SEQUENCE LISTING (50 sequences)

SEQ ID NO: 1 - DNA, 4665 bp, Homo sapiens
agccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatgaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 2 - protein, 1014 aa, Homo sapiens
MVFSVRQCGHVGRTEEVLLTFKIFLVIICLHVVLVTSLEEDTDNSSLSPPPAKLSVVSFAPSSNEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 3 - DNA, 4684 bp, Mus musculus
gccaaccggtcaccgggacccgcagatgcacacggagtttcctccctatttcctctgaacttcctgtcaggatgcttttctctggtgggcagtacagccctgttggcagacctgaagaggttttactgatatacaagatattccttgtcatcatttgttttcatgtcattctcgttacatccctgaaagaaaacggtaattccagtttgttatcaccatctgctgaatcatctcttgtcagtctcatcccctactccaatggtacaccagatgctgcttcagaagttttgtcgactttaaacaaaacagaaaaatctaaaatcactatagtaaaaaccttcaatgcatcaggagtcaaatcccagagaaatatctgcaatttgtcatctctttgcaatgactcagtattttttagaggtgagatagtgtttcaacatgatgaagaccacaatgttacccagaatcaagatacagctaatggcaccttcgctggagtcctgtctctaagtgaactgaagcgatcagagctcaacaaaactctacagaccttaagtgagacttactttatagtgtgtgctacagcagaggcccaaagcacggtaaactgtacattcacagtaaaactcaatgagaccatgaatgtgtgtgccatgatggttactttccaaactgtacagattcggccaatggaacagtgctgctgttccccgaggactccctgcccttcctcaccagaagagttagaaaaactacagtgtgaactgcaggatcccattgtctgtcttgctgatcaaccgcatggcccaccgttatcgtcttccagcaagcctgttgtacctcaggccaccattatttcccatgttgctagtgacttctctttggctgaaccccttgatcatgcccttatgaccccaagcacaccctctctgacacaagaaagtaaccttccatctcctcagcctacgatccccctggcttccagtcctgccactgacttgccagttcaatctgtagtggtctcttctttgcctcaaactgatctttcccacaccctgtcaccggtgcagtcctccattccctctcctaccacaccagccccatctgtccctacagaactggtcaccatcagcacacctcctggtgagacagttgtcaacactagcactgtttctgatctggaagcccaagtatcccagatggagaaagctttgtctttgggtagcttagagcctaatcttgcaggcgaaatggtaaaccgagtcagcaaactccttcactctccacctgccttgctagcccctctagctcaaaggttgctaaaagtggtagatgccattggcttacagctgaatttttcatctacaactatcagtctaacttcaccttctttggctcttgctgtgatcagagtgaatgccagtaatttcaataccacgacttttgcagcccaagacccaacaaatctccaggtttctctggaaaccccacctcctgagaatagtattggtgccattactctgccctcatcactgatgaataatttgccagctaatgatgtagaattggcttcaaggattcagttcaatttctttgaaacacccgccctgtttcaggatccttccctggagaacctcactctgataagctatgtcatatcatcaagtgtcacaaacatgaccatcaagaacttgacaagaaacgtgacagtcgcactgaaacacatcaacccaagtccggatgacttaactgtgaaatgtgtattctgggacttgggcagaaatggtggcaaaggaggttggtcatctgatggctgttccgtcaaagacaagagaatgaatgaaaccatctgtacctgtagccatcttacaagttttggcatcctattggacctatctcgaacatccttaccaccaagtcaaatgatggctctgacatttatcacgtatattggctgtgggctttcatcaatttttctgtcagttactcttgtaacctacatcgcctttgaaaagatccggagggattacccctccaaaatcctcatccagctgtgtgctgccctgcttctgctcaacctgatcttcctcctagactcctggattgcgctgtataatacccgaggtttctgcattgccgtggctgtatttcttcactattttctcttggtctcattcacatggatgggattagaagcattccacatgtacctagcactggtcaaggtgtttaatacttacatccgaaagtacatccttaaattctgcattgttggctggggcataccagctgtggttgtgtccatcgtcctgactatatccccagataactatgggattggatcctatggaaaattccccaatggcacaccagatgacttttgctggatcaacagcaatgtggtgttctatatcacggttgtgggatatttctgtgtgatatttctactgaacgtcagcatgttcatcgtggtcttggttcagctctgtcgaattaaaaagaagaagcagctgggagcccagcgcaaaactagtattcaagacctcaggagtatcgctggcctcacatttttactgggaattacttggggctttgccttctttgcctggggaccagttaatgtcaccttcatgtatctctttgccatctttaacaccttacaagggtttttcatattcattttttactgtgcagcaaaagagaatgtcagaaaacagtggaggcggtatctttgttgtggaaaattacggctggctgaaaattctgactggagtaaaactgctactaatggtttaaagaagcagactgtaaaccaaggagtatccagctcttcaaattccttacagtcaagctgtaactccactaactccaccacactcctagtgaatagtgattgctcagtgcacgcaagcgggaatggcaatgcatctaccgagaggaatggggtttctttcagtgttcagaatggagacgtgtgccttcatgatctcactggcaaacagcatatgtttagtgacaaagaggactcctgcaatggtaaaagccggatagcgctcagaaggacttcgaagcggggaagcttacactttatcgagcaaatgtgattctcttcttctaaaatcaaagcatgatgtggatggtgtgaagtgtccagttttcctttaacacaatgtgagatgtatgaaaagcgctggttttctattcagcaacctcgaaaggaacataagctaaatgagggcaaccagttactatcagaagaaaccacgactggttatggtttctagacgtttgcagtttggtttcttatctctcattttcaaaggcattggtgttaaaaccacagaataagatccaggaaagaaaaatgaaagtttgtttccactcacattttaaagaggctgttatccttgataacatcctaagaaagaaggaaaaaaagaaggaaagaaagaaagaaagagagagagagagagagaaagaaaaaagaaagaaagatgtttacttcagtctgttggtaagtttagttgtgcatgcctttgttgtatataagctaaattctagtgacccacatgtcaaaaaatcttctctttacctttttttatatttattttctactgtgtaaatttattcttttgtagaatcatggttgtgtcgccttctctaatgtgataacttaccagtgacttctaaacctcctttagaagatcatataggatgtgaaattcagaaacttcactgaaatcaagaaataatgataaaggcaatctgaacaattataaatagacaattagctcagaaagtgcctttgagcaatcaagaaaaggtgcccccactgggcagatatagccttgtgagcttatggtttggaaaacagtcgggccacattttagccctgatgatcctcctgggtaccttatgcagggtcctcatgaagggtccttatgcagggaatgacttgtatgactatatgtgacatgaaatagtgataccaatggccagacttcatccctaatgaccagattatagaatctctacaccatggttgttagaggctaaaactgtaatctaaaccatgttaggaaaatcgtgatggtagaaaagatacacactgccacttctcaaatacccagagcctttcaaaagaggggatcagagtaggattacttttccagagtgtatatatggcaaacaaagtaggtctttaaaagacgctgcatgatatgttattgttgcaactgctacattctggtccagtcatacttgtatgtatttttaaaaacatgtcaaatcttgaagagtgtatagtgacaagggggagcagctactgggaagagtgaattatcctgtgatattgttgtttgtatgtaataacatatatccaaccttgattgttcctagttctagagggcctatcctatttctcacctttaccagcttgagtggtgcctcaagcagtgtgattattaggaacatttcaaaatcctttagttaggtctttcactaaggctcccttgcatatatttcaaataaatgttgtatctgagactagccacagtagtaacatctgtttctgtgtgtgctcattgtctttgcaaaattcttctccaaattatttaattattttgtatttatatgttatgattgacaaaactgttaaaaaccaataaaaatcaatttgcagttaaaa

SEQ ID NO: 4 - protein, 1009 aa, Mus musculus
MLFSGGQYSPVGRPEEVLLIYKIFLVIICFHVILVTSLKENGNSSLLSPSAESSLVSLIPYSNGTPDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 5 - DNA, 3054 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcatcaggcgccaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggttttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 6 - protein, 1017 aa, Artificial Sequence
  Description of Artificial Sequence Synthetic polypeptide
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGAKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 7 - DNA, 20 bp, Artificial Sequence
  Description of Artificial Sequence Synthetic primer
ggagcctaacctcgcaggag

SEQ ID NO: 8 - DNA, 23 bp, Artificial Sequence
  Description of Artificial Sequence Synthetic primer
actactttcagcaatctttgagc

SEQ ID NO: 9 - DNA, 25 bp, Artificial Sequence
  Description of Artificial Sequence Synthetic primer
cagactccttcattccccgcctgac

SEQ ID NO: 10 - DNA, 18 bp, Artificial Sequence
  Description of Artificial Sequence Synthetic primer
gcgcccaatacgaccaaa

SEQ ID NO: 11 - DNA, 20 bp, Artificial Sequence
  Description of Artificial Sequence Synthetic primer
ccacatcgctcagacaccat

SEQ ID NO: 12 - DNA, 25 bp, Artificial Sequence
  Description of Artificial Sequence Synthetic primer
gggaaggtgaaggtcggagtcaacg

SEQ ID NO: 13 - RNA, 19 nt, Artificial Sequence
  Description of Artificial Sequence Synthetic oligonucleotide
gguagcuauucaguuacug

SEQ ID NO: 14 - RNA, 21 nt, Artificial Sequence
  Description of Artificial Sequence Synthetic oligonucleotide
gaguaaagauucgaccaauuu

SEQ ID NO: 15 - RNA, 21 nt, Artificial Sequence
  Description of Artificial Sequence Synthetic oligonucleotide
gaguaucgcuggccuuacauu

SEQ ID NO: 16 - RNA, 21 nt, Artificial Sequence
  Description of Artificial Sequence Synthetic oligonucleotide
uaacgugaccuucauguauuu

SEQ ID NO: 17 - RNA, 21 nt, Artificial Sequence
  Description of Artificial Sequence Synthetic oligonucleotide
gacaggagauugaaugaaauu

SEQ ID NO: 18 - DNA, 4911 bp, Artificial Sequence
  Description of Artificial Sequence Synthetic polynucleotide
ggcgcccagcctcgccgccgccgagcgcccgacgcgccacgccgcgctgggcagctggagagctggcggcggagcgggccgggagcggcgggggcggcgccgaggccgcgggcggggggaggagggccgcagccccggagaggggaaaaggcgggccggacggcgccagcagccagccccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcatcaggcgccaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactaccttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggttttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggctcatggtttgacaaacagagtggagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaagatctttaaaaaaaaaaaaaaaaaaaaaaaaaaaaaaaaaaaaaaaaaaaattaaaaaaaaaaaaaa

SEQ ID NO: 19 - DNA, 30 bp, Artificial Sequence
  Description of Artificial Sequence Synthetic primer
caacacaactacctttgtggcccaagaccc

SEQ ID NO: 20 - DNA, 30 bp, Artificial Sequence
  Description of Artificial Sequence Synthetic primer
gggtcttgggccacaaaggtagttgtgttg

SEQ ID NO: 21 - DNA, 37 bp, Artificial Sequence
  Description of Artificial Sequence Synthetic primer
gtttcaacacaactacctttgtggcccaagaccctgc

SEQ ID NO: 22 - DNA, 37 bp, Artificial Sequence
  Description of Artificial Sequence Synthetic primer
gcagggtcttgggccacaaaggtagttgtgttgaaac

SEQ ID NO: 23 - protein, 18 aa, Homo sapiens
CLADHPRGPPFSSSQSIP

SEQ ID NO: 24 - DNA, 907 bp, Artificial Sequence
  Description of Artificial Sequence Synthetic polynucleotide
ggcggcaaaaacattcggcacgaggcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaactnccttgtaagaagatactgataattccagtttgtcaccaccacctgctaaattatctgttgtcagttttgccccctcctccaatgaggttgaaacaacaagcctcaatgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggccccaagcacattaaattgtacattcacaataaaactgaataatacaatgaatgcatgtgctgtaatagctgctttggaaagagtaaagattcgaccaatggaacactgctgctgttctgtcaggataccctgcccttccctccccagaagagatggaaaaagcttcgtgtgacctggaggatcccttgtctgtcttgctgaccatccccgtggcccaccattttcttccagccaatccatccaattggtgcctccgggccctggggcttcccaggtcccccaagctacctcttttgg

SEQ ID NO: 25 - DNA, 926 bp, Artificial Sequence
  Description of Artificial Sequence Synthetic polynucleotide
ggggtttttggcaatatttttaattgcttttaactttttgnttttaacatataaatacaagaaaaattaaataaatcagaaaagaaatttgcaaagacaagtcagcactcacagaaatgtgtattattactatggttagtctgagatccaacattcacttgaaatatatgcaagagaaccttagtgaaagacttaactaaaaggggtttgaaatgttcctaataaggatgtacttgaggcactgctcaagcagatgtaggtaggaagcaagatagaccctgtatataactaggagcaatcaaggctcgatacatgtagcaacaatagcagtgtaggacagttccctgttcccaatagctactccccttgtcactgtgcattcttcaagacacatttaaaaaacaattatagctctggctgggccagagtgtaccagtcataacaatcaacaatgtaccatgcagttgccttttaaagaacggcaatatgatttttttccatatatatgtacctttttaagagaaggaatcctagtctactctcctgttcctaaaggctctggggatttgagaagcggcagtgtatgtacctcatctacgttcacaattttcctgacacatggtctggatgacagttcatccctctctggggccaaggtataaagctaaacatcttggtgcagattctataatttgactcctggggatgccggcacgggatggggttttcatattggatgctgtgttcggctgcccaggccgggcccccacagggggagtggggccaaaaaatggctcttccatcggcctgcctaaccatgaacccctaggcctcggttccgcccatcgggggcattttttcccacttgcttacaggcctcgctgcacctgacgtcggtcctgtgctggtccactttcttatcctgt

SEQ ID NO: 26 - DNA, 2892 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatgaggttgaaacaacaagcctcaatgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccttctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 27 - protein, 963 aa, Artificial Sequence
  Description of Artificial Sequence Synthetic polypeptide
MVFSVRQCGHVGRTEEVLLTFKIFLVIICLHVVLVTSLEEDTDNSSLSPPPAKLSVVSFAPSSNEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLALLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSGNASTERNGVSFSVQNGDVCLHDFTGKQHMFNEKEDSCNGKGRMALRRTSKRGSLHFIEQM

SEQ ID NO: 28 - DNA, 3054 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactccctccaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggtccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggaggaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagaccccaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 29 - protein, 1017 aa, Artificial Sequence
  Description of Artificial Sequence Synthetic polypeptide
MVFSVRQCGHVGRTEEVLLTFKIFLVIICLHVVLVTSLEEDTDNSSLSPPPAKLSVVSFAPSSNGTPEVETTSLNDVTLSLLPPNETEKTKITIVKTFNASGVKPQRNICNLSSICNDSAFFRGEIMFQYDKESTVPQNQHITNGTLTGVLSLSELKRSELNKTLQTLSETYFIMCATAEVQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEEALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDPRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 30 - DNA, 2982 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaatcgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggagcttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcagatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 31 - protein, 993 aa, Artificial Sequence
  Description of Artificial Sequence Synthetic polypeptide
MVFSVRQCGHVGRTEEVLLTFKIFLVIICLHVVLVTSLEEDTDNSSLSPPPDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNRTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRSLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 32 - DNA, 3054 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtctccatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccgtcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggtgcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctcctcttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatgggtgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccgaggagtgtccagctcttcaaattccttacagtctagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 33 - protein, 1017 aa, Artificial Sequence
  Description of Artificial Sequence Synthetic polypeptide
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSVPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLVPNLAGEMINQVSRLLHSPPDMLAPLAQRLLKVVDDIGLQLNFSNTTISLTSSSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWVGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNRGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 34 - DNA, 2982 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgatgttactttaagcttactcccttcaaacgaaacagaaaaaactaaaatcactatagtaaaaaccttcaatgcatcgggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggggtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatcctgctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 35 - protein, 993 aa, Artificial Sequence
  Description of Artificial Sequence Synthetic polypeptide
MVFSVRQCGHVGRTEEVLLTFKIFLVIICLHVVLVTSLEEDTDNSSLSPPPDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILILLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 36 - DNA, 2727 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgatgttactttaagcttactcccttcaaacgaaacagaaaaaactaaaatcactatagtaaaaaccttcaatgcattaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgcttcggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcactccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 37 - protein, 908 aa, Artificial Sequence
  Description of Artificial Sequence Synthetic polypeptide
MVFSVRQCGHVGRTEEVLLTFKIFLVIICLHVVLVTSLEEDTDNSSLSPPPDVTLSLLPSNETEKTKITIVKTFNALGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAASERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEALHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGNASTERNGVSFSVQNGDVCLHDFTGKQHMFNEKEDSCNGKGRMALRRTSKRGSLHFIEQM

SEQ ID NO: 38 - DNA, 3054 bp, Artificial Sequence
  Description of Artificial Sequence Synthetic polynucleotide
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgcacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacagtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 39 - protein, 1017 aa, Artificial Sequence
  Description of Artificial Sequence Synthetic polypeptide
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMHLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 40 - protein, 1017 aa, Artificial Sequence
  Description of Artificial Sequence Synthetic consensus sequence
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 41 - protein, 1017 aa, Artificial Sequence; identical to SEQ ID NO: 40
  Description of Artificial Sequence Synthetic consensus sequence

SEQ ID NO: 42 - protein, 1017 aa, Artificial Sequence; identical to SEQ ID NO: 40
  Description of Artificial Sequence Synthetic consensus sequence

SEQ ID NO: 43 - protein, 1017 aa, Artificial Sequence; identical to SEQ ID NO: 40
  Description of Artificial Sequence Synthetic consensus sequence

SEQ ID NO: 44 - protein, 1017 aa, Artificial Sequence; identical to SEQ ID NO: 40
  Description of Artificial Sequence Synthetic consensus sequence

SEQ ID NO: 45 - protein, 1017 aa, Artificial Sequence; identical to SEQ ID NO: 40
  Description of Artificial Sequence Synthetic consensus sequence

SEQ ID NO: 46 - protein, 1017 aa, Artificial Sequence; identical to SEQ ID NO: 40
  Description of Artificial Sequence Synthetic consensus sequence

SEQ ID NO: 47 - DNA, 3038 bp, Homo sapiens
  CDS (6)..(3038)
ctcgaatggagacagacacactcctgctatgggtactgctgctctgggttccaggttccactggtgatatcgactataaagatgatgacgacaagggatctggtctggaagaagatactgataattccagtttgtcaccaccacctgctaaattatctgttgtcagttttgccccctcctccaatgaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgatatgctggcccctctggctcaaagattgctgaaagtagtggatgacatcgtcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 48 - protein, 1010 aa, Homo sapiens
METDTLLLWVLLLWVPGSTGDIDYKDDDDKGSGLEEDTDNSSLSPPPAKLSVVSFAPSSNEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIVLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 49 - protein, 22 aa, Artificial Sequence
  Description of Artificial Sequence Synthetic peptide
METDTLLLWVLLLWVPGSTGDI

SEQ ID NO: 50 - protein, 8 aa, Artificial Sequence
  Description of Artificial Sequence Synthetic peptide
DYKDDDDK

(19) United States
(12) Patent Application Publication
(10) Pub. No.: US 2009/0202474 A1
(43) Pub. Date: Aug. 13, 2009

(54) EXPRESSION OF ORPHAN GPR64 IN INFLAMMATORY DISEASES

(75) Inventors: Priya CHOCKALINGAM, Arlington, MA (US); Manas K. MAJUMDAR, Audubon, PA (US); Debra D. PITTMAN, Windham, NH (US); Julia BILLIARD, Collegeville, PA (US); Edward Roland LAVALLIE, Harvard, MA (US); Jeffrey L. FELDMAN, Arlington, MA (US); Lisa COLLINS-RACIE, Acton, MA (US); Vishnuvardhan DAESETY, Andover, MA (US); Robert MORAN, Schwenksville, PA (US); Elisabeth A. MORRIS, Sherborn, MA (US); Paul YAWORSKY, Boston, MA (US)

Correspondence Address: WilmerHale/Wyeth, 60 STATE STREET, BOSTON, MA, 02109, US

(73) Assignee: WYETH, Madison, NJ (US)

(21) Appl. No.: 12/273,945

(22) Filed: Nov. 19, 2008

Related U.S. Application Data
(60) Provisional application No. 61/003,630, filed on Nov. 19, 2007.

(51) Int. Cl. A61K 38/20; G01N 33/53; G01N 33/68; C12Q 1/37; C12Q 1/00; A61P 29/00; A61K 38/16; C07H 21/00; C12N 15/63; C12N 5/08; C12N 5/06; C12N 1/19; C12N 1/21

(52) U.S. Cl. 424/85.2; 436/501; 436/86; 435/23; 435/4; 435/24; 514/12; 536/23.1; 435/320.1; 435/366; 435/369; 435/358; 435/325; 435/348; 435/254.2; 435/252.3

(57) ABSTRACT

Methods of screening for agents for treating inflammatory diseases are provided. The methods involve screening for agents that modulate the activity or expression of GPR64, which has been discovered herein to play a role in inflammatory diseases. Methods for treating an inflammatory disease, as well as methods of modulating the activity or expression of GPR64, methods of screening for an inflammatory disease in a subject, pharmaceutical compositions, a nucleic acid variant, and antibodies are also provided.

### CROSS-REFERENCE TO RELATED APPLICATIONS

This application claims the benefit of U.S. Provisional application No. 61/003,630, filed Nov. 19, 2007, the entire contents of which are hereby incorporated by reference herein in their entirety.

### FIELD OF THE INVENTION

This invention relates to the field of therapeutics for inflammatory diseases, including, but not limited to, methods of screening for inflammatory diseases, methods of screening for agents to treat inflammatory diseases, and methods for treating inflammatory diseases.

### BACKGROUND OF THE INVENTION

Receptors used as targets for drug development, primarily from the G-protein coupled receptor class, have led to over half of the currently known drugs. Drews, Nature Biotechnology, 14:1516 (1996). G-protein coupled receptors (“GPCRs”) are a superfamily of transmembrane proteins that are activated by a variety of ligands to mediate signal transduction in many cell types. Marinissen et al., Trends Pharmacol. Sci. 22:368-76 (2001). GPCRs are known to play key roles in signal transduction during diverse normal and disease processes. It has been estimated that 30% of clinically prescribed drugs work as either agonists or antagonists of GPCRs, making them an important family of target proteins. Milligan et al., TIPS, 20: 118-124 (1999).

GPCRs are activated by a variety of ligands, including, but not limited to, peptide and non-peptide neurotransmitters, hormones, growth factors, odorant molecules and light. Additional non-limiting examples of GPCR ligands include biogenic amines (e.g., noradrenaline, dopamine, 5-HT, histamine, and acetylcholine), amino acids and ions (e.g., glutamate, Ca²⁺, and GABA), lipids (e.g., lysophosphatidic acid, platelet-activating factor, prostaglandins, leukotrienes, anandamine, and sphingosine-1-phosphate), peptides and proteins (e.g., angiotensin, bradykinin, thrombin, bombesin, follicle-stimulating hormone, leuteinizing hormone, thyroid-stimulating hormone, and endorphins) and others (e.g., light, odorants, pheromones, nucleotides, opiates, and cannabinoids). Marinissen et al., Trends Pharmacol. Sci. 22:368-76 (2001).

The interaction of GPCRs with heterotrimeric G proteins (which contain α, β, and γ subunits) has been extensively studied. The heterotrimeric G proteins undergo conformational changes resulting in the exchange of GDP for GTP bound to the α-subunit of the G-protein following activation of the receptor. Both the Gα- and the Gβγ-subunits can stimulate effector molecules. Non-limiting examples of such effector molecules include adenylyl and guanylyl cyclases, phosphodiesterases, phospholipase A₂, phospholipase C and phosphoinositide 3-kinases, thereby causing the activation or inhibition of the production of various second messengers, including, but not limited to, cAMP, cGMP, diacylglycerol, inositol (1,4,5)-triphosphate, arachidonic acid and phosphatidic acid, as well as causing increases in intracellular concentrations of Ca²⁺ and opening or closing various ion channels. In addition, activation of GPCRs can result in biochemical responses independent of heterotrimeric G proteins through other molecular mechanisms. Additionally, many biological responses involving GPCRs are not dependent on a single biochemical route. Marinissen et al., Trends Pharmacol. Sci. 22:368-76 (2001).

### SUMMARY OF THE INVENTION

It has been found that GPR64 is upregulated in inflammatory diseases, including, but not limited to, osteoarthritis (“OA”) and rheumatoid arthritis (“RA”), as compared to normal cartilage at both the RNA and protein levels. Specifically, the RNA encoding GPR64 has been found to be increased in both mild and severely affected OA cartilage samples as determined by quantitative real-time RT-PCR. Moreover, the number of cells positive for GPR64 protein in OA cartilage has been found to be increased relative to non-diseased cartilage as determined by immunohistochemistry. GPR64 also showed increased expression in RA joint samples, particularly the capsular tissues, as determined using quantitative PCR. Furthermore, it has been found that GPR64 knockdown repressed IL-1β mediated activation of NFκB signaling as well as repressed the induction of MMP13 mRNA levels. MMP13 is a protease responsible for degradation of cartilage extracellular matrix in OA, and its expression can be positively regulated by activation of NFκB signaling. Together, these data support that modulation, in particular inhibition, of GPR64 is a valuable intervention point for the treatment of inflammatory diseases, such as, for example, OA. Thus, GPR64 has herein been discovered as a target for inflammatory disease therapeutics.

Accordingly, in one aspect of the invention, the invention provides a method of treating a subject having or at risk of developing an inflammatory disease. The method comprises administering to the subject a composition comprising an agent that modulates the activity or expression of GPR64. In some embodiments, the agent decreases the activity or expression of GPR64. In other embodiments, the agent increases the activity or expression of GPR64. In another embodiment, the agent is selected from the group consisting of synthetic small molecules, chemicals, nucleic acids, proteins (including, without limitation, antibodies) and portions thereof. In a specific embodiment, the agent is an siRNA molecule that decreases the activity or expression of GPR64. In one embodiment, the agent binds to GPR64. In another embodiment, the agent is an inhibitor of GPR64 activity or expression. In another embodiment, the agent is an activator of GPR64 activity or expression. In yet another embodiment, the agent interacts with an inhibitor of GPR64 activity or expression, and in still another embodiment, the agent interacts with an activator of GPR64 activity or expression. In some embodiments, the inflammatory disease is selected from the group consisting of arthritis, asthma, inflammatory bowel disease, inflammatory skin disorders, multiple sclerosis, osteoporosis, tendonitis allergic disorders, inflammation in response to an insult to the subject, sepsis, and systematic lupus erythematosus. In one embodiment, the inflammatory disease is OA. In another embodiment, the inflammatory disease is RA.

In another aspect, the invention provides a method of modulating the activity or expression of GPR64 in a subject in need thereof. The method comprises administering to the subject a composition comprising an agent that modulates the activity or expression of GPR64. In one embodiment, the agent decreases the activity or expression of GPR64. In another embodiment, the agent increases the activity or expression of GPR64. In another embodiment, the agent is selected from the group consisting of synthetic small molecules, chemicals, nucleic acids, antibodies, metabolites, proteins and portions thereof. In a specific embodiment, the agent is an siRNA molecule that decreases the activity or expression of GPR64. In one embodiment, the agent binds to GPR64. In another embodiment, the agent is an inhibitor of GPR64 activity or expression. In an additional embodiment, the agent is an activator of GPR64 activity or expression. In yet another embodiment, the agent interacts with an inhibitor of GPR64 activity or expression, and in still another embodiment, the agent interacts with an activator of GPR64 activity or expression. In some embodiments, this method is used to treat a subject having or at risk of developing an inflammatory disease. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the host, sepsis, and systematic lupus erythematosus. In one embodiment, the inflammatory disease is OA. In another embodiment, the inflammatory disease is RA.

In yet another aspect, the invention provides a method of screening for an inflammatory disease in a subject. The screening method comprises: (a) contacting/exposing a sample of tissue from the subject with/to an agent that binds to GPR64, (b) detecting a level of binding of the agent to GPR64 in the sample, and (c) comparing the level of binding of the agent to GPR64 in the sample to a control level. In various embodiments, the level of binding of the agent to GPR64 in the sample is increased relative to the control level. In some embodiments, this increased level of binding is indicative of an inflammatory disease in the subject. In additional embodiments, the level of binding is decreased relative to the control level. In some embodiments, this decreased level of binding is indicative that the subject does not have an inflammatory disease. In another embodiment, the screening method comprises: (a) obtaining a sample of tissue from the subject, (b) preparing a composition of cellular material from the sample, (c) detecting the level of GPR64 protein or RNA in the composition of cellular material, and (d) comparing the level of GPR64 protein or RNA in the composition of cellular material to a control level. In various embodiments, the level of GPR64 protein or RNA in the composition of cellular material is increased relative to a control level. In some embodiments, this increased level of GPR64 protein or RNA is indicative of an inflammatory disease in the subject. In additional embodiments, the level of GPR64 protein or RNA is decreased relative to a control level. In some embodiments, this decreased level of GPR64 protein or RNA is indicative that the subject does not have an inflammatory disease.

In some embodiments, the control level is the level of binding of the agent to GPR64 in a sample from a subject not having or not at risk of developing an inflammatory disease.

In some embodiments, the agent is an antibody or a binding portion thereof. In some embodiments, the agent is an siRNA molecule. In some embodiments, the increase in expression in GPR64 is indicative of an inflammatory disease. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the subject, sepsis, and systematic lupus erythematosus. In one embodiment, the inflammatory disease is OA. In another embodiment, the inflammatory disease is RA.

In another aspect of the invention, the invention provides a method of screening for an increase in expression of GPR64 in a subject. The method comprises: (a) contacting a sample of tissue from the subject with an agent that binds to GPR64, (b) detecting a level of binding of the agent to GPR64 in the sample, and (c) comparing the level of binding of the agent to GPR64 in the sample to a control level. In another embodiment, the screening method comprises: (a) obtaining a sample of tissue from the subject, (b) preparing a composition of cellular material from the sample, (c) detecting the level of GPR64 protein or RNA in the composition of cellular material, and (d) comparing the level of GPR64 protein or RNA in the composition of cellular material to a control level.

In one embodiment, the level of binding of the agent to GPR64 is increased relative to the control level. In one embodiment, the level of binding of the agent to GPR64 is decreased relative to the control level. In another embodiment, the agent is an antibody or a binding portion thereof. In some embodiments, an increase in expression in GPR64 is indicative of an inflammatory disease. In some embodiments, a decrease in expression in GPR64 is indicative that the subject does not have an inflammatory disease. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the host, sepsis, and systematic lupus erythematosus. In one embodiment, the inflammatory disease is OA. In another embodiment, the inflammatory disease is RA.

In another aspect, the invention provides a method of screening for an agent that modulates the activity or expression of GPR64. The method comprises: (a) contacting a sample with a test agent, (b) detecting a level of activity or expression of GPR64 in the presence of the test agent, and (c) comparing the level of activity or expression of GPR64 in the presence of the test agent to a control level. In some embodiments, a level of activity or expression of GPR64 in the sample in the presence of the test agent that is different from the control level is indicative that the test agent is an agent that modulates GPR64 activity or expression. In one embodiment, the level of activity or expression of GPR64 in the presence of the test agent is increased relative to the control level. In another embodiment, the level of activity or expression of GPR64 in the presence of the test agent is decreased relative to the control level. In additional embodiments, the agent modifies GPR64 transcription, GPR64 translation, or the GPR64 signal pathway. In some embodiments, the sample is derived from tissue. In other embodiments, the sample is a cell culture. In still other embodiments, the sample is an amount of isolated GPR64 or an amount of a composition containing GPR64.

In another embodiment, the screening method for an agent that modulates GPR64 comprises: (a) contacting GPR64 with a test agent, (b) detecting a level of activity of GPR64 in the presence of the test agent, and (c) comparing the level of activity of GPR64 in the presence of the test agent to a control level. In some embodiments, a level of activity of GPR64 in the sample in the presence of the test agent that is different from the control level is indicative that the test agent is an agent that modulates GPR64 activity. In one embodiment, the level of activity of GPR64 in the presence of the test agent is increased relative to the control level. In another embodiment, the level of activity of GPR64 in the presence of the test agent is decreased relative to the control level. In some embodiments, the agent modulates the GPR64 signal pathway.

In another embodiment, the method comprises: (a) contacting a cell containing a genetic construct with a test agent, (b) detecting a level of activity or expression of GPR64 in the presence of the test agent, and (c) comparing the level of activity or expression of GPR64 in the presence of the test agent to a control level, wherein the genetic construct comprises at least a portion of a GPR64 gene or a GPR64 promoter. In some embodiments, the genetic construct comprises the GPR64 gene operably-linked to a promoter. In other embodiments, the genetic construct comprises a GPR64 promoter operably-linked to a reporter gene. In some embodiments, the portion of the GPR64 gene comprises SEQ ID NO:5. In some embodiments, the portion of the GPR64 gene consists of SEQ ID NO:5. In some embodiments, a level of activity or expression of GPR64 in the sample in the presence of the test agent that is different from the control level is indicative that the test agent is an agent that modulates GPR64 activity or expression. In one embodiment, the level of activity or expression of GPR64 in the presence of the test agent is increased relative to the control level. In another embodiment, the level of activity or expression of GPR64 in the presence of the test agent is decreased relative to the control level. In additional embodiments, the agent modifies GPR64 transcription, GPR64 translation, or the GPR64 signal pathway.

In another aspect, the invention provides a method of screening for an agent that modulates the activity or expression of GPR64. The method comprises: (a) contacting a sample with a test agent, (b) detecting a level of NFκB pathway signaling, and (c) comparing the level of NFκB pathway signaling in the presence of the test agent to a control level. In some embodiments, a level of NFκB pathway signaling in the presence of the test agent that is different from the control level is indicative that the test agent is an agent that modulates GPR64 activity or expression. In some embodiments, the NFκB pathway in the presence of the test agent is activated relative to the control level. In other embodiments, the NFκB pathway in the presence of the test agent is inhibited relative to the control level. In some embodiments, detecting the level of NFκB pathway signaling comprises identifying the location of a transcription factor (such as, for example, p65 or the NFκB complex) or co-factors related to NFκB activation as being in the nucleus compared to in the cytoplasm. In additional embodiments, detecting the level of NFκB pathway signaling comprises detecting the level of an enzyme that degrades cartilage. In various embodiments, the enzyme that degrades cartilage includes, without limitation, an enzyme selected from the group consisting of matrix metalloproteases (MMPs) and/or aggrecanases. In further embodiments, the enzyme that degrades cartilage is MMP13. In additional embodiments, the enzyme that degrades cartilage is ADAMTS1, ADAMTS4, ADAMTS5, ADAMTS8, ADAMTS9, ADAMTS15, or other cartilage degrading enzymes. In additional embodiments, the agent modifies GPR64 transcription, GPR64 translation, or the GPR64 signal pathway. In some embodiments, the sample is derived from tissue. In other embodiments, the sample is a cell culture. In still other embodiments, the sample is an amount of isolated GPR64 or an amount of a composition containing GPR64.

In another embodiment, the screening method for an agent that modulates GPR64 comprises: (a) contacting GPR64 with a test agent, (b) detecting a level of NFκB pathway signaling in the presence of the test agent, and (c) comparing the level of NFκB pathway signaling in the presence of the test agent to a control level. In one embodiment, the level of NFκB pathway signaling in the presence of the test agent is increased relative to the control level. In another embodiment, the level of NFκB pathway signaling in the presence of the test agent is decreased (inhibited) relative to the control level. In some embodiments, the agent modulates the NFκB pathway.

In another embodiment, the method comprises: (a) contacting a cell containing a genetic construct with a test agent, (b) detecting a level of NFκB pathway signaling in the cell in the presence of the test agent, and (c) comparing the level of NFκB pathway signaling in the presence of the test agent to a control level, wherein the genetic construct comprises at least a portion of a GPR64 gene or a GPR64 promoter. In some embodiments, the genetic construct comprises the GPR64 gene operably-linked to a promoter. In other embodiments, the genetic construct comprises a GPR64 promoter operably-linked to a reporter gene. In some embodiments, the portion of the GPR64 gene comprises SEQ ID NO:5. In some embodiments, the portion of the GPR64 gene consists of SEQ ID NO:5. In some embodiments, a level of NFκB pathway signaling in the presence of the test agent that is different from the control level is indicative that the test agent is an agent that modulates GPR64 activity or expression. In one embodiment, the level of activation of the NFκB pathway in the presence of the test agent is increased relative to the control level. In another embodiment, the level of activation of the NFκB pathway in the presence of the test agent is decreased (inhibited) relative to the control level. In additional embodiments, the agent modifies GPR64 transcription, GPR64 translation, or the GPR64 signal pathway.

In yet another aspect, the invention provides a method of screening for an agent that modulates the activity or expression of GPR64. In one embodiment, the method comprises: (a) contacting a sample with a test agent, (b) detecting a level of activity or expression of MMP13 in the presence of the test agent, and (c) comparing the level of activity or expression of MMP13 in the presence of the test agent to a control level. In some embodiments, a level of activity or expression of MMP13 in the presence of the test agent that is different from the control level is indicative that the test agent is an agent that modulates GPR64 activity or expression. In some embodiments, the level of activity or expression of MMP13 in the presence of the test agent is increased relative to the control level. In additional embodiments, the level of activity or expression of MMP13 in the presence of the test agent is decreased relative to the control level. In additional embodiments, the agent modifies GPR64 and/or MMP13 transcription, GPR64 and/or MMP13 translation, or the GPR64 and/or MMP13 signal pathway. In some embodiments, the sample is derived from tissue. In other embodiments, the sample is a cell culture. In still other embodiments, the sample is an amount of isolated GPR64 or an amount of a composition containing GPR64.

In another embodiment, the screening method for an agent that modulates GPR64 comprises: (a) contacting GPR64 with a test agent, (b) detecting a level of activity or expression of MMP13 in the presence of the test agent, and (c) comparing the level of activity or expression of MMP13 in the presence of the test agent to a control level. In some embodiments, a level of activity or expression of MMP13 in the presence of the test agent that is different from the control level is indicative that the test agent is an agent that modulates GPR64 activity or expression. In one embodiment, the level of activity or expression of MMP13 in the presence of the test agent is increased relative to the control level. In another embodiment, the level of activity or expression of MMP13 in the presence of the test agent is decreased relative to the control level. In some embodiments, the agent modulates the GPR64 and/or MMP13 signal pathway.

In another embodiment, the method comprises: (a) contacting a cell culture containing a genetic construct with a test agent, (b) detecting a level of activity or expression of MMP13 in the presence of the test agent, and (c) comparing the level of activity or expression of MMP13 in the presence of the test agent to a control level, wherein the genetic construct comprises at least a portion of a GPR64 gene or a GPR64 promoter. In some embodiments, the genetic construct comprises the GPR64 gene operably-linked to a promoter. In other embodiments, the genetic construct comprises a GPR64 promoter operably-linked to a reporter gene. In some embodiments, the GPR64 gene comprises the nucleic acid sequence of SEQ ID NO:5 or a portion thereof sufficient to affect the level of activity or expression of MMP13. In some embodiments, the GPR64 gene consists of the nucleic acid sequence of SEQ ID NO:5 or a portion thereof sufficient to affect the level of activity or expression of MMP13. In some embodiments, a level of activity or expression of MMP13 in the presence of the test agent that is different from the control level is indicative that the test agent is an agent that modulates GPR64 activity or expression. In one embodiment, the level of activity or expression of MMP13 in the presence of the test agent is increased relative to the control level. In another embodiment, the level of activity or expression of MMP13 in the presence of the test agent is decreased relative to the control level. In additional embodiments, the agent modifies GPR64 and/or MMP13 transcription, GPR64 and/or MMP13 translation, or the GPR64 and/or MMP13 signal pathway.

In another embodiment, the screening method for an agent that modulates GPR64 comprises: (a) contacting GPR64 with a test agent, (b) detecting a level of activity or expression of ADAMTS1, ADAMTS4, ADAMTS5, ADAMTS8, ADAMTS9, ADAMTS15, or other cartilage degrading enzymes in the presence of the test agent, and (c) comparing the level of activity or expression of ADAMTS1, ADAMTS4, ADAMTS5, ADAMTS8, ADAMTS9, ADAMTS15, or other cartilage degrading enzymes in the presence of the test agent to a control level. In some embodiments, a level of activity or expression of ADAMTS1, ADAMTS4, ADAMTS5, ADAMTS8, ADAMTS9, ADAMTS15, or other cartilage degrading enzymes in the presence of the test agent that is different from the control level is indicative that the test agent is an agent that modulates GPR64 activity or expression. In one embodiment, the level of activity or expression of ADAMTS1, ADAMTS4, ADAMTS5, ADAMTS8, ADAMTS9, ADAMTS15, or other cartilage degrading enzymes in the presence of the test agent is increased relative to the control level. In another embodiment, the level of activity or expression of ADAMTS1, ADAMTS4, ADAMTS5, ADAMTS8, ADAMTS9, ADAMTS15, or other cartilage degrading enzymes in the presence of the test agent is decreased relative to the control level. In some embodiments, the agent modulates the signal pathway of GPR64 and/or ADAMTS1, ADAMTS4, ADAMTS5, ADAMTS8, ADAMTS9, ADAMTS15, or other cartilage degrading enzymes.

In another embodiment, the method comprises: (a) contacting a cell culture containing a genetic construct with a test agent, (b) detecting a level of activity or expression of ADAMTS1, ADAMTS4, ADAMTS5, ADAMTS8, ADAMTS9, ADAMTS15, or other cartilage degrading enzymes in the presence of the test agent, and (c) comparing the level of activity or expression of ADAMTS1, ADAMTS4, ADAMTS5, ADAMTS8, ADAMTS9, ADAMTS15, or other cartilage degrading enzymes in the presence of the test agent to a control level, wherein the genetic construct comprises at least a portion of a GPR64 gene or a GPR64 promoter. In some embodiments, the genetic construct comprises the GPR64 gene operably-linked to a promoter. In other embodiments, the genetic construct comprises a GPR64 promoter operably-linked to a reporter gene. In some embodiments, the GPR64 gene comprises the nucleic acid sequence of SEQ ID NO:5 or a portion thereof sufficient to affect the level of activity or expression of ADAMTS1, ADAMTS4, ADAMTS5, ADAMTS8, ADAMTS9, ADAMTS15, or other cartilage degrading enzymes. In some embodiments, the GPR64 gene consists of the nucleic acid sequence of SEQ ID NO:5 or a portion thereof sufficient to affect the level of activity or expression of ADAMTS4. In some embodiments, a level of activity or expression of ADAMTS1, ADAMTS4, ADAMTS5, ADAMTS8, ADAMTS9, ADAMTS15, or other cartilage degrading enzymes in the presence of the test agent that is different from the control level is indicative that the test agent is an agent that modulates GPR64 activity or expression. In one embodiment, the level of activity or expression of ADAMTS4 in the presence of the test agent is increased relative to the control level. In another embodiment, the level of activity or expression of ADAMTS4 in the presence of the test agent is decreased relative to the control level. In additional embodiments, the agent modifies transcription, translation and/or the signal pathway of GPR64 and/or ADAMTS1, ADAMTS4, ADAMTS5, ADAMTS8, ADAMTS9, ADAMTS15, or other cartilage degrading enzymes.

In another aspect, the invention provides a method of screening for an agent that modulates the activity or expression of ADAMTS1, ADAMTS4, ADAMTS5, ADAMTS8, ADAMTS9, ADAMTS15, or other cartilage degrading enzymes. The method comprises: (a) contacting a sample with a test agent, (b) detecting a level of activity or expression of ADAMTS1, ADAMTS4, ADAMTS5, ADAMTS8, ADAMTS9, ADAMTS15, or other cartilage degrading enzymes in the presence of the test agent, and (c) comparing the level of activity or expression of ADAMTS1, ADAMTS4, ADAMTS5, ADAMTS8, ADAMTS9, ADAMTS15, or other cartilage degrading enzymes in the presence of the test agent to a control level. In some embodiments, a level of activity or expression of ADAMTS1, ADAMTS4, ADAMTS5, ADAMTS8, ADAMTS9, ADAMTS15, or other cartilage degrading enzymes in the presence of the test agent that is different from the control level is indicative that the test agent is an agent that modulates the activity or expression of ADAMTS1, ADAMTS4, ADAMTS5, ADAMTS8, ADAMTS9, ADAMTS15, or other cartilage degrading enzymes. In some embodiments, the level of activity or expression of ADAMTS1, ADAMTS4, ADAMTS5, ADAMTS8, ADAMTS9, ADAMTS15, or other cartilage degrading enzymes in the presence of the test agent is increased relative to the control level. In further embodiments, the level of activity or expression of ADAMTS1, ADAMTS4, ADAMTS5, ADAMTS8, ADAMTS9, ADAMTS15, or other cartilage degrading enzymes in the presence of the test agent is decreased relative to the control level. In additional embodiments, the agent modifies transcription and/or translation of GPR64 and/or ADAMTS1, ADAMTS4, ADAMTS5, ADAMTS8, ADAMTS9, ADAMTS15, or other cartilage degrading enzymes, or the signal pathway of GPR64 and/or ADAMTS1, ADAMTS4, ADAMTS5, ADAMTS8, ADAMTS9, ADAMTS15, and/or other cartilage degrading enzymes. In some embodiments, the sample is derived from tissue. In other embodiments, the sample is a cell culture. In still other embodiments, the sample is an amount of isolated GPR64 or an amount of a composition containing GPR64.

In another aspect, the invention provides a method of identifying a modulator of GPR64. The method comprises (a) over-expressing GPR64 in a mammalian cell, (b) contacting the cell with a test agent, (c) detecting a level of activity or expression of GPR64 in the presence of the test agent, and (d) comparing the level of activity or expression of GPR64 in the presence of the test agent to a control level. In some embodiments, the cell is selected from the group consisting of U2OS, CHO, HEK293, NIH3T3, and COS7. In some embodiments, the method further comprises determining the level of expression of GPR64 in the cell membrane by immunostaining. In some embodiments, the method further comprises monitoring the basal activity of GPR64. In various embodiments, monitoring the basal activity of GPR64 comprises monitoring the level of one or more signaling pathways in cells transfected with GPR64 and comparing to a control level, e.g., a level in cells transfected with an empty vector. In some embodiments, monitoring the basal activity comprises measuring multiple intracellular events. In some embodiments, measuring multiple intracellular events comprises measuring the generation or down-regulation of cAMP, e.g., by CRE-Luc reporter assays or enzyme fragmentation complementation assays; measuring the activation of the MAP Kinase pathway, e.g., by an SRE-Luc reporter analysis; and/or measuring the generation of IP₃, e.g., directly or indirectly, e.g., by measuring changes, e.g., increases, in intracellular concentration of Ca²⁺. In additional embodiments, measuring changes in Ca²⁺ concentration comprises FLIPR technology assays or NFAT-RE-Luc reporter gene assays.

In some embodiments, a level of activity or expression of GPR64 in the cell in the presence of the test agent that is different from the control level is indicative that the test agent is a modulator of GPR64 activity or expression.

In various embodiments, the method further comprises transfecting cells with various doses of GPR64 and determining a dose response. In some embodiments, a high-throughput screen (HTS) is used to identify a modulator of GPR64. In various embodiments, the cell line is stably transfected. In other embodiments, the cell line is transiently transfected. In some embodiments, the cell line is transiently transfected with an amount of GPR64 cDNA around the EC₅₀. In some embodiments, the cell is stably transfected with GPR64 and/or a reporter gene. In various embodiments, the modulator is a small molecule activator and/or inhibitor of basal GPR64 activity levels. In some embodiments, the cell is transfected with a truncated form of GPR64. In additional embodiments, the truncated GPR64 has one or more portions of the extracellular domain deleted or removed.

In other embodiments, the method includes visualizing GPR64 internalization. In some embodiments, the method includes introducing a component of an internalized vesicle into the cell and monitoring it. In some embodiments, this component is an arrestin-GFP fusion protein. In some embodiments, a truncated form of GPR64 is used. In additional embodiments the truncated GPR64 has one or more portion of the extracellular domain deleted or removed.

In still another aspect, the invention features a method of diagnosing an inflammatory disease in a subject suspected of suffering from the inflammatory disease. The method comprises: (a) contacting a sample of tissue from the subject with an agent that binds to GPR64, (b) detecting a level of binding of the agent to GPR64 in the sample, and (c) comparing the level of binding of the agent to GPR64 in the sample to a control level. In another embodiment, the screening method comprises: (a) obtaining a sample of tissue from the subject, (b) preparing a composition of cellular material from the sample, (c) detecting the level of GPR64 protein or RNA in the composition of cellular material, and (d) comparing the level of GPR64 protein or RNA in the composition of cellular material to a control level.

In one embodiment, the level of binding of the agent to GPR64 or the level of GPR64 protein or RNA is increased relative to the control level. In another embodiment, the agent is an antibody or a binding portion thereof. In some embodiments, an increase in the level of binding of the agent to GPR64 or the level of GPR64 protein or RNA is indicative of an inflammatory disease. In another embodiment, the level of binding of the agent to GPR64 or the level of GPR64 protein or RNA is decreased relative to the control level. In some embodiments, a decrease in the level of binding of the agent to GPR64 or the level of GPR64 protein or RNA is indicative that the subject does not have an inflammatory disease. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the host, sepsis, and systematic lupus erythematosus. In one embodiment, the inflammatory disease is OA. In another embodiment, the inflammatory disease is RA.

In another aspect of the invention, a pharmaceutical composition is provided. The pharmaceutical composition comprises an agent that modulates the activity or expression of GPR64 and a pharmaceutically-acceptable carrier. In one embodiment, the agent decreases the activity or expression of GPR64. In another embodiment, the agent increases the activity or expression of GPR64. In some embodiments, the agent is selected from the group consisting of synthetic small molecules, chemicals, nucleic acids, antibodies, metabolites, proteins and portions thereof. In one embodiment, the agent binds to GPR64. In some embodiments, the agent that binds to GPR64 is an antibody. In another embodiment, the agent is an inhibitor of GPR64 activity or expression. In some embodiments, the agent that decreases the activity or expression of GPR64 is an siRNA molecule. In a further embodiment, the agent is an activator of GPR64 activity or expression. In yet another embodiment, the agent interacts with an inhibitor of GPR64 activity or expression, and in still another embodiment, the agent interacts with an activator of GPR64 activity or expression.

In another aspect of the invention, a pharmaceutical composition for treating an inflammatory disease is provided. The pharmaceutical composition comprises an agent that modulates the activity or expression of GPR64 and a pharmaceutically-acceptable carrier. In one embodiment, the agent decreases the activity or expression of GPR64. In another embodiment, the agent increases the activity or expression of GPR64. In some embodiments, the agent is selected from the group consisting of synthetic small molecules, chemicals, nucleic acids, antibodies, metabolites, proteins and portions thereof. In one embodiment, the agent binds to GPR64. In another embodiment, the agent is an inhibitor of GPR64 activity or expression. In another embodiment, the agent is an activator of GPR64 activity or expression. In yet another embodiment, the agent interacts with an inhibitor of GPR64 activity or expression, and in still another embodiment, the agent interacts with an activator of GPR64 activity or expression. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the host, sepsis, and systematic lupus erythematosus. In one embodiment, the inflammatory disease is OA. In another embodiment, the inflammatory disease is RA.

In another aspect, the invention provides a nucleic acid sequence comprising SEQ ID NOS:5, 26, 28, 30, 32, 34, 36, or 38, or a nucleic acid sequence having at least about 70%, at least about 75%, at least about 80%, at least about 85%, at least about 90%, at least about 95%, at least about 96%, at least about 97%, at least about 98%, or at least about 99% sequence identity to SEQ ID NOs:5, 26, 28, 30, 32, 34, 36, or 38. In an additional aspect, the invention provides a nucleic acid sequence consisting essentially of SEQ ID NOS:5, 26, 28, 30, 32, 34, 36, or 38, or a nucleic acid sequence having at least about 70%, at least about 75%, at least about 80%, at least about 85%, at least about 90%, at least about 95%, at least about 96%, at least about 97%, at least about 98%, or at least about 99% sequence identity to SEQ ID NOs:5, 26, 28, 30, 32, 34, 36, or 38. In an additional aspect, the invention provides a nucleic acid sequence consisting of SEQ ID NOS:5, 26, 28, 30, 32, 34, 36, or 38, or a nucleic acid sequence having at least about 70%, at least about 75%, at least about 80%, at least about 85%, at least about 90%, at least about 95%, at least about 96%, at least about 97%, at least about 98%, or at least about 99% sequence identity to SEQ ID NOs:5, 26, 28, 30, 32, 34, 36, or 38. In a further aspect, the invention provides a gene construct comprising SEQ ID NOS:5, 28, 30, 32, 34, 36, or 38, or a nucleic acid sequence having at least about 70%, at least about 75%, at least about 80%, at least about 85%, at least about 90%, at least about 95%, at least about 96%, at least about 97%, at least about 98%, or at least about 99% sequence identity to SEQ ID NOs:5, 26, 28, 30, 32, 34, 36, or 38, and a promoter.

In another aspect, the invention provides an antibody or a binding portion thereof. In some embodiments, the antibody or a binding portion thereof binds to GPR64. In other embodiments the antibody or a binding portion thereof binds to an activator of GPR64 activity or expression. In other embodiments the antibody or a binding portion thereof binds to an inhibitor of GPR64 activity or expression. Such an antibody may be, without limitation, a polyclonal antibody, a monoclonal antibody, a chimeric antibody, a humanized antibody, a genetically-engineered antibody, a bispecific antibody, antibody fragments (including, but not limited to, “Fv,” “F(ab′)₂,” “F(ab),” and “Dab”) and single chains representing the reactive portion of the antibody. Such an antibody includes antibodies belonging to any of the immunoglobulin classes, such as IgM, IgG, IgD, IgE, IgA or their subclasses or mixtures thereof. In another aspect, the invention provides an siRNA molecule that decreases the activity or expression of a GPR64.

In yet another aspect, the invention provides a kit for screening for an inflammatory disease. The kit comprises at least one container for a tissue sample, at least one component for detection of a diagnostic protein and at least one component for quantification of the level of the diagnostic protein. In one embodiment, the diagnostic protein is GPR64, MMP13, ADAMTS1, ADAMTS4, ADAMTS5, ADAMTS8, ADAMTS9, and/or ADAMTS15. In one embodiment, the component for detection comprises an siRNA molecule that targets GPR64. In another embodiment, the component for detection comprises an antibody to GPR64 or an activator or inhibitor of GPR64 activity or expression, or a binding portion of such an antibody. Such an antibody may be, without limitation, a polyclonal antibody, a monoclonal antibody, a chimeric antibody, a humanized antibody, a genetically-engineered antibody, a bispecific antibody, antibody fragments (including, but not limited to, “Fv,” “F(ab′)₂,” “F(ab),” and “Dab”) and single chains representing the reactive portion of the antibody. Such an antibody includes antibodies belonging to any of the immunoglobulin classes, such as IgM, IgG, IgD, IgE, IgA or their subclasses or mixtures thereof. In another embodiment, the kit further comprises a control for comparison. In yet another embodiment, the kit comprises a control sample. In some embodiments, the kit includes an agent used to treat an inflammatory disease.

In another aspect, the invention provides a kit for treating an inflammatory disease. The kit comprises one or more agents used to treat an inflammatory disease. In some embodiments, the kit also comprises components used for screening tissue to determine if a subject has an inflammatory disease.

In additional aspects, the invention provides for the use of one or more of the above compositions, components, modulators and/or kits for the treatment of an inflammatory disease, for the diagnosis of an inflammatory disease, and/or for the identification of modulators of GPR64 activity or expression. In various embodiments, the inflammatory disease is OA. In other embodiments, the inflammatory disease is RA.

The following figures are presented for the purpose of illustration only, and are not intended to be limiting.

### BRIEF DESCRIPTION OF THE DRAWINGS

FIG. 1 is a representation of the nucleotide sequence of human GPR64 mRNA (SEQ ID NO:1) as reported in Genbank (NM_{—}005756), the contents of which are incorporated herein by reference in their entirety.

FIG. 2 is a representation of the amino acid sequence of human GPR64 protein (SEQ ID NO:2) as reported in Genbank (NP_{—}005747), the contents of which are incorporated herein by reference in their entirety.

FIG. 3 is a representation of the nucleotide sequence of murine GPR64 mRNA (SEQ ID NO:3) as reported in Genbank (NM_{—}178712), the contents of which are incorporated herein by reference in their entirety.

FIG. 4 is a representation of the amino acid sequence of murine GPR64 protein (SEQ ID NO:4) as reported in Genbank (NP_{—}848827), the contents of which are incorporated herein by reference in their entirety.

FIG. 5A is a representation of the nucleic acid sequence of a GPR64 variant (SEQ ID NO:5)

FIG. 5B is a representation of the amino acid sequence of a GPR64 variant (SEQ ID NO:6).

FIG. 5C is a representation of a GPR64 variant (SEQ ID NO:6) compared to a reference sequence (NP_{—}005747, SEQ ID NO:2).

FIG. 6 is a representation of a chart showing gene expression changes in RA synovium, OA synovium, and OA cartilage.

FIG. 7A is a representation of a chart showing fold change in expression over normal of GPR64 in mild and severe OA.

FIG. 7B is a representation of a graph showing fold change in expression over normal of GPR64 in mild and severe OA.

FIG. 7C is a representation of normal and OA cartilage samples stained using immunochemistry to show GPR64 protein expression.

FIG. 8 is a representation of a graph showing IL-1β treatment induces NFκB reporter activity in the T/C-28a2-Clone 19 cells.

FIG. 9 is a representation of a graph showing GPR64 knockdown represses IL-1β induced NFκB activity.

FIG. 10 is a representation of a graph showing knockdown of GPR64 represses IL-1β- and TNFα-induced MMP13 mRNA levels in T/C-28a2-Clone 19 cells.

FIG. 11 is a representation of a graph showing that multiple GPR64 siRNAs dramatically knockdown target mRNA levels in the SW1353 cell line.

FIG. 12 is a representation of a graph showing that GPR64 levels do not change following IL-1β or TNFα treatment in SW1353 cells.

FIG. 13 is a representation of a graph showing that MMP13 mRNA levels are induced following IL-1β or TNFα treatment in the SW1353 cell line.

FIG. 14 is a representation of a graph showing that knockdown of GPR64 represses IL-1β induced MMP13 mRNA levels in the SW1353 cell line.

FIG. 15 is a representation of a graph showing that knockdown of GPR64 represses ADAMTS4 mRNA levels following IL-1β treatment.

FIG. 16 is a representation of a graph showing that knockdown of GPR64 in primary human OA chondrocytes represses MMP13 mRNA levels.

FIG. 17 is a representation of the nucleotide sequence of IMAGE clone (30340382) (SEQ ID NO:18).

FIG. 18 is a representation of a western blot analysis of GPR64 protein in OA.

FIG. 19A is a representation of the nucleotide sequence of the unedited Origene clone 5′ end read (SEQ ID NO:24).

FIG. 19B is a representation of the nucleotide sequence of the unedited Origene clone 3′ end read (SEQ ID NO:25).

FIG. 19C is a representation of the nucleotide sequence of a novel human GPR64 variant (SEQ ID NO:26).

FIG. 19D is a representation of the predicted amino acid sequence (SEQ ID NO:27) of the novel human GPR64 variant (SEQ ID NO:26).

FIG. 19E is a representation of a comparison of a reference GPR64 protein sequence (SEQ ID NO:2) versus the novel variant (SEQ ID NO:27).

FIG. 20 is a representation of the nucleotide sequence of a novel human GPR64 clone 2 variant (SEQ ID NO:28).

FIG. 21 is a representation of the predicted amino acid sequence (SEQ ID NO:29) of the novel human GPR64 clone 2 variant.

FIG. 22 is a representation of the nucleotide sequence of a novel human GPR64 clone 5 variant (SEQ ID NO:30).

FIG. 23 is a representation of the predicted amino acid sequence (SEQ ID NO:31) of the novel human GPR64 clone 5 variant.

FIG. 24 is a representation of the nucleotide sequence of a novel human GPR64 clone 11 variant (SEQ ID NO:32).

FIG. 25 is a representation of the predicted amino acid sequence (SEQ ID NO:33) of the novel human GPR64 clone 11 variant.

FIG. 26 is a representation of the nucleotide sequence of a novel human GPR64 clone 13 variant (SEQ ID NO:34).

FIG. 27 is a representation of the predicted amino acid sequence (SEQ ID NO:35) of the novel human GPR64 clone 13 variant.

FIG. 28 is a representation of the nucleotide sequence of a novel human GPR64 clone 20 variant (SEQ ID NO:36).

FIG. 29 is a representation of the predicted amino acid sequence (SEQ ID NO:37) of the novel human GPR64 clone 20 variant.

FIG. 30 is a representation of the nucleotide sequence of a novel human GPR64 variant (SEQ ID NO:38).

FIG. 31 is a representation of the predicted amino acid sequence (SEQ ID NO:39) of the novel human GPR64 variant.

FIG. 32 is a representation of a comparison of reference GPR protein sequence (SEQ ID NO:2) versus novel variants of the invention (SEQ ID NOS:6 and 29).

FIG. 33 is a representation of a comparison of reference GPR protein sequence (SEQ ID NO:2) versus novel variants of the invention (SEQ ID NOS:6, 29 and 31).

FIG. 34 is a representation of a comparison of a reference GPR protein sequence (SEQ ID NO:2) versus novel variants of the invention (SEQ ID NOS:6, 29, 33, and 35, 37, and 42).

FIG. 35 is a representation of an alignment of a reference GPR protein sequence (SEQ ID NO:2) with all full length GPR64 variants disclosed in this application (SEQ ID NOS: 6, 27, 29, 31, 33, 35, 37, and 39). A consensus sequence derived from these GPR sequences is also provided (SEQ ID NO:43).

FIG. 36 is a representation of an alignment of a reference GPR protein sequence (SEQ ID NO:2) with all full length GPR64 variants obtained from naturally isolated cDNAs (SEQ ID NOS: 6, 27, 29, 31, 33, 35, 37, and 39). A consensus sequence derived from these GPR sequences is also provided (SEQ ID NO:44).

FIG. 37 is a representation of an alignment of all full length GPR64 variants from the SW1353 chondrocytic cells (SEQ ID NOS: 29, 33, 35, and 37). A consensus sequence derived from these GPR sequences is also provided (SEQ ID NO:45).

FIG. 38 is a representation of an alignment of all full length GPR64 variants from a primary human chondrocyte (SEQ ID NO:39) as well as the SW1353 chondrocytic cells (SEQ ID NOS: 29, 33, 35, 37, and 39). A consensus sequence derived from these GPR sequences is also provided (SEQ ID NO:46).

FIG. 39 is a representation of the nucleotide (SEQ ID NO:47) and amino acid sequence (SEQ ID NO:48) of the GPR64 expressed in a U2OS osteosarcoma cell line that over-expresses GFP-tagged β-arrestin. The GPR64 protein is expressed with a heterologous signal peptide and a Flag tag.

Other features and advantages of the invention will be apparent from the following detailed description, and from the claims.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein, to refer to one or to more than one (i.e., to at least one) of the grammatical object of the article. By way of example, “an element” means one element or more than one element.

An “isolated” or “purified” polypeptide or protein, e.g., an “isolated antibody,” is purified to a state beyond that in which it exists in nature. For example, the “isolated” or “purified” polypeptide or protein, e.g., an “isolated antibody,” can be substantially free of cellular material or other contaminating proteins from the cell or tissue source from which the protein is derived, or substantially free from chemical precursors or other chemicals when chemically synthesized. In some embodiments, the preparation of antibody protein having less than about 50% of non-antibody protein (also referred to herein as a “contaminating protein”), or of chemical precursors, is considered to be “substantially free.” In other embodiments, about 40%, about 30%, about 20%, about 10% and more preferably about 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation. Proteins or polypeptides referred to herein as “recombinant” are proteins or polypeptides produced by the expression of recombinant nucleic acids.

The term “antibody” as used herein, includes intact antibodies, fragments of antibodies, e.g., Fab, F(ab′)₂ Fd, dAb and scFv fragments, and intact antibodies and fragments that have been mutated either in their constant and/or variable region (e.g., mutations to produce chimeric, partially humanized, or fully humanized antibodies, as well as to produce antibodies with a desired trait). As such, antibodies or fragments thereof are included in the scope of the invention, for example, antibodies or fragments that specifically bind to GPR64 or to an activator or inhibitor of GPR64, and neutralize or inhibit one or more GPR64-associated activities.

The antibody includes an immunoglobulin molecule comprised of four polypeptide chains, two heavy (H) chains and two light (L), chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “binding portion” of an antibody (or “antibody portion”) includes fragments of an antibody that retain the ability to specifically bind to GPR64 or an activator or inhibitor of GPR64, and modulate the GPR64 activity. It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al., (1993) Proc. Natl. Acad. Sci. USA 90:6444-6448; Poljak, R. J. et al., (1994) Structure 2:1121-1123).

Still further, an antibody or binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M. et al., (1995) Human Antibodies and Hybridomas 6:93-101) and use of a cysteine residue, a marker peptide and a C-terminal polyhistidine tag to make bivalent and biotinylated scFv molecules (Kipriyanov, S. M. et al., (1994) Mol. Immunol. 31: 1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and as known in the art. Preferred binding portions are complete domains or pairs of complete domains.

Intact antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit that binds to the antigen, i.e., the binding fragment, or, e.g., the portion of the CH subunit that binds to and/or activates, e.g., an Fc receptor and/or complement.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody), such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An “expression construct” is any recombinant nucleic acid that includes an expressible nucleic acid and regulatory elements sufficient to mediate expression of the expressible protein or polypeptide in a suitable host cell.

The terms “fusion protein,” “fusion polypeptide” and “chimeric protein” are interchangeable and refer to a protein or polypeptide that has an amino acid sequence having portions corresponding to amino acid sequences from two or more proteins. The sequences from two or more proteins may be full or partial (i.e., fragments) of the proteins. Fusion proteins may also have linking regions of amino acids between the portions corresponding to those of the proteins. Such fusion proteins may be prepared by recombinant methods, wherein the corresponding nucleic acids are joined through treatment with nucleases and ligases and incorporated into an expression vector. Preparation of fusion proteins is generally understood by those having ordinary skill in the art.

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “or” is used herein to mean, and is used interchangeably with, the term “and/or,” unless the context clearly indicates otherwise.

The term “percent identical” or “percent identity” refers to sequence identity between two amino acid sequences or between two nucleotide sequences. Percent identity can be determined by comparing a position in each sequence that may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP I program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves the ability to pick up distantly related matches, and is especially tolerant of small gaps and nucleotide sequence errors. Nucleic acid-encoded amino acid sequences can be used to search both protein and DNA databases.

The terms “polypeptide” and “protein” are used interchangeably herein.

The term “recombinant nucleic acid” includes any nucleic acid comprising at least two sequences that are not present together in nature. A recombinant nucleic acid may be generated in vitro, for example by using the methods of molecular biology, or in vivo, for example by, insertion of a nucleic acid at a novel chromosomal location by homologous or non-homologous recombination.

The term “treating” with regard to a subject, refers to improving at least one symptom of the subject's disease or disorder. Treating can be curing the disease or condition or improving it.

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (e.g., a vector may be autonomously replicating in one host cell type and purely integrative in another host cell type). Vectors capable of directing the expression of expressible nucleic acids to which they are operatively linked are referred to herein as “expression vectors.”

The phrase “effective amount” as used herein, means that amount of one or more agent, material, or composition comprising one or more agents described herein that is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the subject, the route of administration, etc. A skilled medical practitioner can readily determine the appropriate dose using methods well known in the medical arts.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “inflammatory disease” is a disease that involves the recruitment of humoral and cellular components of the immune system into tissue. The inflammation process activates numerous cellular and inflammatory cytokine pathways. It involves a complex series of events that include, without limitation, vascular cells, increased permeability and blood flow, exudation of fluids, cell migration and the induction of inflammatory mediators. Non-limiting examples of humoral and cellular components of the immune system recruited into tissue (which can be called cellular infiltrates) are macrophages, mast cells, T-cells, neutrophils, lymphocytes, B-cells and fibroblasts. Non-limiting examples of inflammatory cytokines or chemokines include tumor necrosis factor (“TNF”), interleukin-1 (“IL-1”), interleukin-6 (“IL-6”), interleukin-8 (“IL-8”), IL-18, IL-22, and IL-17. For some inflammatory diseases, the local environment, such as the endothelium, and its signaling pathways are also involved. Inflammatory responses include a broad range of host reaction to a variety of insults, such as, for example, injury, infection, and trauma, and include both innate and adaptive immunity responses. It is the overproduction of mediators that is believed to be associated with a broad range of disorders.

Examples of inflammatory diseases include, but are not limited to, arthritis (including, but not limited to, osteoarthritis, rheumatoid arthritis, spondyloarthropathies, and psoriatic arthritis), asthma (including, but not limited to, atopic asthma, nonatopic asthma, allergic asthma, exercise-induced asthma, drug-induced asthma, occupational asthma and late stage asthma), inflammatory bowel disease (including, but not limited to, Crohn's Disease), inflammatory skin disorders (including, but not limited to, psoriasis, atopic dermatitis, and contact hypersensitivity), multiple sclerosis, osteoporosis, tendonitis, allergic disorders (including, but not limited to, rhinitis, conjunctivitis, and urticaria), inflammation in response to an insult to the host (including, but not limited to, injury or infection), sepsis, and systematic lupus erythematosus.

Inflammatory arthritis represents a family of arthritic diseases characterized by lymphokine-mediated and cytokine-mediated inflammation of the joints. Inflammatory arthritis is often autoimmune in origin but is not limited to this cause. Examples of inflammatory arthritis can include rheumatoid arthritis, osteoarthritis, psoriatic arthritis, and lupus-associated arthritis. The most common form of inflammatory arthritis is rheumatoid arthritis. RA is characterized by persistent inflammation of the joints. Inflammation can eventually lead to cartilage destruction and bone erosion.

By way of non-limiting example, osteoarthritis (“OA”) is an inflammatory disease characterized by the degradation of cartilage extracellular matrix, leading to cartilage damage and erosion. While several catabolic factors and degradative enzymes have been implicated in the degradation process, it is clear that many signal transduction pathways involved are not yet characterized. OA has only recently been shown to have inflammatory and immuno-modulatory, as well as erosive, components. The erosive components are related to the wear and tear or aging of the joint and involves deterioration of the smooth cartilage of the joints. OA is characterized by degenerative changes in the articular cartilage and subsequent new bone formation at the articular margins. OA usually presents as pain, which decreases mobility and appears as thinning cartilage in an X-ray. Joints commonly affected are the knees, hips, spine, finger, base of thumb and base of the big toe. OA is the most common type of arthritis and affected some 20.7 million Americans (i.e., 12.1% of adult Americans) in 1990 and is now estimated to affect some 37 million Americans, trailing only chronic heart disease as the leading cause of Social Security payments due to long-term absence from work (see Lawrence et al., (1998) Arthritis & Rheumatism 41: 778-799).

As an additional non-limiting example, rheumatoid arthritis (“RA”) is a multi-faceted chronic disease (i.e., several disease processes occur in a single tissue). RA comprises inflammatory, angiogenic, neoplastic, immunoregulatory, and matrix erosive activities. RA appears to be an autoimmune disease characterized by joint swelling, deformation and, ultimately, destruction, culminating in severe physical disability (see De Graaf et al., (1963) in The Epidemiology of Chronic Rheumatism, Dellgren and Ball, eds. (Blackwell, Oxford), pp. 446-56; Meenam et al., (1981) Arthritis Rheum., 24:544-50; Gabriel et al., (1999) J. Rheumatol., 26:1269-74; James, (1999) Clin. Exp. Rheumatol., 17:392-93). RA is a progressive systematic inflammatory condition with well-recognized symptoms that include: symmetrical peripheral joint swelling and synovial inflammation which spares the axial skeleton; the presence of rheumatoid factor autoantibodies; increased concentrations of interleukin-6 (IL-6) in serum and synovial fluid; and pregnancy-induced disease remission followed by severe postpartum flares. The inflamed synovium is typically densely crowded with lymphocytes and affects the synovial membrane, which is a structure that is typically one cell layer thick and includes vessels, dendritic cells, T cells, B cells, NK cells, macrophages, as well as clusters of plasma cells. Additionally, there are often a plethora of immunopathological mechanisms at work, including antigen-antibody complexes, polymorphonuclear neutrophils, inflammatory T cells, and activated macrophages. Eventually, these processes occurring in RA, as with OA, result in destruction of the integrity of the joint with resulting deformity and permanent loss of function. A more detailed description of the etiology and physiology of RA can be found in Zvaifler, N., Etiology and Pathogenesis of RA in Arthritis and Allied Conditions, pp. 659-73 (ed. D. M. McCarty).

Modulation of GPR64 Expression or Activity in Inflammatory Diseases

The invention is based upon the unexpected finding that GPR64 is upregulated in inflammatory diseases, including, but not limited to, OA, as compared to normal cartilage at both the RNA and protein levels. Specifically, the RNA encoding GPR64 has been found to be increased in both mild, and severely affected OA cartilage samples as determined by quantitative real-time RT-PCR, and the number of cells positive for GPR64 in OA cartilage has been found to be increased as determined by immunohistochemistry. Additionally, GPR64 showed increased expression in RA joint samples, particularly the capsular tissues, as determined using quantitative PCR. Thus, GPR64 has herein been discovered as a target for inflammatory disease therapeutics.

Further, GPR64 expression may be correlated with the loss of proteoglycan in the extracellular matrix. These findings suggest that GPR64 plays a role in the degradation of the cartilage extracellular matrix and that increased expression of GPR64 is triggered in response to the degradation process.

GPR64 is a G-protein coupled receptor with an unknown ligand. Osterhoff et al., DNA Cell. Biol. 16:379-89 (1997). GPR64 has been found to be expressed in the epididymis, and expressed sequence tags have been isolated from B-cell, lung, testis, embryo, kidney, and placenta libraries. Expression studies were performed to analyze the GPR64 RNA expression in human normal, mild and severely affected OA cartilage and protein expression in human normal and OA cartilage samples. The results indicated that GPR64 expression was increased in both mild and severely affected OA cartilage samples as compared to normal cartilage. Similar results were obtained with regard to the expression of GPR64 in RA samples. Thus, the inventors believe that GPR64 is involved in inflammatory diseases, and, consequently, that an agent that modulates the activity or expression of GPR64 will be effective in treating subjects afflicted with these inflammatory diseases. Consequently, an agent that modulates the activity or expression of GPR64 should be effective to treat inflammatory diseases. “Modulate” as used herein, refers to activating or inhibiting or otherwise regulating or adjusting the level or degree of that which is being modulated. In some embodiments, the increase in GPR64 expression results in the onset of an inflammatory disease. In other embodiments, the increase in GPR64 expression is a response to an inflammatory disease.

As used herein, “activity” refers to the normal functioning of a gene or protein, such as, for example, GPR64, in a cell or cell signaling pathway. For example, it includes activities such as the binding specificity/affinity of an antibody for an antigen, for example, an anti-GPR64 antibody that binds to GPR64 and/or the neutralizing potency of an antibody, for example, an anti-hGPR64 antibody that binds to hGPR64 and inhibits the biological activity of GPR64. In some embodiments, the cell signaling pathway is the NFκB (Nuclear Factor Kappa B) pathway. As used herein, “expression” refers to the level of mRNA or protein in a cell produced from a gene, such as, for example, GPR64, including the level of transcription of the gene or translation of the mRNA.

Further, it has been discovered that the absence of GPR64 modulates the IL-1β/NFκB pathway. The role of GPR64 in chondrocytes and OA was investigated using RNA interference (RNAi) gene knockdown techniques in human chondroctye cell lines as well as primary human chondrocytes. Data indicated that GPR64 knockdown repressed IL-1β mediated activation of NFκB signaling as well as repressed the induction of MMP13 mRNA levels. Together, these data support that inhibition of GPR64 may be a valuable intervention point for the treatment of OA.

The role of GPR64 in NFκB signal transduction in human chondrocytes was investigated using RNA interference in T/C-28a2-Clone19 cells. siRNA reagents against human GPR64 were transfected into cells that were then subsequently treated with IL-1β, and NFκB-luciferase reporter gene activity was measured. It was shown that knockdown of GPR64 significantly represses the activity of the NFκB luciferase reporter gene to levels similar to that of a p65 control. The data showed that repression of GPR64 attenuates IL-1β mediated activation of NFκB signaling.

MMP13 is a protease responsible for degradation of cartilage extracellular matrix in OA. Its expression can be positively regulated by activation of NFκB signaling. MMP13 mRNA levels were monitored following GPR64 siRNA-mediated knockdown. The data confirm that the inhibition of GPR64 results in the repression of MMP13 mRNA levels following the stimulation of the NFκB pathway in human cartilage cells.

The knockdown of GPR64 mRNA was monitored by real-time RT-PCR post siRNA transfection in the human chondrosarcoma cell line SW1353. The data confirms that GPR64 is expressed in a cell line derived from human cartilage. There was a significant reduction in GPR64 mRNA levels confirming the efficacy of the siRNAs. These data show that the siRNA reagents are capable of specifically knocking down GPR64 mRNA levels.

GPR64 mRNA levels were monitored by real-time RT-PCR following treatment of either TNFα or IL-1β in the human chondrosarcoma cell line SW1353. None of the treatment paradigms affected GPR64 mRNA levels, confirming that the repression of NFκB activity following GPR64 mRNA knockdown is strictly due to RNAi-mediated GPR64 knockdown and not to ligand-mediated changes (from TNFα or IL-1β treatment) in endogenous GPR64 mRNA levels.

MMP13 mRNA levels were monitored by real-time RT-PCR following treatment of either TNFα or IL-1β in the human chondrosarcoma cell line SW1353. Both cytokine ligands at either timepoint showed an induction of MMP13 mRNA levels in this human chondrocyte cell line. These data support that activation of NFκB signaling positively regulates MMP13 mRNA levels. They further support that inhibition of NFκB signaling and consequently inhibiting the induction of MMP13 expression, a cartilage matrix destroying enzyme, provide therapeutic intervention points for the treatment of OA.

MMP13 mRNA levels were monitored following GPR64 siRNA-mediated knockdown in SW1353 cells. Three of four GPR64 siRNA reagents tested as well as a pool showed a significant reduction in MMP13 mRNA levels to levels similar to that following RNAi-mediated knockdown of p65, the control. These data show that the inhibition of GPR64 results in the repression of IL-1β-mediated induction of MMP13 mRNA levels in human cartilage cells.

Aggrecanase ADAMTS4 is also a protease whose activity has been implicated in the destruction of cartilage extracellular matrix in osteoarthritic individuals. ADAMTS4 mRNA levels were monitored following GPR64 siRNA-mediated knockdown in SW1353 cells. All four GPR64 siRNA reagents tested as well as the pool showed a significant reduction in ADAMTS4 mRNA levels to levels similar to that following RNAi-mediated knockdown of p65, the control. These data show that the inhibition of GPR64 results in the repression of a second cartilage matrix degradative enzyme that has been associated with OA.

MMP13 mRNA levels were monitored following GPR64 siRNA-mediated knockdown in primary human chondrocytes isolated from surgical biopsy samples of osteoarthritic subjects. Knockdown of GPR64 showed significant repression of MMP13 mRNA levels, to levels superior to that detected with RNAi-mediated knockdown of p65, the control. These data show that the inhibition of GPR64 results in the repression of MMP13 mRNA levels in primary human cartilage cells. Furthermore, these data support the previous observations that were performed in two different human chondrocyte cell lines. Together, these data show that inhibition of GPR64 may be an important therapeutic intervention point for the treatment of OA. Also, these data support that monitoring MMP13 mRNA levels may be a useful assay for screening for compounds that modulate GPR64 activity.

As noted above, embodiments of the invention provide methods of screening for agents for treating an inflammatory disease in a subject. This method can be practiced by screening for an agent that modulates (e.g., inhibits or activates) the activity of GPR64 or that modulates the expression of GPR64. In some embodiments, this method can be practiced by screening for an agent that inhibits the activity or expression of an enzyme that degrades cartilage, such as, for example, MMP13, ADAMTS1, ADAMTS4, ADAMTS5, ADAMTS8, ADAMTS9, and/or ADAMTS15. In some embodiments, the subject is selected from the group consisting of rat, mouse, monkey, cow, horse, pig, rabbit, goat, sheep, dog, cat, and human. In one embodiment, the subject is a human. In some embodiments, the subject is not human.

As used herein, “agent” includes, but is not limited to, synthetic small molecules, chemicals, nucleic acids, such as, for example, antisense oligonucleotides and silencing RNA, peptides, and proteins, such as, for example, hormones, cytokines, antibodies and portions thereof, and receptors and portions thereof. In one aspect, the methods include contacting a sample of tissue, such as, for example, one in which GPR64 is expressed, or contacting GPR64 with a test agent. In one embodiment, the test agent modulates (e.g., inhibits or increases) the activity or expression of GPR64. In another embodiment, the test agent modulates the activity or expression of one or more component of the NFκB signal pathway, such as, for example, localization of a component in the nucleus as compared to the cytoplasm, MMP13, ADAMTS1, ADAMTS4, ADAMTS5, ADAMTS8, ADAMTS9, and/or ADAMTS15. In some embodiments, the test agent inhibits the activity or expression of GPR64 and/or one or more component of the NFκB signal pathway.

Additional assays that could be used for these methods of screening include known assays for GPCR function, including, but not limited to, calcium flux assays or cAMP activity assays, as well known in the art and as described in more detail herein. (See, e.g., FLIPR Calcium Assay Kit, Molecular Devices, Sunnyvale, Calif.; BioVision cAMP Direct Immunoassay Kit, BioVision Research Products, Mountain View, Calif.; CatchPoint cAMP Fluorescent Assay Kit, Molecular Devices, Sunnyvale, Calif.) A “test agent” is a putative “agent,” the modulating ability of which has not yet been confirmed.

Once test agents are screened, they are classified as “agents” if they are shown to modulate activity (for example, by inhibiting or activating or otherwise affecting the signal pathway) or expression (for example, by modulating transcription or translation). Accordingly, in additional embodiments, the agent may modify GPR64 transcription, GPR64 translation, or the GPR64 signal pathway. In some embodiments, the agent down-regulates the GPR64 signal pathway. In additional embodiments, the agent up-regulates the GPR64 signal pathway. In a particular embodiment, the activity or expression of GPR64 is inhibited by the agent. In another embodiment, the activity or expression of GPR64 is activated by the agent. In some embodiments the agent binds to GPR64. In other embodiments, the agent interacts with GPR64. In still other embodiments, the agent binds to or interacts with (such as by chemically modifying) an inhibitor or activator of GPR64 activity or expression. By way of non-limiting example, an agent may bind to and inhibit (or activate) an activator of GPR64 or an agent may bind to and activate (or inhibit) an inhibitor of GPR64 activity.

In additional embodiments, the agent affects the level of activity or expression of a protease. In various embodiments, the protease is an enzyme that degrades cartilage. In further embodiments, the agent affects or modulates the NFκB pathway. In some embodiments, the agent modulates the expression and/or activity of MMP13. In additional embodiments, the agent modulates the expression and/or activity of ADAMTS1, ADAMTS4, ADAMTS5, ADAMTS8, ADAMTS9, ADAMTS15, or other cartilage degrading enzyme. In further embodiments, the agent affects the location of a transcription factor (such as, for example, p65 or the NFκB complex) or co-factors related to NFκB activation, such as, for example, being located in the nucleus as compared to the cytoplasm.

The methods include: contacting or exposing a sample (e.g., of tissue, a cell culture, or an amount of GPR64) with/to a test agent, detecting a level of activity or expression of GPR64 and comparing the level of activity or expression of GPR64 to a control level. The level of activity or expression of GPR64 can be increased or decreased relative to the control level. If the test agent modulates (e.g., inhibits or augments) the activity or expression of the GPR64, then it may be classified as an agent for treating inflammatory disease.

A control level can be determined by any method known in the art. By way of non-limiting example, a control level includes standard levels or normal levels. Such standard levels can be determined by testing the level of GPR64 in a specific tissue (which corresponds to the tissue being tested in the method) from a variety of subjects without an inflammatory disease. An average of these levels can be used as the control level. If tissue from different animals are used, standard levels can be determined for each animal species or for a group of animal species. In addition, in some embodiments, a control level refers to the level measured from the sample to which the experimental element was not applied in an experiment.

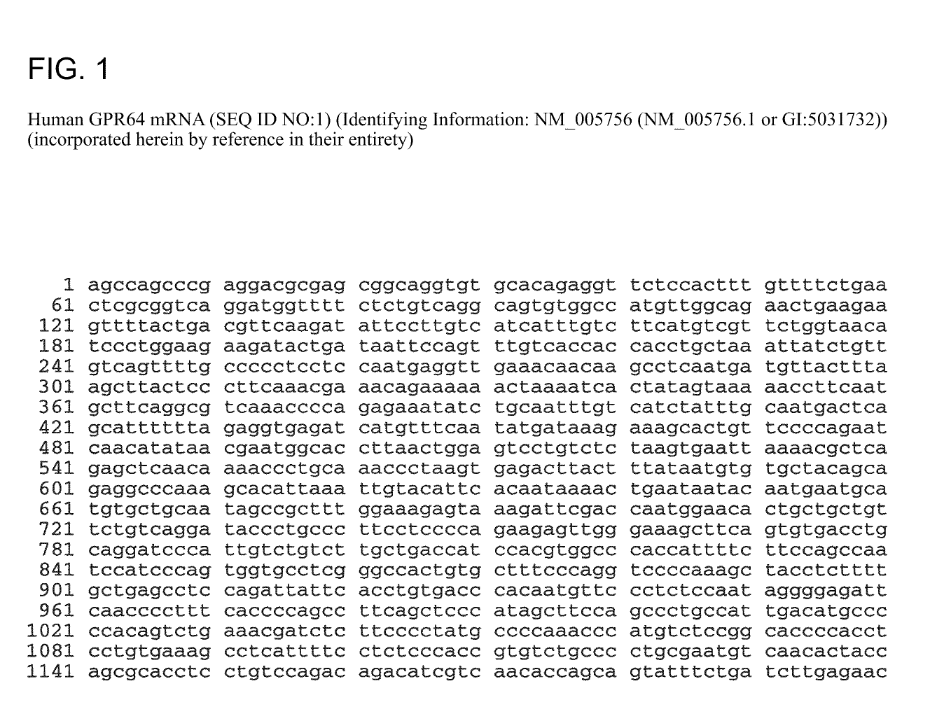

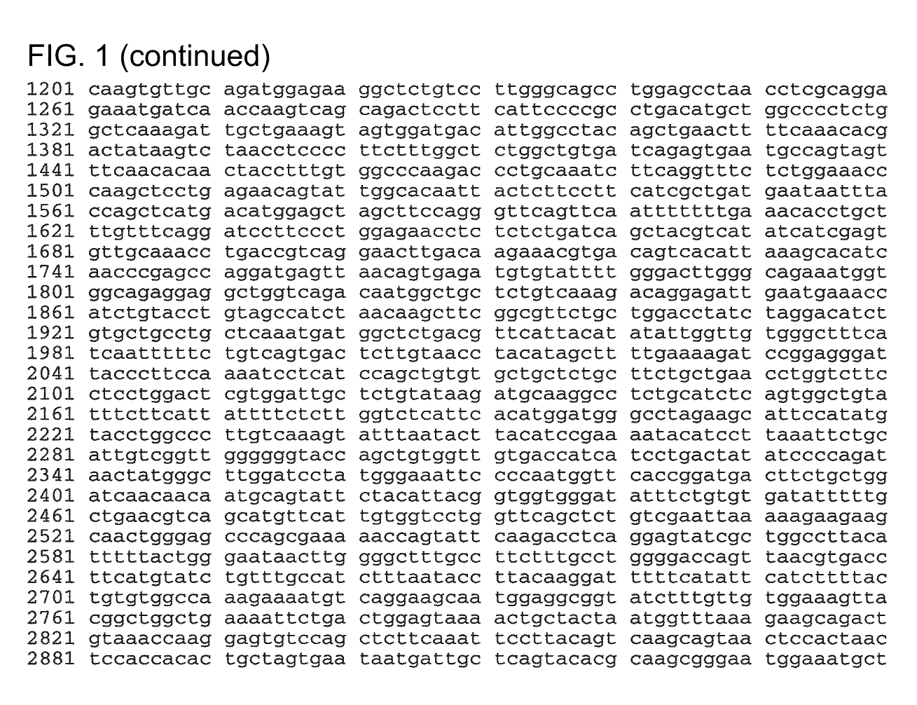

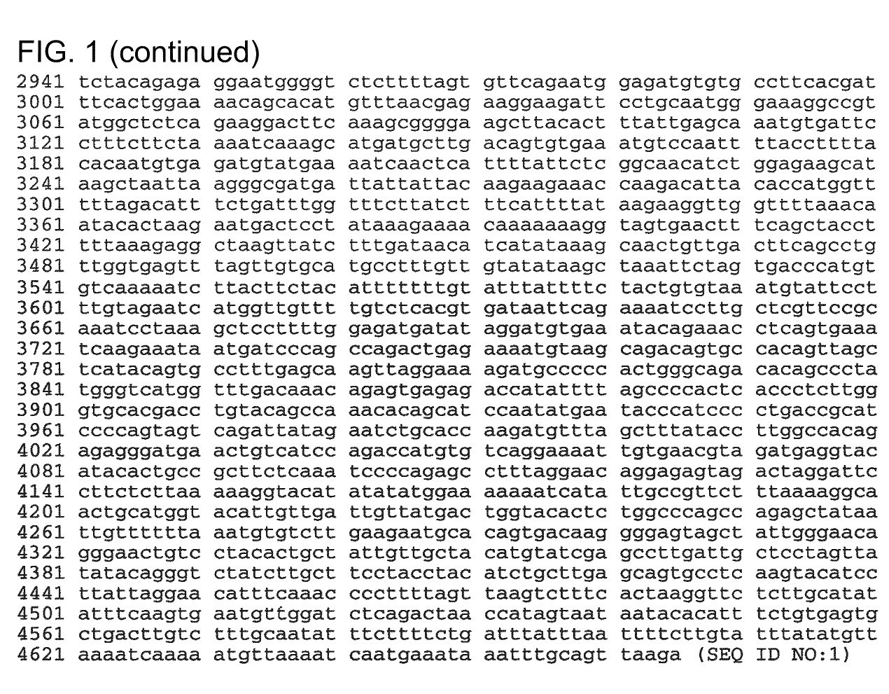

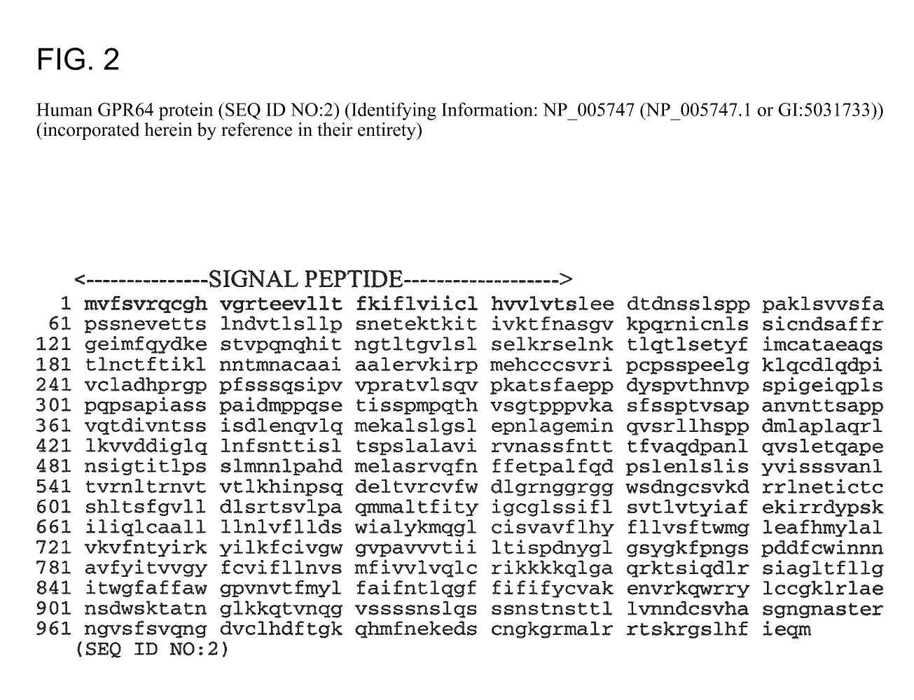

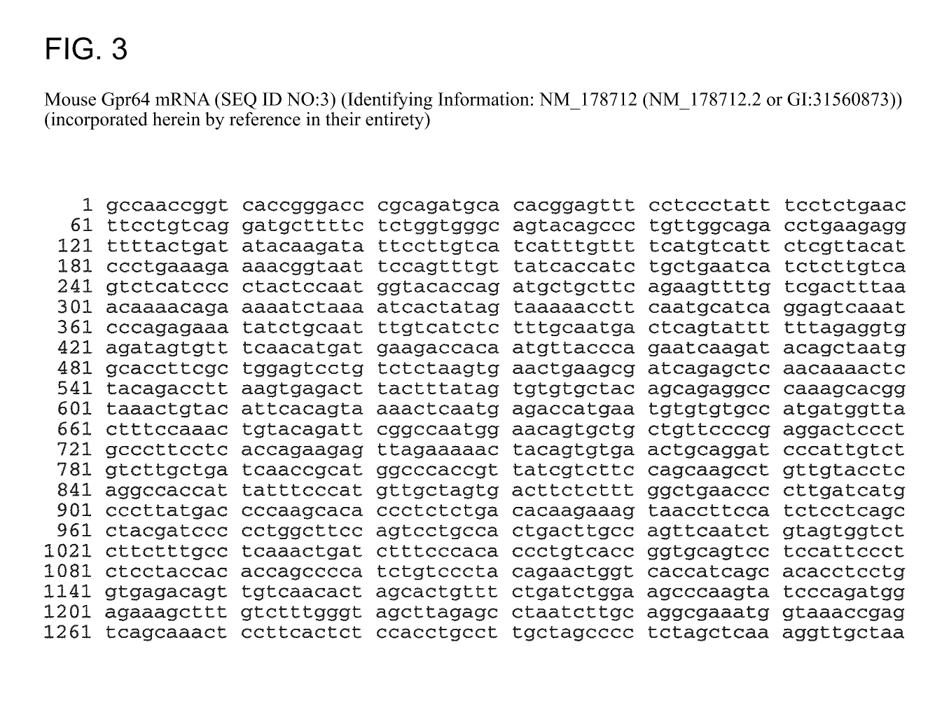

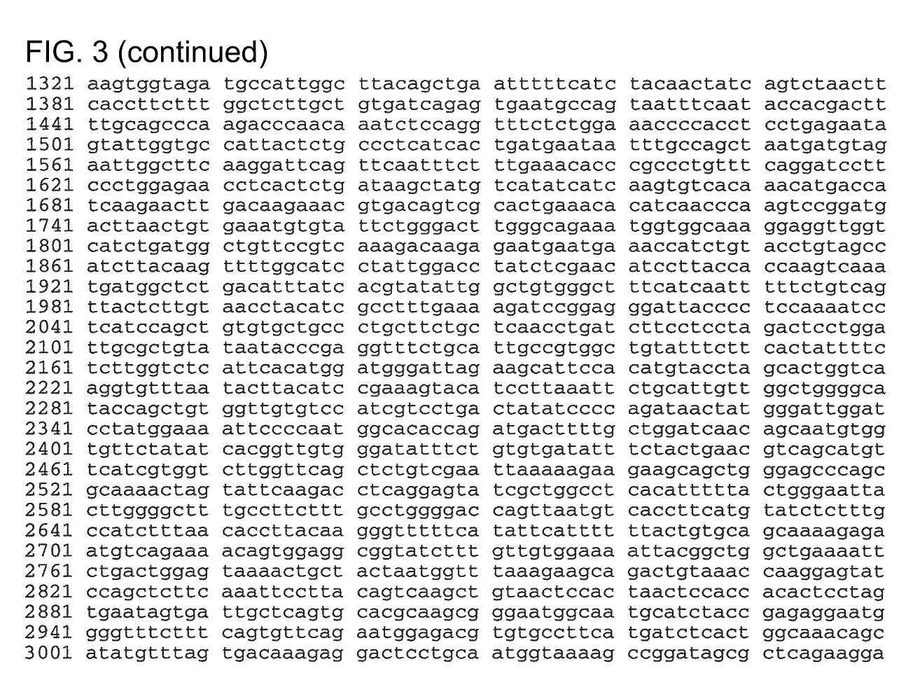

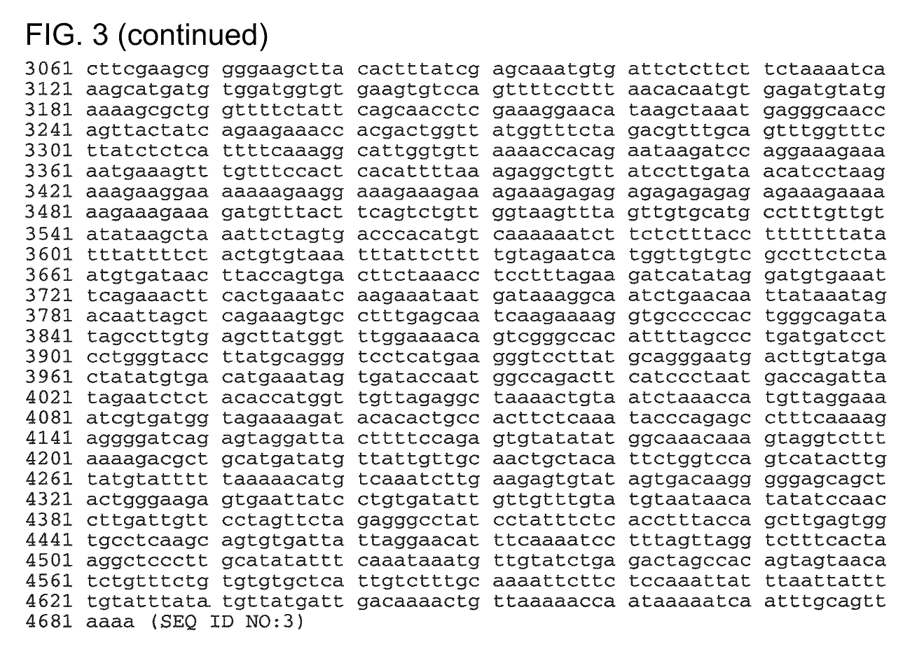

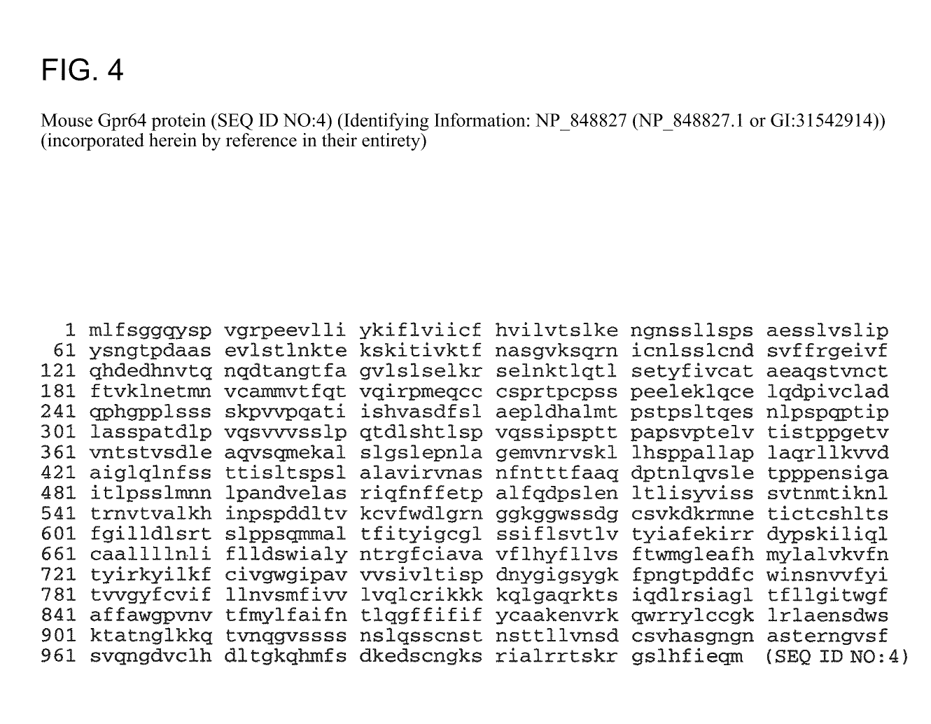

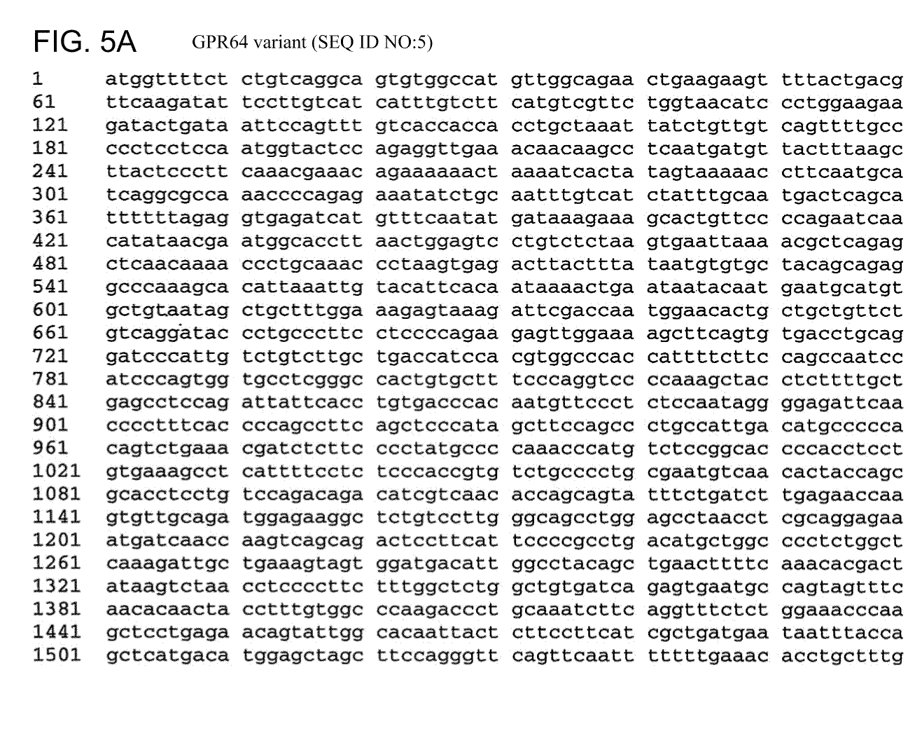

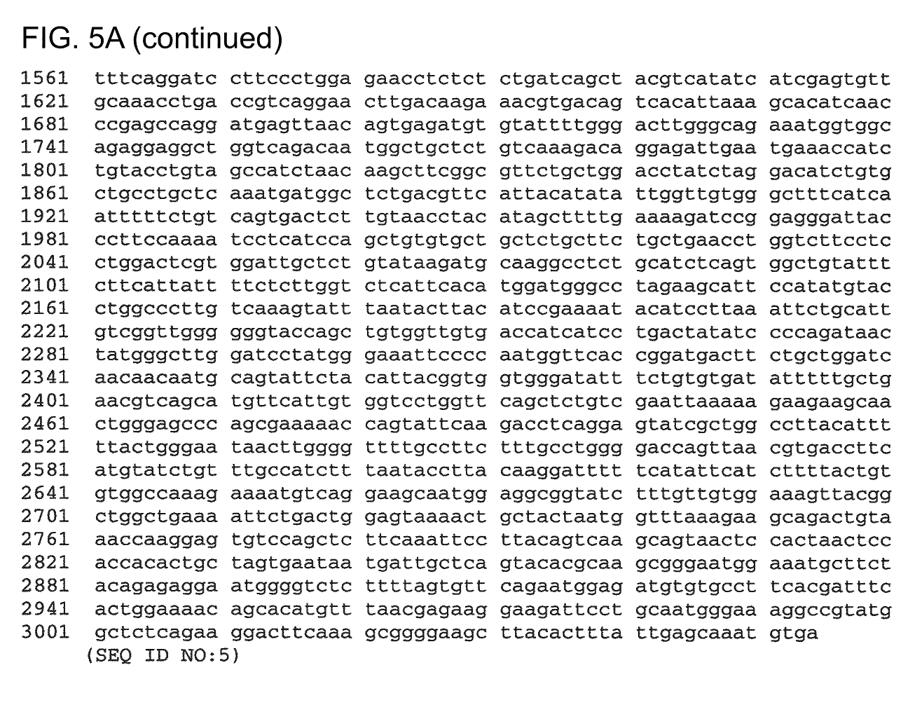

The gene for GPR64 is located at chromosome location Xp22.22. The nucleotide and amino acid sequences of human GPR64 are set forth in SEQ ID NO:1 and SEQ ID NO:2, as provided in FIGS. 1 and 2, respectively. The nucleotide and amino acid sequences of murine GPR64 are set forth in SEQ ID NO:3 and SEQ ID NO:4, as provided in FIGS. 3 and 4, respectively.

The discovery that GPR64 is associated with inducing the symptoms and/or complications of inflammatory diseases renders the sequences of GPR64 useful in methods of identifying agents described herein. Such methods include assaying test agents for the ability to modulate GPR64 activity or expression. Polynucleotides and polypeptides useful in these assays include not only the genes and encoded polypeptides disclosed herein, but also variants thereof that have substantially the same activity as wild-type genes and polypeptides. “Variants”, as used herein, include polynucleotides or polypeptides containing one or more deletions, insertions or substitutions, as long as the variant retains substantially the same activity of the wild-type polynucleotide or polypeptide. With regard to polypeptides, deletion variants are contemplated to include fragments lacking portions of the polypeptide not essential for biological activity, and insertion variants are contemplated to include fusion polypeptides in which the wild-type polypeptide or fragment thereof has been fused to another polypeptide.

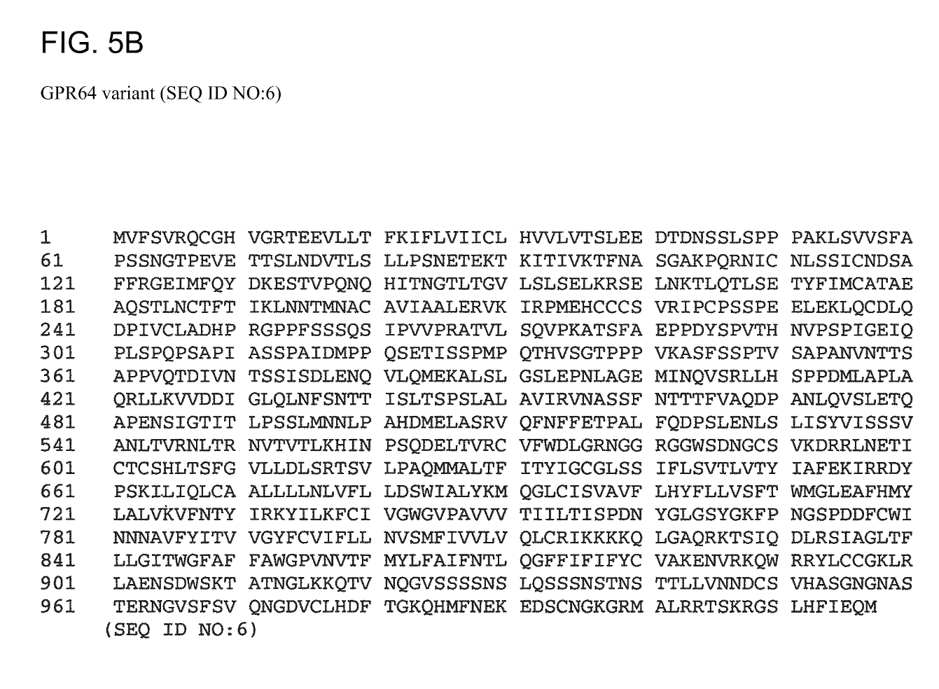

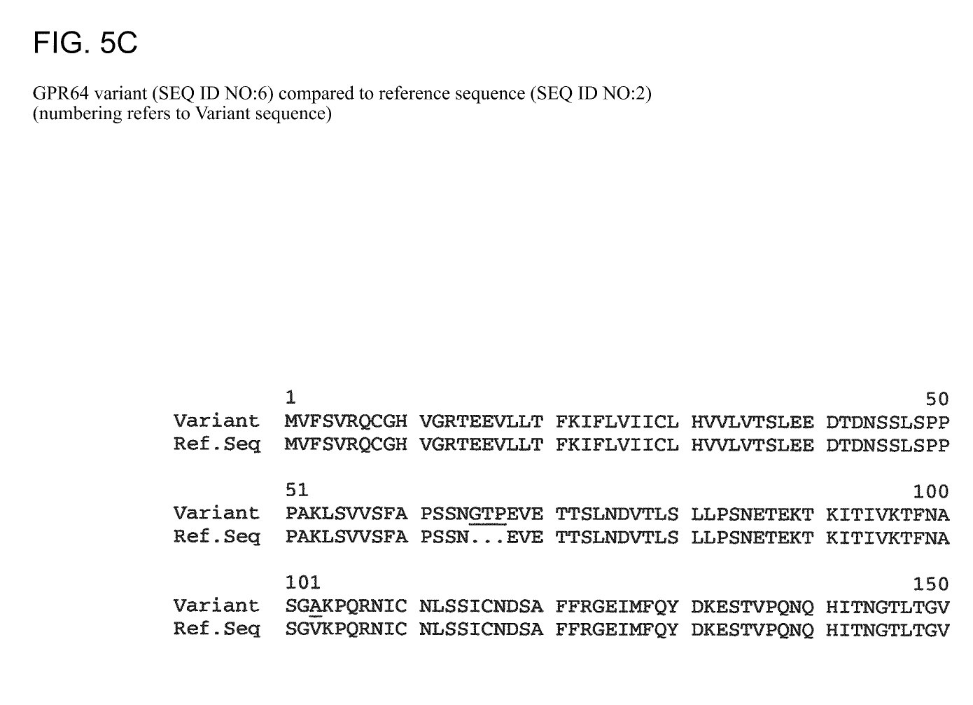

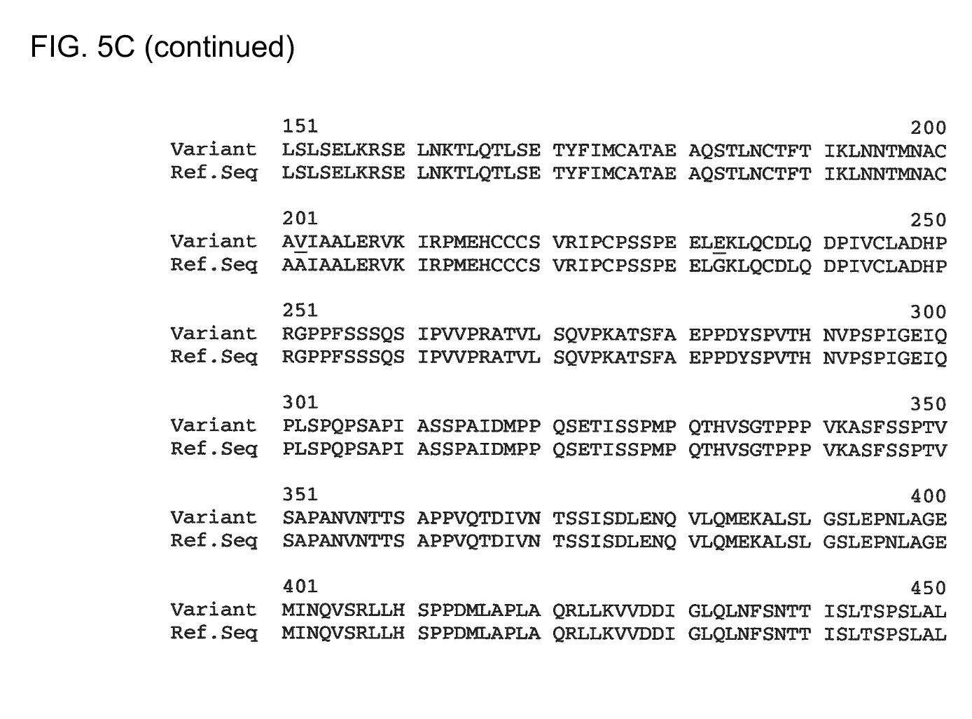

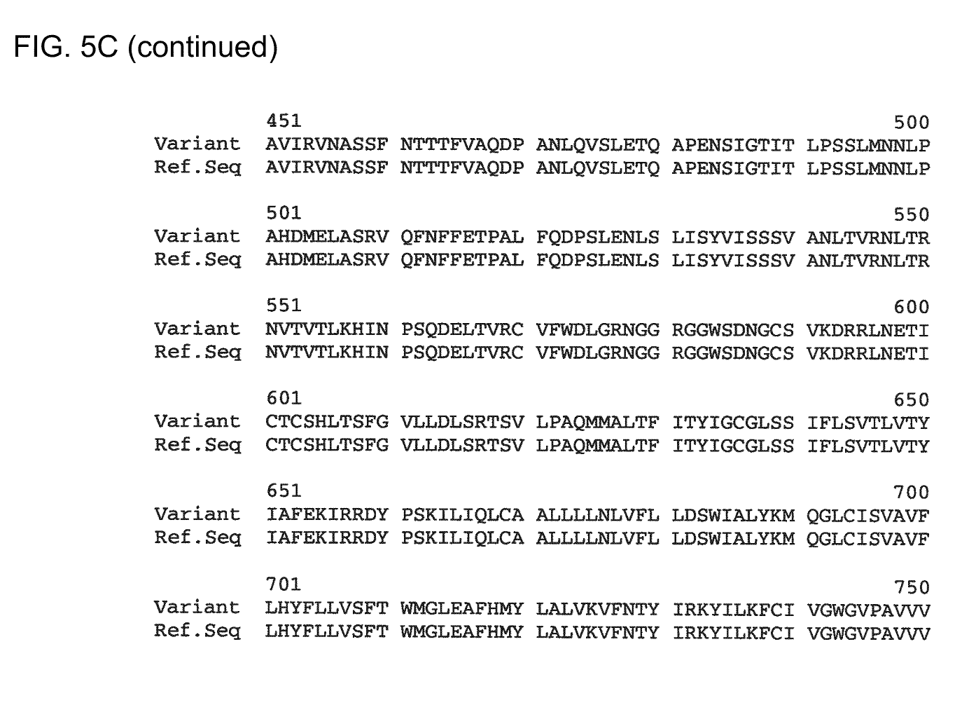

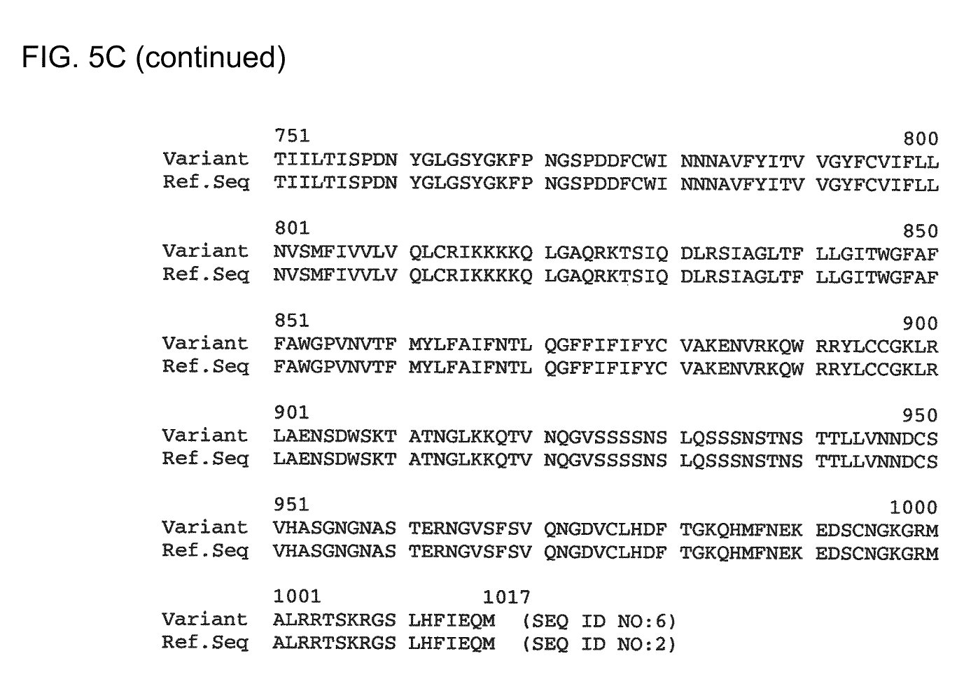

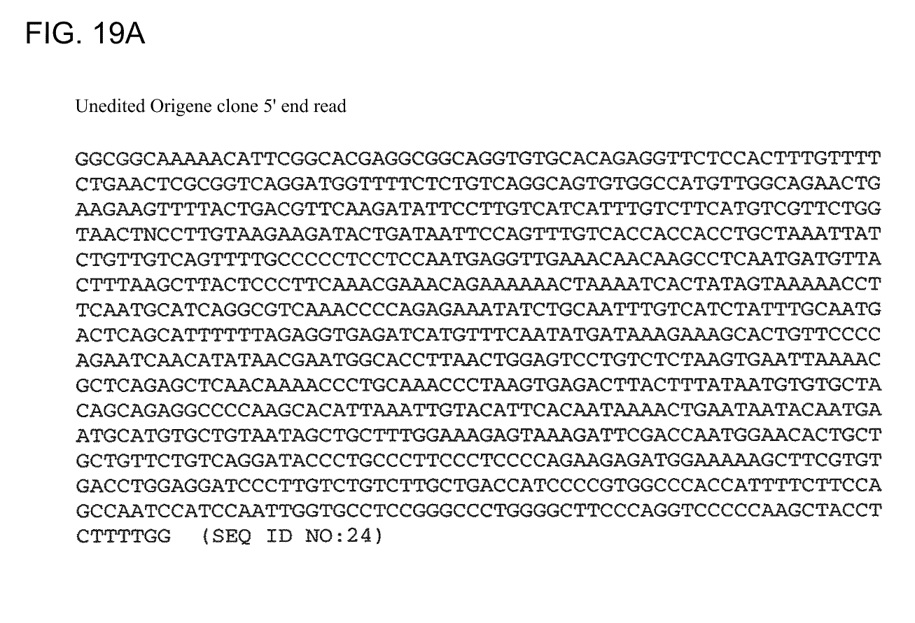

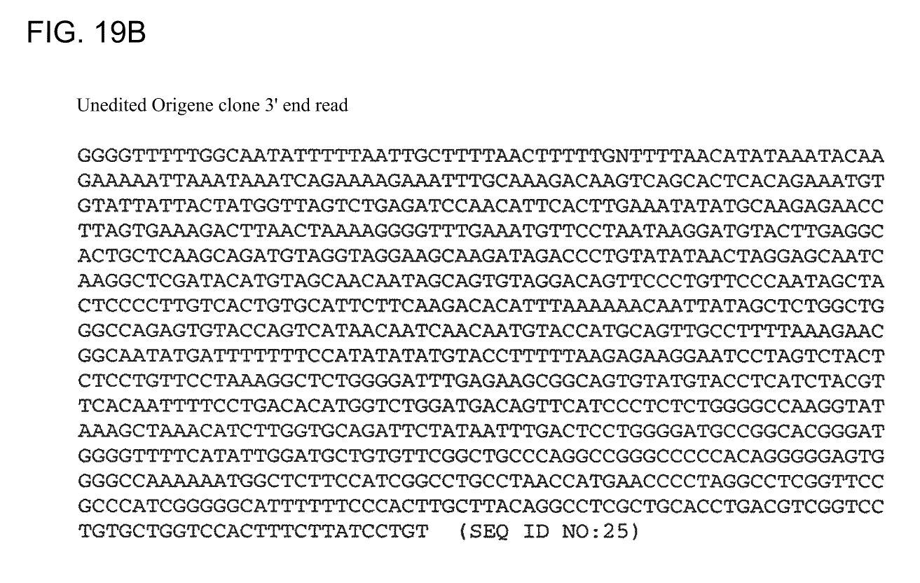

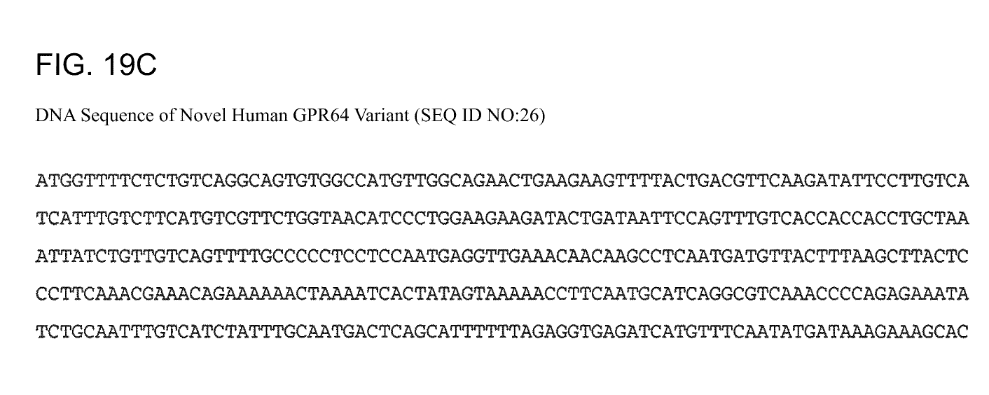

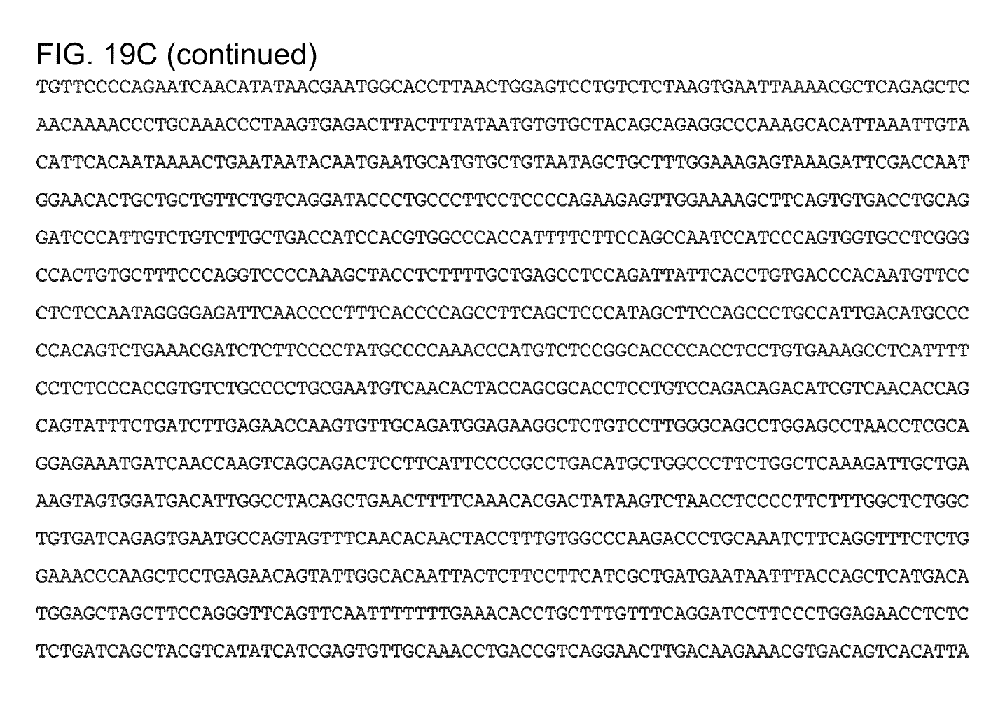

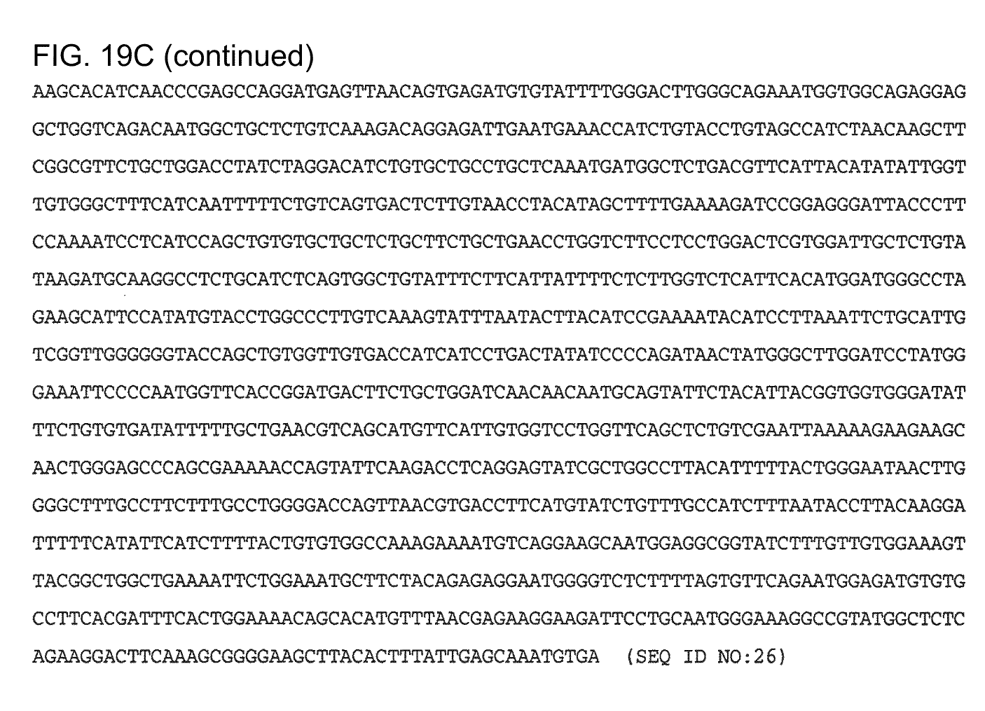

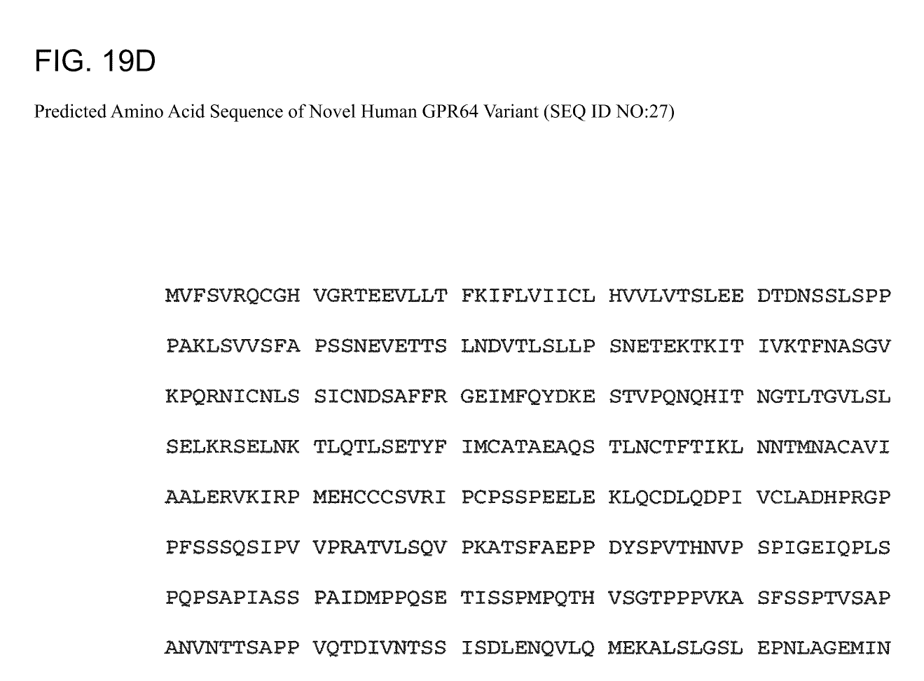

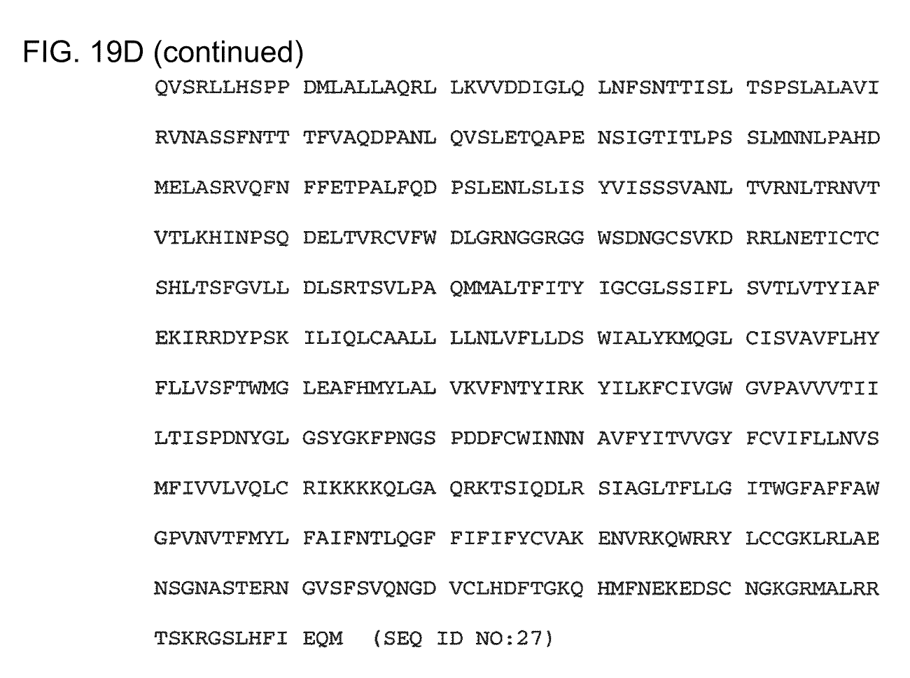

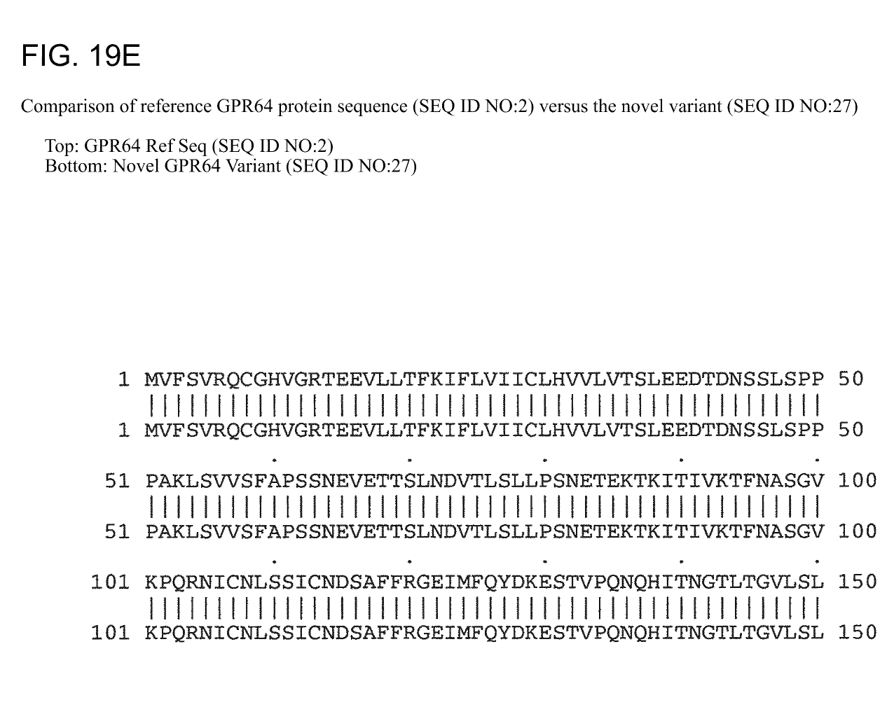

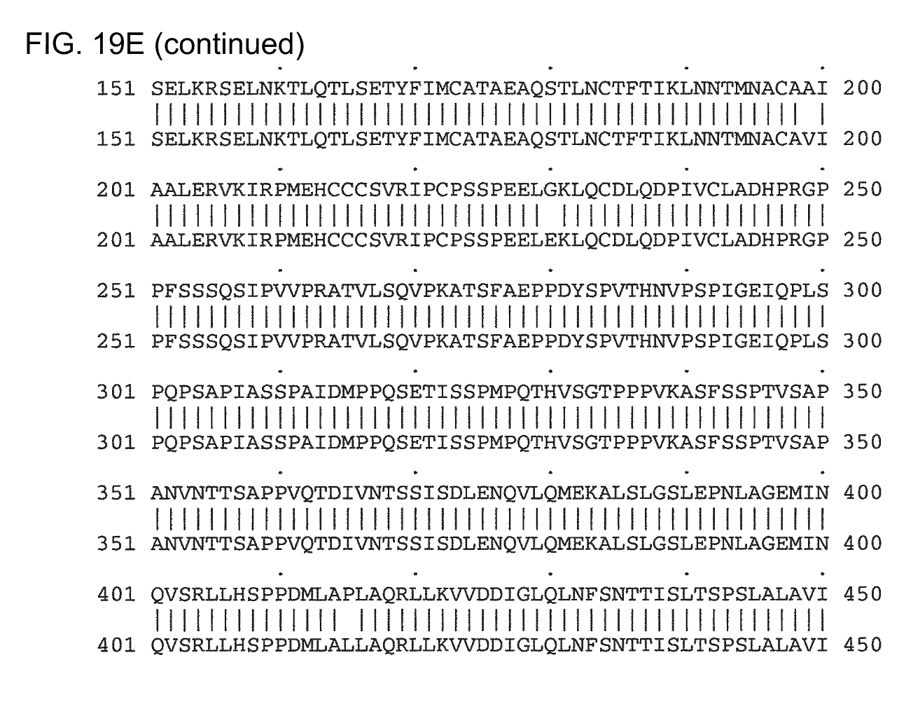

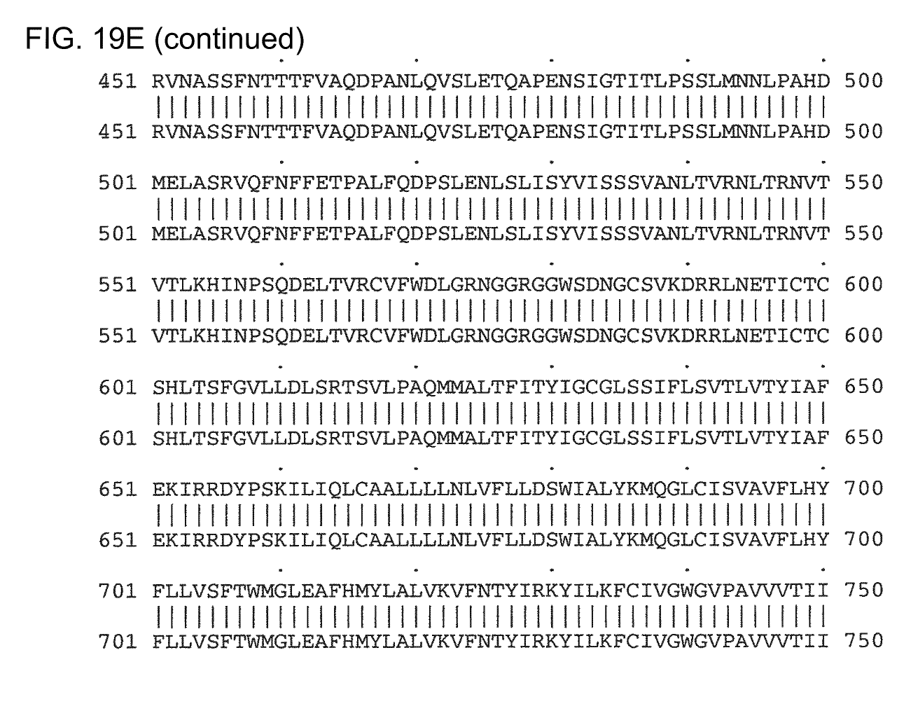

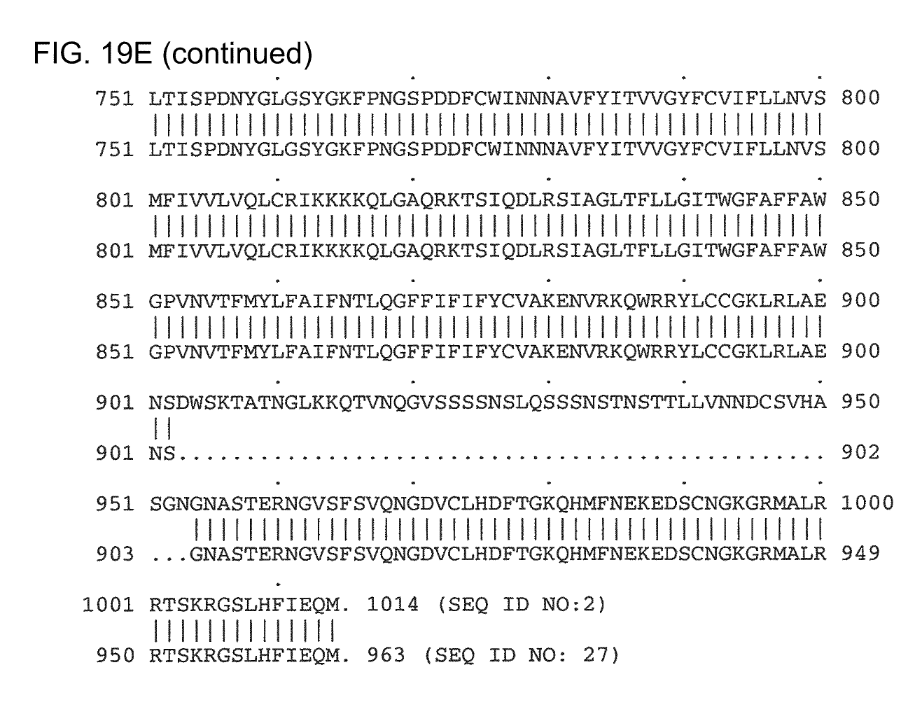

The inventors have discovered new GPR64 variants, which are described herein. A GPR64 variant (nucleic acid sequence SEQ ID NO:5 and amino acid sequence SEQ ID NO:6), which is closer to most reported forms of GPR64, was constructed by site directed mutagenesis and cloning as described in Example 14. A second GPR64 variant was identified as described in Example 16. The nucleotide sequence of this second GPR64 variant is provided in SEQ ID NO:26, and the predicted amino acid sequence is provided in SEQ ID NO:27. Each of these variant amino acid sequences has been compared with reference sequence (NP_{—}005747) (SEQ ID NO:2), as shown in FIG. 5 and FIG. 19E.

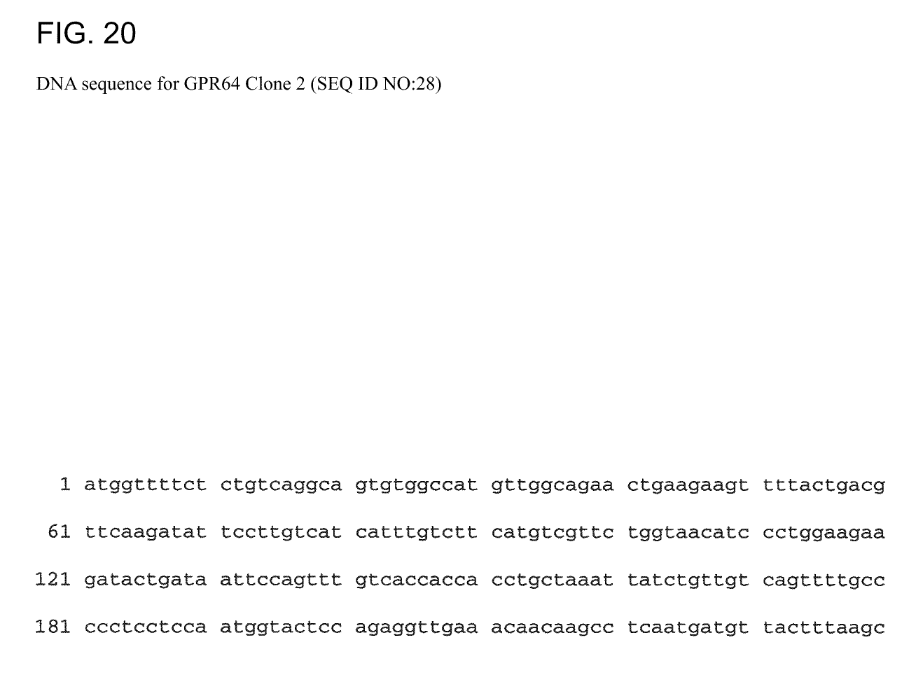

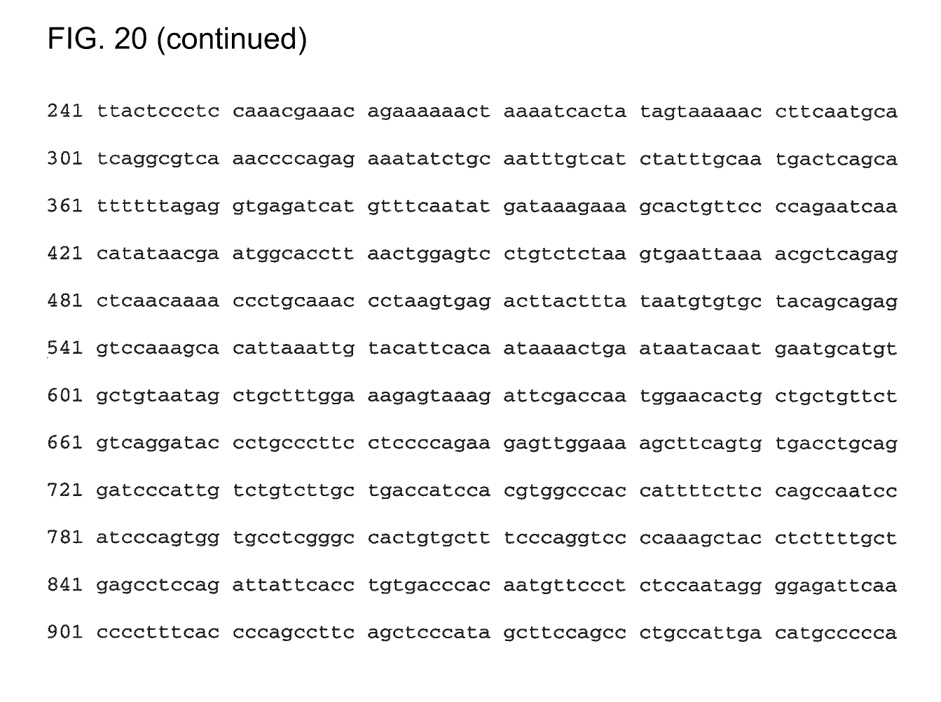

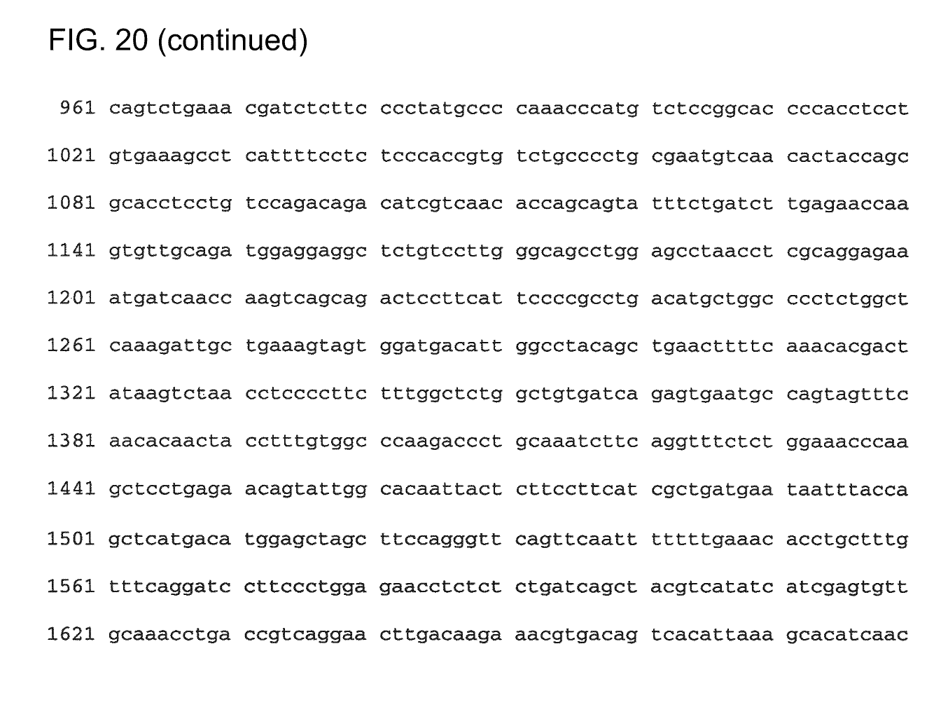

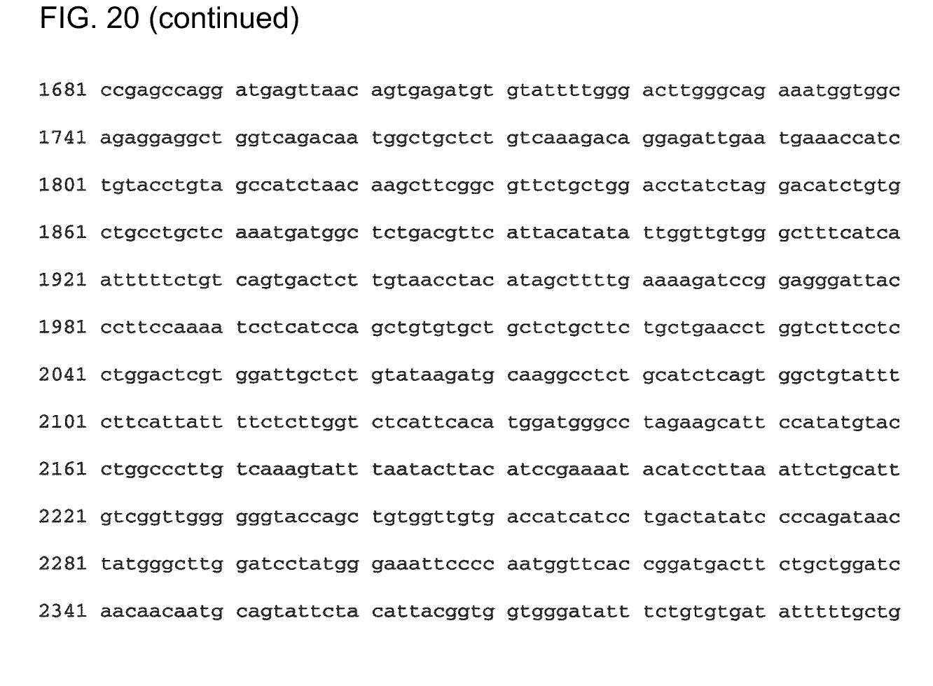

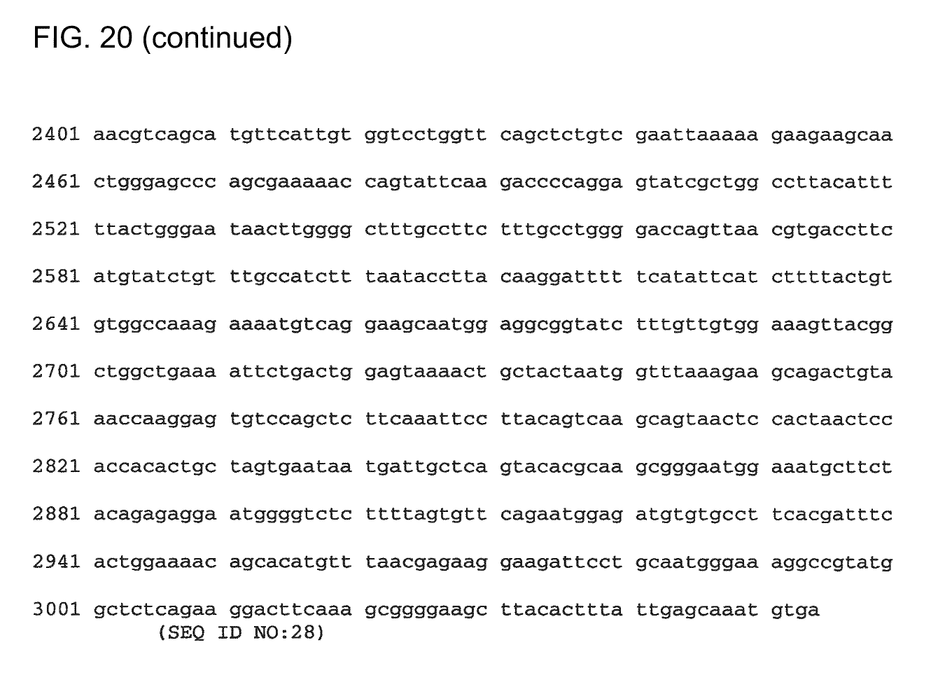

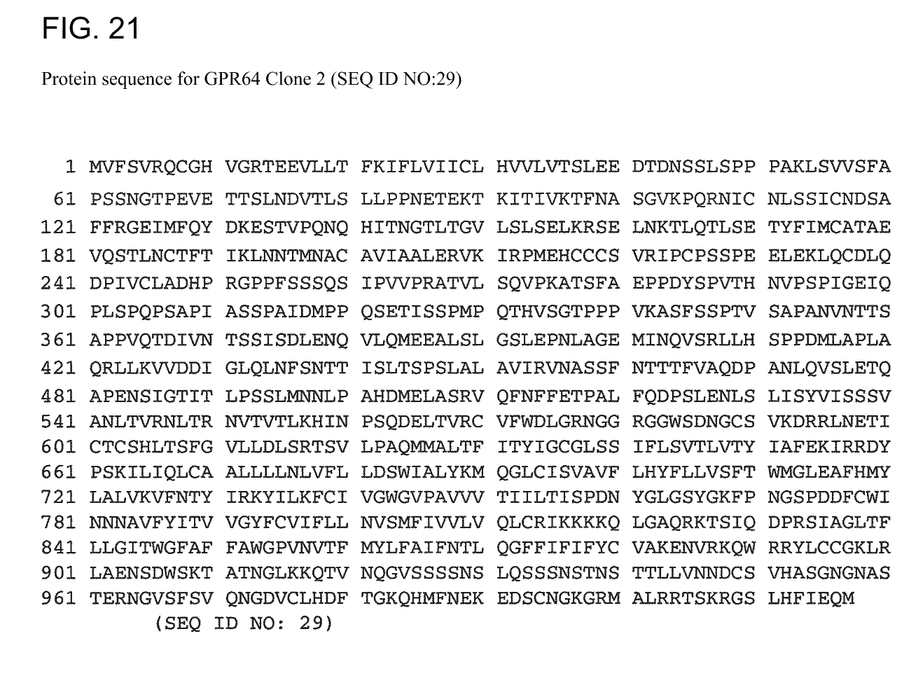

Additional GPR64 variants have been identified by the inventors. The nucleotide sequences of such variants are shown in FIGS. 20, 22, 24, 26, 28 and 30 (SEQ ID NOs:28, 30, 32, 34, 36 and 38, respectively). The predicted amino acid sequences of these variants identified by the inventors are set forth in FIGS. 21, 23, 25, 27, 29 and 31 (SEQ ID Nos:29, 31, 33, 35, 37, and 39, respectively). Each of these sequences is incorporated by reference herein in its entirety.

The variants can be expressed, for example, in U2OS, HEK, and CHO cell lines. Cell-based assays to detect GPR64 activation can be developed using the GPR64 variant prototypes. These GPR64 variants can be used to express GPR64 and for the development of further assays.

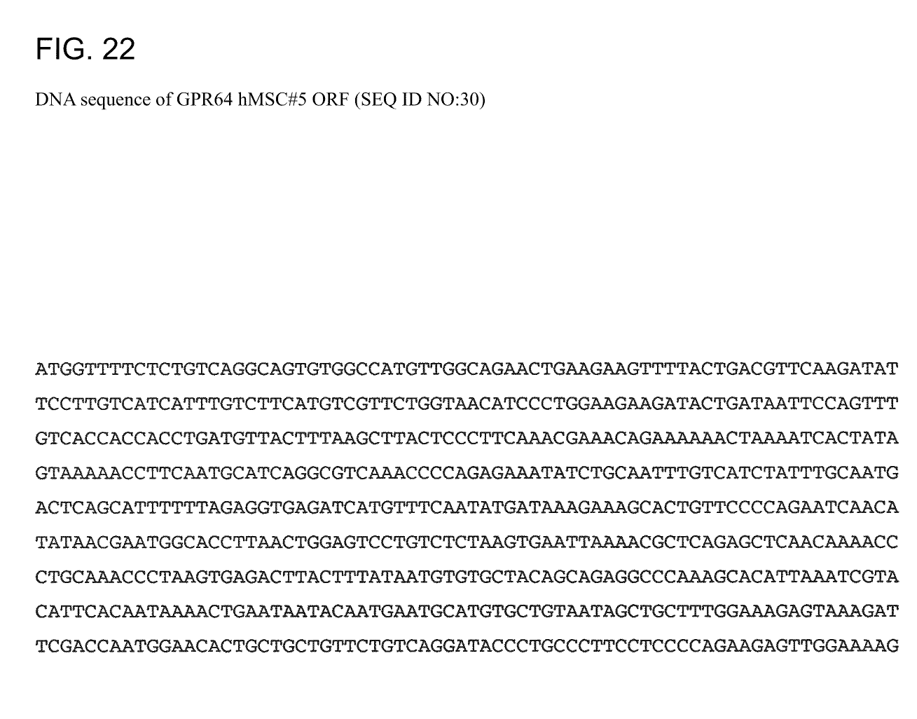

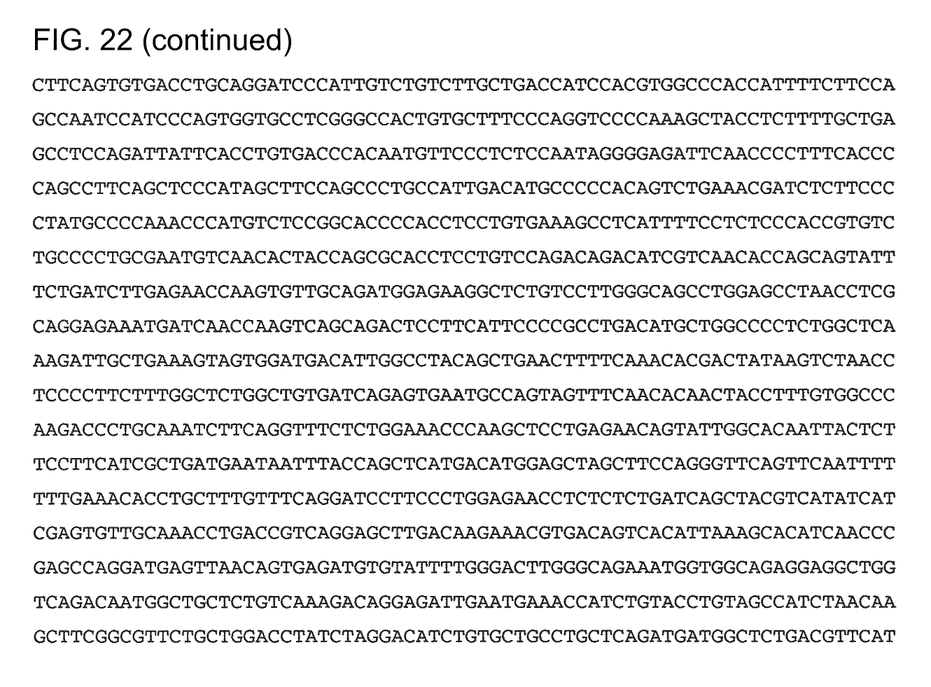

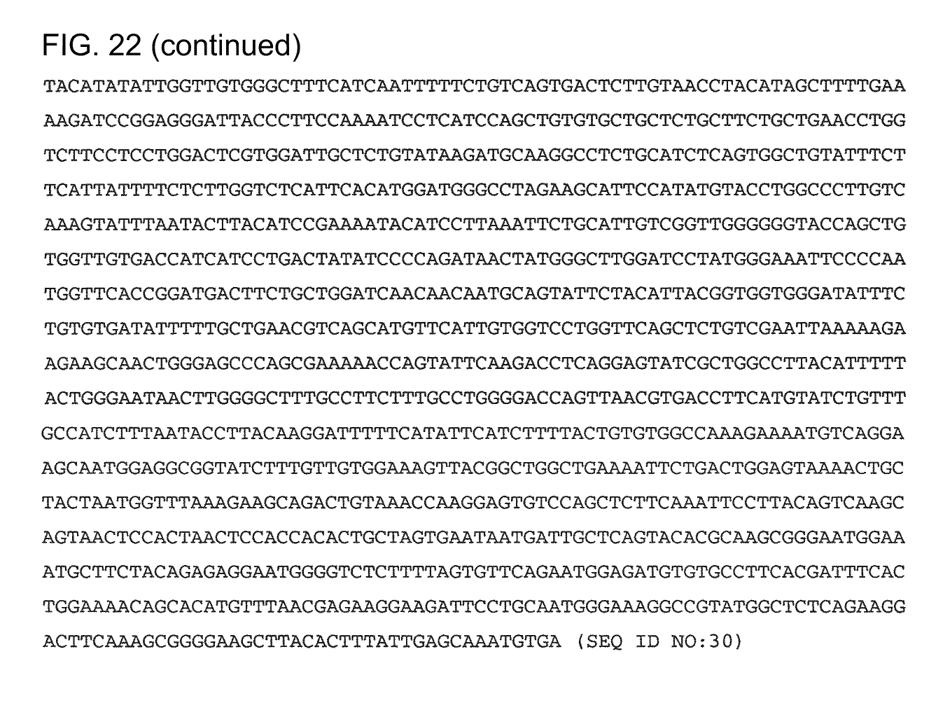

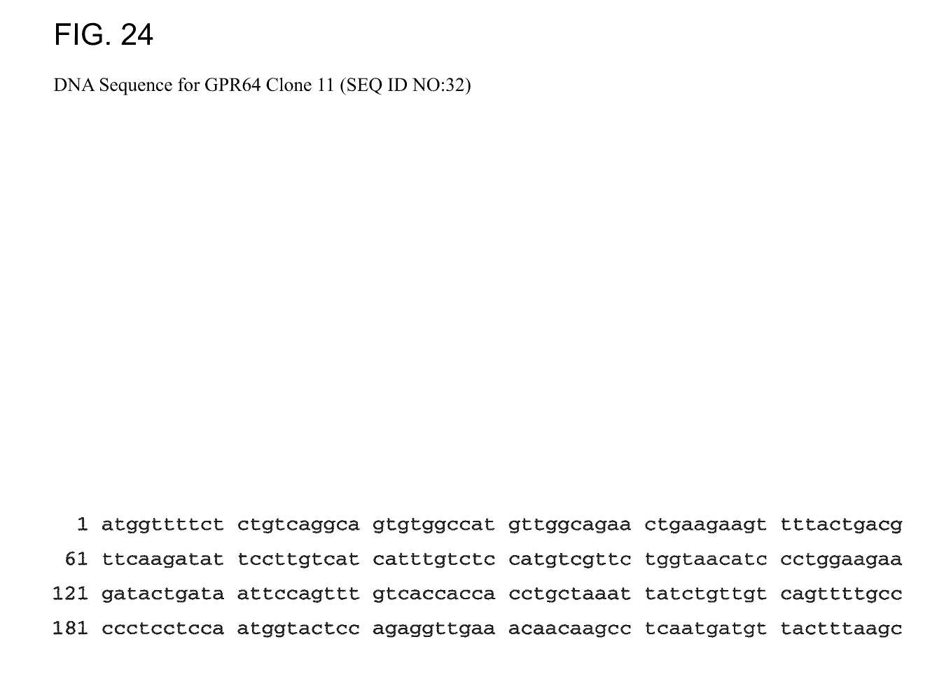

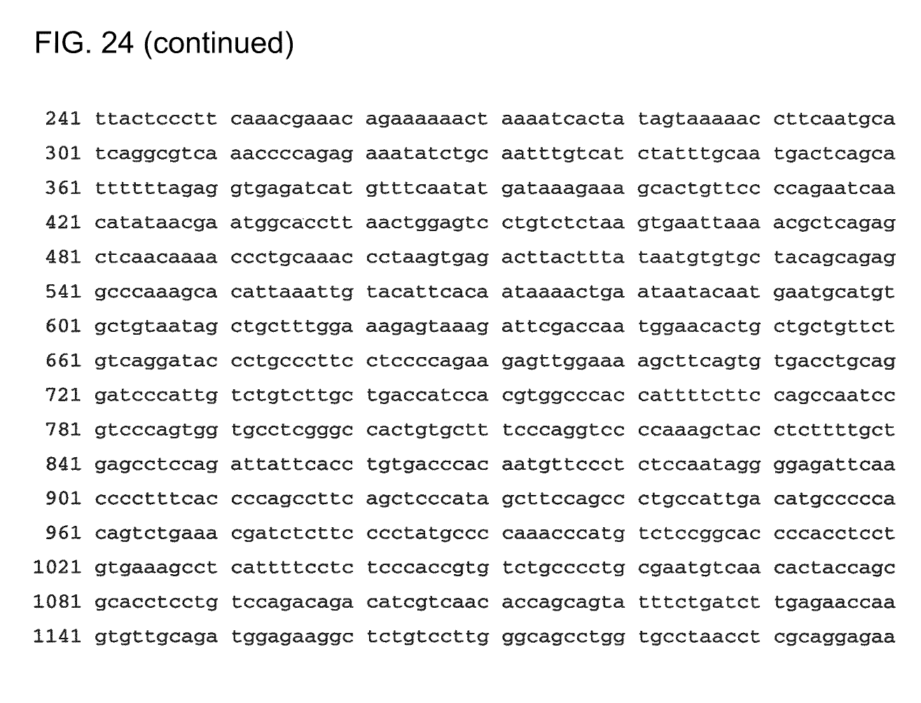

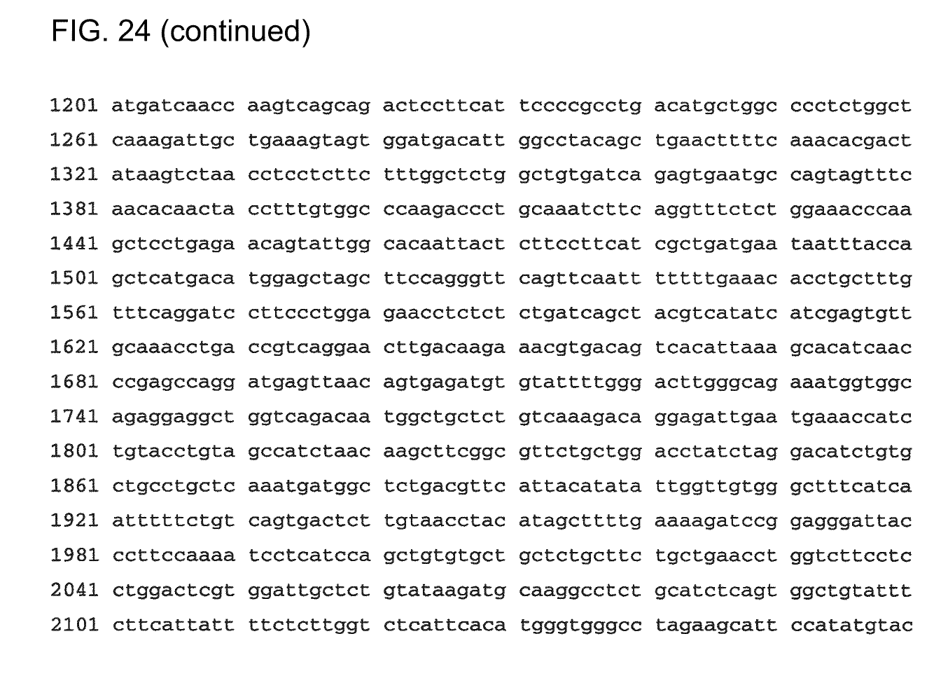

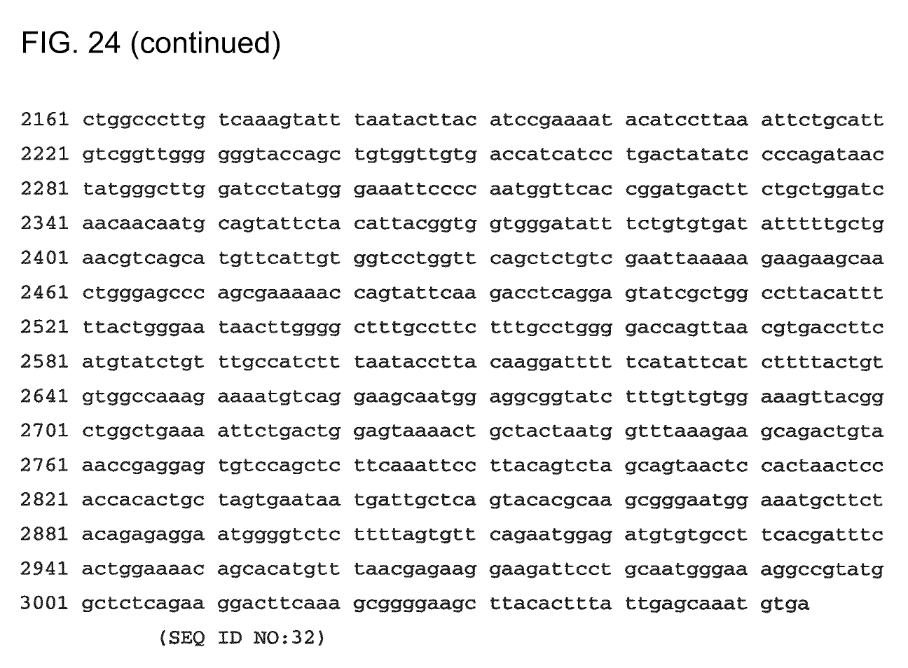

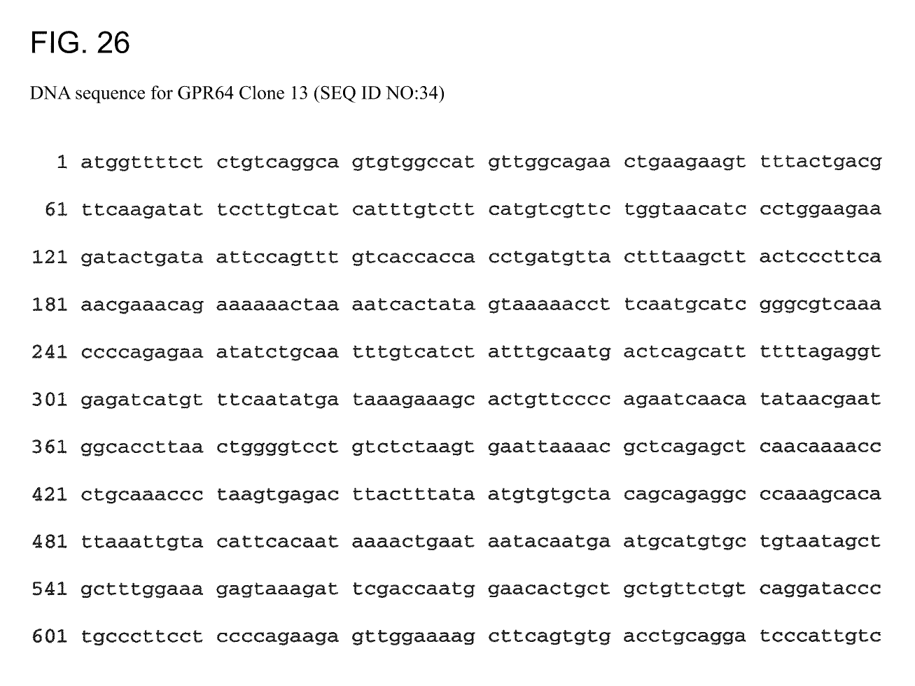

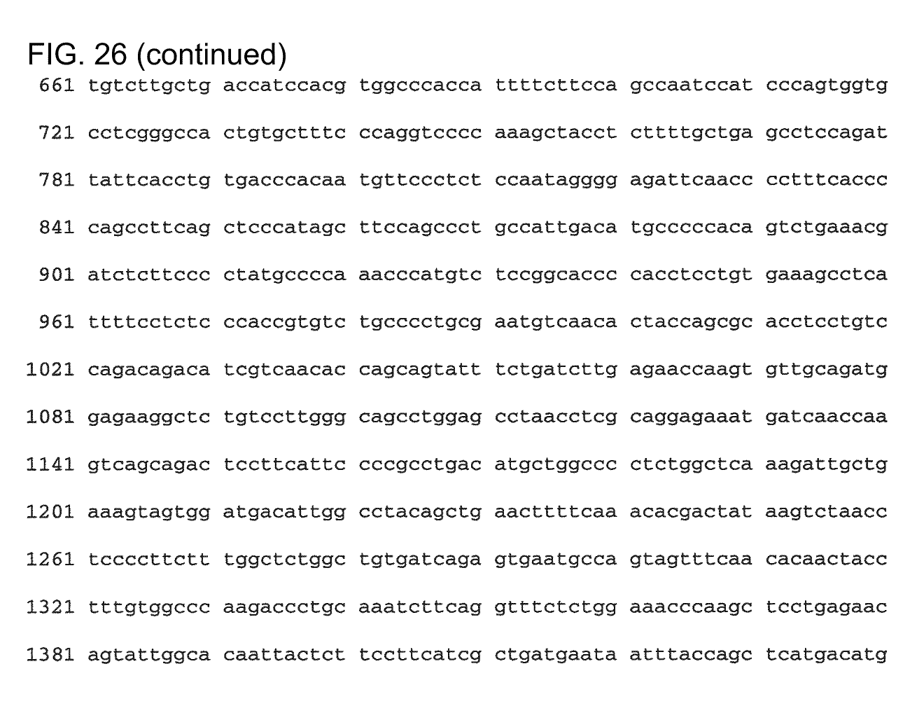

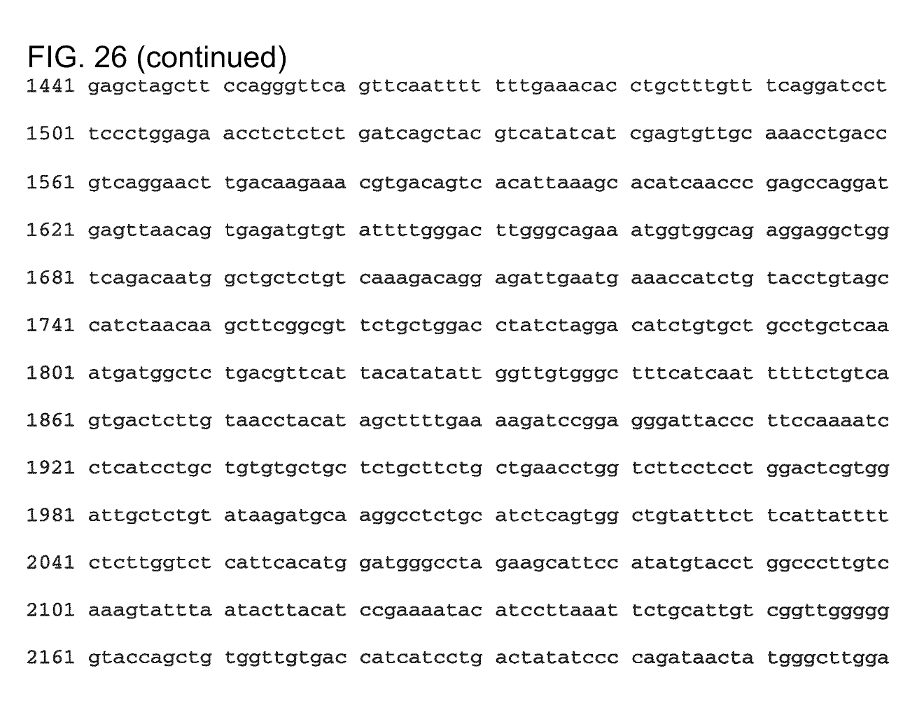

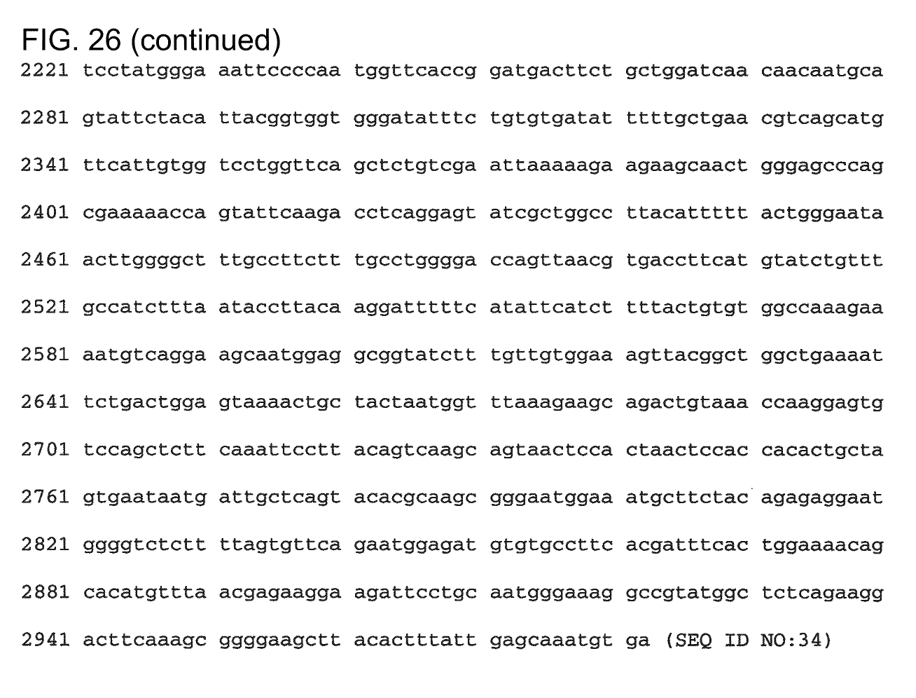

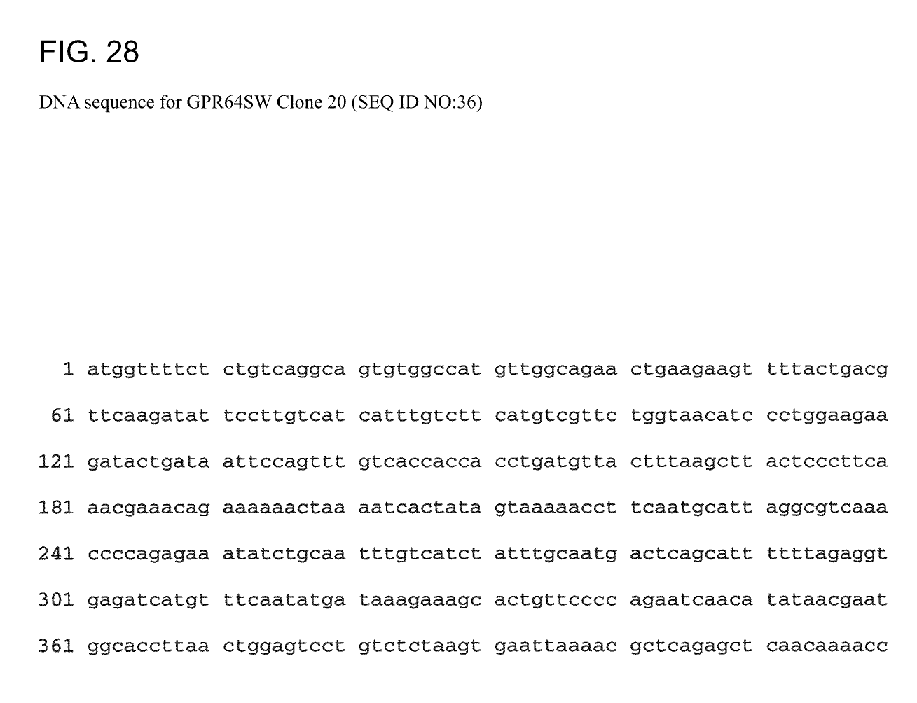

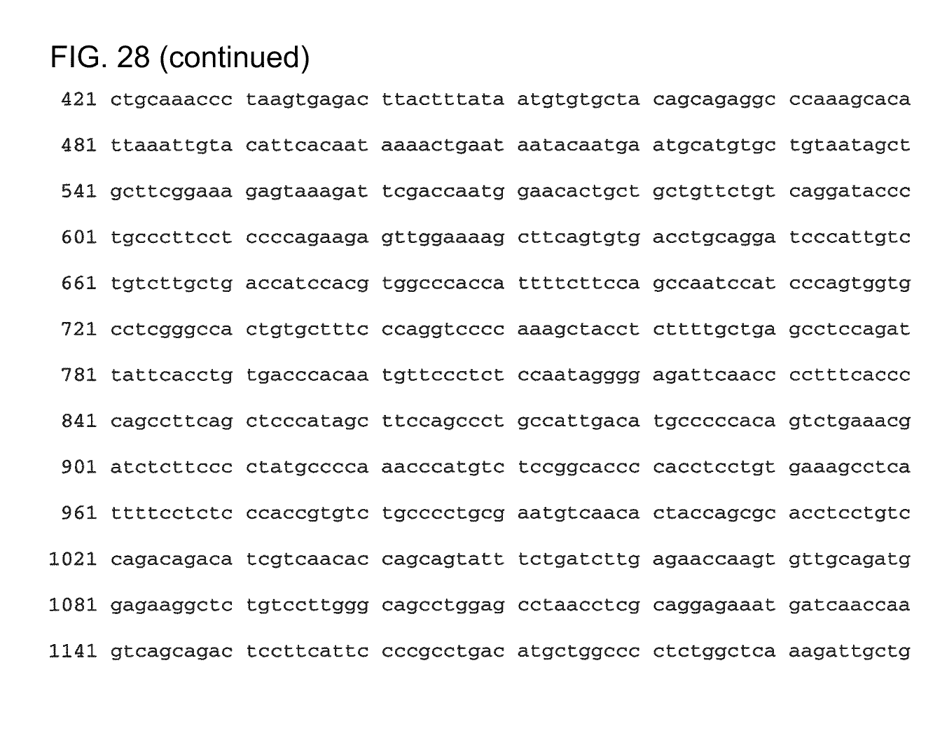

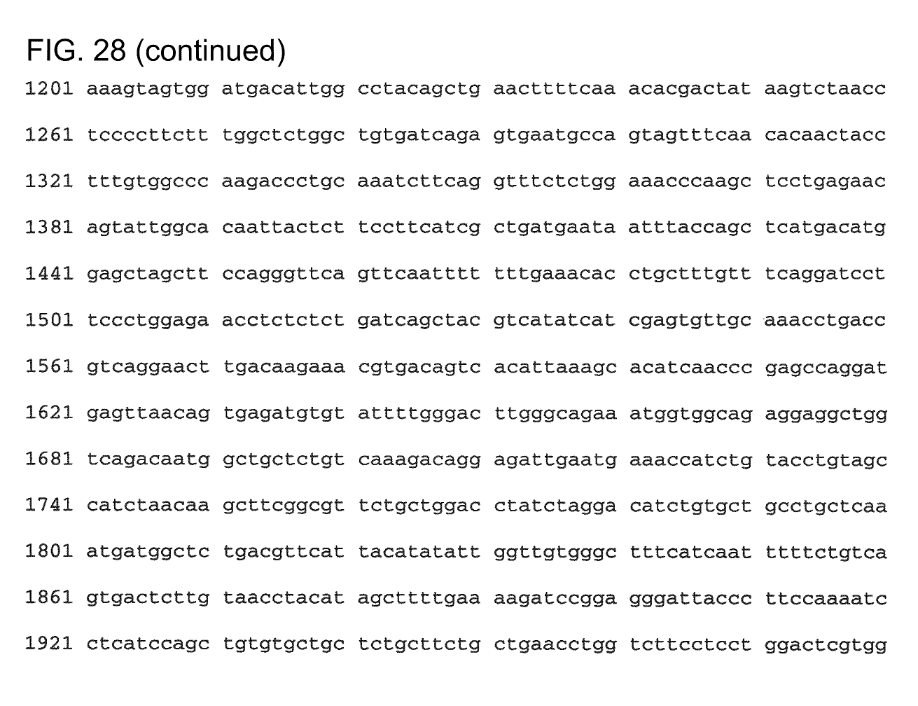

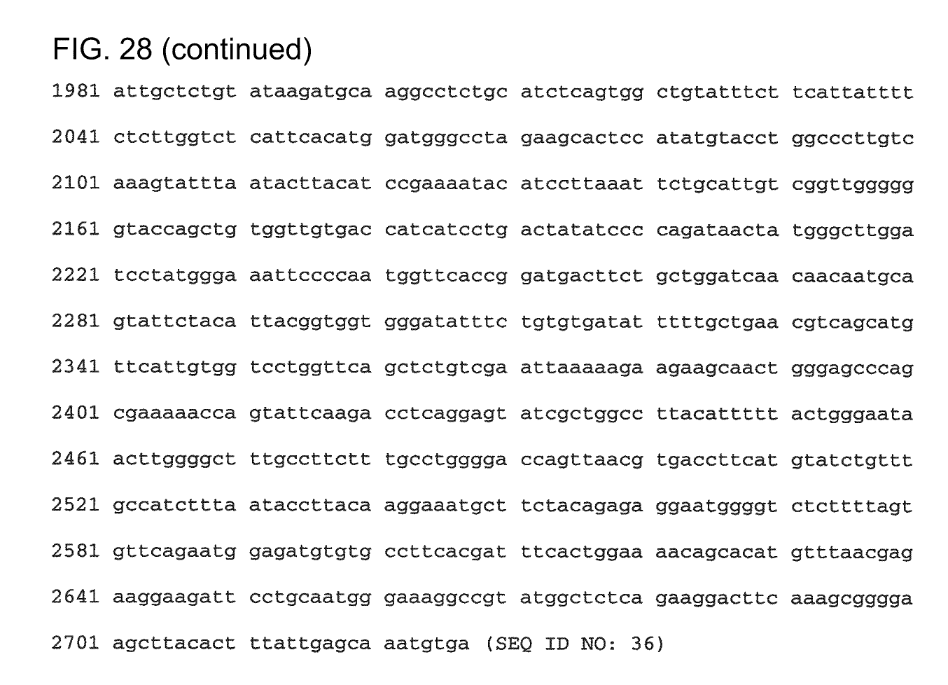

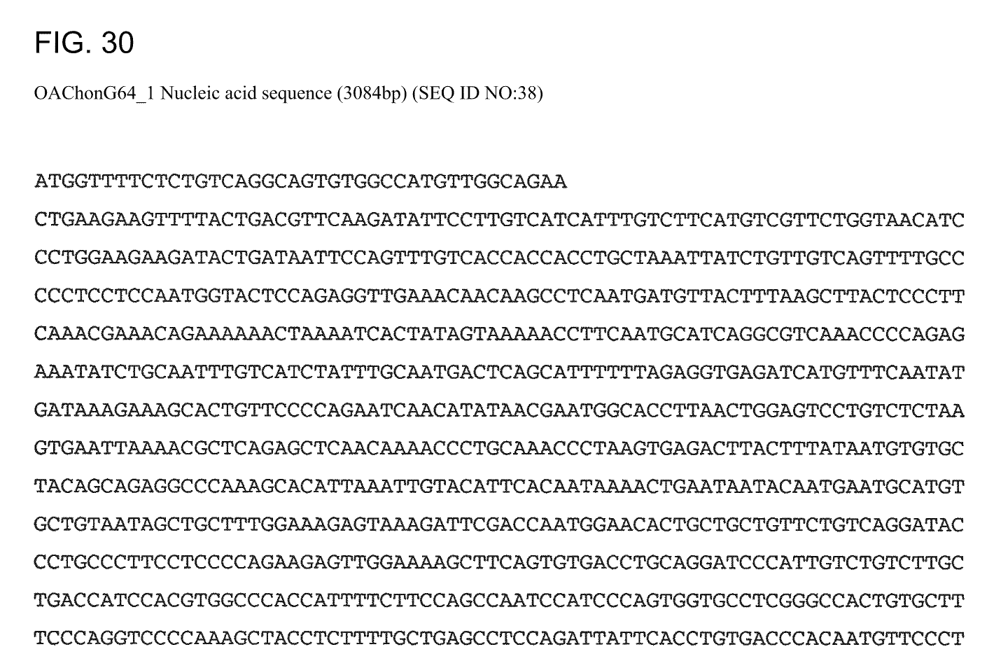

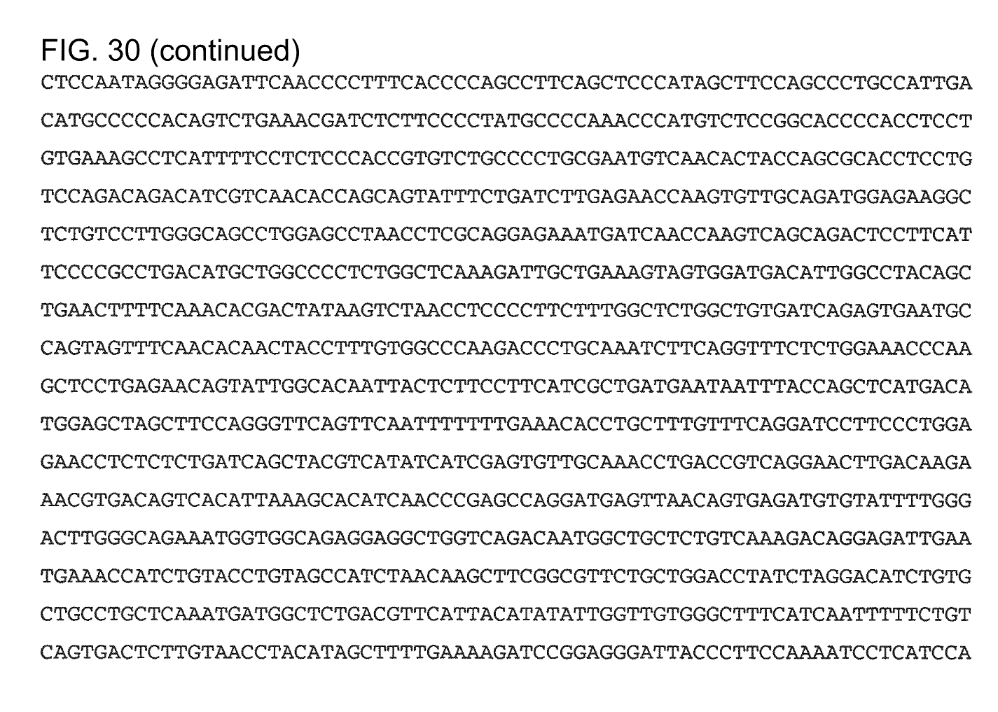

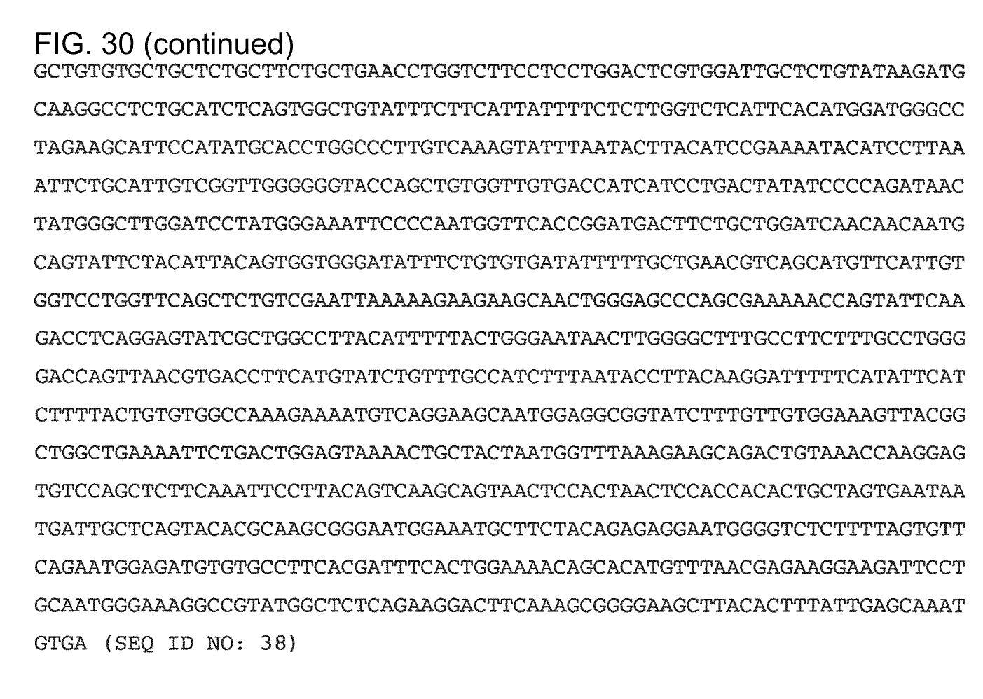

Accordingly, the GPR64 protein utilized in various embodiments of the methods and compositions described herein may be encoded by a nucleotide sequence that has at least about 60%, at least about 70%, at least about 80%, at least about 90%, at least about 95%, at least about 96%, at least about 97%, at least about 98%, or at least about 99%, or 100% similarity or identity to the nucleotide sequence set forth in SEQ ID NO:1 (FIG. 1), SEQ ID NO:3 (FIG. 3), SEQ ID NO:5 (FIG. 5A), SEQ ID NO:26 (FIG. 19C), SEQ ID NO:28 (FIG. 20), SEQ ID NO:30 (FIG. 22), SEQ ID NO:32 (FIG. 24), SEQ ID NO:34 (FIG. 26), SEQ ID NO:36 (FIG. 28) or SEQ ID NO:38 (FIG. 30). Percent identity may be determined, for example, by comparing sequence information using the advanced BLAST computer program, version 2.0.8 or later version, available from the National Institutes of Health.

Additionally, the GPR64 protein may be encoded by nucleotide sequences having substantial similarity to the nucleotide sequence set forth in SEQ ID NO:1 (FIG. 1) SEQ ID NO:3 (FIG. 3), SEQ ID NO:5 (FIG. 5A) SEQ ID NO:26 (FIG. 19C), SEQ ID NO:28 (FIG. 20), SEQ ID NO:30 (FIG. 22), SEQ ID NO:32 (FIG. 24), SEQ ID NO:34 (FIG. 26), SEQ ID NO:36 (FIG. 28) or SEQ ID NO:38 (FIG. 30). “Substantial similarity,” as used herein, means that the nucleotide sequence is sufficiently similar to a reference nucleotide sequence that it will hybridize therewith under moderately stringent conditions. This method of determining similarity is well known in the art to which the invention pertains. Examples of stringency conditions are shown in Table 1 below: highly stringent conditions are those that are at least as stringent as, for example, conditions A-F; moderately stringent conditions are at least as stringent as, for example, conditions G-L; and reduced stringency conditions are at least as stringent as, for example, conditions M-R.

TABLE 1

| Strin- | Poly- | Hybrid | Hybridization | Wash |
|---|---|---|---|---|
| gency | nucleotide | Length | Temperature and | Temperature |
| Condition | Hybrid | (bp)¹ | Buffer² | and Buffer² |
| A | DNA:DNA | >50 | 65° C.; 1X SSC -or- | 65° C.; 0.3X |
| | | | 42° C.; 1X SSC, | SSC |
| | | | 50% formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X |
| | | | 45° C.; 1X SSC, | SSC |
| | | | 50% formamide | |
| D | DNA:RNA | <50 | T_{D}*; 1X SSC | T_{D}*; 1X SSC |
| E | RNA:RNA | >50 | 70° C.; 1X SSC -or- | 70° C.; |
| | | | 50° C.; 1X SSC, | 0.3xSSC |
| | | | 50% formamide | |
| F | RNA:RNA | <50 | T_{F}*; 1X SSC | T_{f}*; 1X SSC |
| G | DNA:DNA | >50 | 65° C.; 4X SSC -or- | 65° C.; 1X |
| | | | 42° C.; 4X SSC, | SSC |
| | | | 50% formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X |
| | | | 45° C.; 4X SSC, | SSC |
| | | | 50% formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X |
| | | | 50° C.; 4X SSC, | SSC |
| | | | 50% formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X |
| | | | 40° C.; 6X SSC, | SSC |
| | | | 50% formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X |
| | | | 42° C.; 6X SSC, | SSC |
| | | | 50% formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X |
| | | | 45° C.; 6X SSC, | SSC |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T bases) + 4(# of G + C bases). For hybrids between 18 and 49 base pairs in length, Tₘ(EC) = 81.5 + 16.6(log₁₀Na⁺) + 0.41(% G + C) − (600/N), where N is the number of bases in the hybrid, and Na⁺ is the concentration of sodium ions in the hybridization buffer (Na+ for 1xSSC = 0.165 M).

Additional examples of stringency conditions for polynucleotide hybridization are provided in Sambrook et al., “Molecular Cloning: A Laboratory Manual”, Chs. 9 & 11, Cold Spring Harbor Laboratory Press, Cold Spring Harbor, N.Y. (1989), and Ausubel et al., eds., Current Protocols in Molecular Biology, §§ 2.10, 6.3-6.4, John Wiley & Sons, Inc. (1995), herein incorporated by reference.

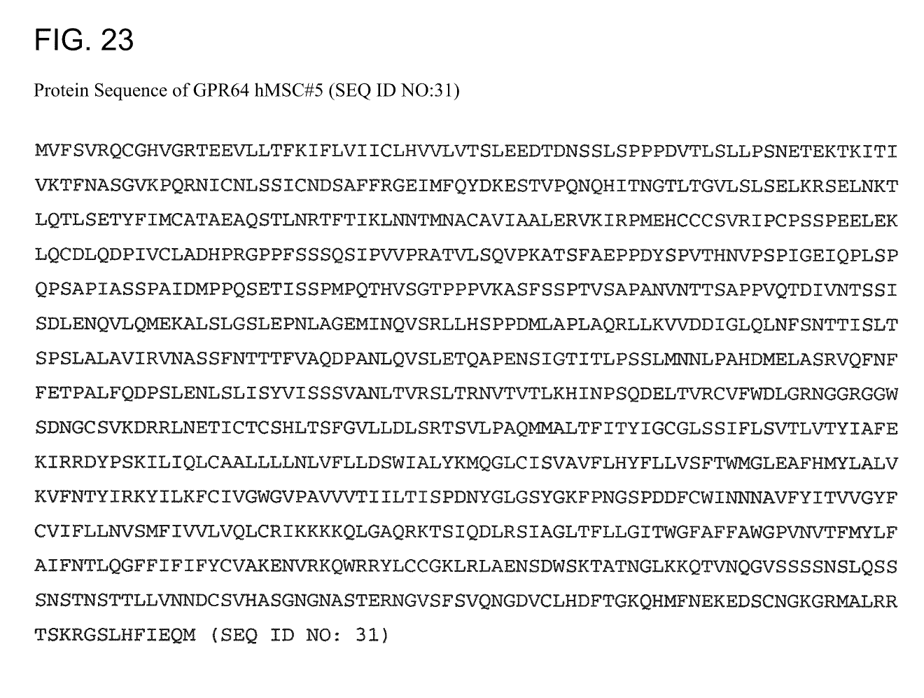

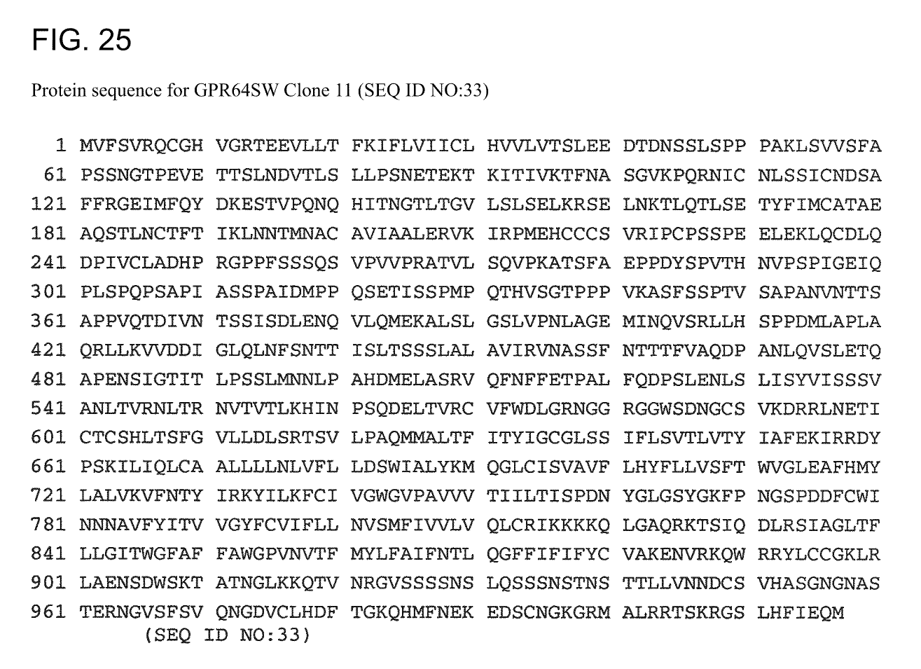

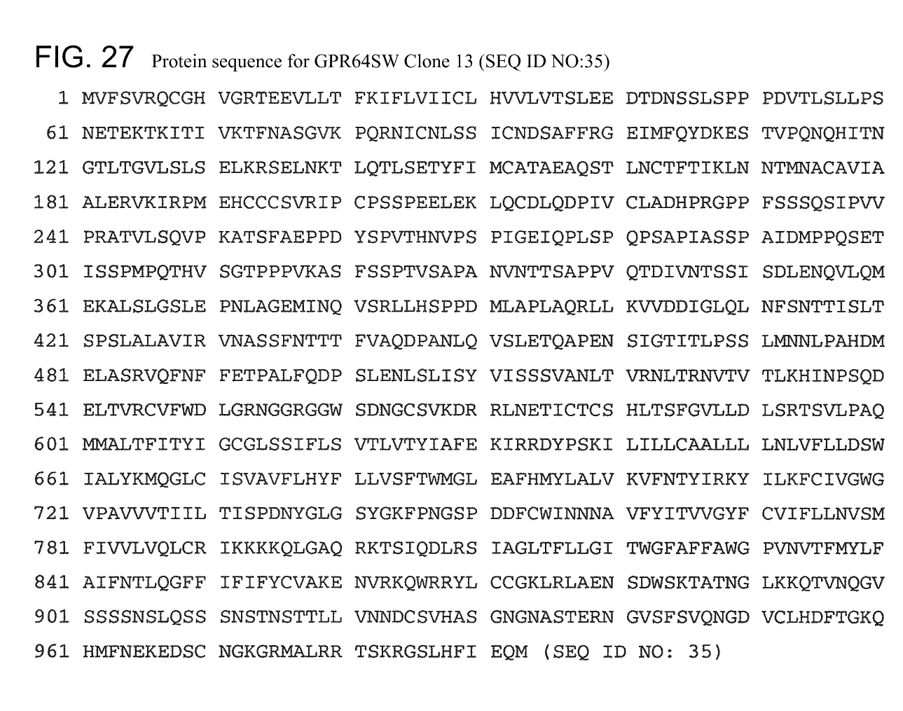

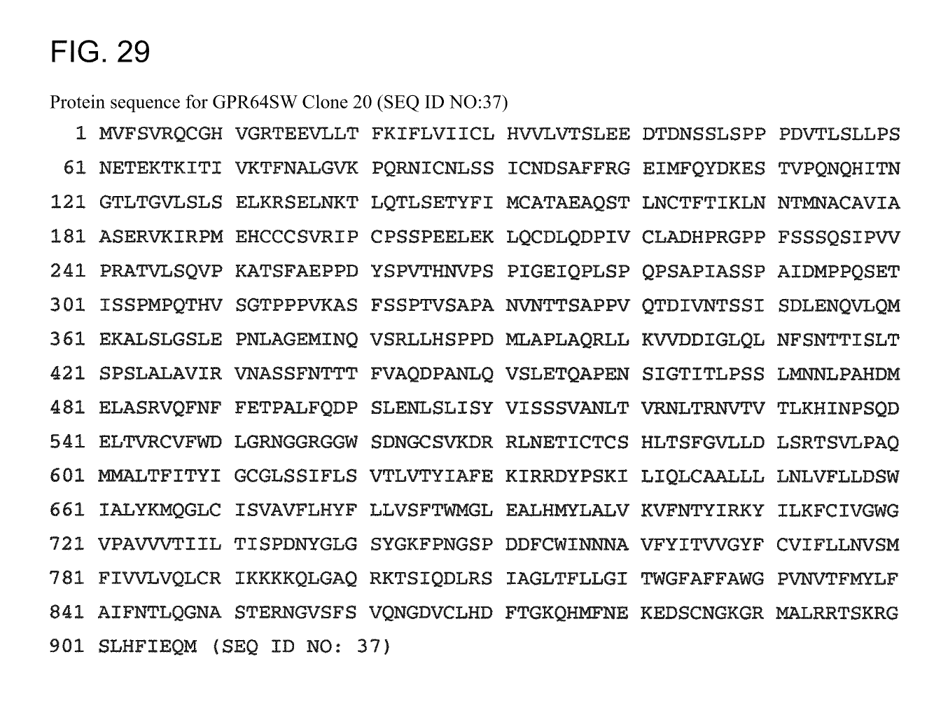

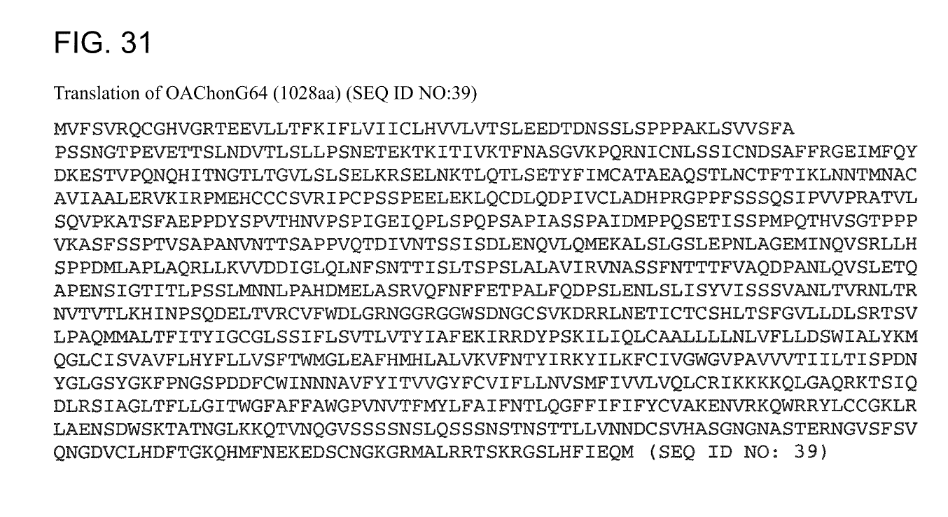

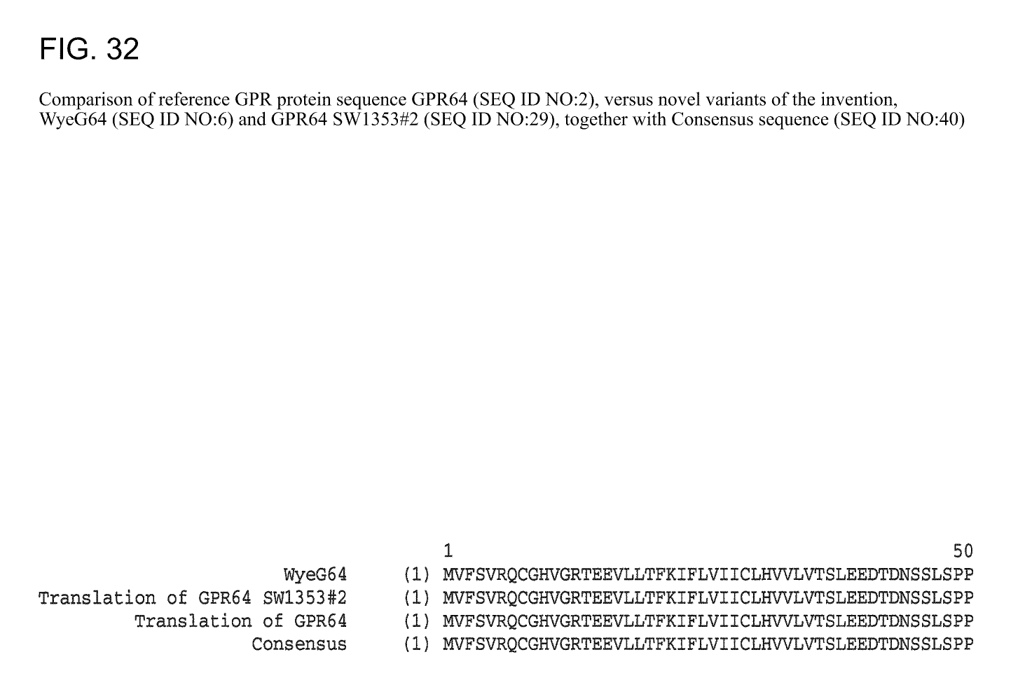

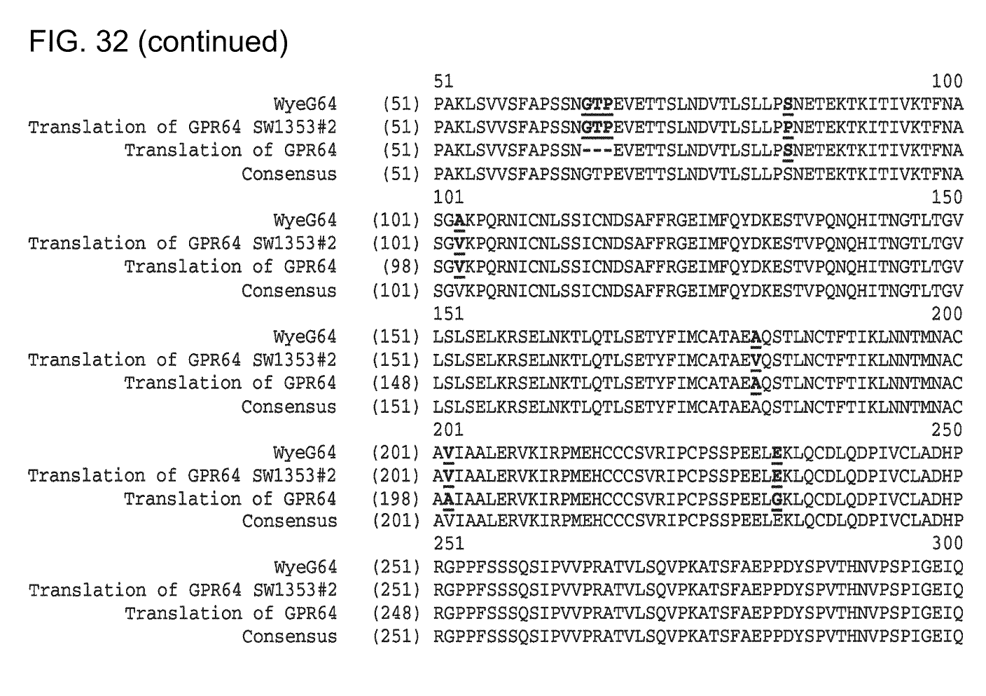

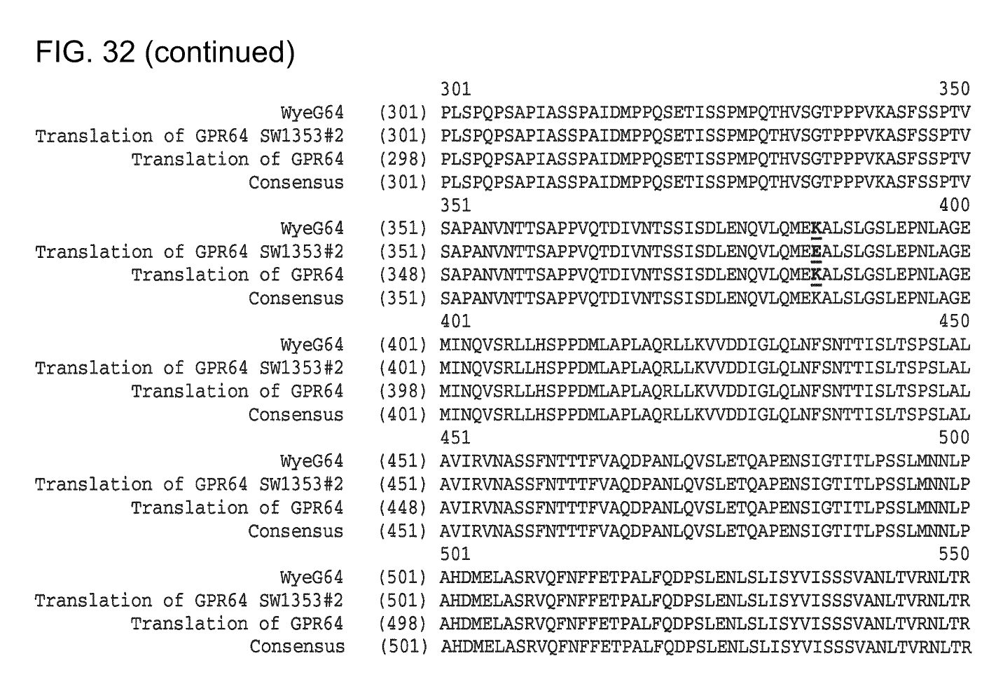

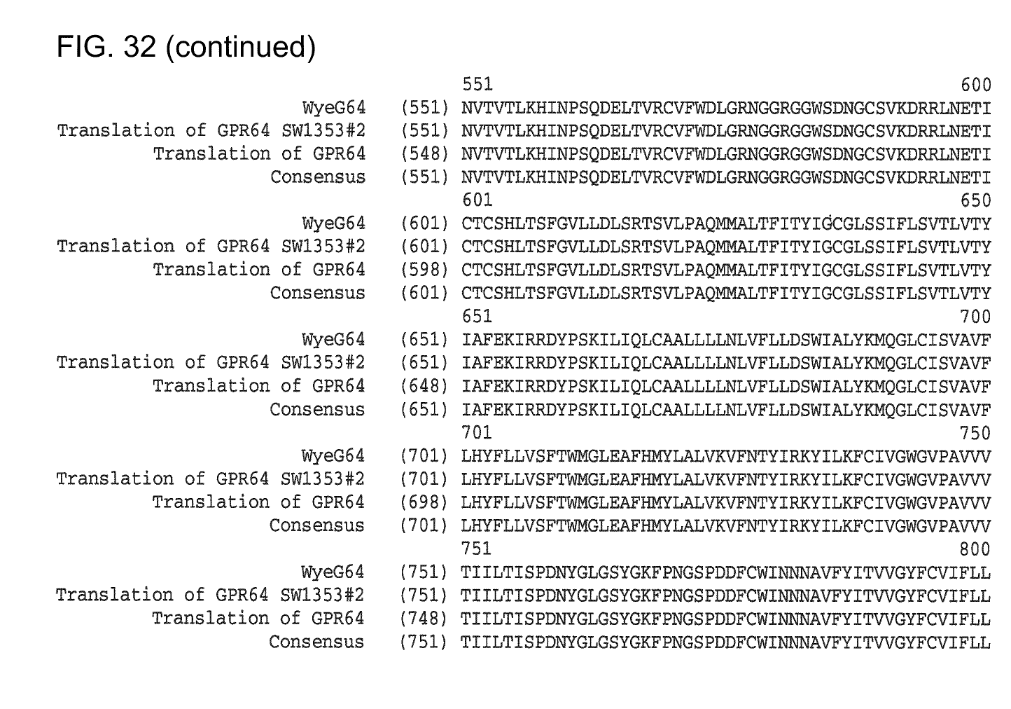

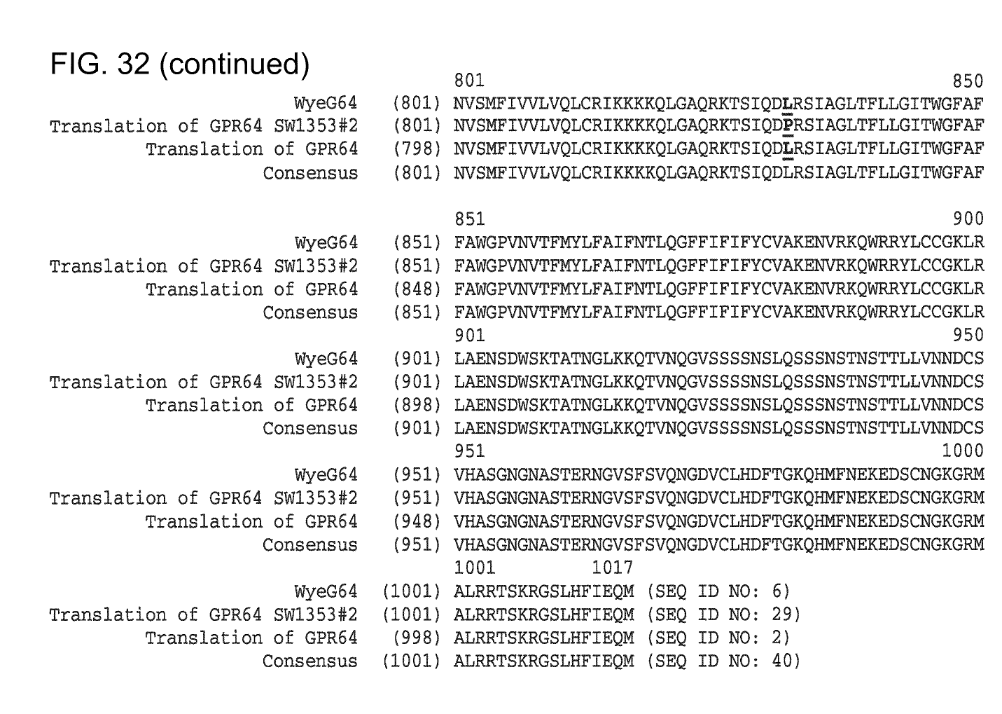

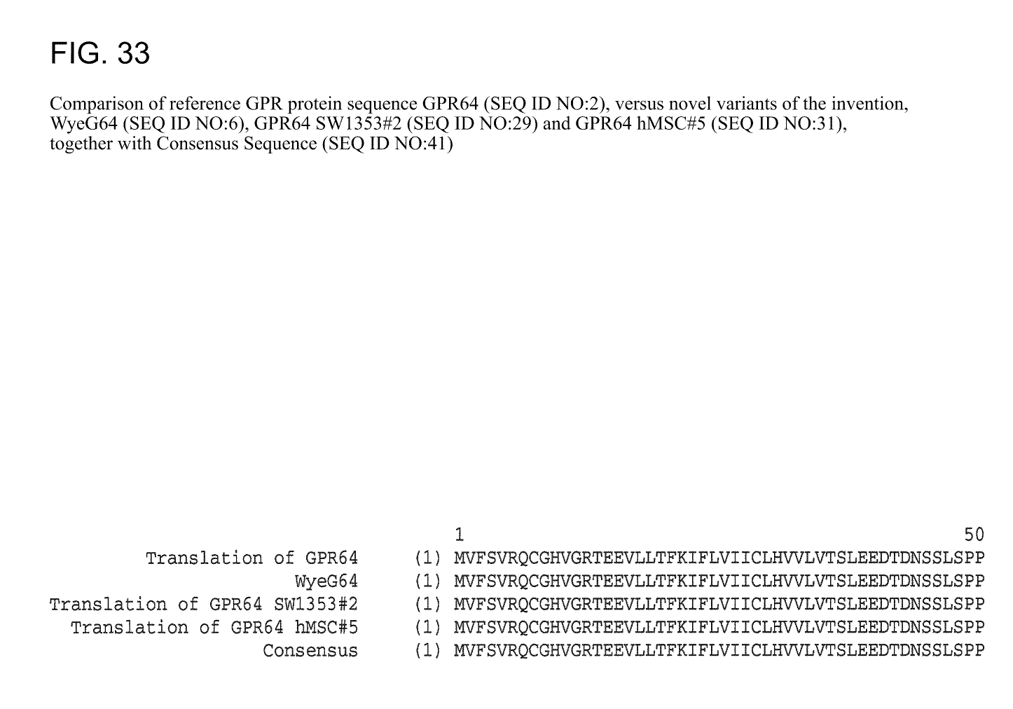

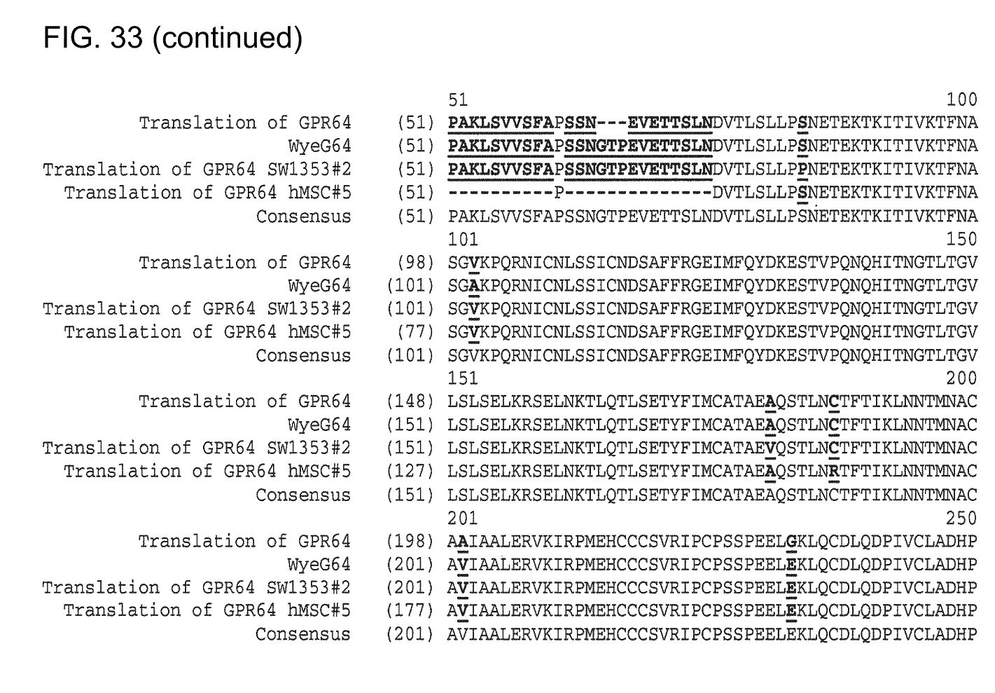

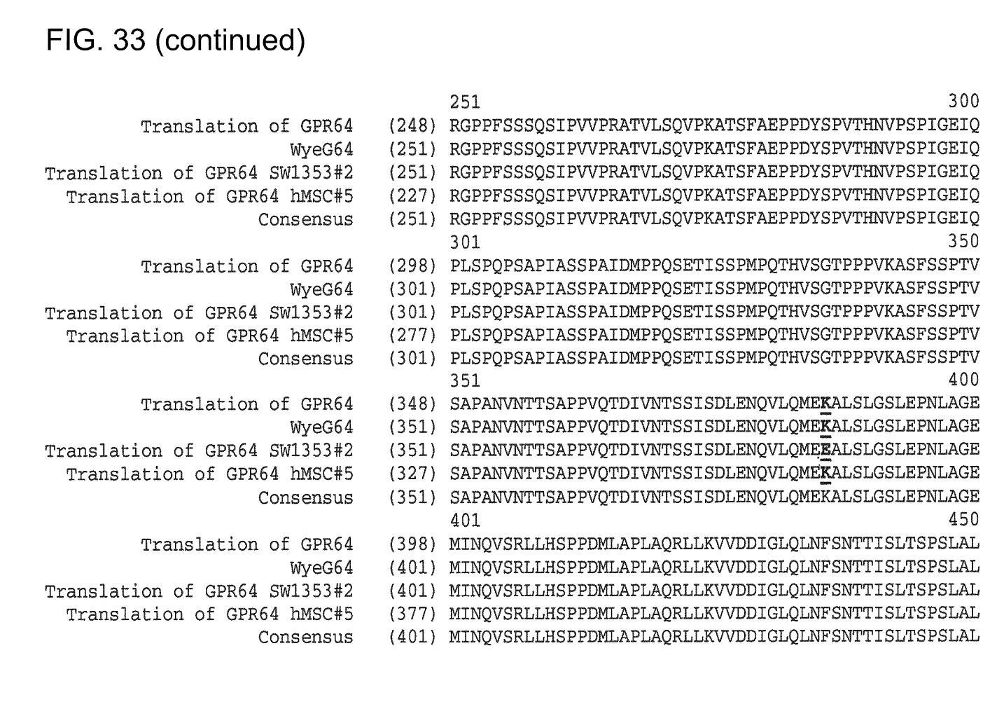

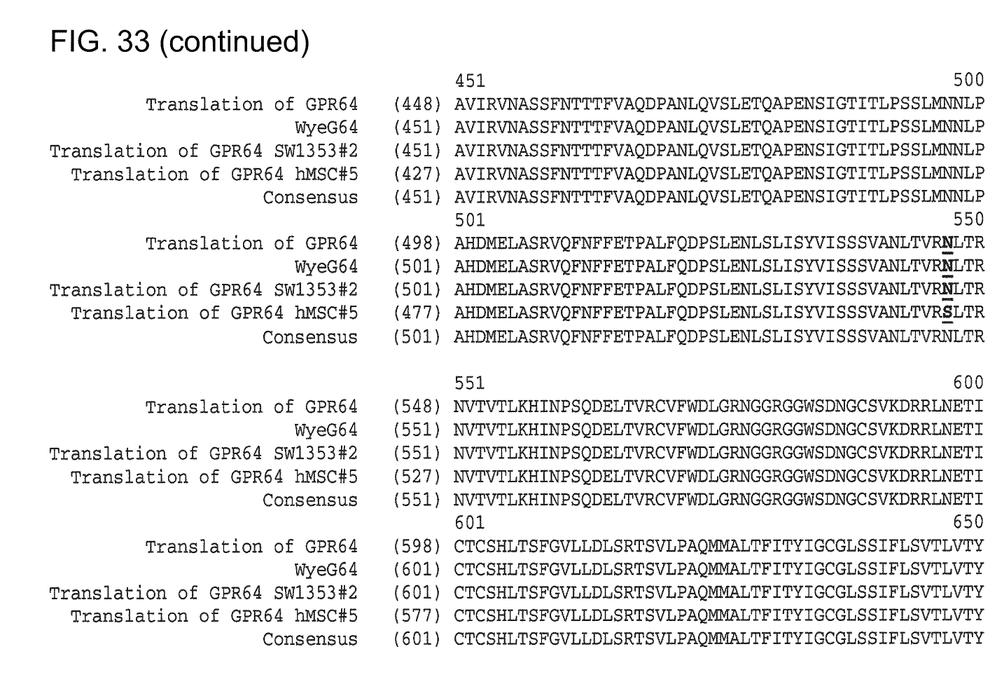

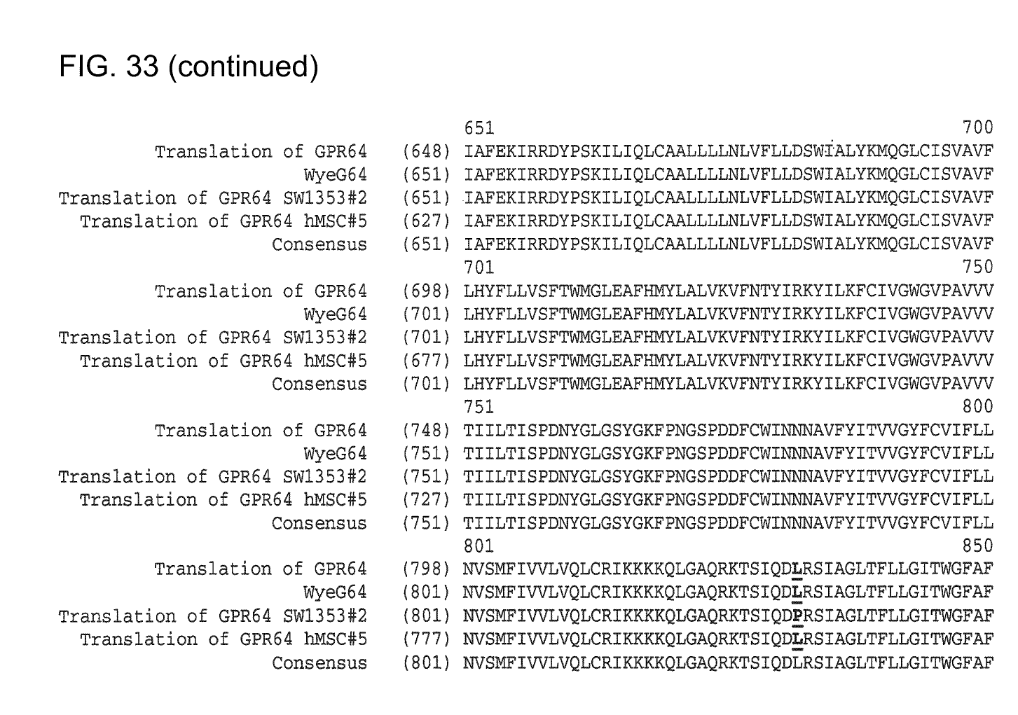

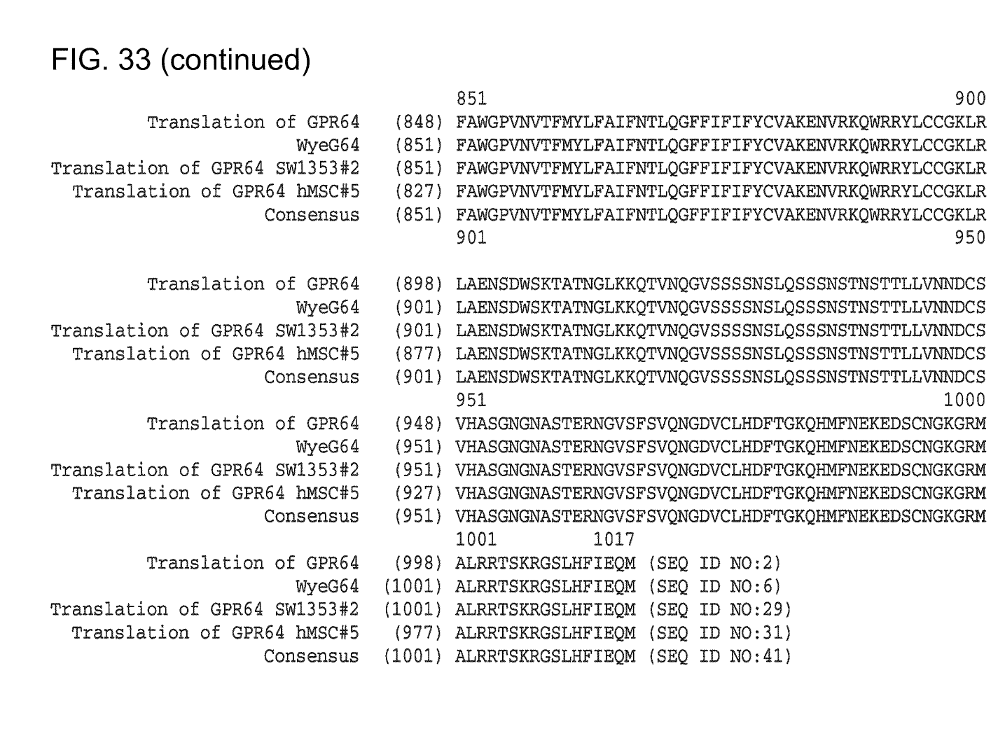

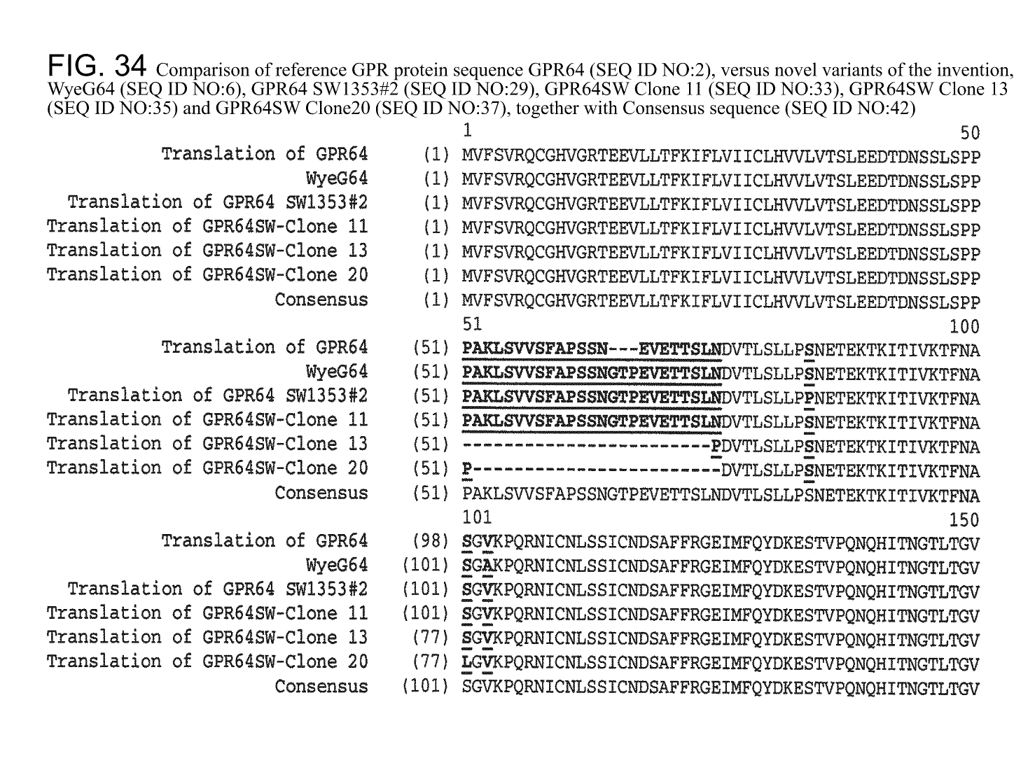

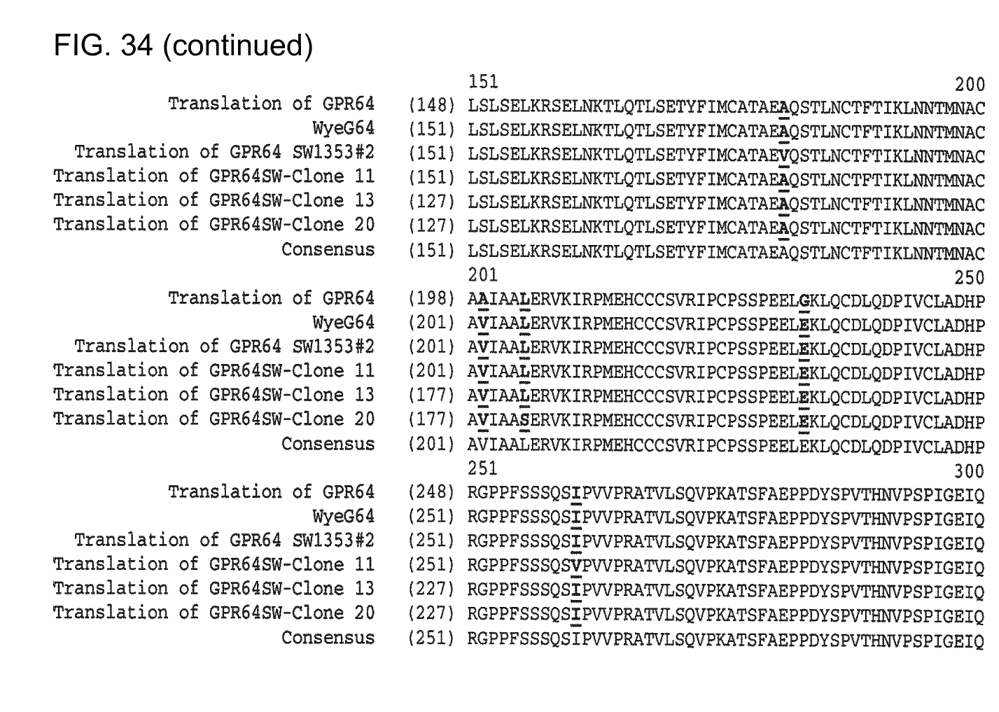

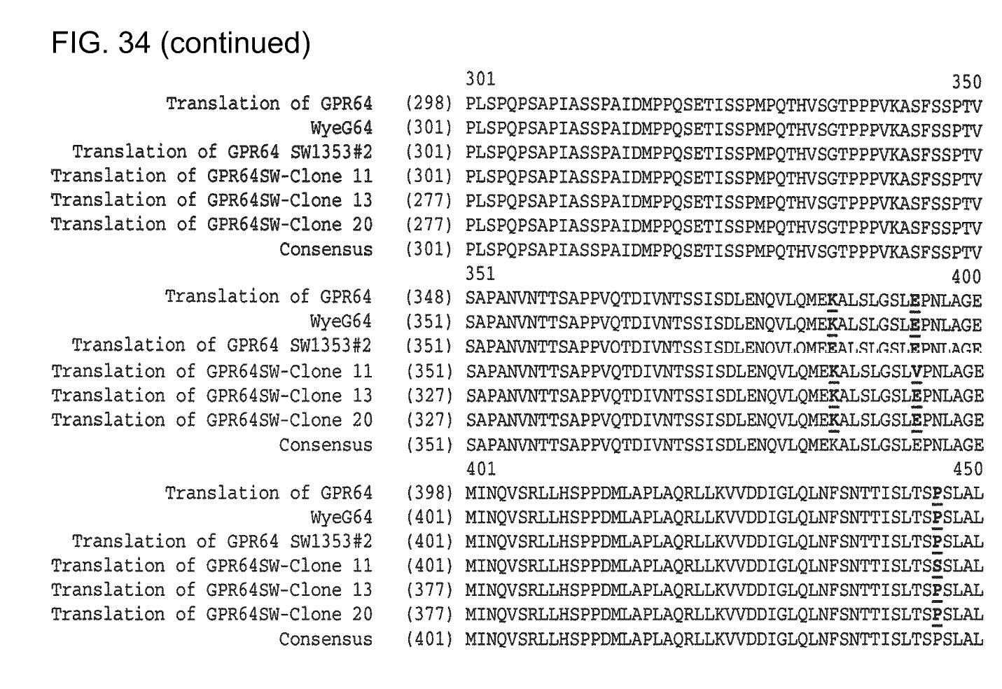

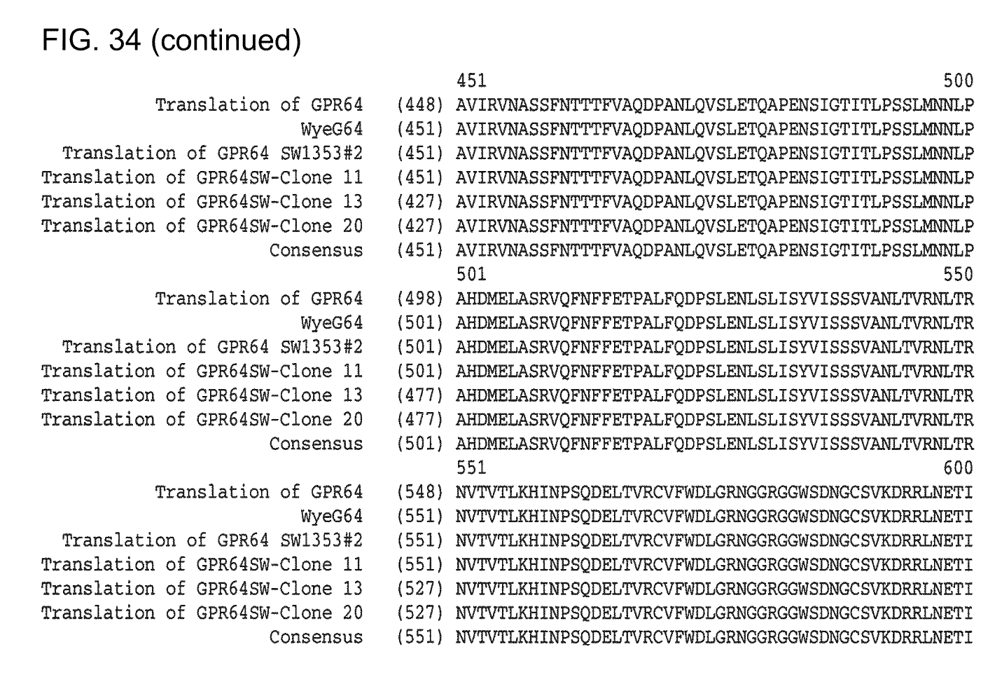

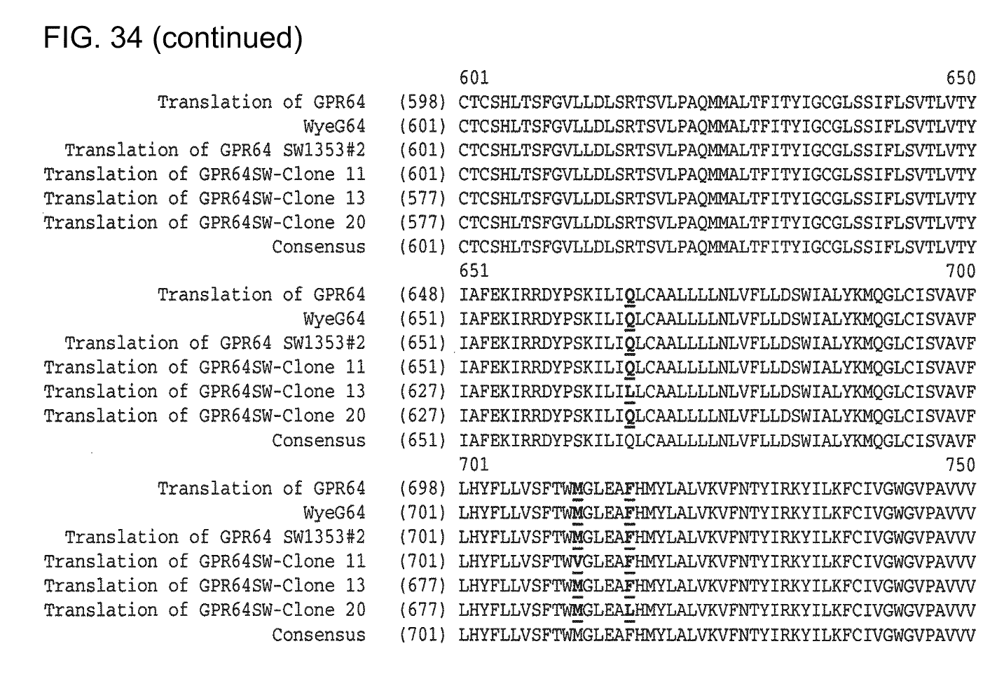

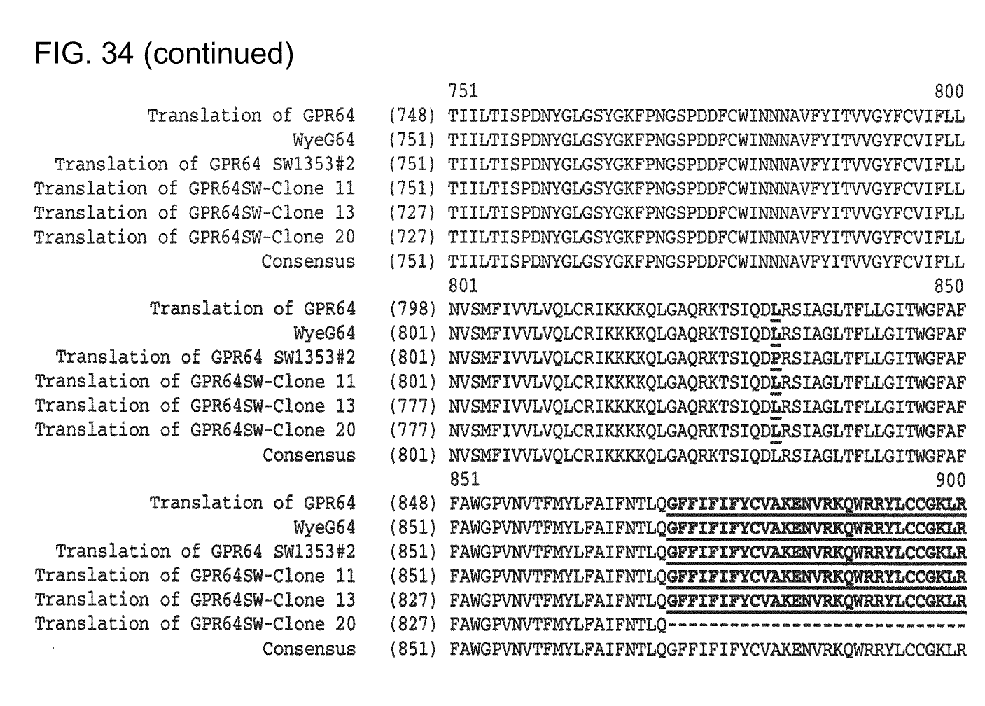

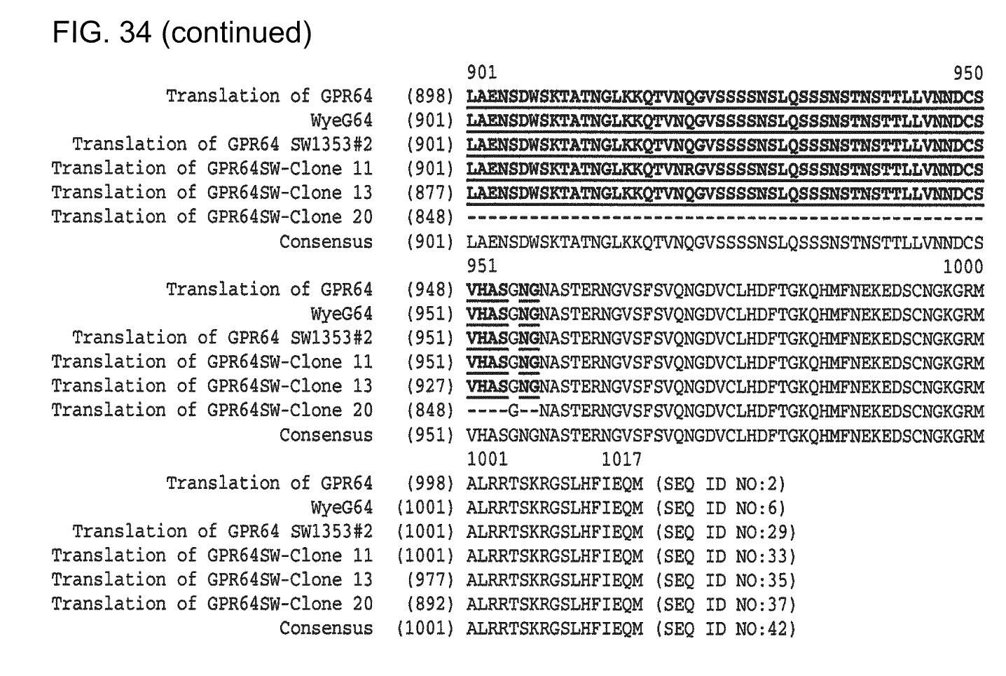

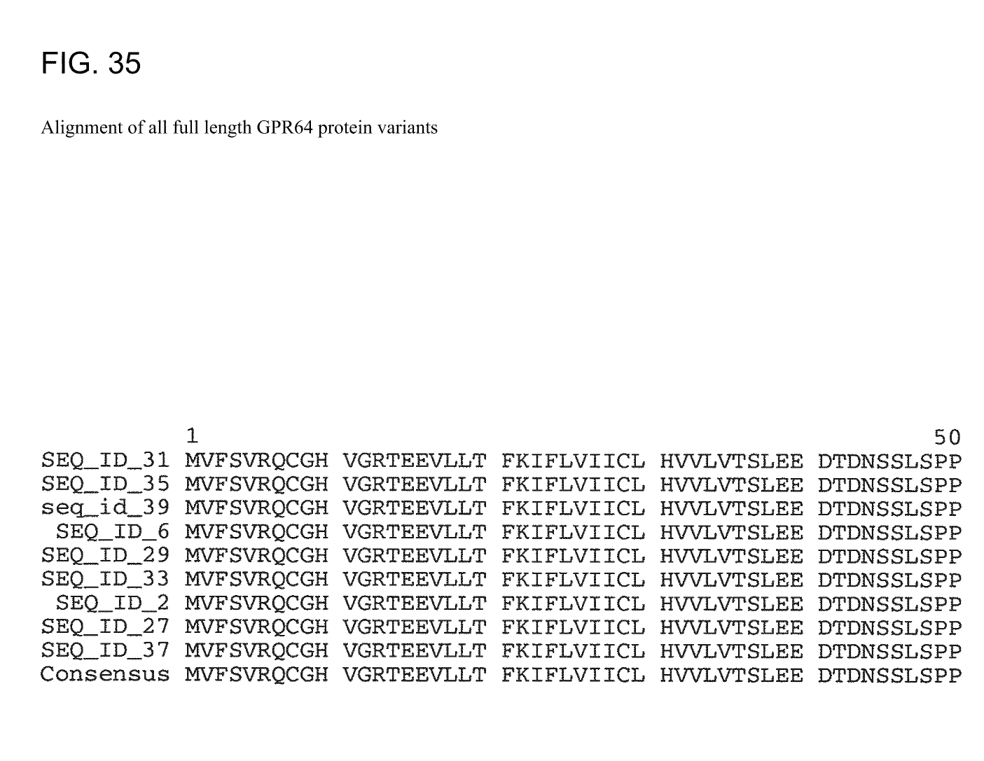

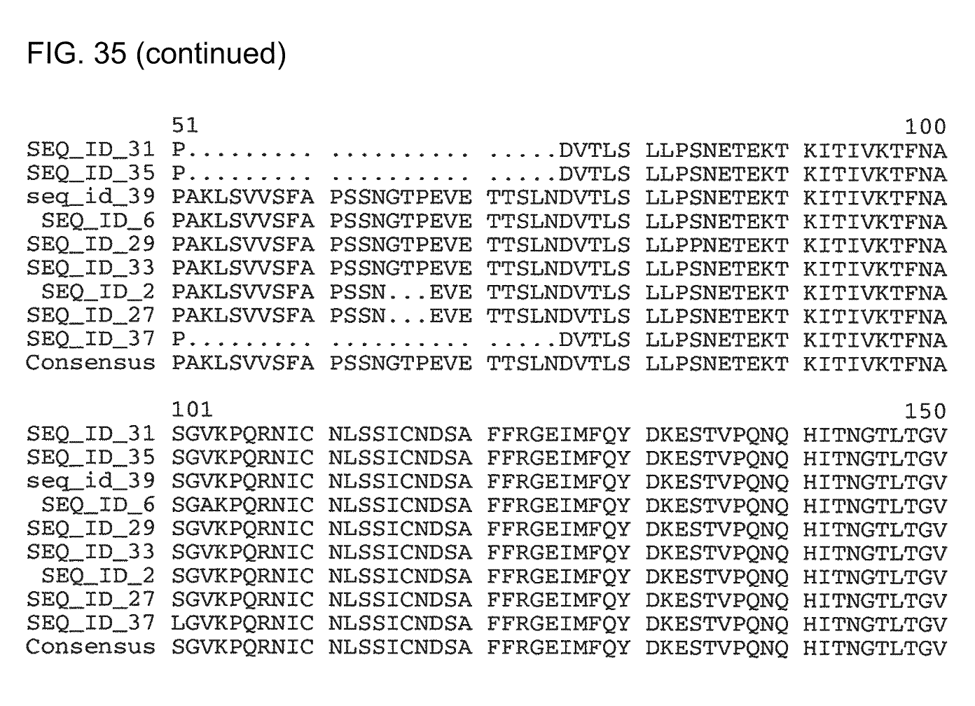

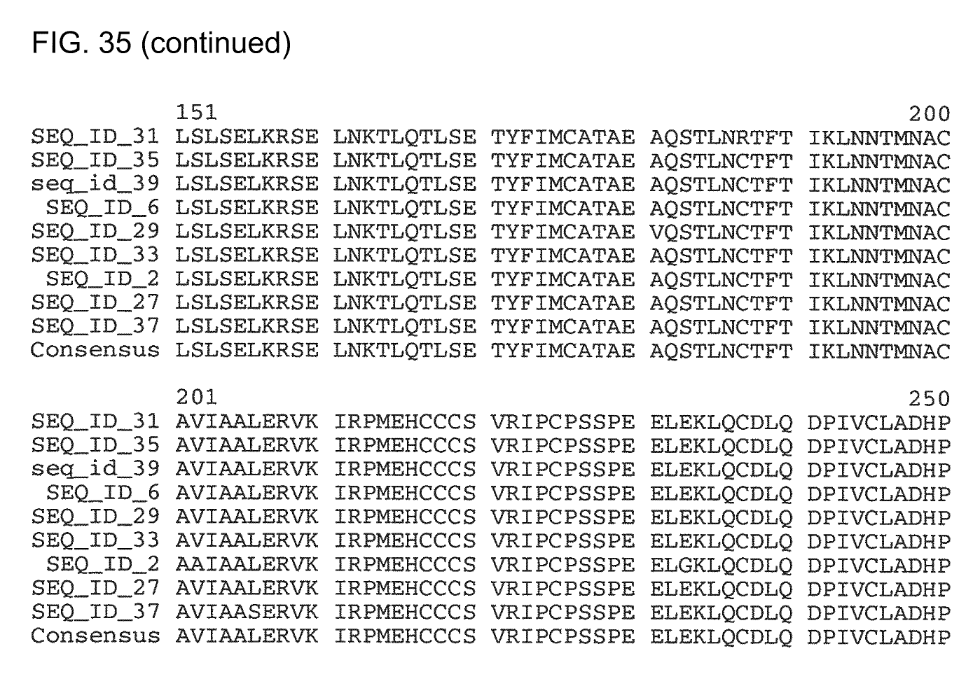

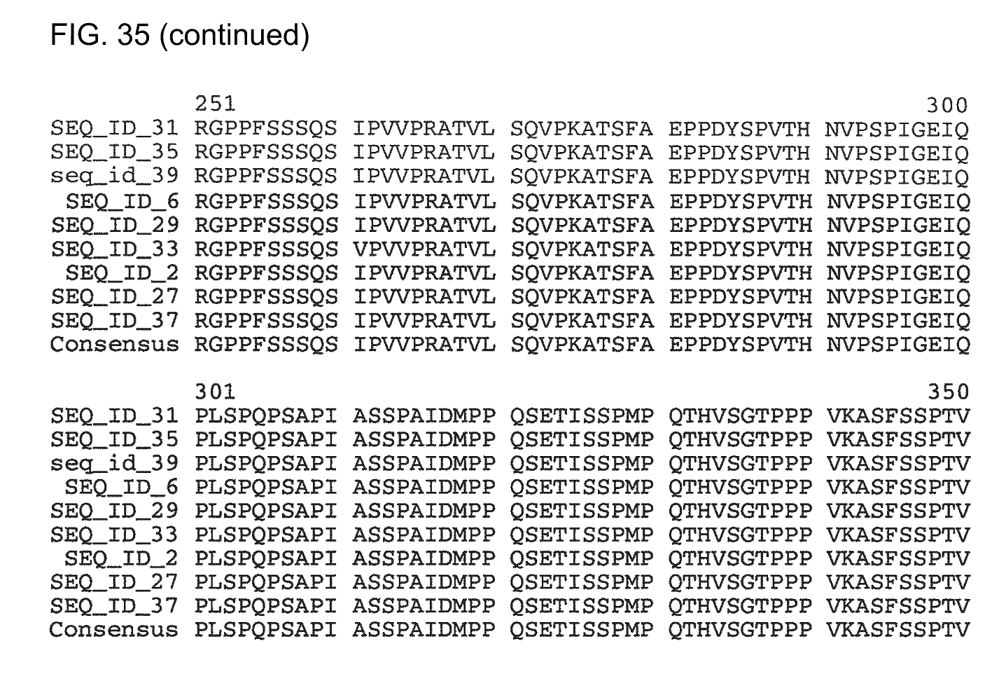

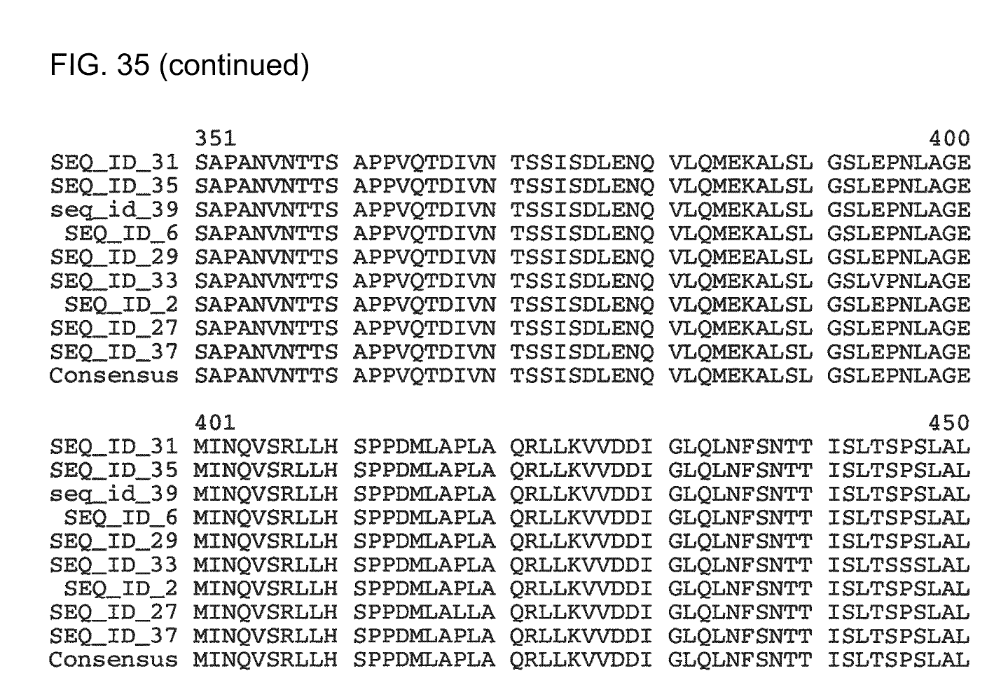

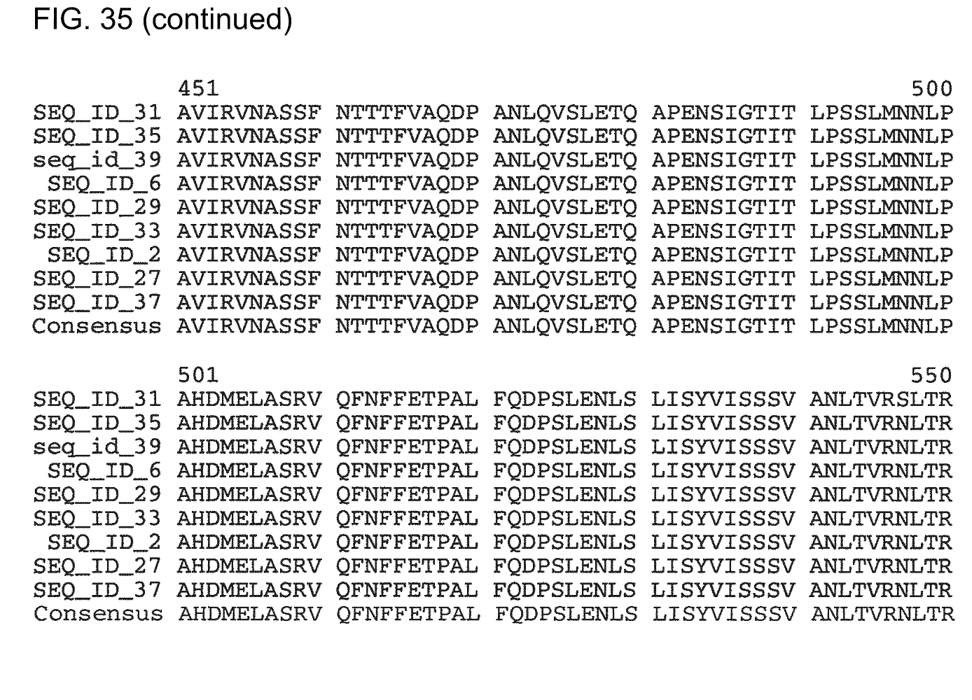

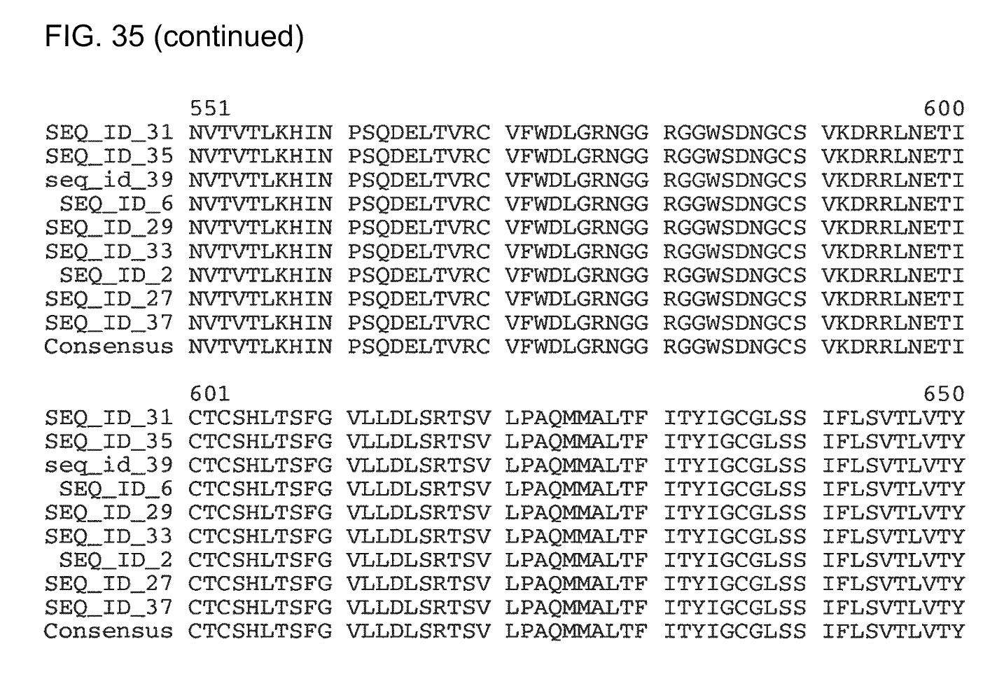

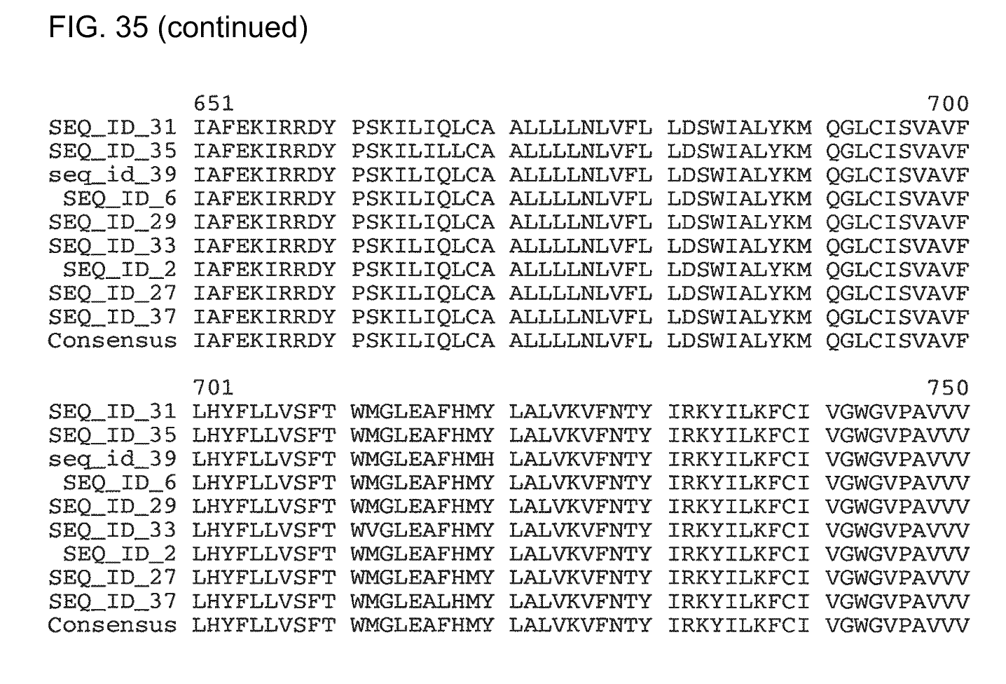

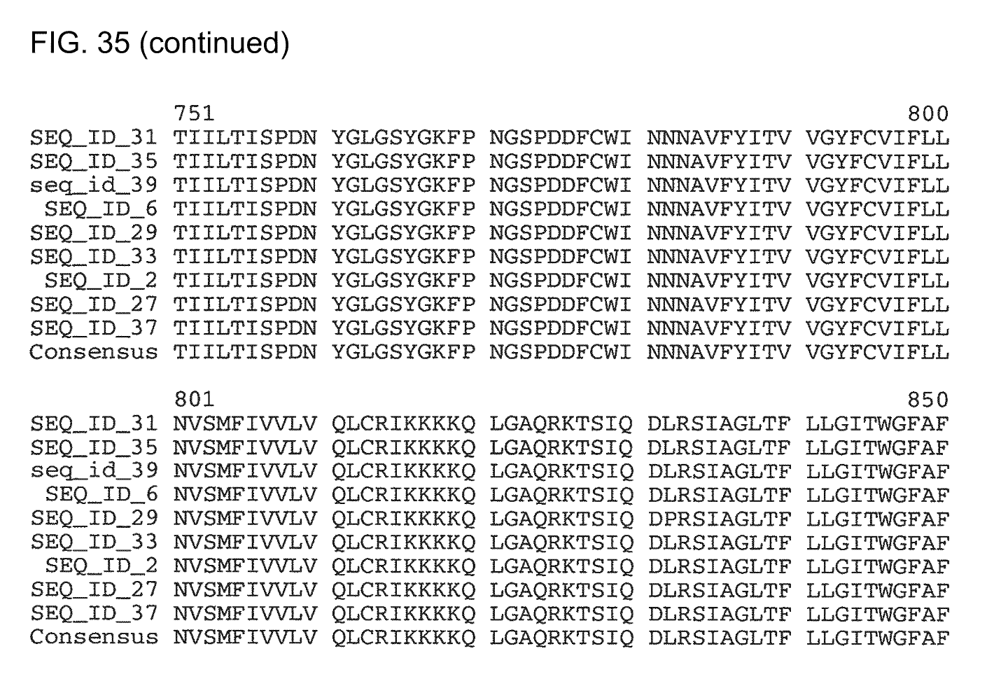

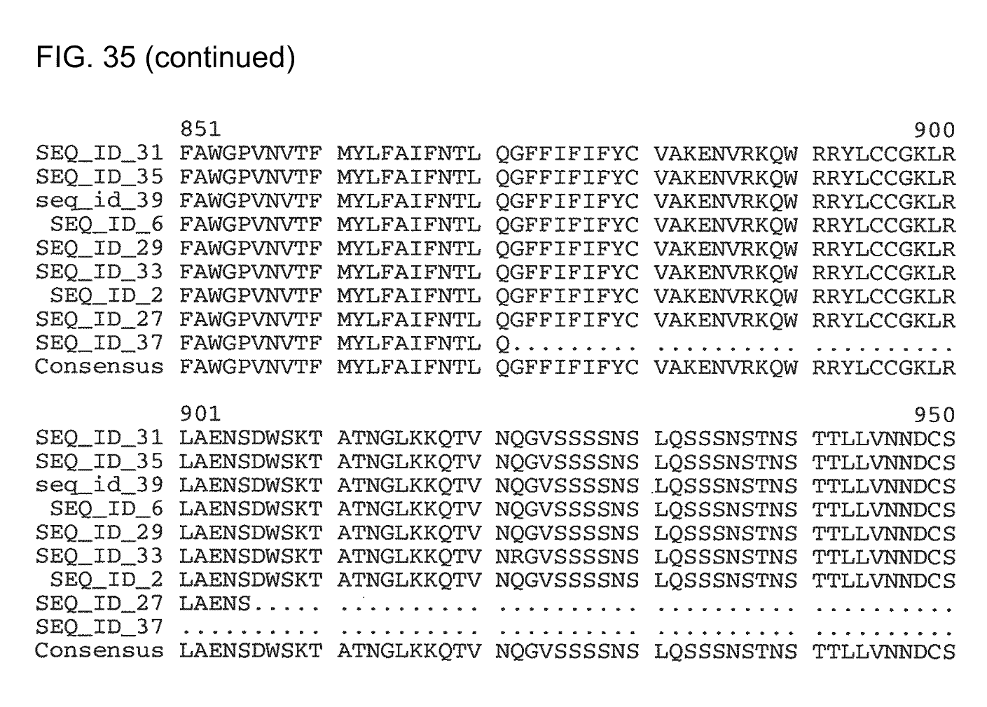

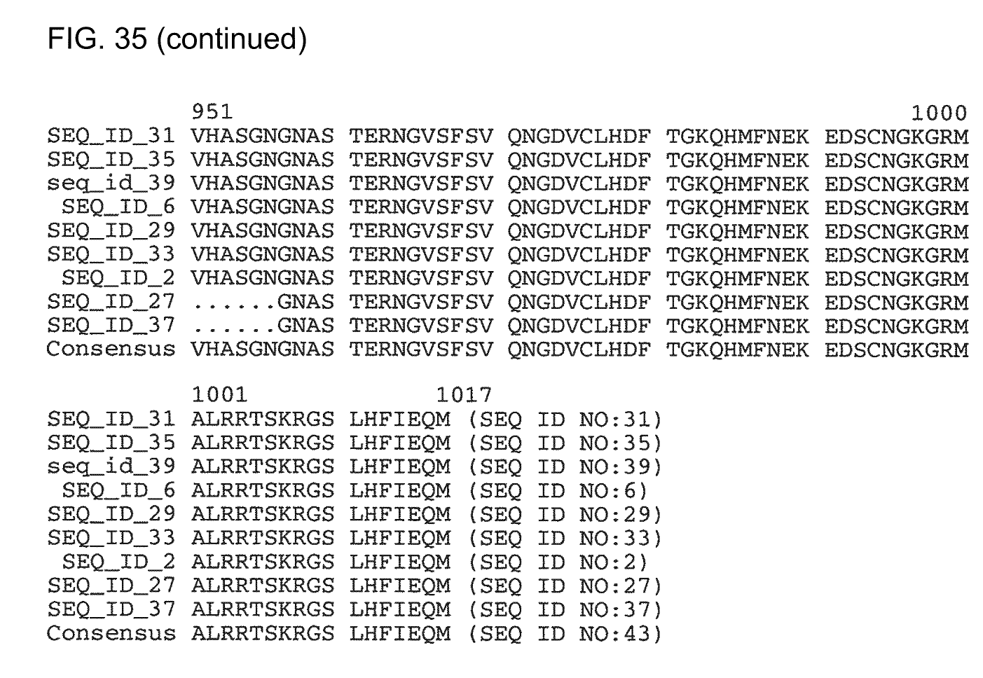

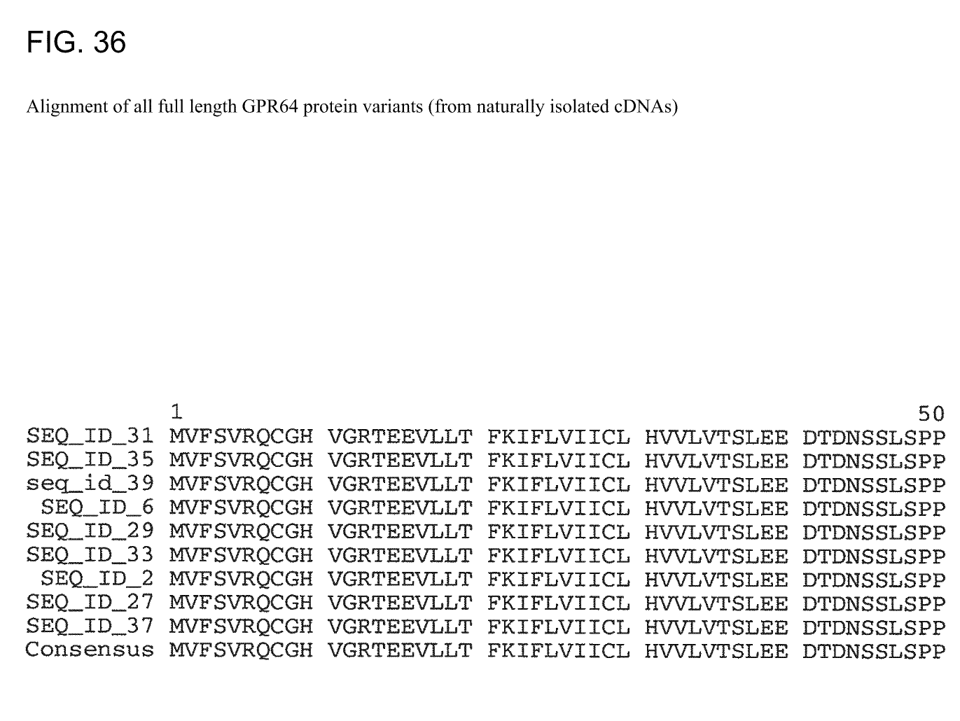

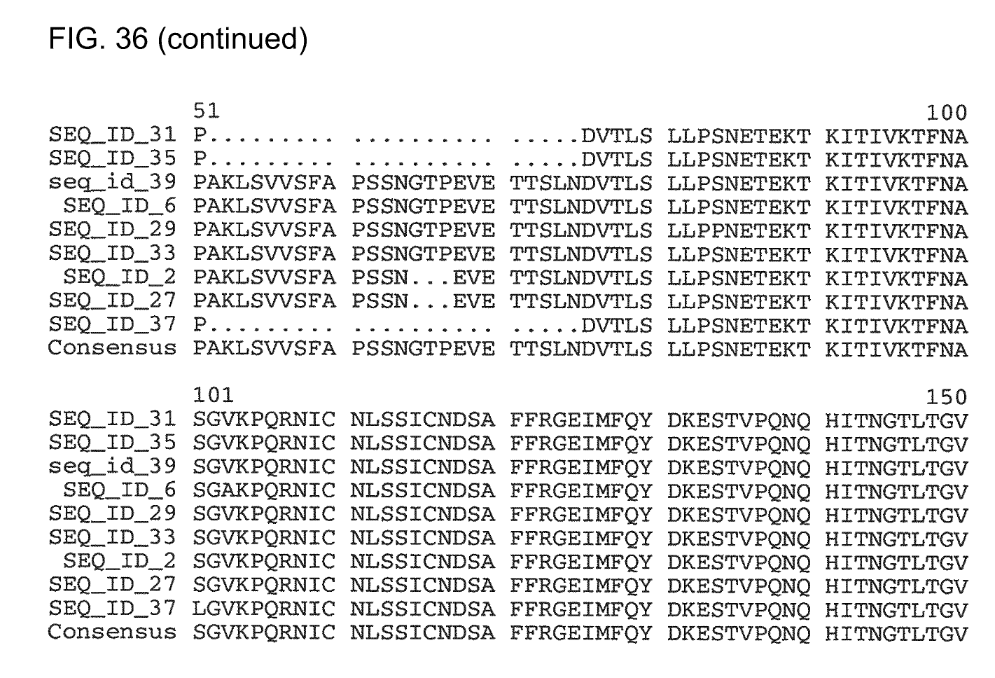

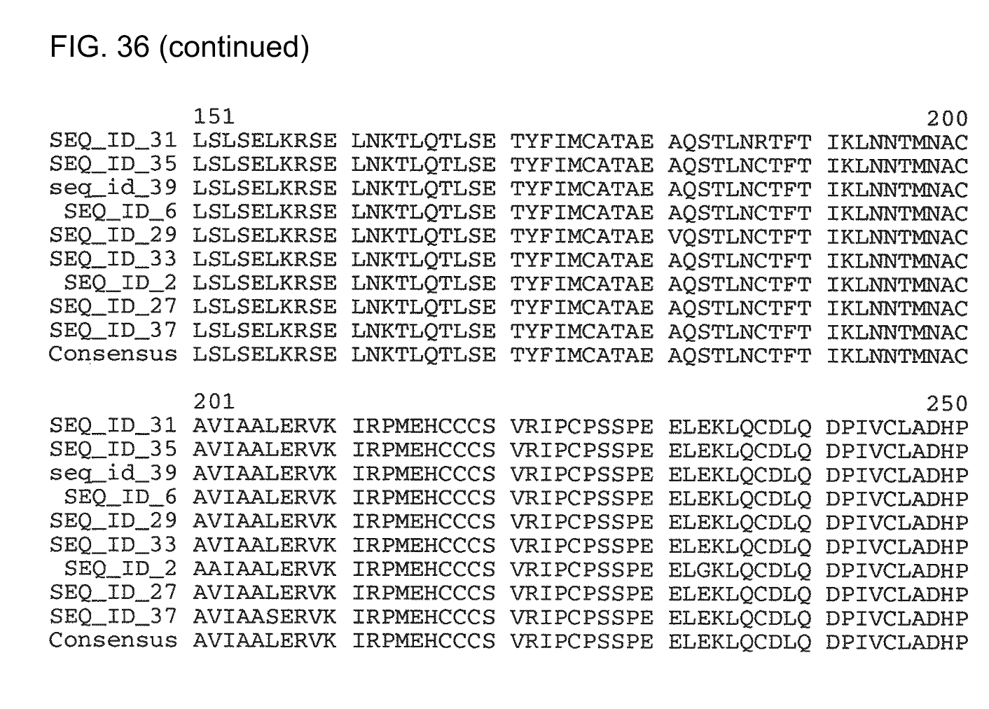

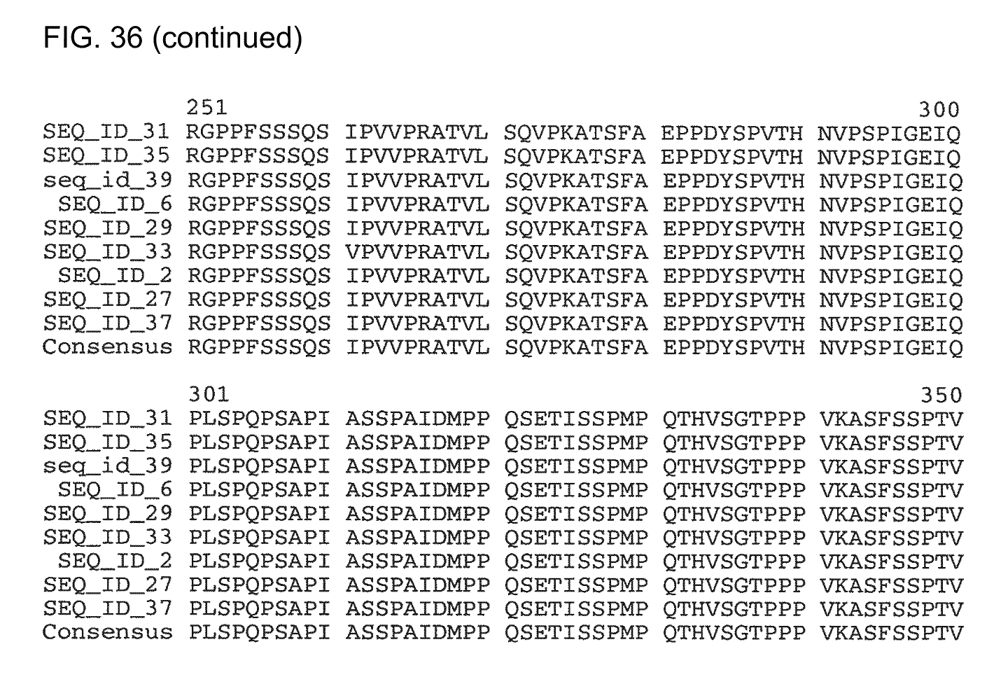

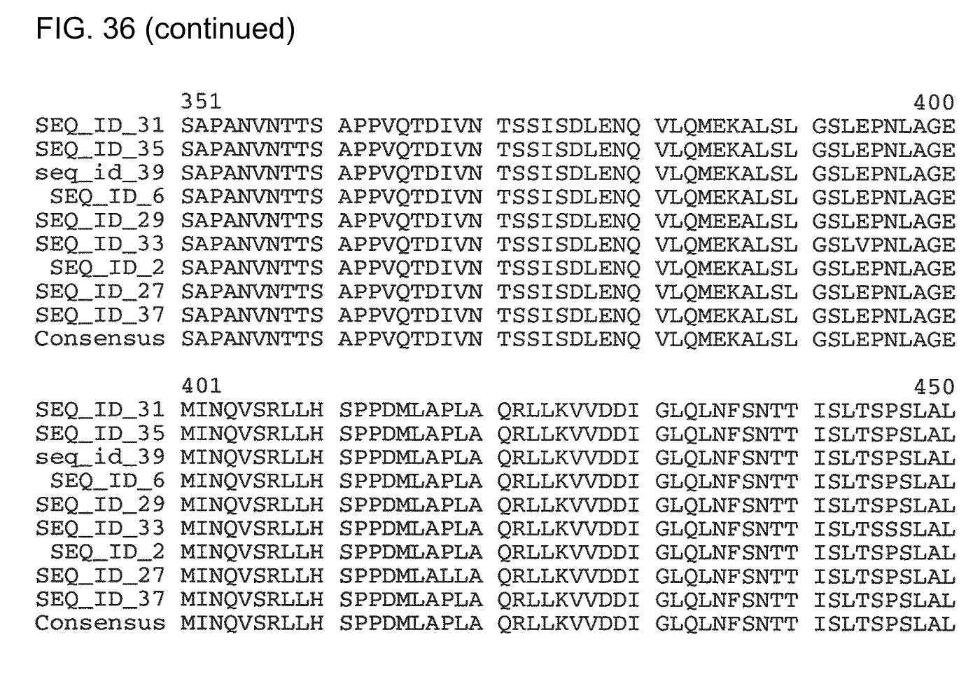

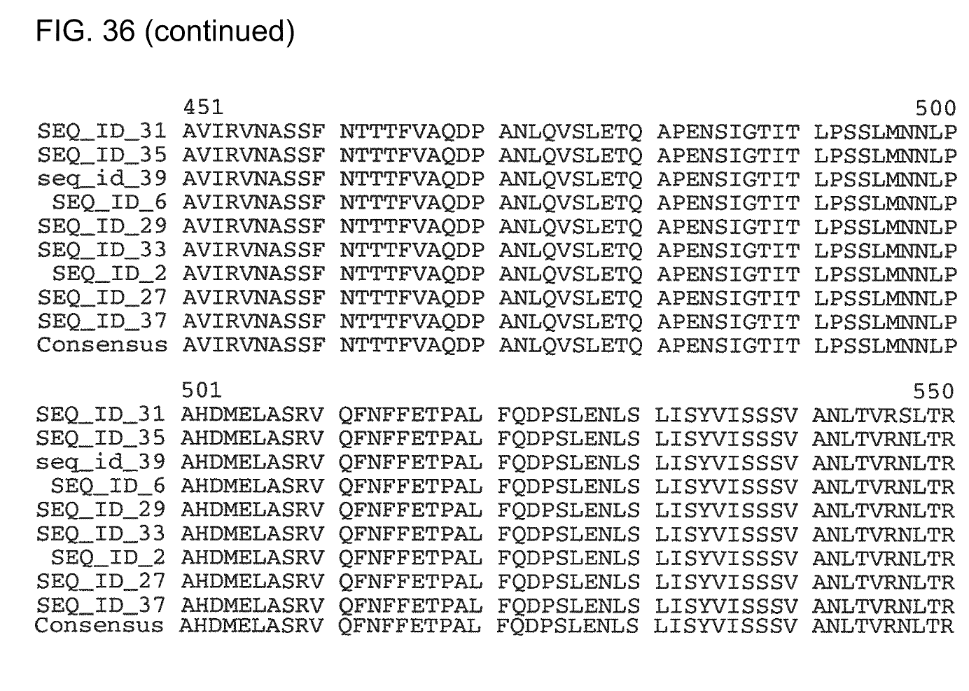

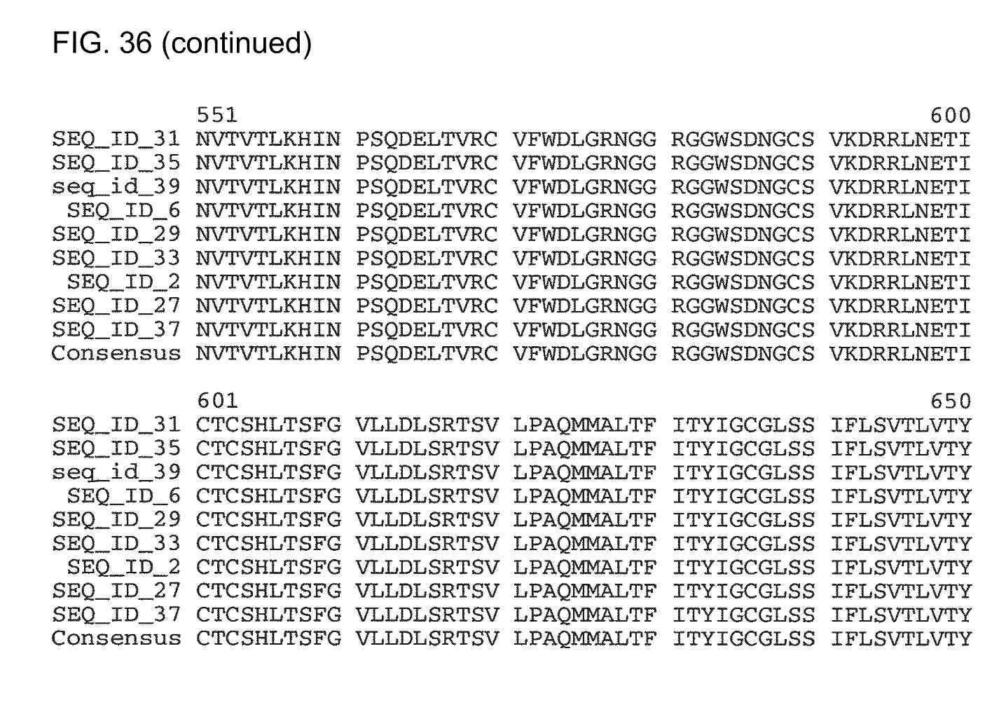

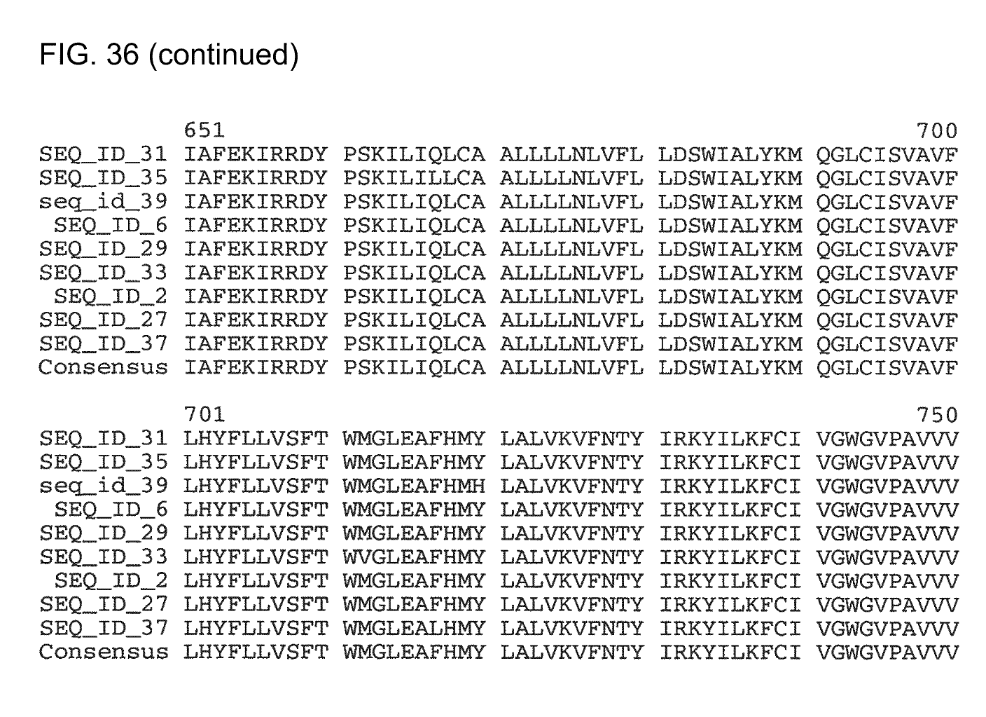

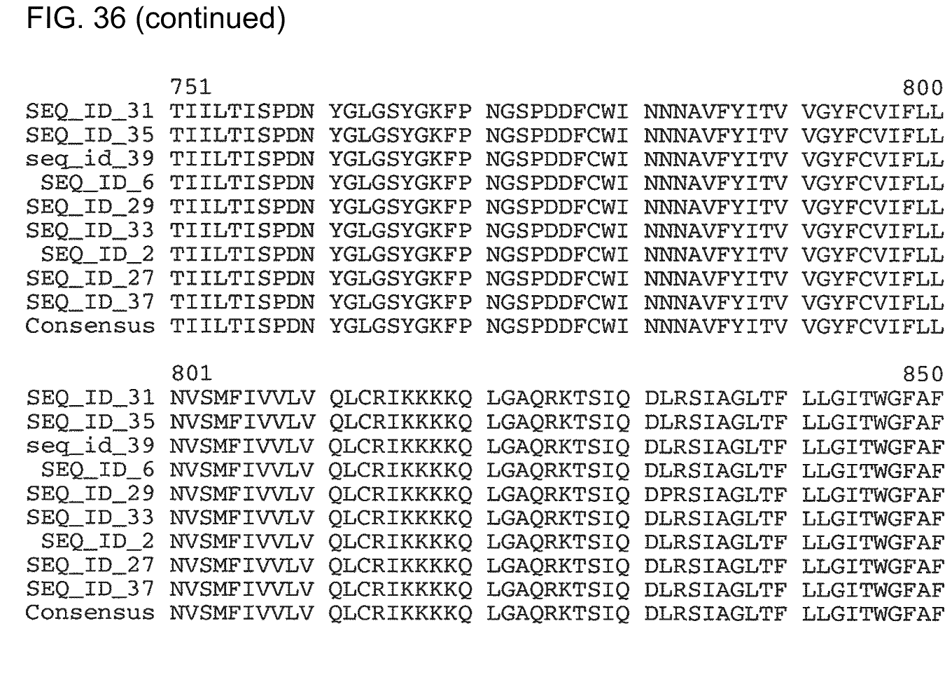

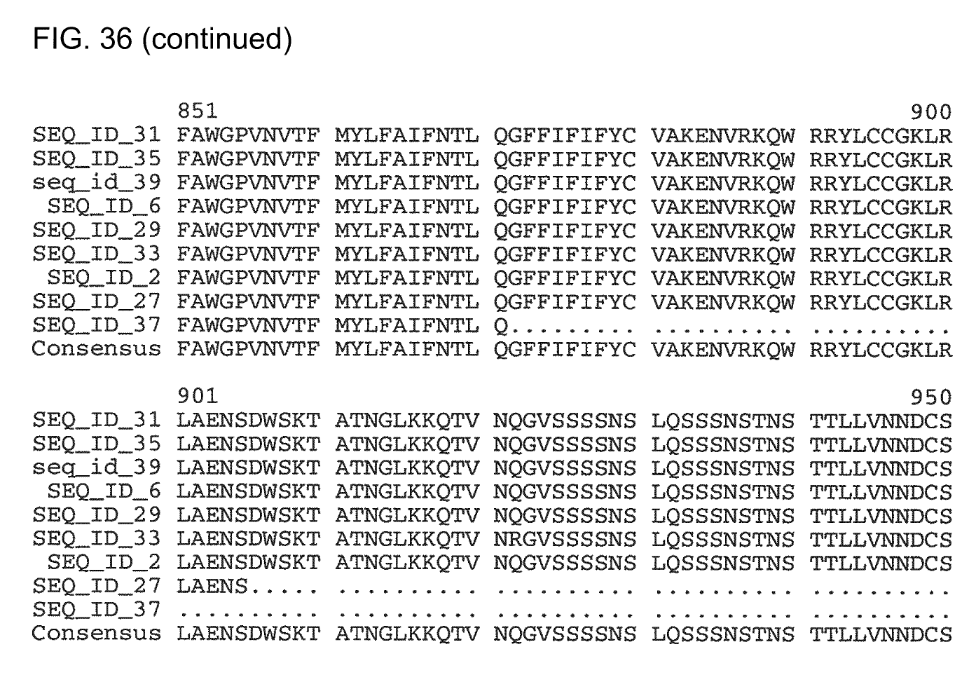

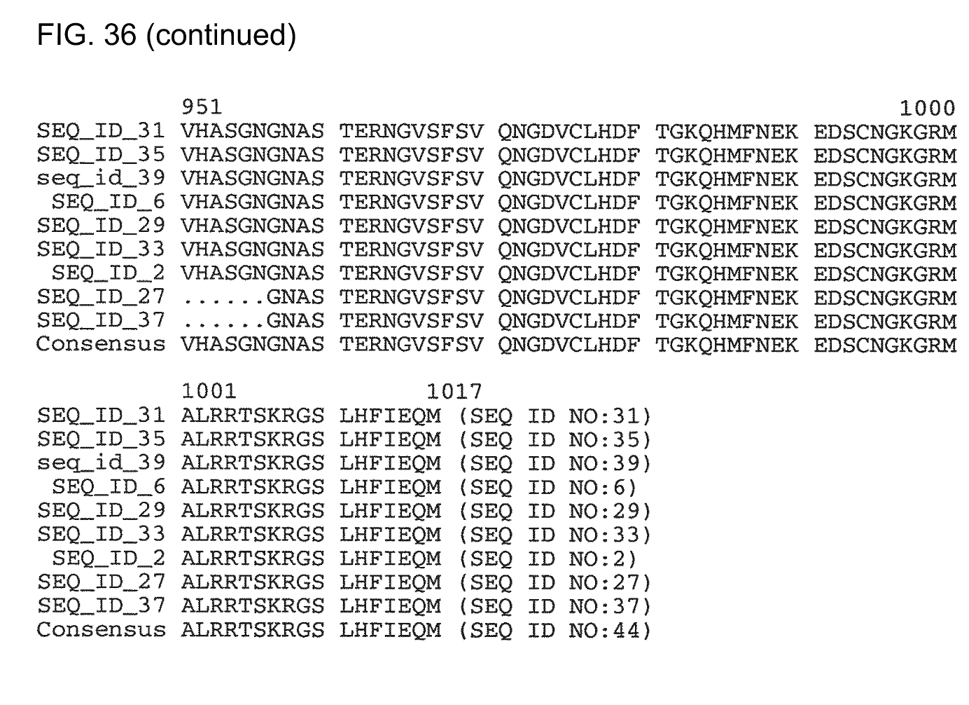

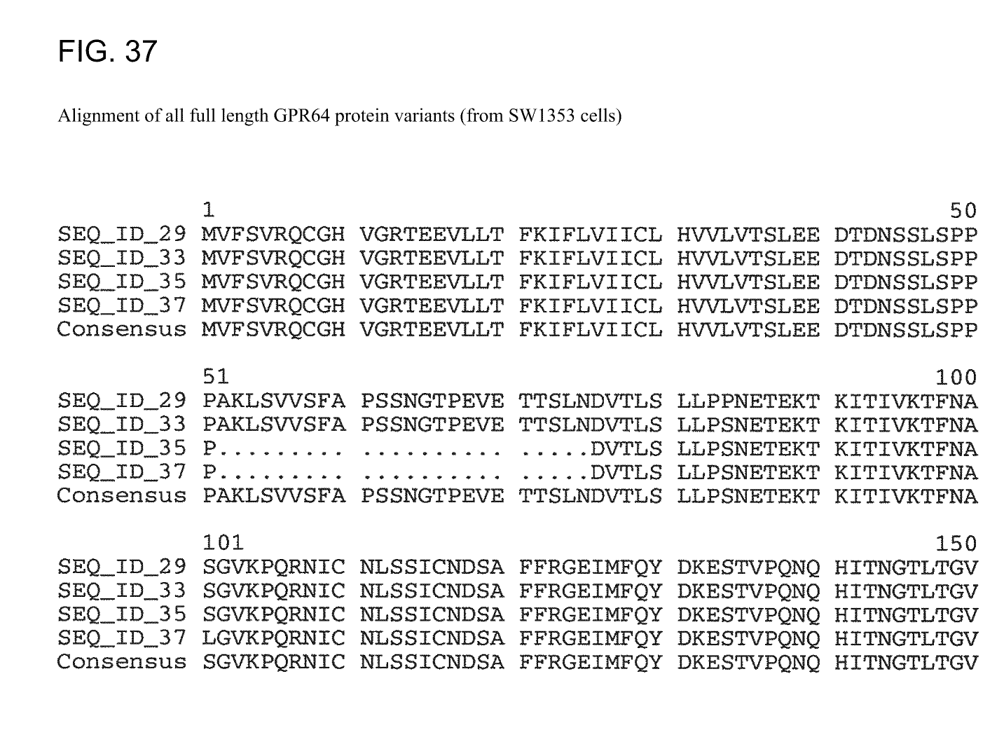

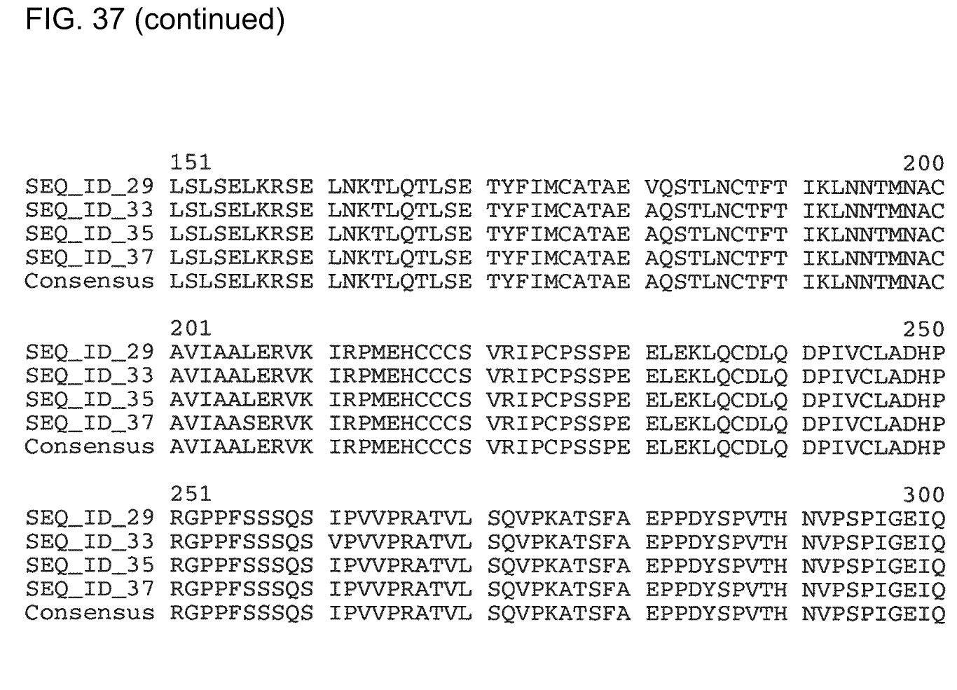

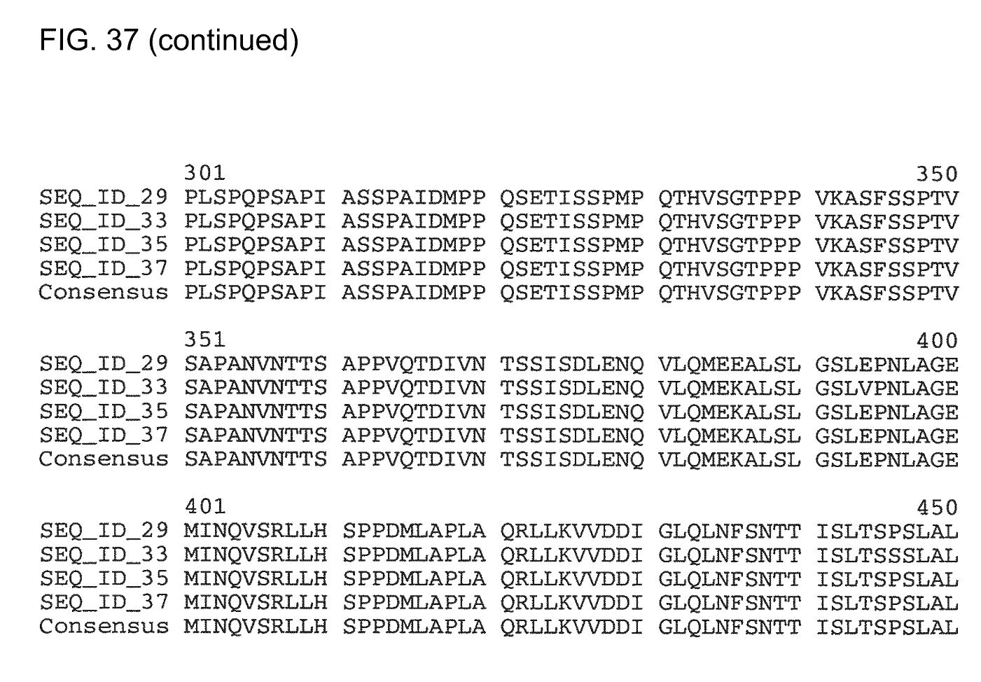

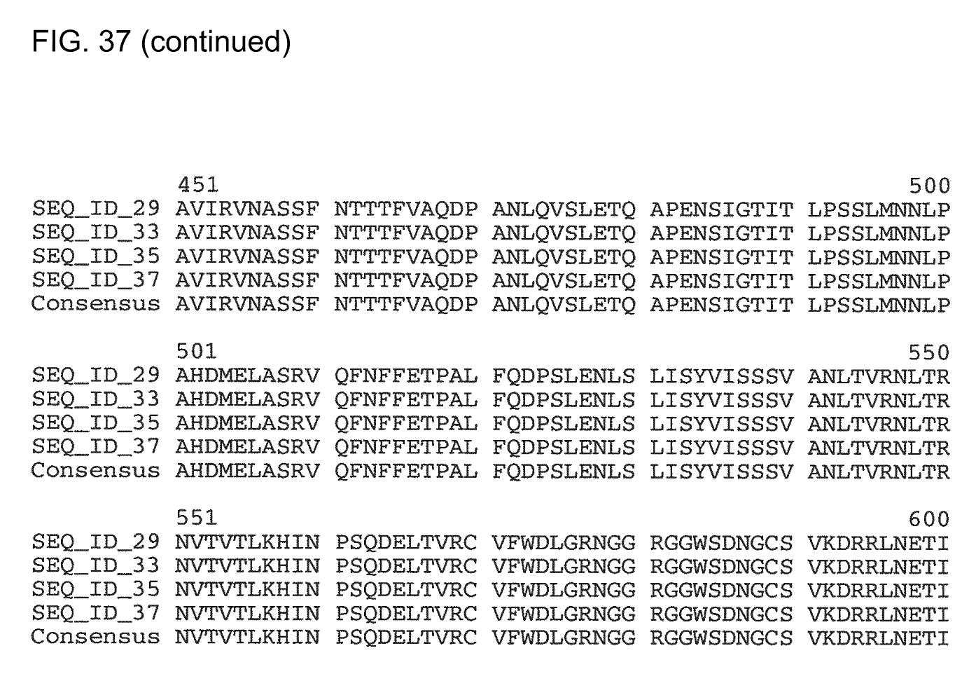

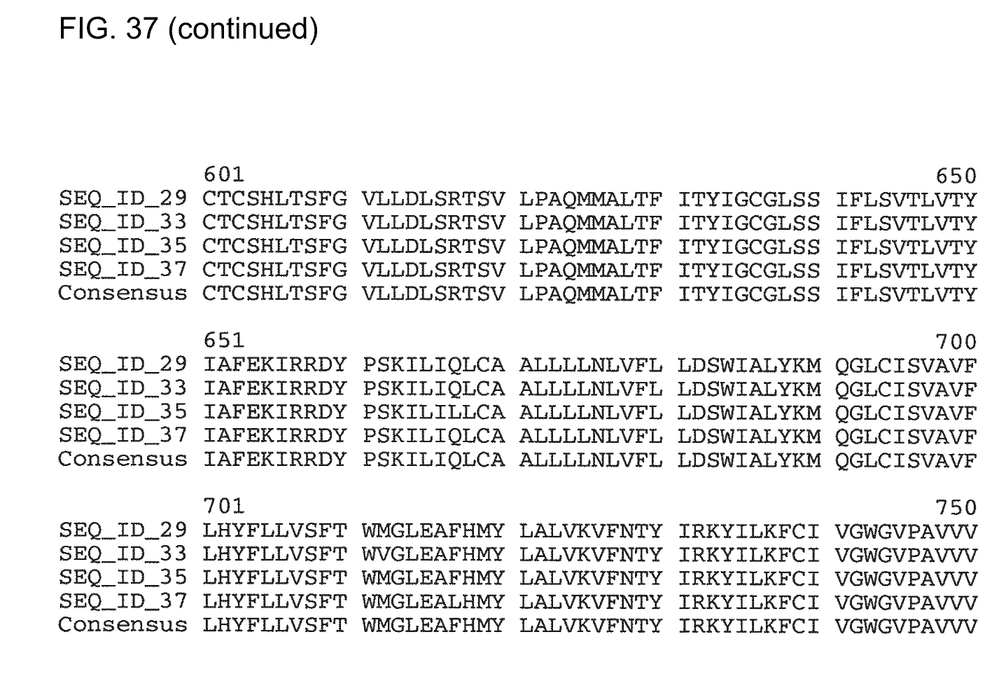

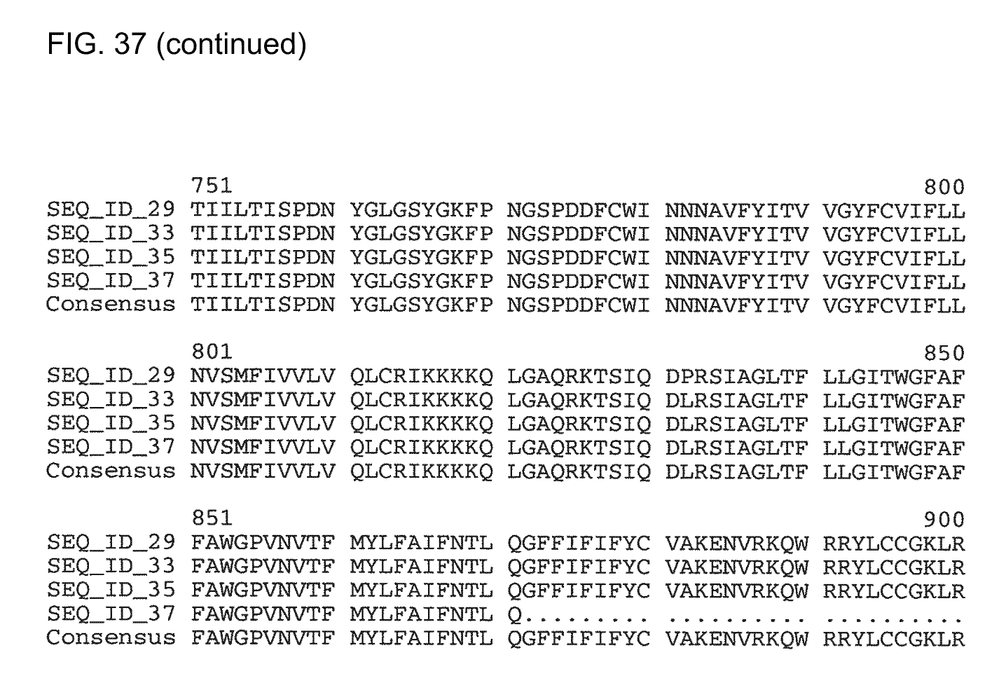

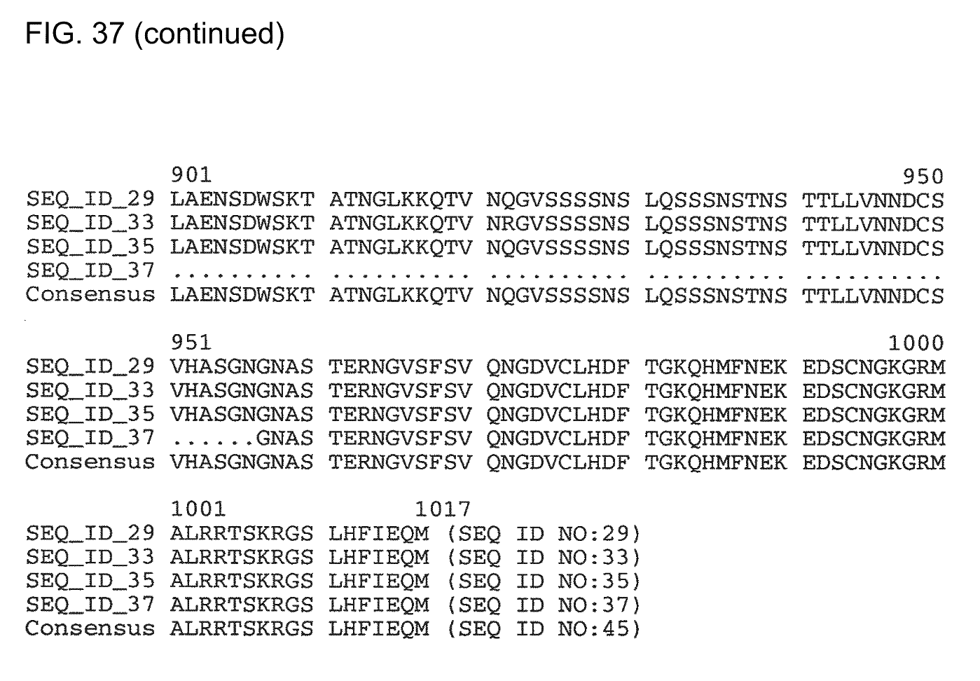

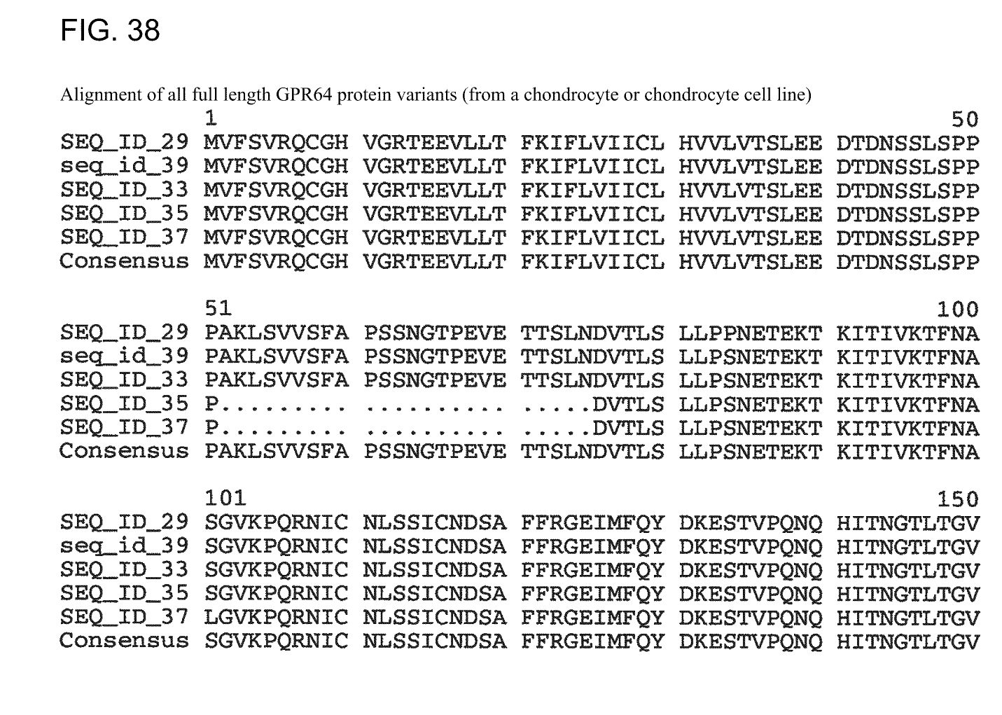

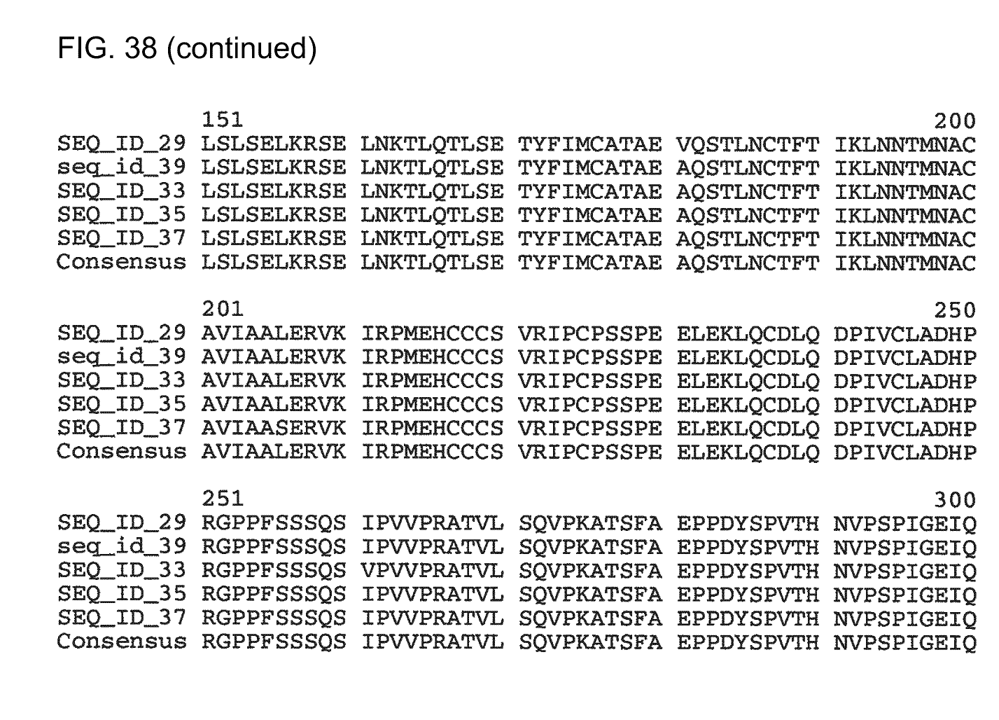

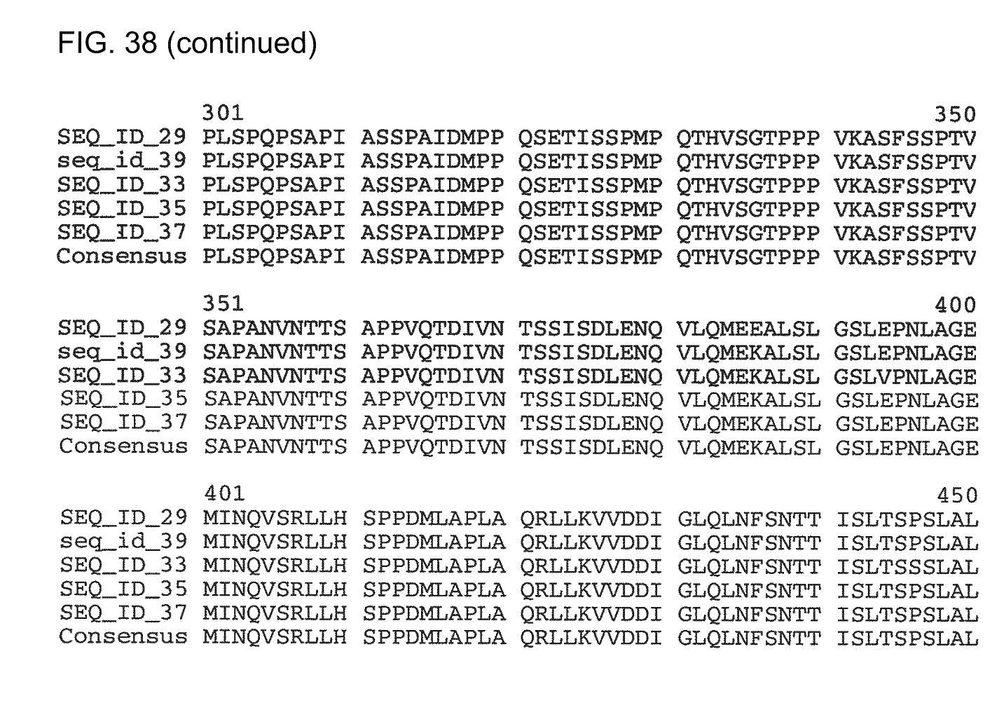

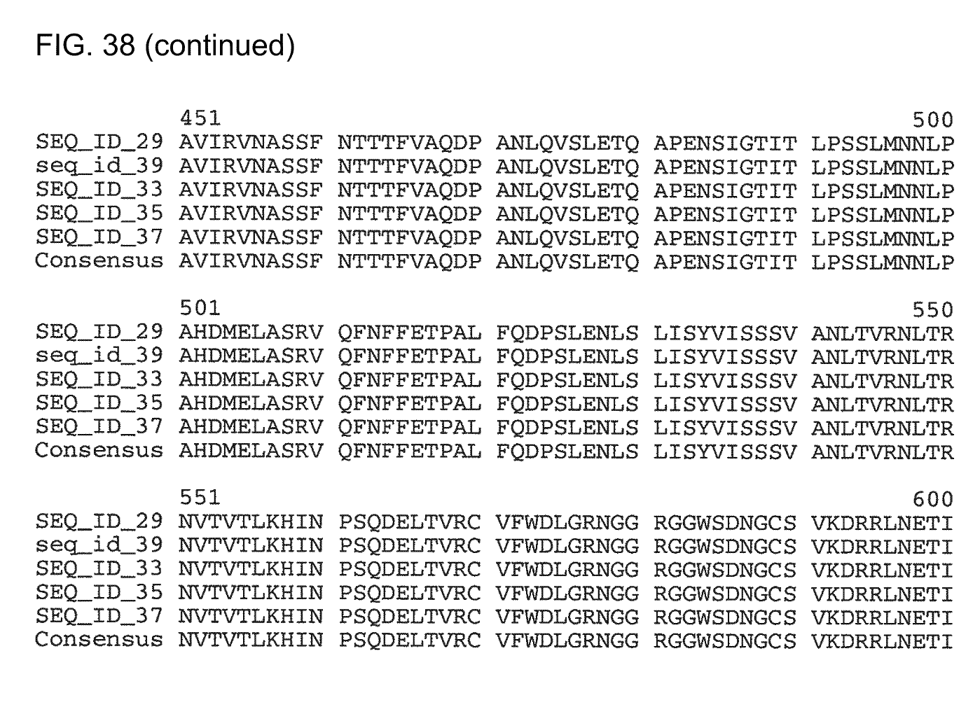

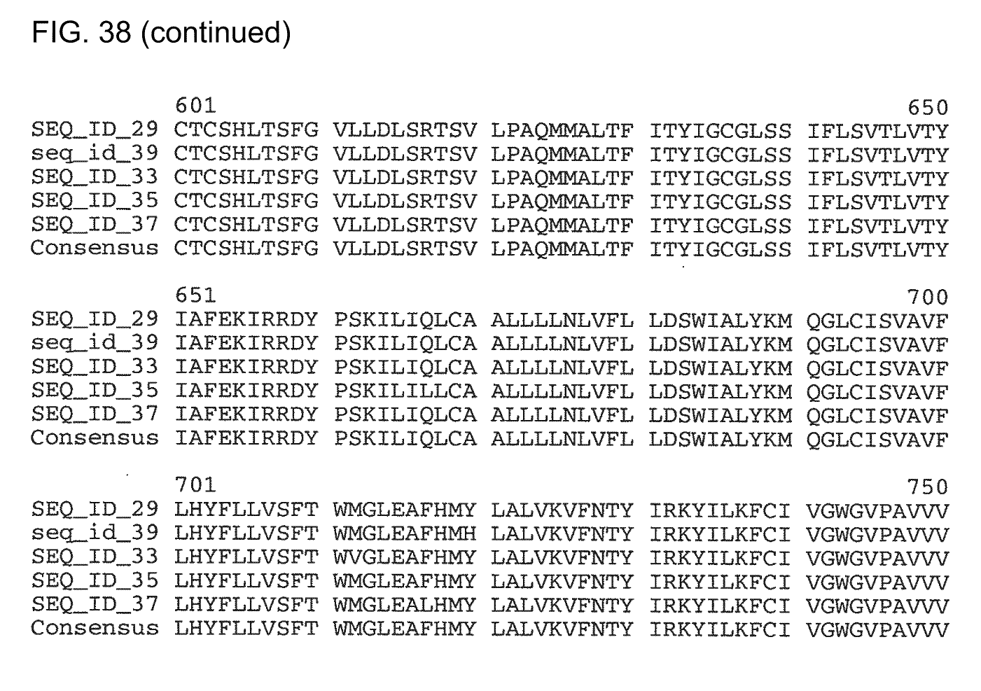

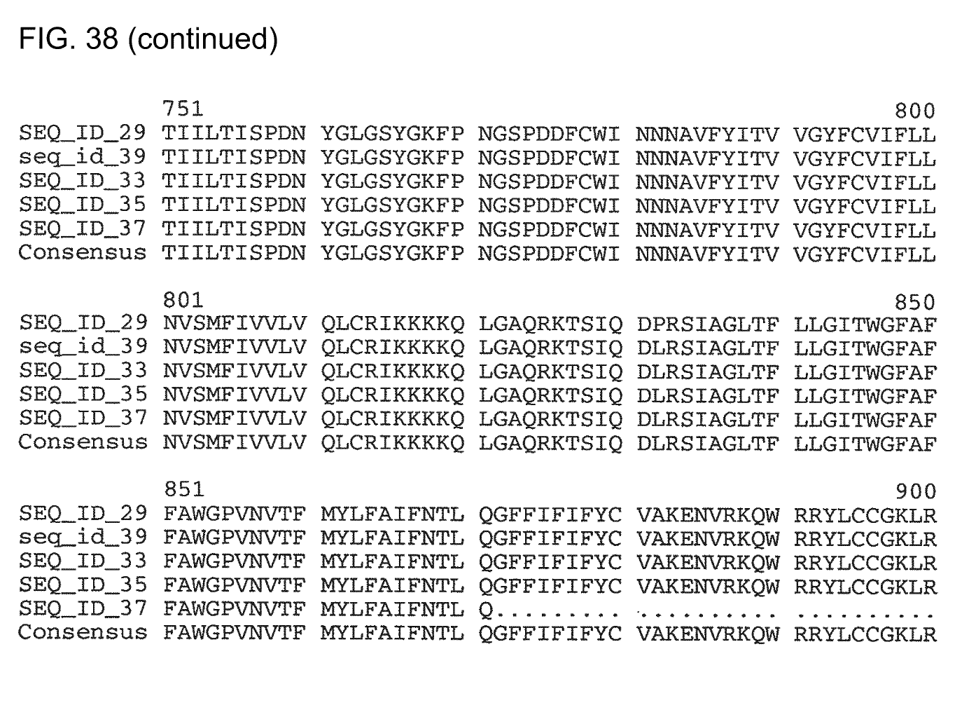

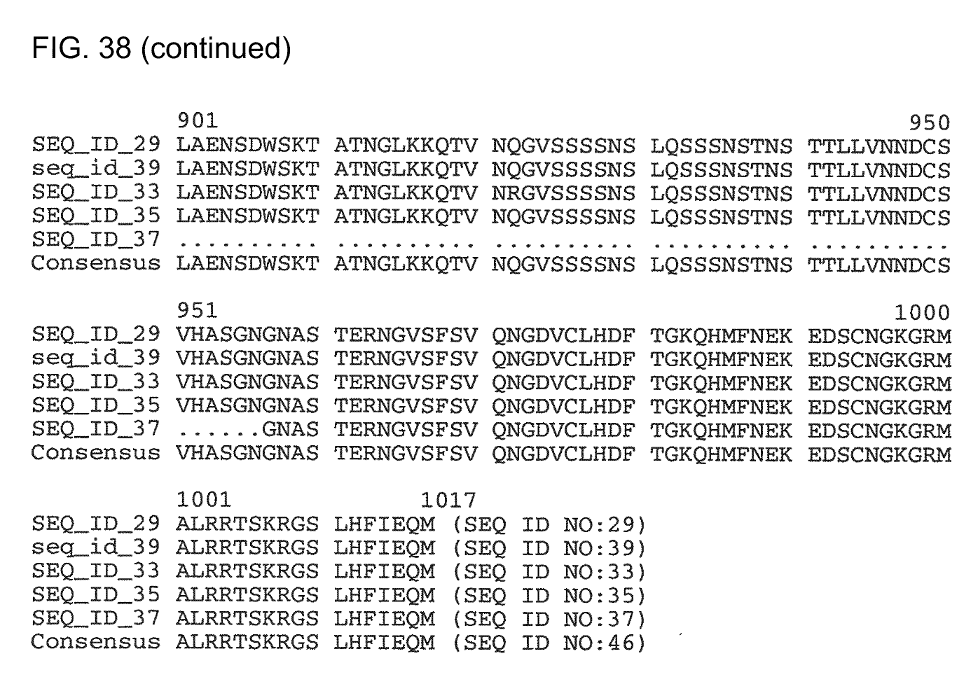

In some embodiments of the methods and compositions described herein, the GPR64 protein may be encoded by an amino acid sequence that has at least about 80%, at least about 90%, at least about 95%, at least about 96%, at least about 97%, at least about 98%, at least about 99%, or 100% similarity or identity to the amino acid sequence set forth in SEQ ID NO:2 (FIG. 2), SEQ ID NO:4 (FIG. 4), SEQ ID NO:6 (FIG. 5B), SEQ ID NO:27 (FIG. 19D), SEQ ID NO:29 (FIG. 21), SEQ ID NO:31 (FIG. 23), SEQ ID NO:33 (FIG. 25), SEQ ID NO:35 (FIG. 27), SEQ ID NO:37 (FIG. 29) or SEQ ID NO:39 (FIG. 31). Percent identity may be determined, for example, by comparing sequence information using the advanced BLAST computer program, version 2.0.8 or later version, available from the National Institutes of Health. In some embodiments, to determine similarity, the amino acid variations are based on conservative substitutions in which the amino acid substituted into the sequence retains similar characteristics, such as, for example, hydrophobicity, hydrophilicity, lipophilicity, size of the side chain, shape of the side chain, and/or charge, as the amino acid which it is replacing.

GPR64 and variants may be produced by methods known to the skilled artisan. For example, a nucleotide sequence encoding a GPR64 or variant may be introduced into a desired host cell. Such a nucleotide sequence may first be inserted into an appropriate recombinant expression vector.

Recombinant expression vectors may be constructed by incorporating the above-recited nucleotide sequences within a vector according to methods well known to the skilled artisan. A wide variety of vectors are known that are useful in the invention. Suitable vectors include plasmid vectors and viral vectors, including retrovirus vectors, adenovirus vectors, adeno-associated virus vectors, and herpes viral vectors. The vectors may include other known genetic elements necessary or desirable for efficient expression of the nucleic acid in a specified host cell, including regulatory elements. For example, the vectors may include a promoter and any necessary enhancer sequences that cooperate with the promoter to achieve transcription of the gene. The nucleotide sequence may be operably-linked to such regulatory elements.

Such a nucleotide sequence is referred to herein as a “genetic construct.” A genetic construct may contain a genetic element on its own or in combination with one or more additional genetic elements, including, but not limited to, genes, promoters, or enhancers. In some embodiments, these genetic elements are operably-linked. In some embodiments, the specific gene at issue (e.g., GPR64) may not be present in the genetic construct, including, but not limited to, a situation in which a GPR64 promoter is operably-linked to a reporter gene.

As used herein, a nucleotide sequence is “operably-linked” to another nucleotide sequence when it is placed in a functional relationship with another nucleotide sequence. For example, if a coding sequence is operably-linked to a promoter sequence, this generally means that the promoter may modulate (e.g., promote) transcription of the coding sequence, or if a ribosome binding site is operably-linked to a coding sequence, this generally means that it is positioned so as to facilitate translation. Operably-linked means that the DNA sequences being linked are typically contiguous and, where necessary to join two protein coding regions, contiguous and in reading frame. However, since enhancers may function when separated from the promoter by several kilobases and intron sequences may be of variable lengths, some nucleotide sequences may be operably-linked but not contiguous or not in reading frame. In some embodiments, linking can be accomplished by ligation at convenient binding sites, or if such sites do not exist, synthetic oligonucleotide adaptors or linkers can be used in accordance with conventional practice.

A wide variety of methods are available for introducing nucleotide sequences encoding GPR64 or variants, and which may be included in a recombinant expression vector, into a host cell. Such methods are known in the art and include, without limitation, mechanical methods, chemical methods, lipophilic methods, and electroporation. Microinjection and use of a gene gun with, for example, a gold particle substrate for the DNA to be introduced is a representative, non-limiting exemplary mechanical method. Use of calcium phosphate or DEAE-Dextran is a representative, non-limiting exemplary chemical method. Non-limiting exemplary lipophilic methods include use of liposomes and other cationic agents for lipid-mediated transfection. Such methods are well known to the art.

A wide variety of host cells may be utilized in embodiments of the invention to produce the desired quantities of GPR64. Such cells include, but are not limited to, eukaryotic and prokaryotic cells, including, without limitation, mammalian cells (including, but not limited to, U2OS, human embryonic kidney cells (such as, for example, HEK293), Chinese Hamster Ovary (CHO) cells and chondrocytes)), insect cells, yeast cells and bacterial cells known to the art.

GPR64 may be isolated and purified by techniques well known to the skilled artisan, including, but not limited to, chromatographic, electrophoretic, and centrifugation techniques. Such methods are known to the art.

In some methods described herein, a sample (e.g., tissue, cell culture, or an amount of GPR64 protein) can be contacted with a test agent for a time period sufficient to inhibit or activate the activity or expression of the GPR64 or variant. This time period and the quantity of sample may vary depending on factors including, but not limited to, the nature of the inhibitor, the activity/expression detection mechanism, and the sample tissue selected. The skilled artisan without undue experimentation may readily determine such times and amounts. An exemplary test agent is one that binds to or otherwise decreases the activity or expression of GPR64, although test agents that inhibit the activity or expression by, for example, binding to a component of the signal pathway, such as an enzyme substrate, or by some other mechanism, are also envisioned. When a sample tissue is used, the type of tissue chosen may vary depending on the specific inflammatory disease being studied. Non-limiting examples of sample tissues include cartilage, synovial fluid, synovium, and bone.

A wide variety of assays may be utilized to determine whether the test agent modulates (e.g., inhibits or activates) the activity or expression of GPR64. For example, the location and/or amount of reactants remaining and/or products formed in reactions and/or interactions involved in the GPR64 signal pathway may be quantified or ascertained. In various embodiments, the location and/or amount of reactants remaining and/or products formed in reactions and/or interactions involved in the NFκB pathway may be quantified or ascertained. Non-limiting examples of such reactions include Taqman, Western, protein phosphorylation, ELISA, cellular localization, and reporter assays. Other reactions include, without limitation, cAMP assay, calcium flux assay, inositol phosphate. To this end, the location of a transcription factor (such as, for example, p65 or the NFκB complex) or co-factors related to NFκB activation may be determined. In other embodiments, the amount of GPR64, MMPs (such as, for example, MMP13) and/or aggrecanases (such as, for example, ADAMTS1, ADAMTS4, ADAMTS5, ADAMTS8, ADAMTS9, and ADAMTS15) remaining, produced, or present after contacting the sample tissue or GPR64 with the test agent may be determined. In some embodiments, this is determined as a function of time. In additional embodiments, this is determined relative to a control level. Various assays may be used to determine the quantity, location, and/or presence of these products and/or reactants.

By way of non-limiting example, such assays include Taqman, Northern blot, Western, ELISA, enzyme activity, immunohistochemistry (1HC), in situ hybridization (ISH), fluorescence resonance energy transfer (FRET), histologic, fluorescence polarization (FP), and cellular translocation assays. These and other applicable assays are known to those of skill in the art.

GPR64 belongs to the family of G protein coupled receptors (GPCRs) based on primary sequence analysis. Based on this analysis, a series of cell-based functional assays can be set up to determine the G protein signaling pathway(s) that GPR64 activates. The recombinant receptor will be stably expressed in mammalian cell lines (such as U2OS, CHO, and HEK293). Receptor expression on the cell surface can be shown by flow cytometry, immunocytochemistry, or FACS analysis using anti-receptor antibodies, or by expressing receptor that contains an extracellular N-terminal short epitope tag (such as c-myc or FLAG) and using the respective anti-tag antibodies in flow cytometry. Levels of second messengers that are usually mobilized by GPCRs, such as, for example, cAMP, cGMP, diacylglycerol, inositol (1,4,5)-triphosphate, phosphatidyl inositol triphosphate, arachidonic acid, and phosphatidic acid, can be measured in cells expressing receptor in comparison to cells transfected with vector only (control cells). These measurements can be carried out using commercially-available kits. If the levels of these second messengers are significantly different between receptor-transfected and control cells, it can be concluded that GPR64 possesses constitutive activity.

To determine whether GPR64's activity is stimulated by small molecules, the receptor can be co-expressed with promiscuous chimeric G proteins, which are known G proteins inside the cell for which it is not known whether they are binding GPR64 (such as, for example, G alpha i; G alpha q; G alpha s; G alpha 12), that are coupled to a readout. These cells can be loaded with a dye and screened against libraries of small molecules with fluorescence-based screening technologies (such as, for example, fluorometric imaging plate reader (FLIPR) or Transfluor Technology™, from Molecular Devices, Sunnyvale, Calif.). The FLIPR Calcium 3 Assay Kit provides a universal method for detecting changes in intracellular calcium concentration in a simple and reliable homogeneous assay format. Transfluor™ is a cell-based fluorescence assay used to screen for G-protein-coupled receptors (GPCRs), ligands, and other potential drugs that regulate GPCRs. The technology is based on the discovery that, upon activation by ligand binding, virtually all GPCRs rapidly undergo deactivation or “desensitization” by a common pathway. An early step in this pathway is the binding of the cytoplasmic protein beta-arrestin to the activated receptor. Beta-arrestin binding deactivates the GPCR signaling and begins the translocation of the receptor into the cell where the ligand is removed and the receptor is recycled back to the cell membrane. By attaching a fluorescent label to beta-arrestin, the location of the receptor-arrestin complex can be monitored. Since desensitization only occurs with an activated receptor, monitoring beta-arrestin translocation and subsequent receptor recycling provides a method to detect the activation of any GPCR. Once small molecule agonists are identified, the same basic method can be used to screen for a small molecule antagonist.

In addition, assays known to one of skill in the art, including, but not limited to, Northern blots (to determine RNA expression levels) and Western blots (to determine protein expression levels) can be used to determine the level of expression of GPR64 by measuring the relative amounts of RNA or protein in the sample compared to a control.

Methods of quantitating GPR64 are known to the art, including use of various immunoassays, such as enzyme-linked immunosorbents assays, quantitative PCR, RT-PCR, and immunohistochemistry. Non-limiting examples of such assays are discussed herein.

A wide variety of test agents may be tested in the screening methods of various embodiments of the invention. For example, small molecule compounds known in the art, including, but not limited to, synthetic small molecules, chemicals, nucleic acids (such as, for example, antisense oligonucleotides and silencing RNA), peptides and proteins (such as, for example, hormones, antibodies, cytokines and chemokines, and portions thereof), may act as test agents. In one non-limiting example, the three-dimensional structure of the active site of GPR64 is determined by crystallizing the complex formed by the receptor and a ligand or inhibitor. Rational drug design can then be used to identify new test agents by making alterations in the structure of a known inhibitor or by designing small molecule compounds that bind to the active site of the enzyme.

In one embodiment, a method of screening for agents for treating inflammatory disease in a subject by screening for an agent that modulates (e.g., inhibits or activates) the activity of GPR64 or that modulates the expression of GPR64 includes contacting a nucleotide sequence encoding a reporter gene product operably-linked to a GPR64 promoter, with a test agent thought to be effective in inhibiting or activating production of GPR64; determining if the test agent inhibits or activates production of the reporter gene product; and classifying the test agent as an agent for treating inflammatory disease if the test agent modulates (e.g., inhibits or activates) production of the reporter gene product. In some embodiments, the subject is selected from the group consisting of rat, mouse, monkey, cow, horse, pig, rabbit, goat, sheep, dog, cat, and human. In one embodiment, the subject is a human. In some embodiments, the subject is not human.

The nucleotide sequence of the GPR64 promoter can be determined by art-recognized methods. Nucleotide sequences having at least about 50%, at least about 70%, at least about 80%, and at least about 90% identity to such sequences and that function as a promoter, for example, to direct expression of a gene encoding GPR64 described herein, can also be used in the methods and compositions described herein. One non-limiting example of such a method is to screen a genomic library (e.g., a YAC human genomic library) for the promoter sequence of interest using SEQ ID NO:1 (FIG. 1) or SEQ ID NO:3 (FIG. 3) as a probe. Another non-limiting example of a method to determine the appropriate promoter sequence is to perform a Southern blot of the human genomic DNA by probing electrophoretically resolved human genomic DNA with a probe (e.g., a probe comprising SEQ ID NO:1 or a portion thereof) and then determining where the cDNA probe (e.g., SEQ ID NO:1 or a portion thereof) hybridizes. Upon determining the band to which the probe hybridizes, the band can be isolated (e.g., cut out of the gel) and subjected to sequence analysis. This allows detection of the nucleotide fragment 5′ of nucleotides 73-75 (i.e., the ATG site) of SEQ ID NO:1. The nucleotide fragment may be between about 500 and about 1000 nucleotides in length or larger. The promoter sequence for murine GPR64 set forth in SEQ ID NO:3 (FIG. 3) may be determined by these methods as well. This allows detection of the nucleotide fragment 5′ of nucleotides 72-74 (i.e., the ATG site) of SEQ ID NO:3. Nucleotide sequences having at least about 70%, at least about 80%, and at least about 90% identity to such sequences and that function as promoter, for example, to direct expression of a gene encoding GPR64 described herein, can also be used in the methods and compositions described herein.

A wide variety of reporter genes may be operably-linked to the GPR64 promoter described above. Such genes may encode, for example, luciferase, β-galactosidase, chloramphenical acetyltransferase, β-glucuronidase, alkaline phosphatase, and green fluorescent protein, or other reporter gene products known to the art.

In an embodiment of the invention, the nucleotide sequence encoding a reporter gene that is operably-linked to a GPR64 promoter is introduced into a host cell. Such a nucleotide sequence may first be inserted into an appropriate recombinant expression vector as previously described herein.

Vectors may include other known genetic elements necessary or desirable for efficient expression of the nucleic acid sequence from the GPR64 promoter in a specified mammalian cell, including regulatory elements. For example, the vectors may include any necessary enhancer sequences that cooperate with the promoter in vivo, for example, to achieve in vivo transcription of the reporter gene. The methods of introducing the nucleotide sequence into a host cell are identical to that previously described for producing GPR64.

A wide variety of host cells may be utilized in the methods described herein. Exemplary host cells include, for example, U2OS, Chinese hamster ovary, 293, COS, Bacillus cells, E. coli, S. cerevisiae, and S. pombe.

Alternatively, the nucleotide sequence encoding all or a portion of the GPR64 gene may be utilized in the vector for the screening methods described herein. In such a case, GPR64 may be isolated and purified by techniques well known to the skilled artisan, including, without limitation, chromatographic, electrophoretic, and centrifugation techniques, as previously described herein and as known in the art. Additionally, GPR64 may be quantified by methods known to the art.

After contacting a nucleotide sequence encoding a reporter gene or a GPR64 gene operably-linked to GPR64 promoter with a test agent thought to be effective in modulating (e.g., inhibiting or activating) expression of GPR64, it is determined if the test agent modulates (e.g., inhibits or activates) production of the reporter gene product. This endpoint may be determined by quantifying either the amount or activity of the reporter gene product. The method of quantification will depend on the reporter gene that is used, but may involve use of an enzyme-linked immunosorbent assay with antibodies to the reporter gene product. Additionally, the assay may measure chemiluminescence, fluorescence or radioactive decay, or other methods known in the art. Assays for determining the activity or amount of the reporter gene products described herein are known to the art. If the test agent modulates (e.g., inhibits or activates) production of the reporter gene product, it is classified as an agent for treating inflammatory diseases.

The above methods and procedures can also be used for various other screening methods. For example, the methods described herein can be used to screen for an inflammatory disease in a subject or to screen for an increase in expression of GPR64 in a subject. By way of non-limiting example, these methods can include exposing a sample of tissue from the subject to an agent that binds to GPR64, MMP13, ADAMTS1, ADAMTS4, ADAMTS5, ADAMTS8, ADAMTS9, and/or ADAMTS15, detecting the level of binding of the agent to GPR64, MMP13, ADAMTS1, ADAMTS4, ADAMTS5, ADAMTS8, ADAMTS9, and/or ADAMTS15 in the sample, and comparing the level of binding of the agent to GPR64, MMP13, ADAMTS1, ADAMTS4, ADAMTS5, ADAMTS8, ADAMTS9, and/or ADAMTS15 in the sample to a control level. In another non-limiting example, the screening method can include obtaining a sample of tissue from the subject, preparing a composition of cellular material from the sample (which in some embodiments may involve various extraction or isolation steps to extract or isolate, for example, RNA or protein from other cellular material), detecting the level of GPR64, MMP13, ADAMTS1, ADAMTS4, ADAMTS5, ADAMTS8, ADAMTS9, and/or ADAMTS15 protein or RNA in the composition of cellular material, and comparing the level of GPR64, MMP13, ADAMTS1, ADAMTS4, ADAMTS5, ADAMTS8, ADAMTS9, and/or ADAMTS15 protein or RNA in the composition of cellular material to a control level. If the level of binding of the agent to GPR64, MMP13, ADAMTS1, ADAMTS4, ADAMTS5, ADAMTS8, ADAMTS9, and/or ADAMTS15, or the level of GPR64, MMP13, ADAMTS1, ADAMTS4, ADAMTS5, ADAMTS8, ADAMTS9, and/or ADAMTS15 protein or RNA is increased relative to the control level, the subject may be classified as having an inflammatory disease. Alternatively, for example, if the level of binding of the agent to GPR64, MMP13, ADAMTS1, ADAMTS4, ADAMTS5, ADAMTS8, ADAMTS9, and/or ADAMTS15, or the level of GPR64, MMP13, ADAMTS1, ADAMTS4, ADAMTS5, ADAMTS8, ADAMTS9, and/or ADAMTS15 protein or RNA is decreased relative to the control level, the subject may be classified as not having an inflammatory disease.

Non-limiting examples of agents useful in this method include antibodies directed against GPR64 as described herein. Non-limiting examples of inflammatory diseases that can be tested by this method include but are not limited to arthritis (including, but not limited to, OA, RA, spondyloarthropathies, and psoriatic arthritis), asthma (including, but not limited to, atopic asthma, nonatopic asthma, allergic asthma, exercise-induced asthma, drug-induced asthma, occupational asthma, and late stage asthma), inflammatory bowel disease (including, but not limited to, Crohn's Disease), inflammatory skin disorders (including, but not limited to, psoriasis, atopic dermatitis, and contact hypersensitivity), multiple sclerosis, osteoporosis, tendonitis, allergic disorders (including, but not limited to, rhinitis, conjunctivitis, and urticaria), inflammation in response to an insult to the host (including, but not limited to, injury or infection), sepsis, and systematic lupus erythematosus. In one embodiment, the inflammatory disease is OA. In another embodiment, the inflammatory disease is rheumatoid arthritis.

In other methods, an inflammatory disease can be diagnosed in a subject suspected of suffering from an inflammatory disease. By way of non-limiting example, this method can include exposing a sample of tissue from the subject to an agent that binds to GPR64, MMP13, ADAMTS1, ADAMTS4, ADAMTS5, ADAMTS8, ADAMTS9, and/or ADAMTS15, detecting a level of binding of the agent to GPR64, MMP13, ADAMTS1, ADAMTS4, ADAMTS5, ADAMTS8, ADAMTS9, and/or ADAMTS15 in the sample, and comparing the level of binding of the agent to GPR64, MMP13, ADAMTS1, ADAMTS4, ADAMTS5, ADAMTS8, ADAMTS9, and/or ADAMTS15 in the sample to a control level. In another non-limiting example, the screening method can include obtaining a sample of tissue from the subject, preparing a composition of cellular material from the sample (which in some embodiments may involve various extraction or isolation steps to extract or isolate, for example, RNA or protein from other cellular material), detecting the level of GPR64, MMP13, ADAMTS1, ADAMTS4, ADAMTS5, ADAMTS8, ADAMTS9, and/or ADAMTS15 protein or RNA in the composition of cellular material, and comparing the level of GPR64, MMP13, ADAMTS1, ADAMTS4, ADAMTS5, ADAMTS8, ADAMTS9, and/or ADAMTS15 protein or RNA in the composition of cellular material to a control level. If the level of binding of the agent to GPR64, MMP13, ADAMTS1, ADAMTS4, ADAMTS5, ADAMTS8, ADAMTS9, and/or ADAMTS15, or the level of GPR64, MMP13, ADAMTS1, ADAMTS4, ADAMTS5, ADAMTS8, ADAMTS9, and/or ADAMTS15 protein or RNA is increased relative to the control level, the subject may be diagnosed as having an inflammatory disease. Alternatively, for example, if the level of binding of the agent to GPR64, MMP13, ADAMTS1, ADAMTS4, ADAMTS5, ADAMTS8, ADAMTS9, and/or ADAMTS15, or the level of GPR64, MMP13, ADAMTS1, ADAMTS4, ADAMTS5, ADAMTS8, ADAMTS9, and/or ADAMTS15 protein or RNA is decreased relative to the control level, the subject may be diagnosed as not having an inflammatory disease.

Non-limiting examples of agents useful in this method include antibodies directed against GPR64 as described herein. Non-limiting examples of inflammatory diseases that can be tested by this method include but are not limited to arthritis (including, but not limited to, OA, RA, spondyloarthropathies, and psoriatic arthritis), asthma (including, but not limited to, atopic asthma, nonatopic asthma, allergic asthma, exercise-induced asthma, drug-induced asthma, occupational asthma and late stage asthma), inflammatory bowel disease (including, but not limited to, Crohn's Disease), inflammatory skin disorders (including, but not limited to, psoriasis, atopic dermatitis, and contact hypersensitivity), multiple sclerosis, osteoporosis, tendonitis, allergic disorders (including, but not limited to, rhinitis, conjunctivitis, and urticaria), inflammation in response to an insult to the host (including, but not limited to, injury or infection), sepsis, and systematic lupus erythematosus. In one embodiment, the inflammatory disease is OA. In another embodiment, the inflammatory disease is rheumatoid arthritis.

Other methods described herein involve treating inflammatory diseases. “Treatment,” “treating,” or “treated,” as used herein, means preventing, reducing or eliminating at least one symptom or complication of the inflammatory disease. Exemplary symptoms and/or complications of such inflammatory diseases include, but are not limited to, pain, edema, swelling, heat, malaise, joint stiffness, and redness. In addition, for OA, additional symptoms that can be reduced or eliminated include, without limitation, degradation of cartilage and subsequent changes in the presence of these degradative products in body fluids. In various embodiments, these methods include administering to a subject in need thereof a composition comprising an agent that modulates the activity or expression of GPR64. In some embodiments, the subject is selected from the group consisting of rat, mouse, monkey, cow, horse, pig, rabbit, goat, sheep, dog, cat, and human. In one embodiment, the subject is a human. In some embodiments, the subject is not human.

In one embodiment, this method comprises administering a therapeutic amount of an agent that decreases the activity or expression of GPR64. In another embodiment this comprises administering a therapeutic amount of an agent that increases the activity or expression of GPR64. A “therapeutic amount” represents an amount of an agent that is capable of inhibiting or decreasing the activity or expression of GPR64 or causes a clinically significant response. The clinical response includes an improvement in the condition treated or in the prevention of the condition. The particular dose of the agent administered according to this invention will, of course, be determined by the particular circumstances surrounding the case, including the agent administered, the particular inflammatory disease being treated, and similar conditions. In some embodiments, the agent binds to GPR64. In one embodiment, the agent is an inhibitor of GPR64. In another embodiment, the agent is an activator of GPR64. In other embodiments, the agent interacts with GPR64. In still other embodiments, the agent binds to or interacts with (such as by chemically modifying) an inhibitor or activator of GPR64 activity or expression. By way of non-limiting example, an agent may bind to and inhibit (or activate) an activator of GPR64 or an agent may bind to and activate (or inhibit) an inhibitor of GPR64 activity.

Agents that modulate (e.g., decrease or increase) the activity or expression of GPR64 include, without limitation, those agents discovered in the screening assays described herein. Additional agents, or inhibitors or activators, include, for example, antibodies against GPR64 or against activators of GPR64 activity or expression. An antibody as used herein, may be, without limitation, a polyclonal antibody, a monoclonal antibody, a chimeric antibody, a humanized antibody, a genetically-engineered antibody, a bispecific antibody, antibody fragments (including, but not limited to, “Fv,” “F(ab′)₂,” “F(ab),” and “Dab”) and single chains representing the reactive portion of the antibody. Such an antibody includes antibodies belonging to any of the immunoglobulin classes, such as IgM, IgG, IgD, IgE, IgA, or their subclasses or mixtures thereof. The invention further includes derivatives of these antibodies, such as those that retain their GPR64-binding activity while altering one or more other properties related to their use as a pharmaceutical agent, e.g., serum stability or efficiency of production.

In various embodiments, such an antibody binds to GPR64, an activator or inhibitor of GPR64 activity or expression, or another component of the GPR64 signal pathway. Binding portions of such antibodies are also included. Methods for production of each of the above antibody forms are well known to the art.

Cells that can be used to synthesize antibodies include animal cells, fungal cells, bacterial cells, or yeast cells after transformation. By way of non-limiting example, hybridoma cells can be produced in a known manner from animals immunized with GPR64 and isolation of their antibody-producing B cells, selecting these cells for GPR64-binding antibodies and subsequently fusing these cells to, for example, human or animal, for example, mouse myeloma cells, human lymphoblastoid cells, or heterohybridoma cells (see, e.g., Kohler et al., (1975) Nature 256: 495-97) or by infecting these cells with appropriate viruses to produce immortalized cell lines.

By way of non-limiting example, human GPR64 monoclonal antibodies may be obtained as follows. Those of skill in the art will recognize that other equivalent procedures for obtaining GPR64 antibodies are also available and are included in various embodiments of the invention.

First, a mammal is immunized with human GPR64. The mammal used for raising anti-human GPR64 antibody is not restricted and may be a primate, a rodent, such as mouse or rat, rabbit, bovine, sheep, goat, or dog.

Next, antibody-producing cells, such as spleen cells, are removed from the immunized animal and are fused with myeloma cells. Myeloma cells are well-known in the art. By way of non-limiting example, p3×63-Ag8-653, NS-0, NS-1, or P3U1 cells may be used. The cell fusion operation may be carried out by a well-known conventional method.

The cells, after being subjected to the cell fusion operation, are then cultured in HAT selection medium so as to select hybridomas. Hybridomas, which produce anti-human monoclonal antibodies, are then screened. This screening may be carried out by, for example, sandwich ELISA (enzyme-linked immunosorbent assay) or the like in which the produced monoclonal antibodies are bound to the wells to which human GPR64 is immobilized. In this case, an antibody specific to the immunoglobulin of the immunized animal, which is labeled with an enzyme, such as peroxidase, alkaline phosphatase, glucose oxidase, beta-D-galactosidase, or the like, may be employed as the secondary antibody. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine, or the like may be used.

By the above-described operation, hybridomas, which produce anti-GPR64 human antibodies, can be selected. The selected hybridomas are then cloned by the conventional limiting dilution method or soft agar method. If desired, to obtain a large number of the cloned hybridomas, the cloned hybridomas may be cultured on a large scale using a serum-containing or a serum-free medium, or may be inoculated into the abdominal cavity of mice and recovered from ascites.

The monoclonal antibodies further include hybrid and recombinant antibodies produced by splicing a variable (including hypervariable) domain of an anti-GPR64 antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)).

Thus, the term “monoclonal” indicates that the character of the antibody obtained is from a substantially homogeneous population of antibodies (i.e., the individual antibodies comprising the population are identical except for possible naturally-occurring mutations that may be present in minor amounts) and is not to be construed as requiring production of the antibody by any particular method. For example, the monoclonal antibodies to be used in accordance with various embodiments of the invention may be made by the hybridoma method first described by Kohler & Milstein, Nature 256:495-497 (1975), or may be made by recombinant DNA methods (See, e.g., U.S. Pat. No. 4,816,567). The “monoclonal antibodies” may also be isolated from phage libraries generated using the techniques described in McCafferty et al., Nature 348:552-554 (1990), for example.

The level of GPR64 in a sample can be detected or quantified using, e.g., an antibody, such as a monoclonal antibody described herein. The detection or quantification of the GPR64 in a sample can be carried out by an immunoassay utilizing the specific binding reaction between the monoclonal antibody of some embodiments of the invention and GPR64. Various immunoassays are well-known in the art and any of them can be employed. Non-limiting examples of the immunoassays include sandwich methods employing the monoclonal antibody and another monoclonal antibody as primary and secondary antibodies respectively, sandwich methods employing the monoclonal antibody and a polyclonal antibody as primary and secondary antibodies, staining methods employing gold colloid, agglutination method, latex method, and chemical luminescence. By way of non-limiting example, the sandwich ELISA can be used. As is well-known, in this method, a primary antibody is immobilized on, for example, the inner wall of a well and then a sample is reacted with the immobilized primary antibody. After washing, a secondary antibody is reacted with the antigen-antibody complex immobilized in the well. After washing, the immobilized secondary antibody is quantified. In some embodiments, an antibody that specifically reacts with human GPR64 is employed as the primary antibody.

The quantification of the secondary antibody may be carried out by reacting a labeled antibody (e.g., enzyme-labeled antibody) specific to the immunoglobulin of the animal from which the secondary antibody was obtained with the secondary antibody and then measuring the label. Alternatively, a labeled (e.g., enzyme-labeled) antibody is used as the secondary antibody, and the quantification of the secondary antibody may be carried out by measuring the label on the secondary antibody.

Antibody fragments can be obtained, for example, by enzymatic means by eliminating the Fc part of the antibody with enzymes, such as papain or pepsin, by chemical oxidation, or by genetic manipulation of the antibody genes. It is also possible and advantageous to use genetically-manipulated, non-truncated fragments. These antibodies or fragments thereof can be used alone or in mixtures.

In some embodiments, the anti-GPR64 antibodies are used in immunotherapy. In this context, immunotherapy means treatment of an inflammatory disease or symptom of an inflammatory disease with an antibody raised against GPR64 proteins. The immunotherapy can be passive or active. Passive immunotherapy is the passive transfer of antibody to a recipient, whereas active immunotherapy is the induction of antibody and/or T-cell responses in a recipient. Induction of an immune response is the result of providing the recipient with an antigen (e.g., GPR64 or DNA encoding it) to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In certain embodiments, the antibody is conjugated to an effector moiety. The effector moiety can be any number of molecules including, but not limited to, detection/labeling moieties, such as radioactive labels or fluorescent labels, and therapeutic moieties (e.g., a chemotherapeutic or cytotoxic agent, an antibiotic, a lipase, a radioisotope emitting beta irradiation). In one aspect, the therapeutic moiety is a small molecule that modulates the activity of the GPR64 protein. In another aspect, the therapeutic moiety modulates the activity of molecules associated with or which are in close proximity to the GPR64 protein.

In other embodiments, the therapeutic moiety is a cytotoxic agent. In this method, targeting the cytotoxic agent to a desired region results in a reduction in the number of inflammatory cells, thereby reducing symptoms associated with the inflammatory disorder. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin, and the like. Cytotoxic agents also include radiochemicals made by conjugating radioisotopes (e.g., I¹²⁵, I¹³¹, Y⁹⁰, and Re¹⁸⁶) to antibodies raised against GPR64, or binding of a radionuclide to a chelating agent that has been covalently attached to the antibody. Targeting the therapeutic moiety to the desired region of the recipient not only serves to increase the local concentration of therapeutic moiety in the afflicted area, but also serves to reduce deleterious side effects that may be associated with the therapeutic moiety.

In one embodiment, the agent that decreases the expression of GPR64 is a nucleic acid. Exemplary nucleic acids include, but are not limited to, a deoxyribonucleic acid or a ribonucleic acid. In one embodiment, the ribonucleic acid has a nucleotide sequence that is complementary to at least a portion of the nucleotide sequence set forth in SEQ ID NO:1 or SEQ ID NO:3, as set forth in FIGS. 1 and 3, encoding GPR64. In another embodiment, the ribonucleic acid has a nucleotide sequence that is complementary to at least a portion of the nucleotide sequence encoding variants of GPR64, as set forth in SEQ ID NO:5 (FIG. 5A), SEQ ID NO:26 (FIG. 19C), SEQ ID NO:28 (FIG. 20), SEQ ID NO:30 (FIG. 22), SEQ ID NO:32 (FIG. 24), SEQ ID NO:34 (FIG. 26), SEQ ID NO:36 (FIG. 28) or SEQ ID NO:38 (FIG. 30). In alternative embodiments, the ribonucleic acid has a nucleotide sequence that is complementary to at least a portion of a nucleotide sequence encoding the amino acid sequence set forth in SEQ ID NO:2 (FIG. 2), SEQ ID NO:4 (FIG. 4), SEQ ID NO:6 (FIG. 5B), SEQ ID NO:27 (FIG. 19D), SEQ ID NO:29 (FIG. 21), SEQ ID NO:31 (FIG. 23), SEQ ID NO:33 (FIG. 25), SEQ ID NO:35 (FIG. 27), SEQ ID NO:37 (FIG. 29) or SEQ ID NO:39 (FIG. 31).

In another embodiment, RNA interference may be used as an inhibitor of GPR64 expression. RNA interference relates to sequence-specific, post-transcriptional gene silencing brought about by double-stranded RNA that is homologous to the silenced gene target. Methods for inhibiting production of a protein utilizing small interfering RNAs are well known to the art, and disclosed in, for example, PCT Publication Numbers WO 01/75164; WO 00/63364; WO 01/92513; WO 00/44895; and WO 99/32619. siRNAs directed to GPR64 have been tested as discussed herein in Example 3.

RNA interference (RNAi) is a process whereby double-stranded RNA (dsRNA) induces the sequence-specific degradation of homologous mRNA in animals and plant cells (Hutvagner and Zamore, 2002, Curr. Opin. Genet. Dev. 12:225-232; Sharp, 2001, Genes Dev. 15:485-490). In mammalian cells, RNAi can be triggered by, for example, without limitation, approximately 21-nucleotide (nt) duplexes of small interfering RNA (siRNA) (Chiu et al., 2002, Mol. Cell. 10:549-561; Elbashir et al., 2001, Nature 411:494-498), or by micro-RNAs (miRNA), functional small-hairpin RNA (shRNA), or other dsRNAs which are expressed in vivo using DNA templates with RNA polymerase III promoters (Zeng et al., 2002, Mol. Cell. 9:1327-1333; Paddison et al., 2002, Genes Dev., 16:948-958; Lee et al., 2002, Nature Biotechnol. 20:500-505; Paul et al., 2002, Nature Biotechnol. 20:505-508; Tuschl, 2002, Nature Biotechnol. 20:440-448; Yu et al., 2002, Proc. Natl. Acad. Sci. USA, 99:6047-6052; McManus et al., 2002, RNA 8:842-850; Sui et al., 2002, Proc. Natl. Acad. Sci. USA 99:5515-5520).

Examples of molecules that can be used to decrease expression of a gene, such as, for example, GPR64, include double-stranded RNA (dsRNA) molecules that can function as siRNAs and that comprise 16-30, for example, 16, 17, 18, 19, 20, 21, 22, 23, 24, 25, 26, 27, 28, 29, or 30 nucleotides in each strand, wherein one of the strands is substantially complementary to, for example, at least about 80% (or more, for example, about 85%, 90%, 95%, or 100%) complementary to, for example, having 3, 2, 1, or 0 mismatched nucleotide(s), a target region, such as, for example, a transcribed region of the nucleic acid of the gene, and the other strand is identical or substantially identical to the first strand. The dsRNA molecules can be chemically-synthesized, or can be transcribed in vitro from a DNA template, or in vivo from an engineered RNA precursor, for example, shRNA. The dsRNA molecules may be designed using methods known in the art (for example, “The siRNA User Guide,” available at rockefeller.edu/labheads/tuschl/siRNA) and can be obtained from commercial sources, for example, Dharmacon, Inc. (Lafayette, Colo.) and Ambion, Inc. (Austin, Tex.). Non-limiting examples of siRNA molecules that can be used to decrease expression of GPR64 include SEQ ID NOS:14, 15, 16, and 17.

Negative control siRNAs generally have the same nucleotide composition as the selected siRNA but without significant sequence complementarity to the targeted genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The siRNAs for use as described herein can be delivered to a cell by methods known in the art and as described herein in using methods such as, for example, transfection utilizing commercially-available kits and reagents. Viral infection, for example, using a lentivirus vector, an adenoviral vector, an adeno-associated viral vector, or a retroviral vector can also be used.

The nucleic acid molecules described herein, including siRNA molecules, can also be labeled using any method known in the art; for instance, the nucleic acid compositions can be labeled with a fluorophore, such as, for example, Cy3, fluorescein, or rhodamine. The labeling can be carried out using a kit, such as, for example, the SILENCER™ siRNA labeling kit (Ambion Austin, Tex.). Additionally, an siRNA can be radiolabeled, such as, for example, using ³H, ³²P, or other appropriate isotope.

An siRNA or other oligonucleotide can also be introduced into the cell by transfection with an heterologous target gene using carrier compositions, such as, for example, liposomes, which are known in the art, such as, for example, Lipofectamine™ 2000 (Invitrogen, Carlsbad, Calif.) as described by the manufacturer for adherent cell lines. Transfection of dsRNA oligonucleotides for targeting endogenous genes can be carried out using Oligofectamine™ (Invitrogen, Carlsbad, Calif.). The effectiveness of the oligonucleotide can be assessed by any of a number of assays following introduction of the oligonucleotide into a cell. These assays include, but are not limited to, Western blot analysis using antibodies that recognize the targeted gene product following sufficient time for turnover of the endogenous pool after new protein synthesis is repressed, and Northern blot analysis to determine the level of existing target mRNA.

Still further compositions, methods, and applications of RNAi technology for use as described herein are provided in U.S. Pat. Nos. 6,278,039, 5,723,750, and 5,244,805. MicroRNA technology is also included, as described in Carthew, Current Opinion in Genetics & Development, 16:1-6 (2006). The descriptions in these references related to RNAi and microRNA technology are incorporated by reference herein.

In some methods described herein, the activity or expression of GPR64 is modulated in a subject. Such methods include administering a composition comprising an agent that modulates the activity or expression of GPR64 to a subject. In some embodiments, the subject is selected from the group consisting of rat, mouse, monkey, cow, horse, pig, rabbit, goat, sheep, dog, cat, and human. In one embodiment, the subject is a human. In some embodiments, the subject is not human. In one embodiment, this comprises administering a therapeutic amount of an agent to a subject in need of such treatment. In some embodiments, the agent decreases the activity or expression of GPR64. In another embodiment, this comprises administering a therapeutic amount of an agent that increases the activity or expression of GPR64. In additional embodiments, the agent can be any agent described herein or discovered by the methods described herein.

In some embodiments, the agent binds to GPR64. In one embodiment, the agent is a modulator (i.e., activator or inhibitor of GPR64). In a particular embodiment, the agent is an inhibitor of GPR64. In other embodiments, the agent interacts with GPR64. In still other embodiments, the agent binds to or interacts with (such as by chemically modifying) an inhibitor or activator of GPR64 activity or expression. By way of non-limiting example, an agent may bind to and inhibit an activator of GPR64 or an agent may bind to and activate an inhibitor of GPR64 activity. In some embodiments, the agent may modify GPR64 transcription, GPR64 translation, or the GPR64 signal pathway. In various embodiments, the agent may modulate the NFκB pathway. By way of non-limiting example, the agent may cause the location of a transcription factor (such as, for example, p65 or the NFκB complex) or co-factors related to NFκB activation to be changed (for example, from the cytoplasm to the nucleus) or the level of an enzyme that degrades cartilage (including, without limitation, MMP13, ADAMTS1, ADAMTS4, ADAMTS5, ADAMTS8, ADAMTS9, or ADAMTS15) to increase.

Such methods of modulating the activity or expression of GPR64 can be used to treat inflammatory diseases. Non-limiting examples of inflammatory diseases that can be treated by this method include but are not limited to arthritis (including, but not limited to, OA, RA, spondyloarthropathies, and psoriatic arthritis), asthma (including, but not limited to, atopic asthma, nonatopic asthma, allergic asthma, exercise-induced asthma, drug-induced asthma, occupational asthma, and late stage asthma), inflammatory bowel disease (including, but not limited to, Crohn's Disease), inflammatory skin disorders (including, but not limited to, psoriasis, atopic dermatitis, and contact hypersensitivity), multiple sclerosis, osteoporosis, tendonitis, allergic disorders (including, but not limited to, rhinitis, conjunctivitis, and urticaria), inflammation in response to an insult to the host (including, but not limited to, injury or infection), sepsis and systematic lupus erythematosus. In one embodiment, the inflammatory disease is OA. In another embodiment, the inflammatory disease is rheumatoid arthritis.

An agent that modulates the activity or expression of GPR64 and a pharmaceutically-acceptable carrier can be provided as a pharmaceutical composition. These compositions are suitable for administration to a subject, including to a human. The pharmaceutical composition can be used for treating an inflammatory disease. Non-limiting examples of inflammatory diseases that can be treated by this method include arthritis (including, but not limited to, osteoarthritis, rheumatoid arthritis, spondyloarthropathies, and psoriatic arthritis), asthma (including, but not limited to, atopic asthma, nonatopic asthma, allergic asthma, exercise-induced asthma, drug-induced asthma, occupational asthma, and late stage asthma), inflammatory bowel disease (including, but not limited to, Crohn's Disease), inflammatory skin disorders (including, but not limited to, psoriasis, atopic dermatitis, and contact hypersensitivity), multiple sclerosis, osteoporosis, tendonitis, allergic disorders (including, but not limited to, rhinitis, conjunctivitis, and urticaria), inflammation in response to an insult to the host (including, but not limited to, injury or infection), sepsis, and systematic lupus erythematosus. In one embodiment, the inflammatory disease is OA. In another embodiment, the inflammatory disease is RA. Such an agent may be any of the agents described herein or discovered by methods described herein. In some embodiments, the agent decreases the activity or expression of GPR64. In some embodiments, the agent binds to GPR64. In other embodiments the agent is an inhibitor or activator of GPR64 activity or expression. In additional embodiments, the agent interacts with an inhibitor of GPR64 activity or expression. In still other embodiments, the agent interacts with an activator of GPR64 activity or expression.

The agents may be administered by a wide variety of routes. Exemplary routes of administration include oral, parenteral, transdermal, colorectal, rectal, and pulmonary administration. For example, the agents may be administered intranasally, intramuscularly, subcutaneously, intraperitonealy, intravaginally, or any combination thereof. For pulmonary administration, nebulizers, inhalers, or aerosol dispensers may be used to deliver the therapeutic agent in an appropriate formulation (e.g., with an aerolizing agent). In addition, the agents may be administered alone or in combination with other agents or known drugs. In combination, agents may be administered simultaneously or each agent may be administered at different times. When combined with one or more known anti-inflammatory drugs, agents, and drugs may be administered simultaneously or the agent can be administered before or after the drug(s).

In one embodiment, the agents are administered in a pharmaceutically-acceptable carrier. Any suitable carrier known in the art may be used (see, e.g., Remington's Pharmaceutical Sciences, pp. 1447-1676 (Alfonso R. Gennaro, ed., 19^{th} ed. 1995)). Carriers that efficiently solubilize the agents are preferred. Carriers include, but are not limited to, a solid, liquid, or a mixture of a solid and a liquid. The carriers may take the form of capsules, tablets, pills, powders, lozenges, suspensions, emulsions, or syrups. The carriers may include substances that act as flavoring agents, lubricants, solubilizers, suspending agents, binders, stabilizers, tablet disintegrating agents, and encapsulating materials. The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein, means a pharmaceutically-acceptable material, composition, or vehicle, such as a liquid or solid filler, diluent, excipient, solvent, or encapsulating material, involved in carrying or transporting the subject agents from one organ, or portion of the body, to another organ, or portion of the body. Such carriers must be suitable for use in contact with the tissues of human beings and animals, as previously described herein. In addition, each carrier must be “acceptable” in the sense of being compatible with the other ingredients of the formulation. Some examples of materials which can serve as pharmaceutically-acceptable carriers include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single-dosage form will vary depending upon the subject being treated, the particular mode of administration, the particular condition being treated, etc. The amount of active ingredient that can be combined with a carrier material to produce a single-dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or timely divided solid carriers, or both, and then, if necessary, shaping the product.

In solid dosage forms of the invention for oral administration (capsules, tablets, pills, dragees, powders, granules, and the like), the active ingredient is mixed with one or more additional ingredients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof, and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

In powders, the carrier is a finely-divided solid, which is mixed with an effective amount of a finely-divided agent. Powders and sprays can contain, in addition to a compound of this invention, excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Tablets for systemic oral administration may include one or more excipients as known in the art, such as, for example, calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with one or more disintegrating agents (e.g., maize, starch, or alginic acid, binding agents, such as, for example, gelatin, collagen, or acacia), lubricating agents (e.g., magnesium stearate, stearic acid, or talc), inert diluents, preservatives, disintegrants (e.g., sodium starch glycolate), surface-active and/or dispersing agent. A tablet may be made by compression or molding, optionally with one or more accessory ingredients.

In solutions, suspensions, emulsions or syrups, an effective amount of the agent is dissolved or suspended in a carrier, such as sterile water or an organic solvent, such as aqueous propylene glycol. Other compositions can be made by dispersing the agent in an aqueous starch or sodium carboxymethyl cellulose solution or a suitable oil known to the art. The liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants, such as wetting agents, emulsifying and suspending agents, sweetening, flavoring, coloring, perfuming, and preservative agents.

Suspensions, in addition to the active compound, may contain suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions for rectal or vaginal administration may be presented as a suppository, which may be prepared by mixing one or more compounds of the invention with one or more suitable non-irritating excipients or carriers comprising, for example, cocoa butter, polyethylene glycol, a suppository wax or a salicylate, and which is solid at room temperature but liquid at body temperature and, thus, will melt in the rectum or vaginal cavity and release the agents.

Formulations suitable for vaginal administration also include pessaries, tampons, creams, gels, pastes, foams, or spray formulations containing such carriers as are known in the art to be appropriate.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants that may be required.

Ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

The agents are administered in a therapeutic amount to a subject in need of such treatment. Such an amount is effective in treating inflammatory diseases. This amount may vary, depending on the activity of the agent utilized, the nature of the inflammatory disease, and the health of the subject. The term “therapeutically-effective amount” is used to denote treatments at dosages effective to achieve the therapeutic result sought. Furthermore, a skilled practitioner will appreciate that the therapeutically-effective amount of the agent may be lowered or increased by fine-tuning and/or by administering more than one agent, or by administering an agent together with an anti-inflammatory compound (e.g., NSAIDS, DMARDS, and steroids). Therapeutically-effective amounts may be easily determined, for example, empirically by starting at relatively low amounts and by step-wise increments with concurrent evaluation of beneficial effect. (e.g., reduction in symptoms). The actual effective amount will be established by dose/response assays using methods standard in the art (Johnson et al., Diabetes. 42:1179, (1993)). As is known to those in the art, the effective amount will depend on bioavailability, bioactivity, and biodegradability of the compound.

A therapeutically-effective amount is an amount that is capable of modulating the expression or activity of GPR64 in a subject. Accordingly, the amount will vary with the subject being treated. Administration of the compound may be hourly, daily, weekly, monthly, yearly, or a single event. For example, the effective amount of the compound may comprise from about 1 μg/kg body weight to about 100 mg/kg body weight. In one embodiment, the effective amount of the compound comprises from about 1 μg/kg body weight to about 50 mg/kg body weight. In a further embodiment, the effective amount of the compound comprises from about 10 μg/kg body weight to about 10 mg/kg body weight.

When one or more agents or anti-inflammatory compounds are combined with a carrier, they may be present in an amount of about 1 weight percent to about 99 weight percent, the remainder being composed of the pharmaceutically-acceptable carrier.

In some instances, one or more agents described herein can be administered to a subject in combination with another therapy for an inflammatory disease, such as those known in the art. For example, therapies for RA include non-steroidal anti-inflammatory drugs (NSAIDS, aspirin, ibuprofen, naproxen, COX-2 inhibitors, or combinations thereof), corticosteroids, hydroxychloroquine, gold, methotrexate, sulfasalazine, penicillamine, cyclophosphamide and cyclosporin or disease modifying drugs (DMARDS), such as anti-TNF therapies.

The compositions described herein can be included in kits that can be used for screening tissue to determine if a subject, including, but not limited to, a subject, has an inflammatory disease. Such kits can include one or more of the following: at least one container for a tissue sample, at least one component for detection of GPR64 (including, but not limited to, an antibody to GPR64 or a binding portion thereof), at least one component for quantification or visualization of the level of GPR64, at least one container for mixing the above components, either alone or with a sample tissue, a control level for comparison, and a control sample to determine whether the screening method is working properly. Such a kit may also include instructions directing the use of these materials. In another embodiment, a kit may include an agent used to treat an inflammatory disease with or without such above-mentioned materials that may be present to determine if a subject has an inflammatory disease.

The invention is further illustrated by the following examples. The examples are provided for illustrative purposes only. They are not to be construed as limiting the scope or content of the invention in any way.

### Example 1

### Determination of Differential Expression of GPR64

To determine genes differentially expressed in RA and OA, 42 samples of human synovia from 17 patients diagnosed with RA, 6 samples of human synovia from 4 OA patients, and 8 samples of normal human synovia (non-involved tissues from trauma patients undergoing amputation) from 3 patients were compared using Affymetrix® GeneChip™ (Santa Clara, Calif.) analysis.

The RA samples came from joint synovia and tenosynovia. Tenosynovia is from the synovial sheath around the tendons of the flexor or extensor compartments of the metacarpal phalanges. The joint synovia samples were grossly diagnosed as “capsular” (where the pannus is fully-contained in the synovial capsule) or “erosive” (where osteoclasts in the pannus have made contact with bone and caused destruction of bone matrix). The tenosynovia samples were grossly diagnosed as either “encapsulating” (where the pannus is a nodule of tissue attached to the tendon) or “invasive” (where the pannus has invaded the tendonous fibers and is disrupting the tissue).

OA synovial samples came from joint synovia, and normal samples came from either the ankle joints or the tenosynovial sheath surrounding the tendons of the metatarsal phalanges.

Total RNA was isolated from human synovial samples. Samples were lysed in tissue lysis buffer (RNAgents Kit, Promega, Madison, Wis.). Total RNA was isolated with a modification of the manufacturer's recommendations. Briefly, RNA was precipitated with the addition of isopropanol and washed twice with cold 75% ethanol. The pellet was dissolved in RNeasy minikit sample lysis buffer, and RNA was purified according to the manufacturer's recommendations (Qiagen, Valencia, Calif.). RNA was purified from cultured cells with the use of an RNeasy minikit, according to the manufacturer's recommendations (Qiagen, Valencia, Calif.). Total RNA was quantitated from a measure of UV absorption at 260 nm. An aliquot of total RNA was resolved with the use of agarose gel electrophoresis, and RNA integrity was assessed from a visual comparison of the relative intensities of the 18S and 28S rRNA bands. For all samples, the intensity of the 28S rRNA band exceeded that of the 18S band.

Synovia were subjected to analysis with the use of oligonucleotide microarrays. Double-stranded cDNA was prepared from 5-10 mg of total RNA with the use of the SuperScript Choice kit (Invitrogen, Carlsbad, Calif.) and 33 pmoles of oligo-dT primer containing a T7 RNA polymerase promoter (Proligo, LLC, Boulder, Colo.). First strand cDNA synthesis was initiated with the addition of the following kit components: first strand buffer at 1×, DTT at 10 mM, dNTPs at 500 mM, Superscript RT II at 400 U, and RNAse inhibitor at 40 U. The reaction proceeded at 47° C. for 1 hour. Second strand synthesis proceeded with the addition of the following kit components: second strand buffer at 1×, additional dNTPs at 200 mM, E. coli DNA polymerase I at 40 U, E. coli RNaseH at 2 U, and E. coli DNA ligase at 10 U. The reaction proceeded at 15.8° C. for 2 hours. T4 DNA polymerase (New England Biolabs, Beverly, Mass.), at a final concentration of 6 U, was added for the last five minutes of the second strand reaction. Doubled-stranded cDNA was purified with the use of a solid-phase, reversible immobilization technique (Byrne, M. C., Whitley, M. Z., and Follettie, M., T. (2000). Preparation of mRNA for Expression Monitoring. In “Current Protocols in Molecular Biology” (F. M. Ausubel, R. Brent, R. E. Kingston, D. D. Moore, J. G. Seidman, J. A. Smith, and K. Struhl, Eds.), pp. 22.2.1-22.2.13. John Wiley & Sons, Inc., Hoboken, N.J.) and collected in a volume of 20 ml of 10 mM Tris acetate, pH 7.8.

Purified cDNA (10 ml) was used in an in vitro transcription reaction, with the use of the Bioarray High Yield RNA Transcript labeling kit, according to the manufacturer's protocol (Enzo, Farmingdale, N.Y.). Biotin-labeled, antisense cRNA was purified with the use of a RNeasy mini kit as suggested by the manufacturer (Qiagen, Valencia, Calif.). The cRNA yield was determined from a measure of UV absorption at 260 nm.

To improve hybridization efficiencies, 15 mg of cRNA was incubated in fragmentation buffer (40 mM Tris-acetate, pH 8.1, 100 mM KOAc, 30 mM MgOAc) at 94° C. for 35 min. The fragmented cRNA probes were used to create a GeneChip hybridization solution as suggested by the manufacturer (Affymetrix, Santa Clara, Calif.). It should be noted that the hybridization solutions also contained a mix of eleven prokaryotic RNAs, each at a different concentration, which were used to create an internal standard curve for each chip and interpolate the frequencies of detected genes. Hybridization solutions were pre-hybridized to two glass beads (Fisher Scientific, Pittsburgh, Pa.) at 45° C. overnight. The hybridization solution was removed to a clean tube and heated for 1-2 min at 95° C. and microcentrifuged on high for 2 minutes to pellet insoluble debris. Labeled cRNA solutions were hybridized to Affymetrix HG_U95Av2 and Hg_U95B (Santa Clara, Calif.) chips. (OA cartilage samples were hybridized to the Hg_U95Av2 only). The RNAs hybridized to the chips were scanned on a Hewlett-Packard GeneArray Scanner, Model G2500A (Palo Alto, Calif.). Analyses of the data from the scans were performed using the GeneChip™ 3.1 program (Affymetrix, Santa Clara, Calif.) and GeneSpring™ (Silicon Genetics, Redwood, Calif.). Initial data processing was performed using the GeneChip 3.1 program and gene frequencies were determined using bacterial RNAs spiked-in at different levels to provide a standard curve. Other methods of determining gene frequencies are well known in the art.

RA and OA synovial samples were analyzed. Three different types of analysis were performed on the RA samples by comparing different subsets of the diseased samples to corresponding controls. For these comparisons, expression values for each gene, in each RA sample, were divided by the average expression value of the corresponding gene in the selected control samples. Three types of comparisons were made. (i) The first comparison involved all RA synovial samples (42) normalized to the average of all 8 control synovia; in this comparison, differences between diseased and control tissues were measured. (ii) The second comparison involved joint synovial RA samples (16) normalized to the average of 4 normal joint controls, and RA tenosynovial samples (26) normalized to the average of 4 normal tenosynovial samples. Here comparisons were made between two sites of disease: the joint and the tendon. (iii) The third comparison involved encapsulating tenosynovial RA samples (14) or invasive tenosynovial RA samples (12) separately normalized to 4 normal tenosynovial samples, capsular (13) or erosive (3) joint RA samples normalized to the average of 4 normal joint synovial samples. In the third comparison, differences in gross pathologies within the two disease sites were compared; in this case, aggressive pathologies in the tendon (invasive) and joint (erosive) were compared to apparently less aggressive pathologies: encapsulated in tendon and capsular in the joint as determined and described by the surgeons (Jain, et al., Arthritis Rheum 44: 1754-60 (2001)).

Data for genes that showed increased expression were filtered for an Absolute Decision of “Present” (a “call” made by the GeneChip program based upon an analysis of the probes for each gene) and a Frequency>5 PPM in >50% of the diseased samples in each type of analysis. An average fold change cutoff of 1.5× was applied to the data and then exported to Excel™ (Microsoft, Redmond, Wash.) for further analysis. Genes having a fold change of at least 2× in greater than 20% of the diseased samples and at least 1.5× in at least 50% of the samples in any of the above analyses were designated as genes with increased expression compared to non-diseased.

Data for genes that showed decreased expression were filtered for an Absolute Decision of “Present” and a Frequency>5 PPM in >50% of the normal samples in each type of analysis. An average fold change cutoff of −15× was applied to the diseased sample data and then exported to Excel (Microsoft, Redmond, Wash.). Genes with a fold change of at most −2× in greater than 20% of the diseased samples and less than −1.5× in at least 50% of the diseased samples were designated as genes with decreased expression compared to non-diseased samples.

Six samples of synovia from a total of 4 patients diagnosed with OA were analyzed using expression profiling on the Hg_U95Av2 chips. The normal joint controls described above were used as controls for these samples as well. Samples from two of the patients failed the RNA quality criteria because the ratios of frequency of the 5′ ends compared to the 3′ ends of beta actin and GAPDH fell below set criteria. This fact indicated that the RNA from these two samples might be degraded and that frequency values could appear lower than actual expression levels. Analyses were performed both indicating and excluding these two samples because the remaining four samples represented only two patients. Data for genes that showed increased or decreased expression were filtered as described for the RA synovia.

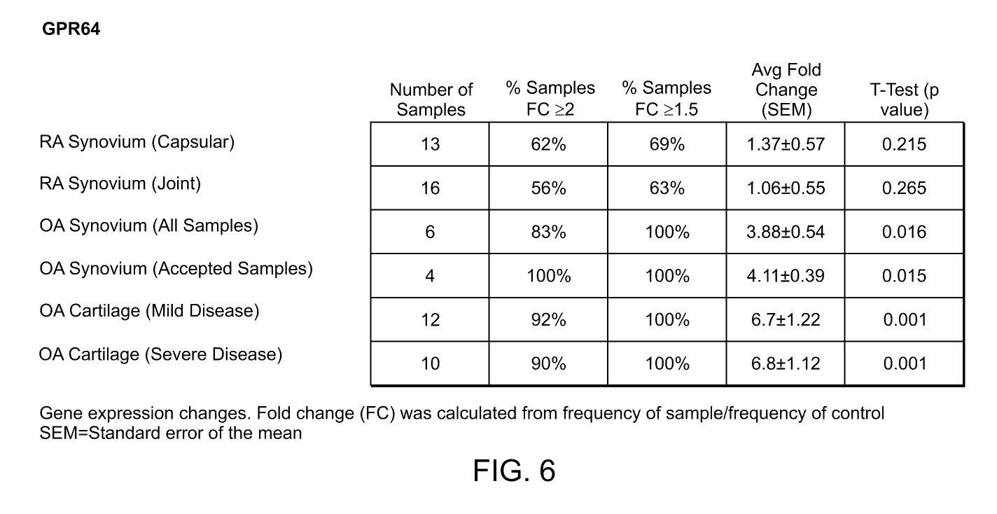

These analyses represent genes that are up-regulated or down-regulated in the synovia of RA and OA patients relative to the corresponding tissues of non-diseased patients. The different analyses performed point to potential specificities of certain genes for particular sites of disease and/or severity of disease, which can lead to the identification of potential targets for therapeutic intervention. In this example, the orphan GPR64 expression was increased. The modulation of the activity of this protein could be beneficial for inflammatory diseases. This data is shown in the table in FIG. 6.

The data demonstrate that GPR64 expression is increased in patients with inflammatory diseases, such as OA and RA.

### Example 2

### GPR64 Expression in OA Cartilage

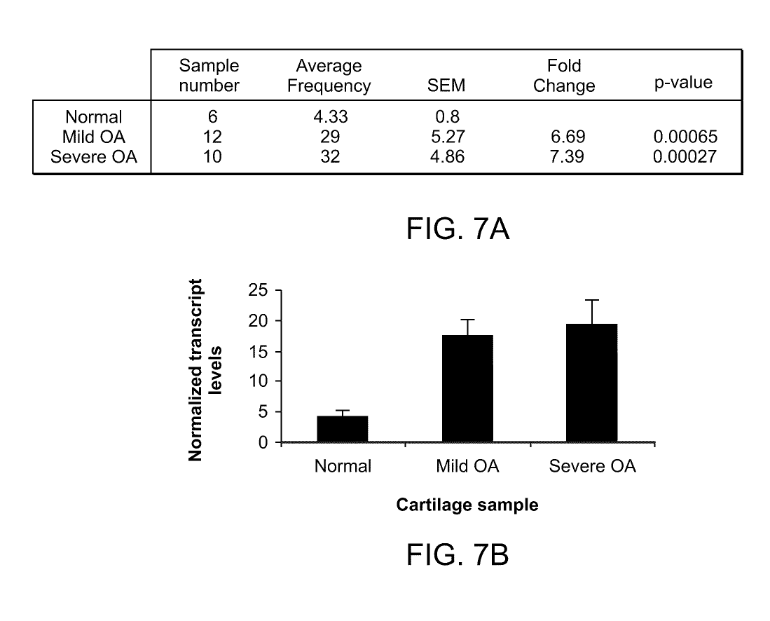

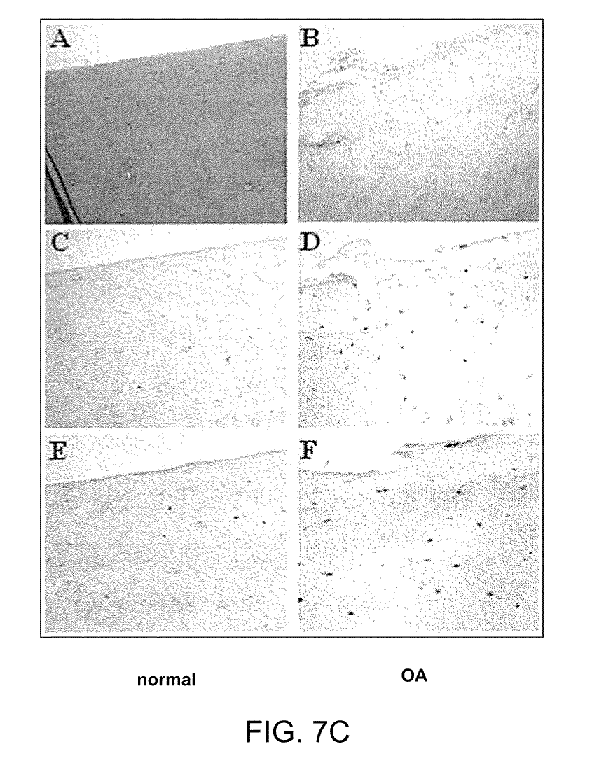

OA cartilage samples came from knee replacement patients. The areas of the cartilage that showed little damage were termed “mild” disease tissue, and areas of the cartilage with increased damage were termed “severe” disease tissue. Twelve mild and eleven severe cartilage samples were compared separately to the six normal cartilage controls. Data for genes that showed increased or decreased expression was filtered based upon a minimum fold change of greater than or equal to 2.5, and a p value less than 0.05 (FIG. 7A).

The expression of GPR64 was strongly increased in all OA cartilage samples. For RNA expression analysis, human normal, mild and severely affected OA cartilage samples were harvested after signed consent (New England Baptist Hospital, Boston, Mass.) and flash-frozen in liquid nitrogen. Frozen tissues were pulverized and RNA isolated utilizing guanidinium isothiocyante extraction and RNeasy kit (Qiagen, Valencia, Calif.). Agilent systems (Palo Alto, Calif.) were used to assess RNA quality. Quantitative real time RT-PCR was carried out utilizing primers and probes [5′-primer-ggagcctaacctcgcaggag (SEQ ID NO:7); 3′ primer-actactttcagcaatctttgagc (SEQ ID NO:8); probe-cagactccttcattccccgcctgac (SEQ ID NO:9) specific for human GPR64 and the human GAPDH gene GCGCCCAATACGACCAAA (SEQ ID NO:10), CCACATCGCTCAGACACCAT (SEQ ID NO:11), and GGGAAGGTGAAGGTCGGAGTCAACG (SEQ ID NO:12) for normalization.

Quantitative RT-PCR experiments performed on 6 donors for each sample type indicated that GPR64 expression (normalized against GAPDH and averaged) was increased in both mild and severely affected OA cartilage samples, compared to normal cartilage. Change in expression levels over normal was similar for both mild and severely affected OA samples with increases of 4.4 fold and 4.8 fold, respectively. This data is depicted in FIG. 7B. Expression levels were averaged from 6 donors for each cartilage type and error bars indicate standard error.

Immunochemistry was utilized to determine expression of GPR64 protein in normal and OA cartilage samples. Human normal and OA cartilage samples were fixed for 24 hours in 4% paraformaldehyde, embedded in paraffin and sectioned for immunohistochemistry. Tissues were stained with a polyclonal anti-GPR64 antibody (LifeSpan BioSciences, Seattle, Wash.) utilizing the DAKO Envision+system (DakoCytomation California Inc., Carpinteria, Calif.) and counterstained with Mayer's alum-hematoxylin. The extent of matrix degradation in each tissue sample was assessed by Safranin-O, which stains proteoglycan in the extracellular matrix, staining on adjacent sections (FIG. 7C). Cartilage samples from a minimum of 4 donors for each sample type were analyzed.

Normal (A, C, and E) and OA cartilage (B, D, and F) tissues were stained with Safranin-O (A and B) and anti-GPR64 (C and D). Panels E and F are magnified images of C and D. Safranin-O (compare A and B) staining showed that there is significant loss of proteoglycan by OA cartilage. Staining with anti-GPR64 (compare C and D) indicated that the number of cells positive for GPR64 increased in OA cartilage compared to normal cartilage. Therefore, increase in the number of cells positive for GPR64 correlated with loss of proteoglycan in the cartilage matrix as seen from Safranin-O staining.

### Example 3

### Example of Knockdown of GPR64

The role of GPR64 in chondrocytes and OA was investigated using RNA interference (RNAi) gene knockdown techniques in human chondroctye cell lines as well as primary human chondrocytes. Data indicated that GPR64 knockdown repressed IL-1β mediated activation of NFκB signaling as well as repressed the induction of MMP13 mRNA levels. Together, these data support that inhibition of GPR64 is a valuable intervention point for the treatment of OA.

### Example 3A

### Monitoring NFκB Activity in a Human Chondrocyte Cell Line: Generation of T/C-28a2-Clone19

NFκB is a downstream target of several signaling pathways including TNFα and IL-1β. A cell-based assay was developed based on cells containing NFκB response elements coupled to a luciferase reporter gene. Reporter gene activity can be induced upon treatment with either IL-1β or TNFα. Furthermore, molecules that inhibit NFκB signaling will not activate the response elements or repress a ligand-mediated induction and therefore, will result in diminished or no luciferase activity.

Vectors pIRESpuro3 (BD-Clontech, Cat. #6986, Palo Alto, Calif.) and pNFκB-Luc (BD-Clontech, Cat. #6053, Palo Alto, Calif.) were used for this purpose. When using the pIRESpuro3 vector, the antibiotic exerts selective pressure on the whole expression cassette; thus, a high dose of antibiotic will select for cells expressing a high level of the gene of interest. pNFκB-Luc is designed to measure the binding of the transcription factors to the κ enhancer, which then initiates transcription of the luciferase reporter gene, providing a direct measurement of activation of this pathway. These were co-transfected into human chondrocyte cell lines (T/C-28a2 and C-28/I2). The clones that survived selection were isolated.

Primary Screening: The positive clones were screened by a luciferase reporter assay (Promega, Madison, Wis.) after IL-1β (10 ng/ml) induction. 49 positive clones each from T/C-28a2 cell line and C-28I2 cell line were screened. 29 T/C clones and 11 C clones responded to IL-1β induction with a signal/background ratio of 5 or more in the luciferase reporter assay (Promega, Madison, Wis.).

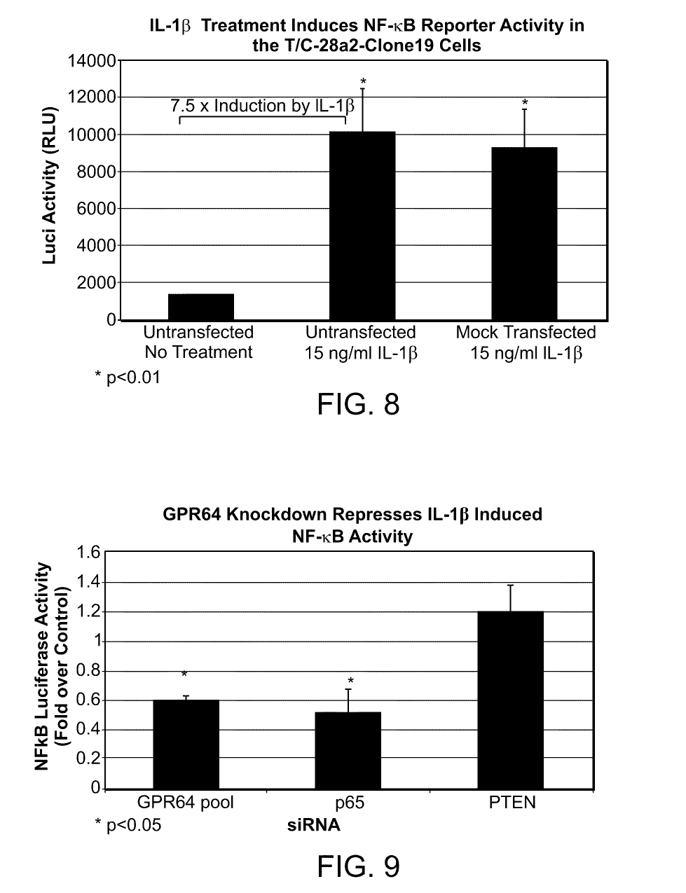

Secondary Screening: The clones that were selected were further screened using either TNFα (5 ng/ml and 20 ng/ml) or IL-1β (5 ng/ml and 20 ng/ml). C28I2 clones did not respond very well in the secondary screening. Further characterization was pursued only with T/C28a2 clones. In the secondary screening, T/C NFκB Clone #19 was selected based on its highest response and dose dependent response to both TNFα and IL-1β as compared to other clones at the same cell density. Following a 4 hour treatment with 15 ng/ml IL-1β (catalog #201-LB, R&D Systems, Minneapolis, Minn.), an approximately 7.5 fold induction of reporter gene activity was detected (see FIG. 8). Mock transfecting the cells with 0.5% Lipofectamine 2000 (catalog #11668-019, Invitrogen, Carlsbad, Calif.), did not alter this response. (These results are shown in FIG. 8).

### Example 3B

### Knockdown of GPR64 Represses IL-1β-Mediated NFκB Activity in the Human Chondrocyte Cell Line T/C-28a2-Clone19

The role of GPR64 in NFκB signal transduction in human chondrocytes was investigated using RNA interference in the T/C-28a2-Clone19 cells. In these experiments, siRNA reagents against human GPR64 were transfected into cells that were then subsequently treated with 15 ng/ml IL-1β (R&D Systems, Minneapolis, Minn.). NFκB-luciferase reporter gene activity was measured following 4 hours of treatment.

Briefly, T/C-28a2-Clone19 cells were seeded in 50 μl at 40,000 cells/well in a 96-well poly-lysine coated plate (catalog #356651, Promega, Madison, Wis.) and cultured in DMEM/F12 50:50 media (catalog #10-092-CV, Cellgro Herndon, Va.) supplemented with 10% FBS (Invitrogen, Carlsbad, Calif.). The cells were plated together with 50 μl of Optimem (catalog #31985-070, Invitrogen, Carlsbad, Calif.) containing 1% Lipofectamine 2000 (Invitrogen) and 5 nM siRNA (Dharmacon, Lafayette, Colo.). As a result, each well contained 5% FBS, 0.5% Lipofectamine 2000, and 2.5 nM siRNA final concentrations. The following day, the media was replaced with DMEM/F12 50:50 with 10% FBS. 48 hours post-transfection, the media was replaced with serum-free DMEM/F12 50:50 supplemented with 15 ng/ml IL-1β (R&D Systems, Minneapolis, Minn.) for 4 hours. Cell viability was monitored using the WSTassay according to the manufacturer's specifications (catalog #1664807, Roche, Indianapolis, Ind.). The assay is based on the cleavage of the tetrazolium salt WST-1 producing a soluble formazan salt. This conversion only occurs in viable cells. Some wells were treated with 500 ug/ml Etoposide (catalog#341206, Calbiochem, San Diego, Calif.), a potent inducer of cell death, as a control for this cell viability assay. Luciferase activity was monitored following incubation in a cell lysis buffer (catalog #E153A, Promega, Madison, Wis.) and luciferase substrate (catalog #E1501, Promega, Madison, Wis.) according to the manufacturer's protocol. Activity was monitored on a Victor 3 plate reader. As controls, cells were either mock transfected (no siRNA) or transfected with non-specific, siRNA sequences including: 5′-GGUAGCUAUUCAGUUACUG-3′ (SEQ ID NO:13); NSPV (catalog #D-001206-05, Dharmacon, Lafayette, Colo.); NSPVI (catalog #D-001206-06, Dharmacon, Lafayette, Colo.); NSPVIII (catalog #D-001206-08, Dharmacon, Lafayette, Colo.); NSPIX (catalog #D-001206-09, Dharmacon, Lafayette, Colo.); NSPX (catalog #D-001206-10, Dharmacon, Lafayette, Colo.); or NSPXI (catalog #D-001206-11, Dharmacon, Lafayette, Colo.). siRNA sequences for GPR64 knockdown were: GPR64-9 (catalog #D-003812-09, Dharmacon, Lafayette, Colo.) 5′-GAGUAAAGAUUCGACC AAUUU-3′ (SEQ ID NO:14); GPR64-10 (catalog #D-003812-10, Dharmacon, Lafayette, Colo.) 5′-GAGUAUCGCUGGCCUUACAUU-3′ (SEQ ID NO:15); GPR64-11 (catalog #D-003812-11, Dharmacon, Lafayette, Colo.) 5′-UAACGUGACCUUCAUGUAUUU-3′ (SEQ ID NO:16); and GPR64-12 (catalog #D-003812-12, Dharmacon, Lafayette, Colo.) 5′-GACAGGAGAUUGAAUGAAAUU-3′ (SEQ ID NO:17). In addition, an equal mixture of these 4 siRNA sequences was tested as GPR64 SMARTpool (catalog #D-003812-02, Dharmacon, Lafayette, Colo.). Additional controls included a pool of siRNAs against p65 (catalog #M003533-01, Dharmacon, Lafayette, Colo.), which is a component of NFκB; and a pool of siRNAs against PTEN (catalog #M-003023-01, Dharmacon, Lafayette, Colo.). PTEN has been implicated as a negative regulator of NFκB signaling (Vasudevan et al., (2004) Mol. Cell. Biol. 24, 1007-21). As a result, its knockdown may show a potentiation of IL-1β mediated activation of the NFκB reporter gene. Data were analyzed as a ratio of luciferase activity to WST reading to control for any effect of differences in cell number. The data were then expressed as a fold change over the average for all non-specific siRNA controls, which was set to 1.

FIG. 9 shows that knockdown of GPR64 significantly repressed the activity of the NFκB luciferase reporter gene to levels similar to that of the p65 control. Knockdown of PTEN did show a modest induction of the reporter gene. Strikingly, the data shows that repression of GPR64 attenuated IL-113 mediated activation of NFκB signaling. This assay also may be suitable for a screen to identify modulators of GPR64, including small molecule inhibitors.

### Example 3C

### Multiple GPR64 siRNA Reagents Repress IL-1β- and TNFα-Induced MMP13 mRNA Levels in the Human Chondrocyte T/C-28a2-Clone19 Cell Line

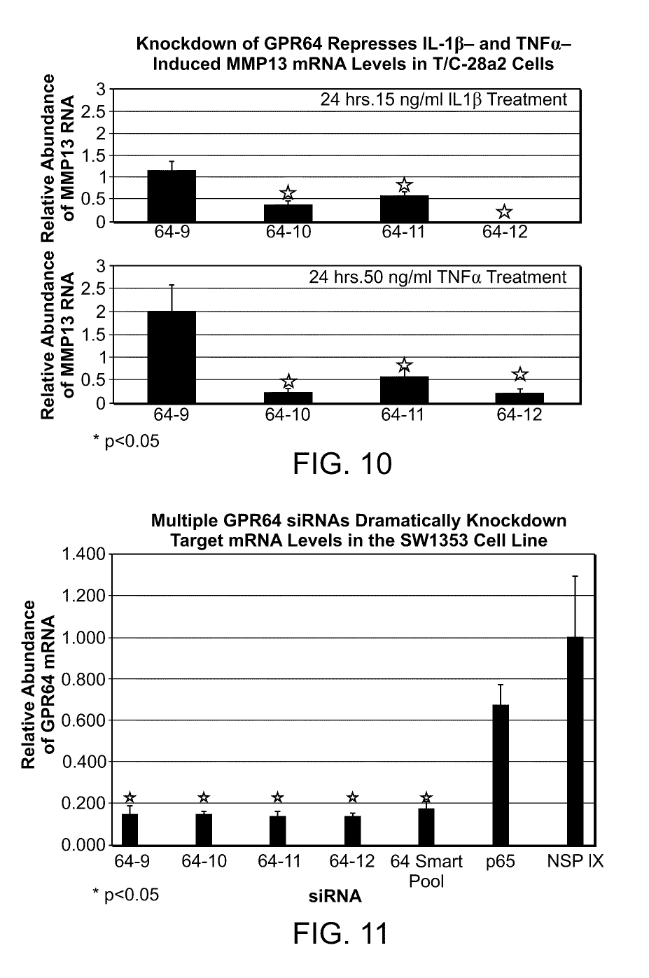

MMP13 is a major protease responsible for degradation of cartilage extracellular matrix in OA. Its expression can be positively regulated by activation of NFκB signaling. MMP13 mRNA levels were monitored following GPR64 siRNA-mediated knockdown. The T/C-28a2-Clone19 cells were seeded in 50 μl at 40,000 cells/well in a 96-well poly-lysine coated plate (catalog #356651, Promega, Madison, Wis.) and cultured in DMEM/F12 50:50 media (catalog #10-092-CV, Cellgro, Herndon, Va.) supplemented with 10% FBS. The cells were plated together with 50 μl of Optimem (catalog #31985-070, Invitrogen, Carlsbad, Calif.) containing 1% Lipofectamine 2000 (Invitrogen, Carlsbad, Calif.) and 50 nM siRNA (Dharmacon, Lafayette, Colo.). As a result, each well contained 5% FBS, 0.5% Lipofectamine 2000 and 25 nM siRNA final concentrations. Specific siRNAs were as described in Example 3B. The following day, the media was replaced with DMEM/F12 50:50 with 10% FBS. 48 hours post-transfection, the media was replaced with serum-free DMEM/F12 50:50 supplemented with 15 ng/ml IL-113 (R&D Systems, Minneapolis, Minn.) or 50 ng/ml TNFα (R&D Systems, Minneapolis, Minn.) for 24 hours. Cells were washed twice in PBS. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (MMP13-Assay-on-Demand catalog #Hs00233992 from ABI). Gene expression was monitored relative to that of the housekeeping gene GAPDH (ABI, Foster City, Calif., cat#4326317E; used at a final concentration of 1×). Relative gene expression levels of MMP13 following GPR64 siRNA knockdown are shown in FIG. 10. All data is presented as fold change relative to expression levels detected in cells transfected with the non-specific, scrambled siRNA NSPIX where the level was set to 1 (white line, FIG. 10). Three of the four GPR64 siRNA reagents (GPR64-10, GPR64-11 and GPR64-12) showed a significant reduction in MMP13 mRNA levels following either IL-1β or TNFα treatment. These data confirm that the inhibition of GPR64 results in the repression of MMP13 mRNA levels following the stimulation of the NFκB pathway in human cartilage cells. Again, these data show that inhibition of GPR64 may be an important therapeutic intervention point for the treatment of OA. Also, these data support that monitoring MMP13 mRNA levels may be a useful assay for screening for compounds that modulate GPR64 activity.

### Example 3D

### Multiple GPR64 siRNA Reagents Knockdown GPR64 mRNA Levels

The knockdown of GPR64 mRNA was monitored by real-time RT-PCR 48 hours post siRNA transfection. The human chondrosarcoma cell line SW1353 was seeded in 50 μl at 30,000 cells/well in a 96-well poly-lysine coated plate (catalog #356651, Promega, Madison, Wis.) and cultured in DMEM/F12 50:50 media (catalog #10-092-CV, Cellgro Herndon, Va.) supplemented with 10% FBS. The cells were plated together with 50 μl of Optimem (catalog #31985-070, Invitrogen, Carlsbad, Calif.) containing 1% Lipofectamine 2000 (Invitrogen, Carlsbad, Calif.) and 50 nM siRNA (Dharmacon, Lafayette, Colo.). As a result, each well contained 5% FBS, 0.5% Lipofectamine 2000, and 25 nM siRNA final concentrations. Specific siRNAs were as described in Example 3B. The following day, the media was replaced with DMEM/F12 50:50 with 10% FBS. 48 hours post-transfection, media was removed, and cells were washed twice in PBS. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (GPR64-Assay-on-Demand catalog #Hs00173773 from ABI). Gene expression was monitored relative to that of the housekeeping gene GAPDH (ABI, Foster City, Calif., cat#4326317E; used at a final concentration of 1×). Relative gene expression levels of GPR64 following GPR64 siRNA knockdown are shown in FIG. 11. All data is presented as fold change relative to expression levels detected in cells transfected with the non-specific, scrambled siRNA NSPIX where the level was set to 1 (white line, FIG. 11). The data confirms that GPR64 is expressed in a cell line derived from human cartilage. All four GPR64 siRNA reagents as well as the pool showed a significant reduction in GPR64 mRNA levels, confirming the efficacy of the siRNAs (p<0.05 by t-test; FIG. 11). These data show that the siRNA reagents are capable of specifically knocking down GPR64 mRNA levels.

### Example 3E

### GPR64 mRNA Levels Do Not Change Following Either TNFα or IL-1β Treatment in Human Chondrosarcoma Cells

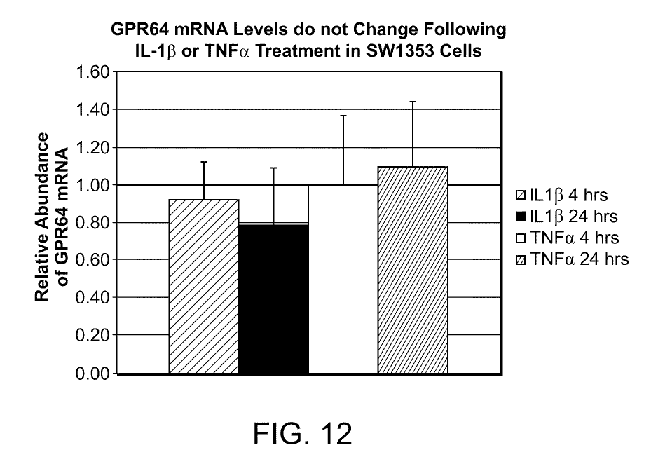

GPR64 mRNA levels were monitored by real-time RT-PCR following treatment of either TNFα or IL-1β. The human chondrosarcoma cell line SW1353 was seeded in 100 μl at 30,000 cells/well in a 96-well poly-lysine coated plate (catalog #356651, Promega, Madison, Wis.) and cultured in DMEM/F12 50:50 media (catalog #10-092-CV, Cellgro, Herndon, Va.) supplemented with 10% FBS. 48 hours post-seeding, the media was replaced with serum-free DMEM/F12 50:50 supplemented with either 15 ng/ml IL-1β (R&D Systems, Minneapolis, Minn.) or 50 ng/ml TNFα (catalog #210-TA, R&D Systems, Minneapolis, Minn.). Treatments proceeded for either 4 or 24 hours. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (GPR64-Assay-on-Demand catalog #Hs00173773 from ABI). Gene expression was monitored relative to that of the housekeeping gene GAPDH (ABI, Foster City, Calif., cat#4326317E; used at a final concentration of 1×). Relative gene expression levels of GPR64 following TNFα or IL-1β treatment are shown in FIG. 12. All data is presented as fold change relative to expression levels detected in untreated cells (set to 1; dark line on FIG. 12). None of the treatment paradigms affected GPR64 mRNA levels, confirming that the repression of NFκB activity following GPR64 mRNA knockdown (shown in FIG. 9) is strictly due to RNAi-mediated GPR64 knockdown and not to ligand-mediated changes (from TNFα or IL-1β treatment) in endogenous GPR64 mRNA levels.

### Example 3F

### MMP13 mRNA Levels are Induced Following Either TNFα or IL-1β Treatment in Human Chondrosarcoma Cell

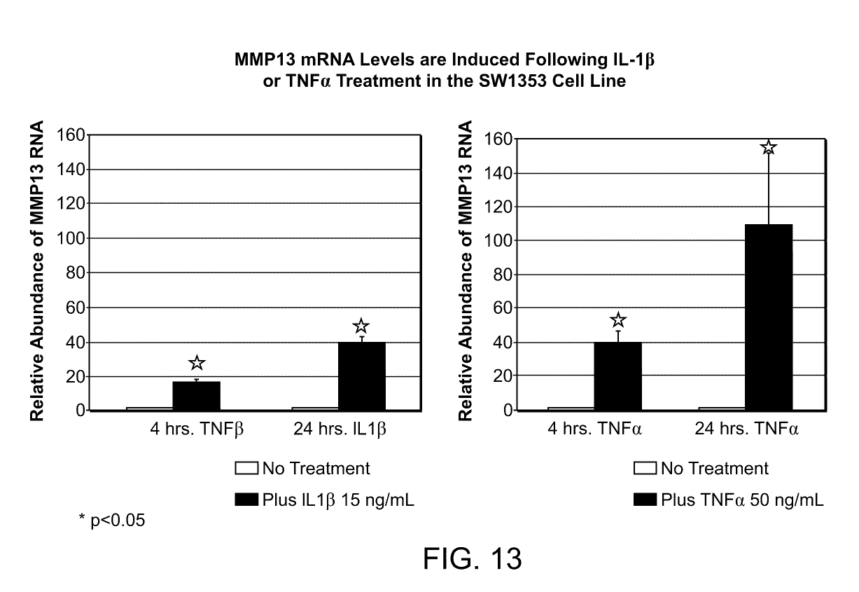

As discussed above, MMP13 is a major protease responsible for degradation of cartilage extracellular matrix in OA. Its expression can be positively regulated by activation of NFκB signaling. MMP13 mRNA levels were monitored by real-time RT-PCR following treatment of either TNFα or IL-1. The human chondrosarcoma cell line SW1353 was seeded in 100 μl at 30,000 cells/well in a 96-well poly-lysine coated plate (catalog #356651, Promega, Madison, Wis.) and cultured in DMEM/F12 50:50 media (catalog #10-092-CV, Cellgro, Herndon, Va.) supplemented with 10% FBS. 48 hours post-seeding, the media was replaced with serum-free DMEM/F12 50:50 supplemented with either 15 ng/ml IL-1β (R&D Systems, Minneapolis, Minn.) or 50 ng/ml TNFα (catalog #210-TA, R&D Systems, Minneapolis, Minn.). Treatments proceeded for either 4 or 24 hours. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (MMP13-Assay-on-Demand catalog #Hs00233992 from ABI). Gene expression was monitored relative to that of the housekeeping gene GAPDH (ABI, Foster City, Calif. cat#4326317E; used at a final concentration of lx). Relative gene expression levels of GPR64 following TNFα or IL-113 treatment are shown in FIG. 12. All data is presented as fold change relative to expression levels detected in untreated cells. Both cytokine ligands at either timepoint showed a very dramatic and significant induction of MMP13 mRNA levels in this human chondrocyte cell line, as shown in FIG. 13. These data support that activation of NFκB signaling positively regulates MMP13 mRNA levels. They further support that inhibition of NFκB signaling and consequently inhibiting the induction of MMP13 expression, a cartilage matrix destroying enzyme, may provide important therapeutic intervention points for the treatment of OA.

### Example 3G

### Multiple GPR64 siRNA Reagents Repress IL-1β-Induced MMP13 mRNA Levels in Human Chondrosarcoma Cells

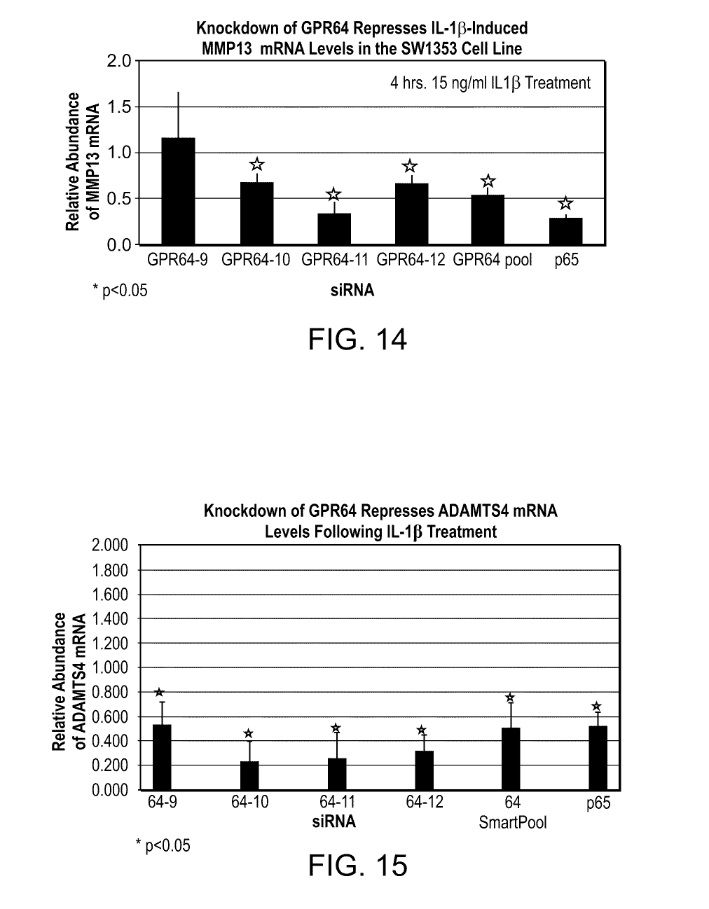

MMP13 mRNA levels were monitored following GPR64 siRNA-mediated knockdown. The human chondrosarcoma cell line SW1353 was seeded in 50 μl at 30,000 cells/well in a 96-well poly-lysine coated plate (catalog #356651, Promega, Madison, Wis.) and cultured in DMEM/F12 50:50 media (catalog #10-092-CV, Cellgro, Herndon, Va.) supplemented with 10% FBS. The cells were plated together with 50 μl of Optimem (catalog #31985-070, Invitrogen, Carlsbad, Calif.) containing 1% Lipofectamine 2000 (Invitrogen, Carlsbad, Calif.) and 50 nM siRNA (Dharmacon, Lafayette, Colo.). As a result, each well contained 5% FBS, 0.5% Lipofectamine 2000 and 25 nM siRNA final concentrations. Specific siRNAs were as described in Example 3B. The following day, the media was replaced with DMEM/F12 50:50 with 10% FBS. 48 hours post-transfection, the media was replaced with serum-free DMEM/F12 50:50 supplemented with 15 ng/ml IL-1β (R&D Systems, Minneapolis, Minn.) for 4 hours. Cells were washed twice in PBS. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (MMP13-Assay-on-Demand catalog #Hs00233992 from ABI). Gene expression was monitored relative to that of the housekeeping gene GAPDH (ABI, Foster City, Calif. cat#4326317E; used at a final concentration of lx). Relative gene expression levels of MMP13 following GPR64 siRNA knockdown are shown in FIG. 14. All data is presented as fold change relative to expression levels detected in cells transfected with the non-specific, scrambled siRNA NSPIX where the level was set to 1 (white line, FIG. 14). Three of the four GPR64 siRNA reagents (GPR64-10, GPR64-11, and GPR64-12) as well as the pool showed a significant reduction in MMP13 mRNA levels to levels similar to that following RNAi-mediated knockdown of p65, the control. These data show that the inhibition of GPR64 results in the repression of IL-1β-mediated induction of MMP13 mRNA levels in human cartilage cells. Again, these data show that inhibition of GPR64 may be an important therapeutic intervention point for the treatment of OA. Also, these data support that monitoring MMP13 mRNA levels may be a useful assay for screening for compounds that modulate GPR64 activity.

### Example 3H

### Multiple GPR64 siRNA Reagents Repress Aggrecanase (ADAMTS4) mRNA Levels in Human Chondrosarcoma Cells

ADAMTS4 is a protease whose activity has been implicated in the destruction of cartilage extracellular matrix in osteoarthritic individuals. ADAMTS4 mRNA levels were monitored following GPR64 siRNA-mediated knockdown. The human chondrosarcoma cell line SW1353 was seeded in 50 μl at 30,000 cells/well in a 96-well poly-lysine coated plate (catalog #356651, Promega, Madison, Wis.) and cultured in DMEM/F12 50:50 media (catalog #10-092-CV, Cellgro, Herndon, Va.) supplemented with 10% FBS (Invitrogen, Carlsbad, Calif.). The cells were plated together with 50 μl of Optimem (catalog #31985-070, Invitrogen, Carlsbad, Calif.) containing 1% Lipofectamine 2000 (Invitrogen, Carlsbad, Calif.) and 50 nM siRNA (Dharmacon, Lafayette, Colo.). As a result, each well contained 5% FBS, 0.5% Lipofectamine 2000 and 25 nM siRNA final concentrations. Specific siRNAs were as described in Example 3B. The following day, the media was replaced with DMEM/F12 50:50 with 10% FBS. 48 hours post-transfection, the media was replaced with serum-free DMEM/F12 50:50 supplemented with 15 ng/ml IL-1β (R&D Systems, Minneapolis Minn.) for 4 hours. Cells were washed twice in PBS. RNA was purified using the RNAcapture kit (cat#21-GP02-1, RNAture, Irvine, Calif.). Real-time RT-PCR was performed using 5 μl of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (ADAMTS4-Assay-on-Demand catalog #Hs00192708 from ABI). Gene expression was monitored relative to that of the housekeeping gene GAPDH (ABI, Foster City, Calif., cat#4326317E; used at a final concentration of 1×). Relative gene expression levels of ADAMTS4 following GPR64 siRNA knockdown are shown in FIG. 15. All data is presented as fold change relative to expression levels detected in cells transfected with the non-specific, scrambled siRNA NSPIX where the level was set to 1 (white line, FIG. 15). All four GPR64 siRNA reagents (GPR64-9, GPR64-10, GPR64-11, and GPR64-12) as well as the pool showed a significant reduction in ADAMTS4 mRNA levels to levels similar to that following RNAi-mediated knockdown of p65, the control. These data show that the inhibition of GPR64 results in the repression of a second cartilage matrix degradative enzyme that has been associated with OA. Again, these data show that inhibition of GPR64 may be an important therapeutic intervention point for the treatment of OA. Also, these data support that monitoring ADAMTS4 mRNA levels may be a useful assay for screening for compounds that modulate GPR64 activity.

### Example 31

### Knockdown of GPR64 Represses MMP13 mRNA Levels in Primary Human Chondrocytes Obtained from Osteoarthritic Patients

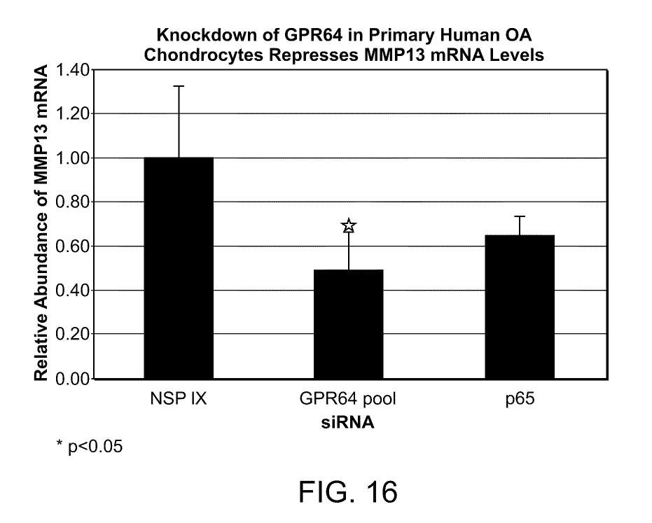

MMP13 mRNA levels were monitored following GPR64 siRNA-mediated knockdown. Primary human chondrocytes were isolated from surgical biopsy samples of osteoarthritic patients. Cells were seeded in 300 μl at 600,000 cells/well in a 24-well plate and cultured in growth media: DMEM/F12 50:50 media (catalog #10-092-CV, Cellgro, Herndon, Va.) supplemented with 10% FBS. The following day, the media was removed and replaced with 250 μl of the growth media and 50 μl of Optimem (catalog #31985-070, Invitrogen, Carlsbad, Calif.) containing 2.5% Ribojuice (catalog #71115-4, Novogen, San Diego, Calif.) and 50 nM siRNA (Dharmacon, Lafayette, Colo.). As a result, each well contained 8.3% FBS, 0.42% Ribojuice and 25 nM siRNA final concentrations. Specific siRNAs were as described in Example 3B. The following day, the media was replaced with DMEM/F12 50:50 with 10% FBS. 48 hours post-transfection, the media was removed and the cells were washed twice in PBS. RNA was purified using the RNEasy kit (cat#74106, Qiagen, Valencia, Calif.). Real-time RT-PCR was performed using 100 ng of RNA per 25 μl reaction in 1×QRT-PCR mastermix (Eurogenetec, Philadelphia, Pa.; cat#RT-QPRT-032×). Primers and probes were purchased from Applied Biosystems (ABI, Foster City, Calif.) and used at a final concentration of 1× (MMP13-Assay-on-Demand catalog #Hs00233992 from ABI). Gene expression was monitored relative to that of the housekeeping gene GAPDH (ABI, Foster City, Calif., cat#4326317E; used at a final concentration of 1×). Relative gene expression levels of MMP13 following GPR64 siRNA knockdown are shown in FIG. 16. All data is presented as fold change relative to expression levels detected in cells transfected with the non-specific, scrambled siRNA NSPIX where the level was set to 1 (white line, FIG. 16). Knockdown of GPR64 showed significant repression of MMP13 mRNA levels, to levels superior to that detected in RNAi-mediated knockdown of p65, the control. These data show that the inhibition of GPR64 results in the repression of MMP13 mRNA levels in primary human cartilage cells. Furthermore, these data support the previous observations presented in FIGS. 8-15 that were performed in two different human chondrocytes cell lines. The data presented in FIG. 16 demonstrate that, in primary human cells, the same results were observed: inhibition of GPR64 repressed the expression of the OA, disease-associated gene, MMP13. Together, these data show that inhibition of GPR64 may be an important therapeutic intervention point for the treatment of OA. Also, these data support that monitoring MMP13 mRNA levels may be a useful assay for screening for compounds that modulate GPR64 activity.

### Example 4

### Screening Assay for Modulators of GPR64 Activity

In order to identify small molecule modulators of GPR64, an assay system is set up to measure activity of this G protein-coupled receptor. First, GPR64 is transiently over-expressed in U2OS, CHO, HEK293, 293T, NIH3T3, COS7, or other mammalian cell line, and its membrane expression is verified by immunostaining. Next, the basal activity of the receptor is examined by monitoring several signaling pathways in cells transfected with GPR64 versus cells expressing the empty vector. Since the coupling of GPR64 has not been determined to date, the basal activity is determined by measuring multiple intracellular events, including, but not limited to, the following: 1) measuring the generation or down-regulation of cAMP by CRE-Luc reporter assays or enzyme fragmentation complementation assays; 2) measuring the activation of the MAP Kinase pathway by an SRE-Luc reporter analysis; and/or 3) measuring the generation of IP₃ directly or indirectly through increase in intracellular concentration of Ca²⁺. The changes in Ca²⁺ concentration are assessed by the FLIPR technology or by NFAT-RE-Luc reporter gene approach. Once the signaling event most responsive to GPR64 is identified, the dose response is determined using increasing amounts of GPR64 cDNA. A cell line is also generated by stably over-expressing GPR64 and/or a reporter gene. A stable or transiently transfected cell line is then used in an HTS to identify small molecule activators and/or inhibitors of the basal GPR64 activity. If transient transfection is used, the amount of GPR64 cDNA transfected is around EC50 to maximize the chances of identifying the response modulators.

An alternative approach includes visualizing GPR64 internalization. This is accomplished by introducing into the cell and monitoring an arrestin-GFP fusion protein, a component of the internalized vesicle.

The assays described above are modified by using a truncated form of GPR64 missing various portions of the extracellular domain to identify modulators binding elsewhere in the molecule.

MMP-13 and ADAMTS4 are assayed using a FRET based high throughput method. For GPR64 translocation experiments, Transfluor Technology™ (Molecular Devices, Sunnyvale, Calif.) is used. Transfluor™ is a cell-based fluorescence assay used to screen for G-protein-coupled receptors (GPCRs) ligands and other potential drugs that regulate GPCRs. The technology is based on the discovery that, upon activation by ligand binding, virtually all GPCRs rapidly undergo deactivation or “desensitization” by a common pathway. An early step in this pathway is the binding of the cytoplasmic protein beta-arrestin to the activated receptor. Beta-arresting binding deactivates the GPCR signaling and begins the translocation of the receptor into the cell where the ligand is removed and the receptor is recycled back to the cell membrane. By attaching a fluorescent label to beta-arrestin, the location of the receptor-arrestin complex is monitored. Since desensitization only occurs with an activated receptor, activation of any GPCR is detected by monitoring beta-arrestin translocation and subsequent receptor recycling.

### Example 5

### Example of Screening Assay for Inhibitor of GPR64 Activity

The portion of the gene encoding the substrate-binding domain of human GPR64 is cloned into a bacterial expression vector, transformed into E. coli, and the protein is purified from bacterial cultures by column chromatography utilizing standard molecular biology and biochemistry methods. The partially purified preparation is assayed for GPR64 activity by bringing it in contact with a substrate. Test agents are screened by their ability to modulate (e.g., inhibit) the reaction, as determined by altered (e.g., decreased) amount of the GPR64-substrate interaction, such as binding, or by product formed as a function of time relative to control reactions. In some cases, cell-based assays, such as, for example, those described in Example 4, are also used to screen for inhibitors of GPR64 activity.

### Example 6

### Example of Screening Assay for Inhibitor of GPR64 Expression Involving GPR64 Promoter

A GPR64 promoter is linked to a reporter gene, for example, a luciferase gene. Activation of the reporter gene is demonstrated by using a GPR64 inducer, indicating transcriptional specificity. Test agents are screened to identify those that block the induced reporter gene activity.

### Example 7

### Example of Screening Assay for Inhibitor of GPR64 Expression

A tissue sample or cartilage extract culture is treated with a test agent. The tissue sample or cartilage extract culture is then treated with an antibody to GPR64 (or a binding portion thereof), and levels of antibody binding are detected. Alternatively, the tissue sample or cartilage extract is treated with an antibody to MMP13, ADAMTS1, ADAMTS4, ADAMTS5, ADAMTS8, ADAMTS9, or ADAMTS15 (or an antigen-binding fragment/portion thereof) and levels of antibody binding detected. These levels are compared to the control level for normal tissue of the same sample type or the same cartilage extract culture type. The levels are also compared to those of a control tissue sample or cartilage extract culture that are not treated with the test agent. A decrease in GPR64 expression levels indicates that the test agent is an inhibitor agent.

### Example 8

### Example of Screening Assay for an Activator of GPR64 Expression

A tissue sample or cartilage extract culture is treated with a test agent. The test agent is a known cytokine involved in inflammatory cytokine pathways, such as, but not limited to, TNF, IL-1, IL-6, IL-9 IL-18, and IL-22. The tissue sample or cartilage extract culture is then treated with an antibody to GPR64 (or a binding portion thereof), and levels of antibody binding are detected. Alternatively, the tissue sample or cartilage extract is treated with an antibody to MMP13, ADAMTS1, ADAMTS4, ADAMTS5, ADAMTS8, ADAMTS9, or ADAMTS15 (or antigen-binding fragments/portions of the antibody) and levels of antibody binding detected. These levels are compared to the control level for normal tissue or the cartilage extract culture of the same sample type. The levels are also compared to those of a control tissue sample or cartilage extract that are not treated with the test agent. An increase in GPR64 expression levels indicates that the test agent is an activator agent.

### Example 9

### Example of Screening Assay for an Activator of GPR64 Activity

The portion of the gene encoding the activator-binding domain of human GPR64 is cloned into bacterial expression vector, transformed into E. coli and the protein purified from bacterial cultures by column chromatography utilizing standard molecular biology and biochemistry methods. The partially purified preparation is assayed for GPR64 activity by bringing it in contact with a substrate. Test agents are screened by their ability to modulate (e.g., activate) the reaction as determined by altered (e.g., increased) amount of the GPR64-activator interaction, such as binding, or product formed as a function of time relative to control enzyme reactions. In some cases, cell-based assays, such as, for example, those described in Example 4, are also used to screen for activators of GPR64 activity.

### Example 10

### Example of Screening Assay for an Activator of GPR64 Expression Involving GPR64 Promoter

A GPR64 promoter is linked to a reporter gene, for example, a luciferase gene. Activation of the reporter gene is demonstrated by a GPR64 inducer, indicating transcriptional specificity. Test agents are screened to identify those that activate the induced reporter gene activity.

### Example 11

### Example of Screening Assay for OA Using Determination of RNA Expression Levels of GPR64

Samples of human normal cartilage and cartilage from a patient possibly afflicted with OA are harvested after signed consent and flash frozen in liquid nitrogen. Frozen tissues are pulverized and RNA is isolated utilizing guanidinium isothiocyante and RNeasy kit (Qiagen, Valencia, Calif.). Agilent systems are used to assess RNA quality. Quantitative real time RT-PCR is carried out utilizing primers and probes specific for human GPR64 (see, e.g., SEQ ID NOS: 7, 8, and 9) and the human GAPDH gene for normalization. (Alternatively, primers and probes (as in Example 3) specific for MMP13 and/or ADAMTS1, ADAMTS4, ADAMTS5, ADAMTS8, ADAMTS9, or ADAMTS15 are used.)

The RNA expression levels from the affected OA cartilage sample are compared to the control normal level. An increase in GPR64 expression (or MMP13 and/or ADAMTS1, ADAMTS4, ADAMTS5, ADAMTS8, ADAMTS9, or ADAMTS15) indicates that the patient is afflicted with OA. A decrease in GPR64 expression (or MMP13 and/or ADAMTS1, ADAMTS4, ADAMTS5, ADAMTS8, ADAMTS9, or ADAMTS15) indicates that the patient is not afflicted with OA.

### Example 12

### Example of Screening Assay for OA Using Determination of Protein Expression Levels of GPR64

Samples of human normal cartilage and cartilage from a patient possibly afflicted with OA are harvested after signed consent. The samples are fixed for 24 hours in 4% paraformaldehyde, embedded in paraffin and sectioned for immunohistochemistry. Tissues are stained with a polyclonal anti-GPR64 antibody (LifeSpan BioSciences, Seattle, Wash.) utilizing the DAKO Envision+system (DakoCytomation California Inc., Carpinteria, Calif.) according to the manufacturer's instructions, and are counterstained with Mayer's alum-hematoxylin. (Alternatively, antibodies specific for MMP13 and/or ADAMTS1, ADAMTS4, ADAMTS5, ADAMTS8, ADAMTS9, or ADAMTS15 can be used.) The extent of matrix degradation in each tissue sample is also assessed by Safranin-O staining, which stains proteoglycan in the extracellular matrix, on adjacent sections.

The protein expression levels from the affected OA cartilage sample are compared to the control normal level. An increase in GPR64 (or MMP13 and/or ADAMTS1, ADAMTS4, ADAMTS5, ADAMTS8, ADAMTS9, or ADAMTS15) expression indicates that the patient is afflicted with OA. A decrease in GPR64 expression (or MMP13 and/or ADAMTS1, ADAMTS4, ADAMTS5, ADAMTS8, ADAMTS9, or ADAMTS15) indicates that the patient is not afflicted with OA.

### Example 13

### Example of Treating an Inflammatory Disease with a GPR64 Inhibitor

A therapeutically-effective amount of a known GPR64 inhibitor is administered to a subject diagnosed with an inflammatory disease. A control group also exhibiting symptoms of the inflammatory disease is treated with a placebo control. Administration is by a single treatment or treatment over a course of days. Subjects are evaluated for symptoms related to the inflammatory disease. Exemplary symptoms and/or complications of such inflammatory diseases include, but are not limited to, pain, edema, swelling, heat, malaise, joint stiffness, and redness. In addition, for OA, additional symptoms that are reduced or eliminated include, without limitation, degradation of cartilage and subsequent changes in the presence of these degradative products in body fluids. Effective treatment is determined by a reduction in symptoms compared to the control group.

### Example 14

### Example of GPR64 Mutagenesis

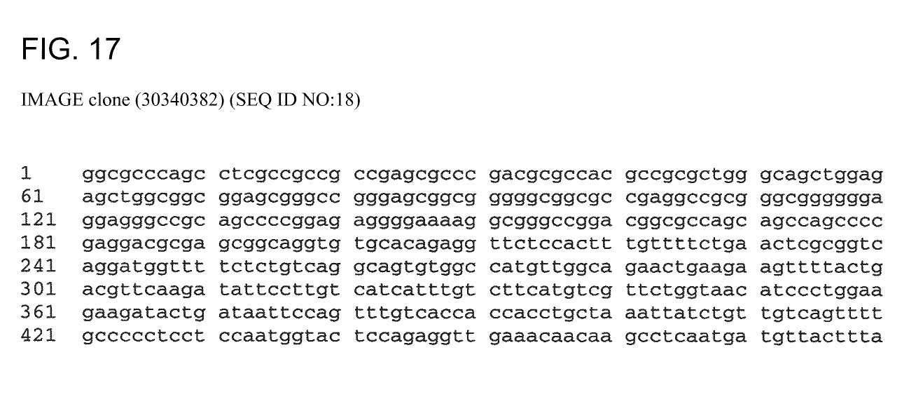

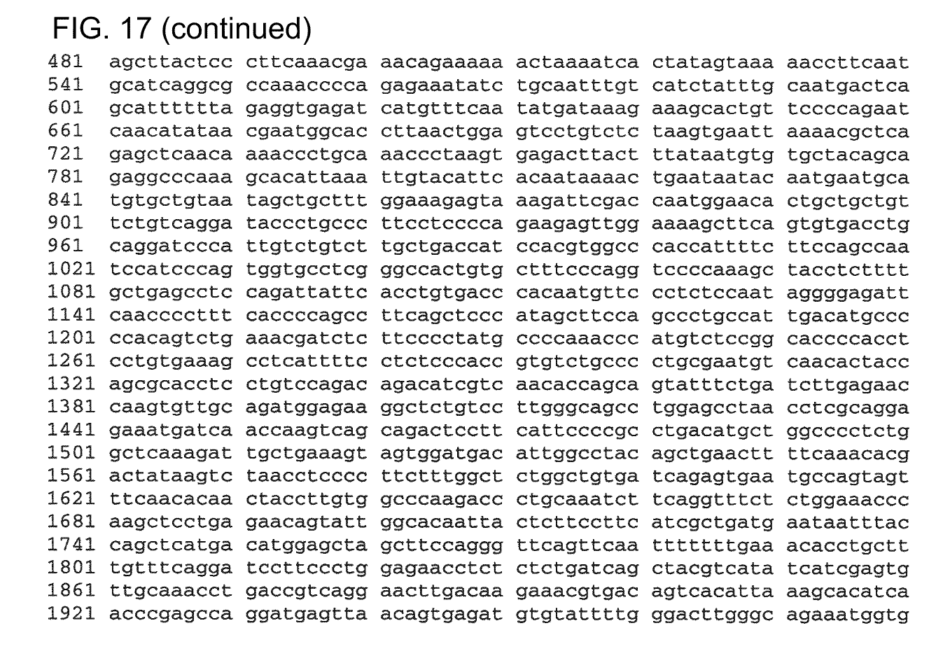

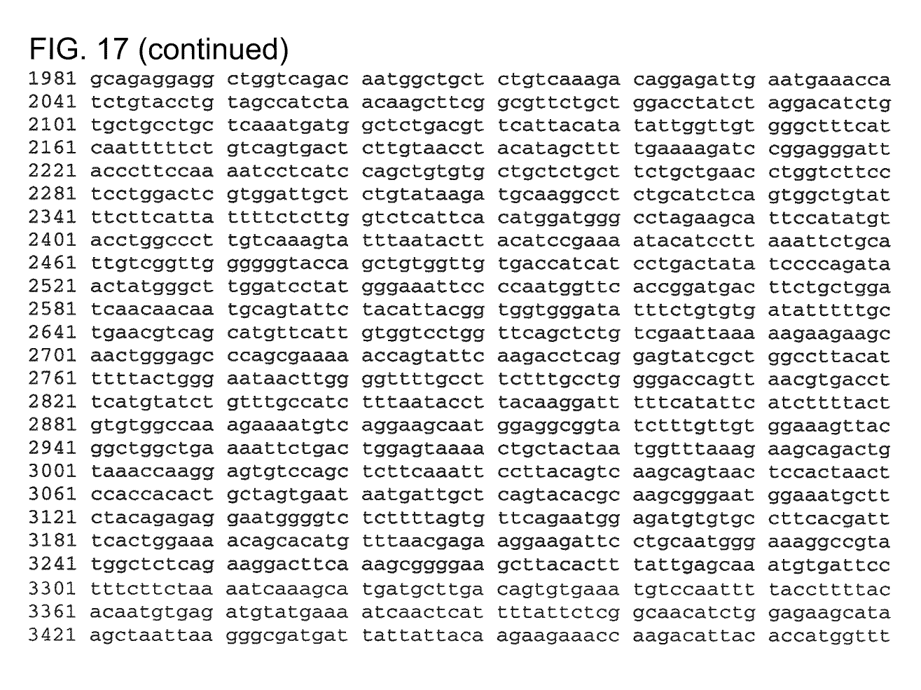

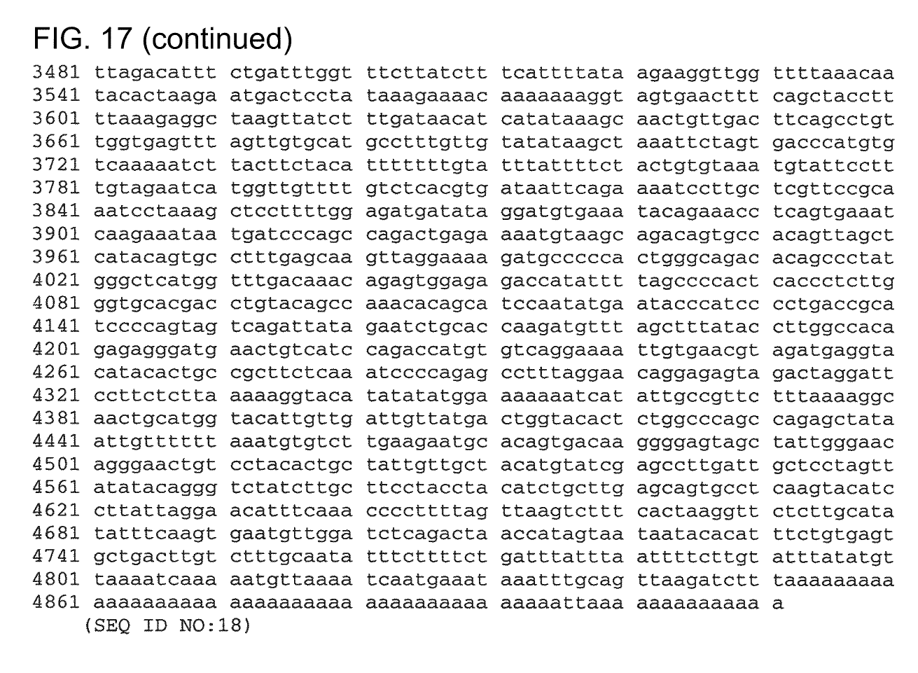

An IMAGE clone (Clone ID: 30340382; SEQ ID NO:18 shown in FIG. 17) that has a frame shift error was mutated to correct the error by using suitable oligonucleotides. The clone containing frame shift error was purchased from Open Biosystems (Huntsville, Ala.). Two sets of oligonucleotides were used to correct the reading frame (making two changes). The primers used for the mutagenesis were: 5′-CAACACAACTACCTTTGTGGCCCAAGACCC-3′ (SEQ ID NO:19); 5′-GGGTCTTGGGCCACAAAGGTAGTTGTGTTG-3′ (SEQ ID NO:20); 5′-GTTTCAACACAACTACCTTTGTGGCCCAAGACCCTGC-3′ (SEQ ID NO:21); and 5′-GCAGGGTCTTGGGCCACAAAGGTAGTTGTGTTGAAAC-3′ (SEQ ID NO:22). Following PCR and transformation according to QuickChange manufacturer's recommendations (Stratagene, La Jolla, Calif.) protocol, several potential colonies were observed from both sets of oligonucleotides. Sequence data from plasmid DNA from these clones confirm the presence of the introduced change. The nucleic acid sequence is designated SEQ ID NO:5, and the amino acid sequence is designated SEQ ID NO:6.

### Example 15

### Western Blot Analysis of GPR64 in Human Cartilage Extracts

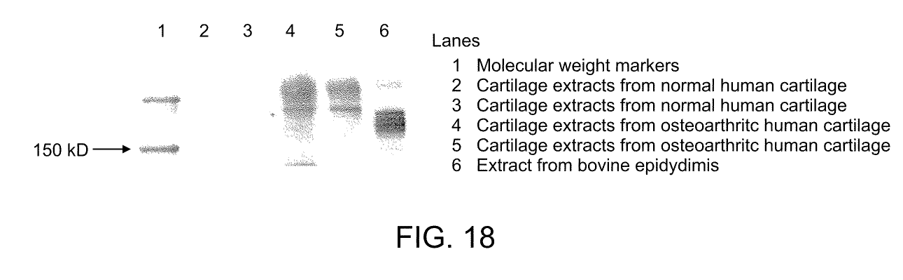

For the western blot analysis of GPR64 protein up-regulation in OA, total protein from normal and OA cartilage was precipitated with acetone. Protein precipitate was washed with 300 mM guanidine hydrochloride and dissolved in phosphate buffer containing 1% Triton X-100 and 0.5% deoxycholate. Protein samples were diluted with sample loading buffer to a final concentration of 20 mM DTT. 20 μg of protein from each sample was loaded onto a 4-12% SDS-PAGE gel. Bovine epididymal extract was loaded as a positive control. Proteins were transferred to polyvinyliden-difluoride (PVDF) membranes using wet transfer method. Immunodetection of proteins was carried out by standard procedures, employing serum from immunized rabbits at a dilution of 1:2000. The antibodies were raised against peptide sequence: CLADHPRGP PFSSSQSIP (SEQ ID NO:23). Immunopositive bands were detected employing anti-rabbit horse-radish peroxidase-conjugated antibody (1:5000) combined with HRP substrate system and exposure to autoradiography film. The results are shown in FIG. 18.

### Example 16

### Identification of Novel Sequence Variants of GPR64

A plasmid containing at least a partial fragment of the human GPR64 gene was purchased from Origene, Inc. (Rockville, Md.) as catalog #TC108549. The clone was identified as having some homology to GPR64 (RefSeq NM_{—}005756) based on unedited DNA sequence reads from each end of the insert. The reported 5-prime and 3-prime end reads of the plasmid were obtained and are represented in SEQ ID NO:24 and SEQ ID NO:25, respectively (as shown in FIGS. 19A and 19B, respectively).

Upon purchase of this plasmid, the full insert was determined by DNA sequencing to encode a novel variant of the human GPR64 gene (SEQ ID NO:26, shown in FIG. 19C). The predicted amino acid sequence of SEQ ID NO:26 was determined and is shown in FIG. 19D, as SEQ ID NO:27. A comparison of a reference GPR64 protein sequence (SEQ. ID NO:2) versus the novel variant (SEQ ID NO:27), shown in FIG. 19E, revealed a 51 amino acid deletion in the novel variant (SEQ ID NO:27). Thus, this novel variant may confer unique biological activities when expressed in a cell, or when subjected to an agonist or antagonist.

Additional novel variants of the human GPR64 gene were identified and sequenced in accordance with the methods described herein, with the nucleotide sequences being as shown as SEQ ID NO:1 (FIG. 1), SEQ ID NO:3 (FIG. 3), SEQ ID NO:5 (FIG. 5A), SEQ ID NO:28 (FIG. 20), SEQ ID NO:30 (FIG. 22), SEQ ID NO:32 (FIG. 24), SEQ ID NO:34 (FIG. 26), SEQ ID NO:36 (FIG. 28) and SEQ ID NO:38 (FIG. 30). The predicted amino acid sequences are provided as SEQ ID NO:2 (FIG. 2), SEQ ID NO:4 (FIG. 4), SEQ ID NO:6 (FIG. 5B), SEQ ID NO:29 (FIG. 21), SEQ ID NO:31 (FIG. 23), SEQ ID NO:33 (FIG. 25), SEQ ID NO:35 (FIG. 27), SEQ ID NO:37 (FIG. 29) and SEQ ID NO:39 (FIG. 31).

### Example 17

### A Tool to Screen for Modulators of GPR64

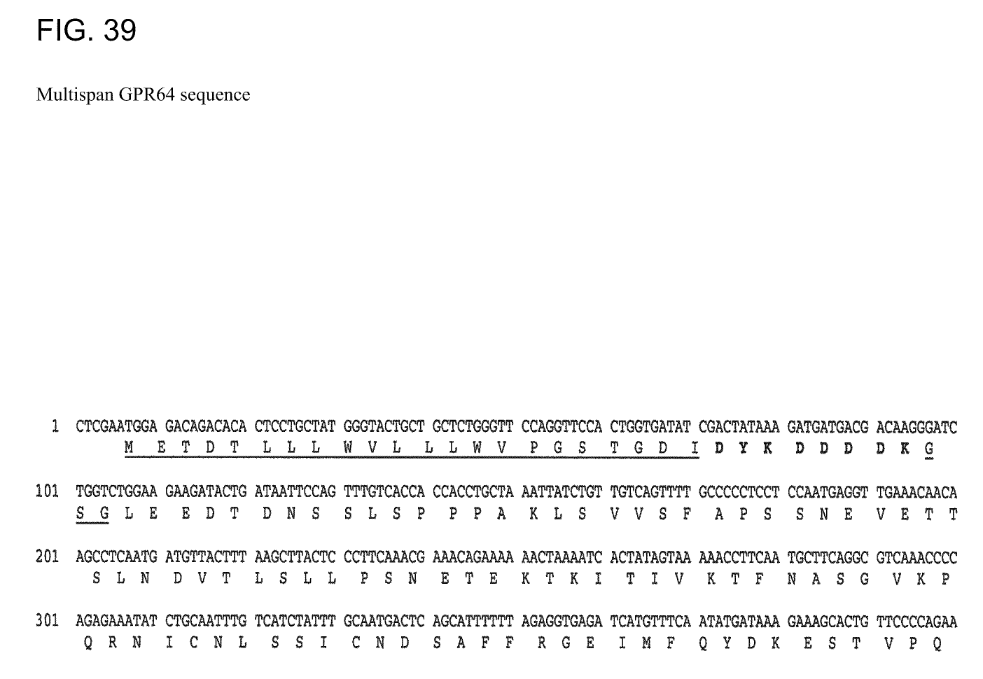

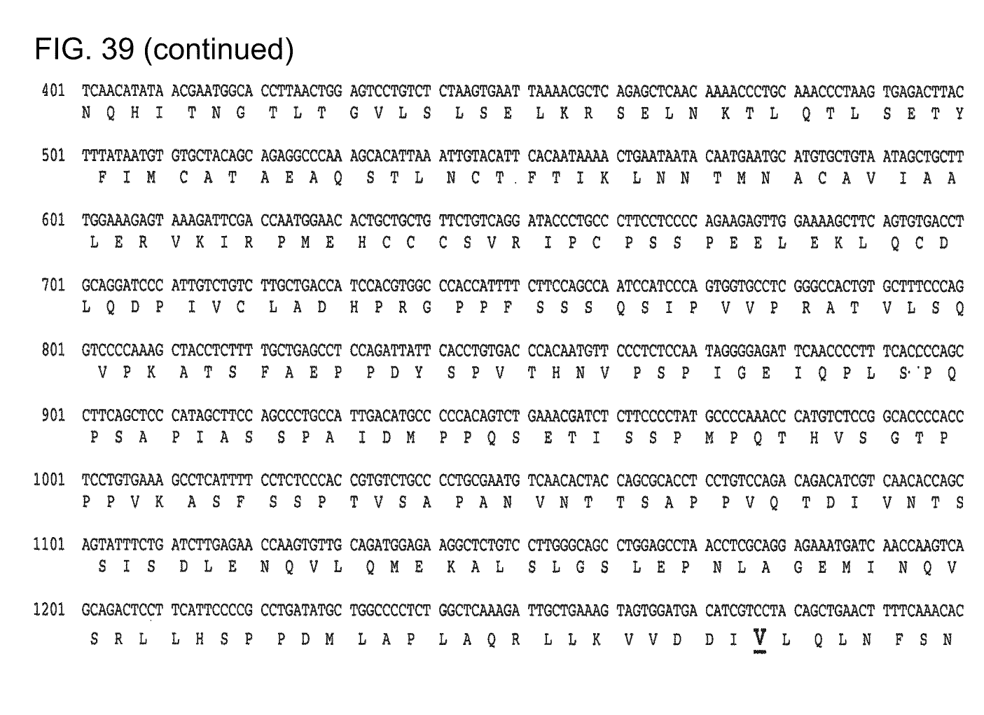

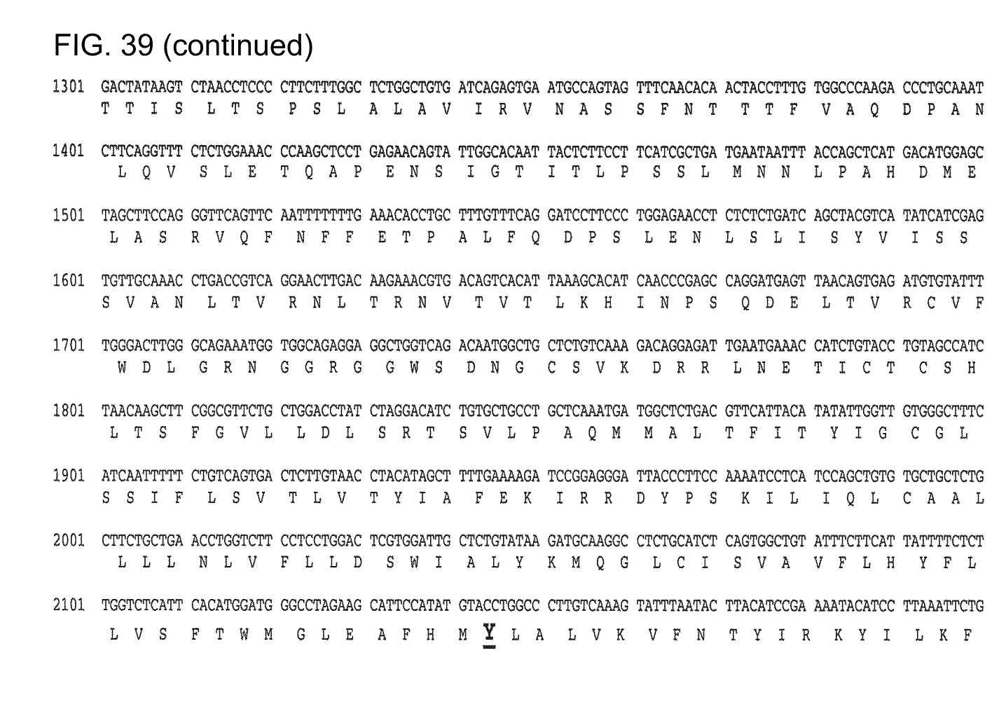

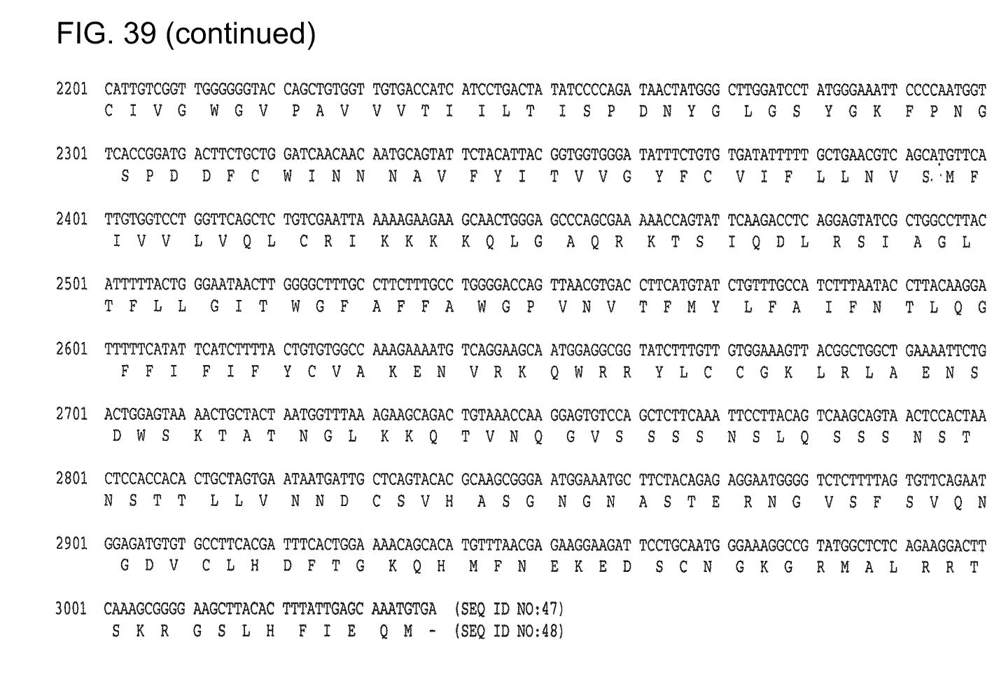

A U2OS cell line that expresses human osteoarthritic cartilage sequence with the following changes: 1) conservative amino acid substitution at position 424 (Val for Gly) and 2) a polymorphism at position 713 (Tyr for His) was constructed by Multispan as a tool to screen for modulators of GPR64. The GPR64 protein was expressed with a heterologous signal peptide (Multispan leader sequence: METDTLLLWVLLLWVPGSTGDI (SEQ ID NO:49)), a Flag tag (DYKDDDDK (SEQ ID NO:50)), and a linker (GSG). The sequence is shown in FIG. 39 and assigned SEQ ID NO:48. The cell line uses U2OS osteosarcoma cells over-expressing GFP-tagged beta-arrestin (licensed from Molecular Devices). This cell line is used in the screening for modulators of GPR64 using the GRK-LITe assay, i.e., ligand independent GPR internalization (Transflour technology licensed from Molecular Devices).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a subject having or at risk for an inflammatory disease, comprising
contacting a sample from the subject with an agent that binds to GPR64;
detecting a level of binding of the agent to GPR64 in the sample; and
comparing the level of binding of the agent to GPR64 in the sample to a control level;
wherein a level of binding of the agent to GPR64 in the sample that is increased relative to the control is indicative that the subject has or is at risk for the inflammatory disease.

2. The method of claim 1, wherein the agent is an antibody.

3. The method of claim 1, wherein the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the subject, sepsis, and systematic lupus erythematosus.

4. The method of claim 1, wherein the inflammatory disease is osteoarthritis or rheumatoid arthritis.

5. A method of identifying an agent that modulates the activity or expression of GPR64, comprising:
contacting a sample with a test agent;
detecting a level of activity or expression of GPR64 in the sample in the presence of the test agent; and
comparing the level of activity or expression of GPR64 in the presence of the test agent to a control level,
wherein a level of activity or expression of GPR64 in the presence of the agent that is different from the control level is indicative that the test agent is an agent that modulates the activity or expression of GRP64.

6. The method of claim 5, wherein a level of activity or expression of GPR64 in the presence of the agent that is increased relative to the control level is indicative that the test agent is an agent that modulates the activity or expression of GRP64.

7. The method of claim 5, wherein a level of activity or expression of GPR64 in the presence of the agent that is decreased relative to the control level is indicative that the test agent is an agent that modulates the activity or expression of GRP64.

8. The method of claim 5, wherein detecting the level of activity or expression of GPR64 comprises measuring the level of one or more of Aggrecanase activity, Aggrecanase expression, MMP activity, MMP expression, and NFκB signaling.

9. A method of identifying an agent that modulates the activity or expression of GPR64, comprising:
contacting a sample with a test agent;
detecting a level of NFκB pathway signaling in the sample in the presence of the test agent; and
comparing the level of NFκB pathway signaling in the presence of the test agent to a control level,
wherein a level of NFκB pathway signaling in the presence of the agent that is different from the control level is indicative that the test agent is an agent that modulates the activity or expression of GRP64.

10. The method of claim 9, wherein detecting the level of NFκB pathway signaling comprises evaluating the level of a transcription factor in the nucleus relative to the level of the transcription factor in the cytoplasm of a cell.

11. The method of claim 9, wherein detecting the level of NFκB pathway signaling comprises detecting the level of activity or expression of an enzyme that degrades cartilage.

12. The method of claim 11, wherein the enzyme is MMP13, ADAMTS1, ADAMTS4, ADAMTS5, ADAMTS8, ADAMTS9, or ADAMTS15.

13. A method of identifying an agent that modulates the activity or expression of GPR64, comprising:
contacting a sample with a test agent;
detecting a level of activity or expression of MMP13 in the sample in the presence of the test agent; and
comparing the level of activity or expression of MMP13 in the presence of the test agent to a control level,
wherein a level of activity or expression of MMP13 in the presence of the agent that is different from the control level is indicative that the test agent is an agent that modulates the activity or expression of GRP64.

14. A method of treating a subject having or at risk of developing an inflammatory disease, comprising administering to the subject an agent that modulates the activity or expression of GPR64, thereby treating the inflammatory disease.

15. An isolated polynucleotide comprising the nucleic acid sequence of SEQ ID NO:5, SEQ ID NO:26, SEQ ID NO:28, SEQ ID NO:30, SEQ ID NO:32, SEQ ID NO:34, SEQ ID NO:36, or SEQ ID NO:38.

16. An isolated polynucleotide comprising a nucleic acid encoding the amino acid sequence of SEQ ID NO:2, SEQ ID NO:4, SEQ ID NO:6, SEQ ID NO:27, SEQ ID NO:29, SEQ ID NO:31, SEQ ID NO:33, SEQ ID NO:35, SEQ ID NO:37, or SEQ ID NO:39.

17. An isolated nucleic acid having at least 90% sequence identity to a nucleic acid sequence encoding a polypeptide having the amino acid sequence of SEQ ID NO:2, SEQ ID NO:4, SEQ ID NO:6, SEQ ID NO:27, SEQ ID NO:29, SEQ ID NO:31, SEQ ID NO:33, SEQ ID NO:35, SEQ ID NO:37, or SEQ ID NO:39, wherein the isolated nucleic acid encodes a polypeptide that inhibits the activity or expression of GPR64.

18. An isolated nucleic acid encoding a polypeptide having at least 90% sequence identity to the amino acid sequence of SEQ ID NO:2, SEQ ID NO:4, SEQ ID NO:6, SEQ ID NO:27, SEQ ID NO:29, SEQ ID NO:31, SEQ ID NO:33, SEQ ID NO:35, SEQ ID NO:37, or SEQ ID NO:39, wherein the polypeptide inhibits the activity or expression of GPR64.

19. A vector comprising the nucleic acid of any one of claims 15-18.

20. The vector of claim 19, wherein the nucleic acid is operably-linked to a control sequence recognized by a host cell transformed with the vector.

21. A host cell comprising the vector of claim 20.

22. The host cell of claim 21, wherein the cell is a U2OS osteosarcoma cell, a human embryonic kidney cell, a Chinese Hamster Ovary (CHO) cell, a chondrocyte, an insect cell, a yeast cell, or a bacterial cell.
